(19) United States
(12) Patent Application Publication
(10) Pub. No.: US 2010/0061982 A1
(43) Pub. Date: Mar. 11, 2010

(54) 3-SUBSTITUTED-1H-INDOLE, 3-SUBSTITUTED-1H-PYRROLO[2,3-B]PYRIDINE AND 3-SUBSTITUTED-1H-PYRROLO[3,2-B]PYRIDINE COMPOUNDS, THEIR USE AS MTOR KINASE AND PI3 KINASE INHIBITORS, AND THEIR SYNTHESES

(75) Inventors: Semiramis Ayral-Kaloustian, Tarrytown, NY (US); Tarek Suhayl Mansour, New City, NY (US); Hwei-Ru Tsou, New City, NY (US); Nan Zhang, Bayside, NY (US); Aranapakam Mudumbai Venkatesan, Rego Park, NY (US); Li Di, West Windsor, NJ (US); Edward Harvel Kerns, Skillman, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/556,833

(22) Filed: Sep. 10, 2009

Related U.S. Application Data
(60) Provisional application No. 61/095,787, filed on Sep. 10, 2008.

(51) Int. Cl. A61K 39/395; C07D 405/06; A61K 31/40; C07D 403/06; A61K 31/497; C07D 498/02; A61K 31/5386

(52) U.S. Cl. 424/130.1; 548/454; 514/414; 544/373; 514/254.09; 544/105; 514/230.5

(57) ABSTRACT

The invention relates to 3-substituted-1H-indole, 3-substituted-1H-pyrrolo[2,3-b]pyridine, and 3-substituted-1H-pyrrolo[3,2-b]pyridine compounds of the Formula 1:

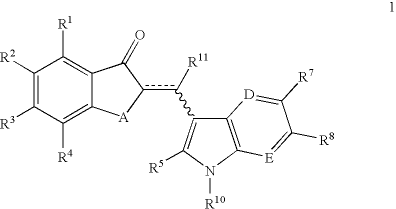

or a pharmaceutically acceptable salt thereof, wherein the constituent variables are as defined herein, compositions comprising the compounds, and methods for making and using the compounds.

### FIELD OF THE INVENTION

The invention relates to 3-substituted-1H-indole, 3-substituted-1H-pyrrolo[2,3-b]pyridine, and 3-substituted-1H-pyrrolo[3,2-b]pyridine compounds, compositions comprising a compound of the present invention, methods of synthesizing compounds of the present invention, and methods for treating mTOR-related diseases comprising the administration of an effective amount of a compound of the present invention. The invention also relates to methods for treating PI3K-related diseases comprising the administration of an effective amount of a compound of the present invention.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery. Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2μ, and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,′: 4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate, and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the α isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. U.S. patent application Ser. Nos. 12/473,605, filed May 28, 2009 and 12/473,658, filed May 28, 2009 disclose compounds that have PI3K and/or mTOR inhibitory activity. Each of the above applications is incorporated by reference herein in its entirety.

The instant invention relates to new compounds that have PI3K and/or mTOR inhibitory activity, and/or that act as prodrugs to provide compounds having PI3K and/or mTOR inhibitory activity.

### SUMMARY OF THE INVENTION

In one aspect, the invention provides compounds of the formula 1:

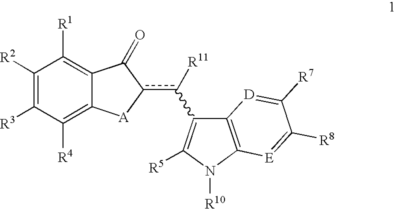

or a pharmaceutically acceptable salt thereof, wherein the constituent variables are as defined below.

In other aspects, the invention provides compounds of formula 2:

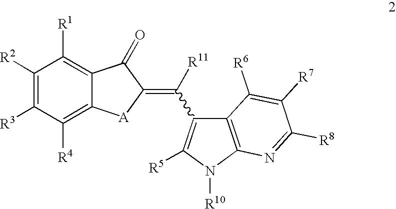

or a pharmaceutically acceptable salt thereof, wherein the constituent variables are as defined below.

In other aspects, the invention provides compounds of formula 3:

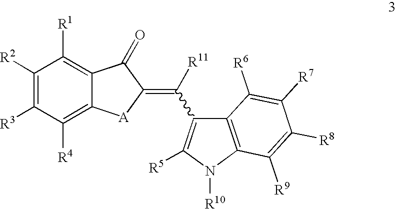

or a pharmaceutically acceptable salt thereof, wherein the constituent variables are as defined below.

In other aspects, the invention provides compositions comprising a compound of the invention, and methods for making compounds of the invention. In further aspects, the invention provides methods for inhibiting PI3K and mTOR in a subject, and methods for treating PI3K-related and mTOR-related disorders in a mammal in need thereof.

In other aspects, the invention provides further methods of synthesizing the compounds or pharmaceutically acceptable salts of compounds of the present formulas 1-3.

### DETAILED DESCRIPTION OF THE INVENTION

In one aspect, the invention provides compounds of the Formula 1:

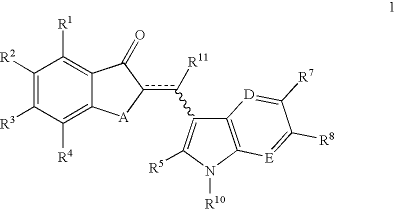

or a geometric isomer thereof, or a pharmaceutically acceptable salt thereof, wherein:

A is oxygen or sulfur;

. . . represents an optional second carbon-to-carbon bond;

D is C—R⁶ or N;

E is C—R⁹ or N;

R¹, R², R³, and R⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; OR²⁰; or hydroxyl;
wherein at least one of R¹-R⁴ is OR²⁰, wherein each R²⁹ is independently selected from C(O)R¹², CO₂R¹², CONR¹²R¹³, P(O)(OR¹²)(OR¹³), P(O)R¹²(OR¹³),
C(R¹²R¹³)OR¹⁴, and C(R¹²R¹³)NR¹⁴R²¹;
with the proviso that R¹² cannot be H when R²⁹ is C(O)R¹² or CO₂R¹²;

R¹², R¹³, R¹⁴ and R²¹ are each independently H; C₁-C₆alkyl optionally substituted with from 1 to 3 substituents independently selected from OH, —NH₂, (C₁-C₆alkyl)NH—, (C₁-C₆alkyl)(C₁-C₆alkyl)N—, C₆-C₁₀ aryl, (C₁-C₆alkyl)oxycarbonyl, and C₁-C₉ heteroaryl; perfluoro(C₁-C₆)alkyl; C₁-C₉heteroaryl optionally substituted with from 1 to 3 substituents independently selected from C₁-C₆alkyl, halo, and perfluoro(C₁-C₆)alkyl; C₆-C₁₄aryl optionally substituted with from 1 to 3 substituents independently selected from C₁-C₆alkyl, halo, and perfluoro(C₁-C₆)alkyl; C₂-C₈ heterocyclyl; or C₃-C₈cycloalkyl;

or when R²⁹ is CONR¹²R¹³, R¹² and R¹³ taken together with the N they are attached to form a 3-10 membered heterocyclyl with 1-3 hetero atoms selected from N, O and S, wherein the 3-10 membered heterocyclyl is optionally substituted with 1-3 substituents selected from straight or branched C₁-C₆alkyl optionally substituted with fluorine, C₆-C₁₄aryl, C₃-C₈cycloalkyl, C₃-C₈heterocyclyl, CN, ═O, NO₂, (CH₂)ₙO(C₁-C₈alkyl), (CH₂)ₙ—NH₂, (C₁-C₈alkyl)NH—(CH₂)ₙ— and (C₁-C₆alkyl)(C₁-C₈alkyl)N—(CH₂)ₙ—.

wherein n is 0 or 1;

R⁵ is H; C₁-C₈alkyl; C₆-C₁₄aryl; C₃-C₈cycloalkyl; halo; C₁-C₉heteroaryl; C₁-C₈heterocyclylalkyl; C₁-C₈ perfluoroalkyl-; R¹⁵R¹⁶NC(O)—; (C₁-C₈alkoxy)carbonyl; or CO₂H;

R¹⁵ and R¹⁶ are each independently H; C₁-C₈alkyl optionally substituted with from 1 to 3 substituents independently selected from —NH₂, (C₁-C₈alkyl)NH—, (C₁-C₈alkyl)(C₁-C₈alkyl)N—, and C₁-C₉heteroaryl; C₁-C₉heteroaryl; C₈-C₁₄aryl optionally substituted with from 1 to 3 substituents independently selected from C₁-C₈alkyl, halo, and perfluoro(C₁-C₈)alkyl; or C₃-C₈cycloalkyl;

or R¹⁵ and R¹⁶ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₈-C₁₄aryl)-, —S—, —SO—, —S(O)₂—, or —O—;

R⁶-R⁹ are each independently: (a) H; (b) C₁-C₆alkoxy-; (c) C₁-C₆alkyl-optionally substituted by C₈-C₁₄aryl-; (d) C₂-C₈alkenyl-optionally substituted by C₈-C₁₄aryl-; (e) C₂-C₈alkynyl-optionally substituted by C₈-C₁₄aryl-; (f) (C₁-C₆alkyl)amido-; (g) C₁-C₈alkylcarboxy-; (h) (C₁-C₆alkyl)carboxyamido-; (i) (C₁-C₆alkyl)SO₂—; (j) C₆-C₁₄aryl-optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy-, (v) (C₁-C₆alkyl)carboxyamido-, (vi) C₁-C₆alkoxy-optionally substituted by C₁-C₆alkoxy or C₁-C₉heteroaryl, (vii) (C₁-C₆alkoxy)carbonyl-, (viii) (C₆-C₁₄aryl)oxy-, (ix) C₃-C₆cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) C₂N—; (k) (C₆-C₁₄aryl)alkyl-O—; (l) halo; (m) C₁-C₉heteroaryl optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy, (v) (C₁-C₆alkyl)carboxyamido-, (vi) C₁-C₆alkoxy-optionally substituted by C₁-C₆alkoxy- or C₁-C₉heteroaryl-, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₈alkyl- or C₁-C₆hydroxylalkyl-, (xiii) hydroxyl, (xiv) C₁-C₆hydroxylalkyl-, (xv) C₁-C₆perfluoroalkyl-, (xvi) C₁-C₆perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (n) hydroxyl; (o) C₁-C₉ heterocyclyl-optionally substituted by: (i) C₁-C₆alkyl-, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) hydroxyl, or (iv) R¹⁷R¹⁸N—; (p) C₁-C₆ perfluoroalkyl-; (q) NC—; (r) (C₁-C₆alkoxy)carbonyl-; (s) HOOC—; or (t) C₂N—;

R¹⁷ and R¹⁸ are each independently H; C₁-C₆alkyl optionally substituted with from 1 to 3 substituents independently selected from C₁-C₆alkoxy, —NH₂, (C₁-C₆alkyl)NH—, (C₁-C₆alkyl)(C₁-C₆alkyl)N—, C₆-C₁₄aryl, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;

or R¹⁷ and R¹⁸ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R¹⁹ is C₁-C₆alkyl or C₆-C₁₄aryl;

or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;

R¹⁰ is H; C₁-C₆alkyl optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl C₁-C₉heteroaryl, C₃-C₈cycloalkyl, C₁-C₆haloalkyl-, C₁-C₆aminoalkyl-, —OC(O)(C₁-C₆alkyl), C₁-C₆-carboxyamidoalkyl-, NO₂, and C₁-C₉ heterocyclyl such as aziridinyl, azetidinyl, pyrrolidinyl, piperidinyl, azepanyl, or piperazinyl, each C₁-C₉ heterocyclyl optionally substituted with C₁-C₆alkyl; C₂-C₁₀ alkenyl; C₆-C₁₄aryl; C₃-C₈cycloalkyl; C₁-C₉heteroaryl; or C₁-C₆heterocyclylalkyl group optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, C₁-C₆alkyl, —C(O)OH, —C(O)O(C₁-C₆alkyl), —C(O)(C₁-C₆alkyl), 4- to 7-membered monocyclic heterocycle, C₆-C₁₄aryl C₁-C₉heteroaryl, C₁-C₆heterocyclylalkyl, and C₃-C₈cycloalkyl;

or R⁵ and R¹⁹ taken together with the atoms connecting them form a fused C₅-C₈ heterocyclic ring containing 2-3 hetero atoms selected from N, O, and S, and optionally substituted with halogen, hydroxy, O—C₁-C₆ alkoxy, CN, ═O, C₁-C₆alkyl, NO₂, NH₂, NHC₁-C₆alkyl, N(C₁-C₆alkyl)₂, C(O)C₁-C₆alkyl, CO₂C₁-C₆alkyl, CONH₂, CONHC₁-C₆alkyl, or CON(C₁-C₆alkyl)₂; and

R¹¹ is H or C₁-C₆alkyl.

In one embodiment, A is oxygen.

In one embodiment, R² is H.

In one embodiment, R⁴ is H.

In one embodiment, R⁵ is H.

In one embodiment, R⁶ is C₆-C₁₄aryl, optionally independently substituted with from 1 to 3 substituents as specified in formula 1.

In one embodiment, R⁷ is H.

In one embodiment, R⁸ is H.

In one embodiment, R²⁰ is C(O)R¹².

In one embodiment, R²⁰ is CO₂R¹².

In one embodiment, R²⁰ is CONR¹²R¹³. In an example of this embodiment, R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1 herein.

In one embodiment, R²⁰ is P(O)(OR¹²)(OR¹³).

In one embodiment, R²⁰ is P(O)R¹²(OR¹³).

In one embodiment, R¹¹ is C₁-C₆alkyl optionally substituted as defined in formula 1.

In one embodiment, R¹¹ is methyl.

In one embodiment, R¹¹ is H.

In one embodiment, R⁵═R⁷═R⁸═H and R¹⁰ is CH₃.

In one embodiment, R⁶ is C₆-C₁₄aryl, optionally independently substituted with from 1 to 3 substituents as specified in formula 1, D is C—R⁶, E is N or C—R⁹, and R¹¹ is H.

In one embodiment, R²═R⁴═R⁵═R⁷═R⁸═R¹¹H, R⁶ is C₆-C₁₄aryl, optionally independently substituted with from 1 to 3 substituents as specified in formula 1, D is C—R⁶, E is N or C—R⁹, and R¹⁰ is CH₃.

In one embodiment of the invention, R¹ and R³ are each OR²⁰ and R² and R⁴ are each H. In one example of this embodiment, each R²⁰ is CO₂R¹², wherein each R¹² is C₁-C₆alkyl optionally substituted as defined in formula 1 or each R¹² is C₆-C₁₄ aryl optionally substituted as defined in formula 1. In another example, each R²⁰ is CONR¹²R¹³, wherein R¹² and R¹³ are each C₆-C₁₄ aryl or each C₁-C₆alkyl wherein each alkyl or aryl is optionally substituted as defined for formula 1, or one of R¹² and R¹³ is C₆-C₁₄ aryl and the other of R¹² and R¹³ is C₁-C₆alkyl wherein the aryl or alkyl is optionally substituted as defined for formula 1, for example with a C₁-C₆alkyloxycarbonyl, or one of R¹² and R¹³ is hydrogen and the other of R¹² and R¹³ is C₁-C₆alkyl wherein the alkyl is optionally substituted as defined for formula 1, for example with a C₁-C₆alkyloxycarbonyl, or for each CONR¹²R¹³R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1 In another example, each R²⁰ is COR¹², wherein each R¹² is C₆-C₁₄ aryl or each R¹² is C₁-C₆alkyl or each R¹² is C₂-C₈ heterocyclyl wherein the alkyl or aryl or heterocyclyl is optionally substituted as defined in formula 1. In another example, each R²⁰ is P(O)(OR¹²)(OR¹³), wherein R¹² and R¹³ are each C₁-C₆alkyl optionally substituted as defined in formula 1 or each hydrogen.

In another embodiment of the invention, R³ is OR²⁰ and R¹, R², and R⁴ are each H or OH,

provided that no more than one of R¹, R², and R⁴ can be OH. In one example of this embodiment, R²⁰ is COR¹², wherein R¹² is C₁-C₆alkyl or C₆-C₁₄ aryl wherein the alkyl or aryl is optionally substituted as defined in formula 1 In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl wherein each alkyl or aryl is optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen.

In another example, R²⁰ is P(O)R¹²(OR¹³), wherein R¹² is C₆-C₁₄ aryl optionally substituted as defined in formula 1 and R¹³ is hydrogen.

In another embodiment of the invention, R¹ is OR²⁰ and R³, R², and R⁴ are each H or OH, provided that no more than one of R², R³, and R⁴ can be OH. In one example of this embodiment, R²⁰ is COR¹², wherein R¹² is C₁-C₆alkyl or C₆-C₁₄ aryl wherein the alkyl or aryl is optionally substituted as defined in formula 1. In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl wherein the alkyl or aryl is optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen. In another example, R²⁰ is P(O)R¹²(OR¹³), wherein R¹² is C₆-C₁₄ aryl optionally substituted as defined in formula 1 and R¹³ is hydrogen.

In another embodiment of the invention, R² is OR²⁰ and R³, R¹, and R⁴ are each H or OH, provided that no more than one of R³, R¹, and R⁴ can be OH. In one example of this embodiment, R²⁰ is CONR¹²R¹³, wherein one of R¹² and R¹³ is hydrogen and the other of R¹² and R¹³ is C₁-C₆alkyl optionally substituted as defined in formula 1.

In another embodiment of the invention, R¹⁰ is selected from hydrogen; (C₁-C₆)alkyl optionally substituted with di(C₁-C₆)alkylamino or with C₁-C₉ heterocyclyl such as piperazinyl which is optionally substituted with C₁-C₆alkyl;

In another embodiment of the invention, R⁷ is selected from hydrogen, (C₁-C₆)alkoxy,

In another embodiment of the invention, R⁵ is selected from hydrogen, (C₆-C₁₄)aryl, C1-C₆alkyl,

In another embodiment of the invention, R⁶ is selected from hydrogen, (C₆-C₁₄)aryl, or C₁-C₉ heterocyclyl such as (8-oxa-3-azabicyclo[3.2.1]oct-3-yl).

In another aspect, the invention provides compounds of the Formula 2:

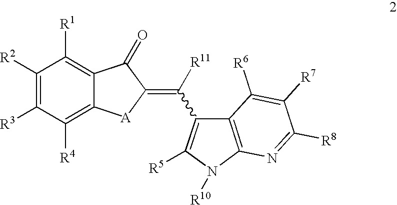

or a geometric isomer thereof or a pharmaceutically acceptable salt thereof, wherein:

A is oxygen or sulfur;

R¹, R², R³, and R⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; OR²⁰; or hydroxyl;

wherein at least one of R¹-R⁴ is OR²⁰, wherein each R²⁰ is independently selected from C(O)R¹², CO₂R¹², CONR¹²R¹³, P(O)(OR¹²)(OR¹³), P(O)R¹²(OR¹³),
C(R¹²R¹³)OR¹⁴, and C(R¹²R¹³)NR¹⁴R²¹;
with the proviso that R¹² cannot be H when R²⁰ is C(O)R¹² or CO₂R¹²;

R¹², R¹³, R¹⁴ and R²¹ are each independently H; C₁-C₆alkyl optionally substituted with from 1 to 3 substituents independently selected from OH, —NH₂, (C₁-C₆alkyl)NH—, (C₁-C₆alkyl)(C₁-C₆alkyl)N—, C₆-C₁₀ aryl, (C₁-C₆alkyl)oxycarbonyl, and C₁-C₉ heteroaryl; perfluoro(C₁-C₆)alkyl; C₁-C₉heteroaryl optionally substituted with from 1 to 3 substituents independently selected from C₁-C₆alkyl, halo, and perfluoro(C₁-C₆)alkyl; C₆-C₁₄aryl optionally substituted with from 1 to 3 substituents independently selected from C₁-C₆alkyl, halo, and perfluoro(C₁-C₆)alkyl; C₂-C₈ heterocyclyl; or C₃-C₈cycloalkyl;

or when R²⁰ is CONR¹²R¹³, R¹² and R¹³ taken together with the N they are attached to form a 3-10 membered heterocyclyl with 1-3 hetero atoms selected from N, O and S, wherein the 3-10 membered heterocyclyl is optionally substituted with 1-3 substituents selected from straight or branched C₁-C₆alkyl optionally substituted with fluorine, C₆-C₁₄aryl, C₃-C₈cycloalkyl, C₃-C₈heterocyclyl, CN, ═O, NO₂, (CH₂)ₙO(C₁-C₆alkyl), (CH₂)ₙ—, —NH₂, (C₁-C₆alkyl)NH—(CH₂)ₙ—, and (C₁-C₆alkyl)(C₁-C₆alkyl)N—(CH₂)ₙ—;

wherein n is 0 or 1;

R⁵ is H; C₁-C₆alkyl; C₆-C₁₄aryl; C₃-C₈cycloalkyl; halo; C₁-C₉heteroaryl; C₁-C₆heterocyclylalkyl; C₁-C₆ perfluoroalkyl-; R¹⁵R¹⁶NC(O)—; (C₁-C₆alkoxy)carbonyl; or CO₂H;

R¹⁵ and R¹⁶ are each independently H; C₁-C₆alkyl optionally substituted with from 1 to 3 substituents independently selected from —NH₂, (C₁-C₆alkyl)NH—, (C₁-C₆alkyl)(C₁-C₆alkyl)N—, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;

or R¹⁵ and R¹⁶ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R⁶—R⁸ are each independently: (a) H; (b) C₁-C₆alkoxy-; (c) C₁-C₆alkyl-optionally substituted by C₆-C₁₄aryl-; (d) C₂-C₆alkenyl-optionally substituted by C₆-C₁₄aryl-; (e) C₂-C₆alkynyl-optionally substituted by C₆-C₁₄aryl-; (f) (C₁-C₆alkyl)amido-; (g) C₁-C₆alkylcarboxy-; (h) (C₁-C₆alkyl)carboxyamido-; (i) (C₁-C₆alkyl)SO₂—; (j) C₆-C₁₄aryl-optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy-, (v) (C₁-C₆alkyl)carboxyamido-, (vi) C₁-C₆alkoxy-optionally substituted by C₁-C₆alkoxy or C₁-C₉heteroaryl, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (k) (C₆-C₁₄aryl)alkyl-O—; (l) halo; (m) C₁-C₉heteroaryl optionally substituted with from 1 to 3 substituents independently selected from: (i) C₁-C₈acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy, (v) (C₁-C₆alkyl)carboxyamido-, (vi) C₁-C₆alkoxy-optionally substituted by C₁-C₆alkoxy- or C₁-C₉heteroaryl-, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (n) hydroxyl; (o) C₁-C₉ heterocyclyl-optionally substituted by: (i) C₁-C₆alkyl-, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) hydroxyl, or (iv) R¹⁷R¹⁸N—; (p) C₁-C₆ perfluoroalkyl-; (q) NC—; (r) (C₁-C₆alkoxy)carbonyl-; (s) HOOC—; or (t) O₂N—;

R¹⁷ and R¹⁸ are each independently H; C₁-C₆alkyl optionally substituted with from 1 to 3 substituents independently selected from C₁-C₆alkoxy, —NH₂, (C₁-C₆alkyl)NH—, (C₁-C₆alkyl)(C₁-C₆alkyl)N—, C₆-C₁₄aryl, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;

or R¹⁷ and R¹⁸ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R¹⁹ is C₁-C₆alkyl or O₆—C₁₄aryl;

or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;

R¹⁰ is H; C₁-C₆alkyl optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, NO₂, and C₁-C₉ heterocyclyl such as aziridinyl, azetidinyl, pyrrolidinyl, piperidinyl, azepanyl, or piperazinyl, each C₁-C₉ heterocyclyl optionally substituted with C₁-C₆alkyl; C₂-C₁₀ alkenyl; C₆-C₁₄aryl; C₃-C₈cycloalkyl; C₁-C₉heteroaryl; or C₁-C₆heterocyclylalkyl group optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, C₁-C₆alkyl, —C(O)OH, —C(O)O(C₁-C₆alkyl), —C(O)(C₁-C₆alkyl), 4- to 7-membered monocyclic heterocycle, C₆-C₁₄aryl C₁-C₉heteroaryl, C₁-C₆heterocyclylalkyl, and C₃-C₈cycloalkyl;

or R⁵ and R¹⁰ taken together with the atoms connecting them form a fused C₅-C₈ heterocyclic ring containing 2-3 hetero atoms selected from N, O, and S, and optionally substituted with halogen, hydroxy, O—C₁-C₆ alkoxy, CN, ═O, C₁-C₆alkyl, NO₂, NH₂, NHC₁-C₆alkyl, N(C₁-C₆alkyl)₂, C(O)C₁-C₆alkyl, CO₂C₁-C₆alkyl, CONH₂, CONHC₁-C₆alkyl, or CON(C₁-C₆alkyl)₂; and

R¹¹ is H or C₁-C₆alkyl.

In one embodiment, A is oxygen.

In one embodiment, R² is H.

In one embodiment, R⁴ is H.

In one embodiment, R⁵ is H.

In one embodiment, R⁶ is C₆-C₁₄aryl, optionally independently substituted with from 1 to 3 substituents as specified in formula 1.

In one embodiment, R⁷ is H.

In one embodiment, R⁸ is H.

In one embodiment, R²⁰ is C(O)R¹².

In one embodiment, R²⁰ is CO₂R¹².

In one embodiment, R²⁰ is CONR¹²R¹³. In an example of this embodiment, R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1 herein.

In one embodiment, R²⁰ is P(O)(OR¹²)(OR¹³).

In one embodiment, R²⁰ is P(O)R¹²(OR¹³).

In one embodiment, R¹⁰ is C₁-C₆alkyl optionally substituted as defined in formula 1.

In one embodiment, R¹⁰ is methyl.

In one embodiment, R¹¹ is H.

In one embodiment, R⁵═R⁷═R⁸═H and R¹⁰ is CH₃.

In one embodiment, R⁶ is C₆-C₁₄aryl, optionally independently substituted with from 1 to 3 substituents as specified in formula 1, and R¹¹ is H.

In one embodiment, R², R⁴, R⁵, R⁷, R⁸, R¹¹H, R⁶ is C₆-C₁₄aryl, optionally independently substituted with from 1 to 3 substituents as specified in Formula 1, and R¹⁰ is CH₃.

In one embodiment of the compound of formula 2, R¹ and R³ are each OR²⁰ and R² and R⁴ are each H. In one example of this embodiment, each R²⁰ is CO₂R¹², wherein each R¹² is C₁-C₆alkyl optionally substituted as defined in formula 1 or each R¹² is C₆-C₁₄ aryl optionally substituted as defined in formula 1. In another example, each R²⁰ is CONR¹²R¹³ are each C₆-C₁₄ aryl or each C₁-C₆alkyl, wherein each alkyl or aryl is optionally substituted as defined for formula 1, or one of R¹² and R¹³ is C₆-C₁₄ aryl and the other of R¹² and R¹³ is C₁-C₆alkyl, wherein the alkyl or aryl is optionally substituted as defined for formula 1, for example with a C₁-C₆alkyloxycarbonyl, or one of R¹² and R¹³ is hydrogen and the other of R¹² and R¹³ is C₁-C₆alkyl wherein the alkyl is optionally substituted as defined for formula 1, for example with a C₁-C₆alkyloxycarbonyl, or for each CONR¹²R¹³R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1. In another example, each R²⁰ is COR¹², wherein each R¹² is C₆-C₁₄ aryl or each R¹² is C₁-C₆alkyl or each R¹² is C₂-C₈ heterocyclyl wherein the alkyl or aryl or heterocyclyl is optionally substituted as defined in formula 1. In another example, each R²⁰ is P(O)(OR¹²)(OR¹³), wherein R¹² and R¹³ are each C₁-C₆alkyl optionally substituted as defined in formula 1 or each hydrogen.

In another embodiment of the compound of formula 2, R³ is OR²⁰ and R¹, R², and R⁴ are each H or OH, provided that no more than one of R¹, R², and R⁴ can be OH. In one example of this embodiment, R²⁰ is COR¹², wherein R¹² is C₁-C₆alkyl or C₆-C₁₄ aryl wherein the alkyl or aryl is optionally substituted as defined in formula 1. In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl wherein each alkyl or aryl is optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen.

In another example, R²⁰ is P(O)R¹²(OR¹³), wherein R¹² is C₆-C₁₄ aryl optionally substituted as defined in formula 1 and R¹³ is hydrogen.

In another embodiment of the compound of formula 2, R¹ is OR²⁰ and R³, R², and R⁴ are each H or OH, provided that no more than one of R², R³, and R⁴ can be OH. In one example of this embodiment, R²⁰ is COR¹², wherein R¹² is C₁-C₆alkyl or C₆-C₁₄ aryl wherein the alkyl or aryl is optionally substituted as defined in formula 1. In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl wherein the alkyl or aryl is optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen. In another example, R²⁰ is P(O)R¹²(OR¹³), wherein R¹² is C₆-C₁₄ aryl optionally substituted as defined in formula 1 and R¹³ is hydrogen.

In another embodiment of the compound of formula 2, R² is OR²⁰ and R³, R¹, and R⁴ are each H or OH, provided that no more than one of R³, R¹, and R⁴ can be OH. In one example of this embodiment, R²⁰ is CONR¹²R¹³, wherein one of R¹² and R¹³ is hydrogen and the other of R¹² and R¹³ is C₁-C₆alkyl optionally substituted as defined in formula 1.

In another embodiment of the compound of formula 2, R¹⁰ is selected from hydrogen; (C₁-C₆)alkyl optionally substituted with di(C₁-C₆)alkylamino or with C₁-C₉ heterocyclyl such as piperazinyl which is optionally substituted with C₁-C₆alkyl;

In another embodiment of the compound of formula 2, R⁷ is selected from hydrogen, (C₁-C₆)alkoxy,

In another embodiment of the compound of formula 2, R⁵ is selected from hydrogen, (C₆-C₁₄)aryl, C₁-C₆alkyl,

In another embodiment of the compound of formula 2, R⁶ is selected from hydrogen, (C₆-C₁₄)aryl, or C₁-C₆ heterocyclyl such as (8-oxa-3-azabicyclo[3.2.1]oct-3-yl).

In another aspect, the invention provides compounds of the Formula 3:

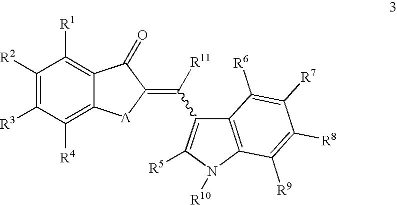

or a geometric isomer thereof or a pharmaceutically acceptable salt thereof, wherein:

A is oxygen or sulfur;

R¹, R², R³, and R⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; OR²⁰; or hydroxyl;
wherein at least one of R₁-R₄ is OR²⁰, wherein each R²⁰ is independently selected from C(O)R¹², CO₂R¹², CONR¹²R¹³, P(O)(OR¹²)(OR¹³), P(O)R¹²(OR¹³),
C(R¹²R¹³)OR¹⁴, and C(R¹²R¹³)NR¹⁴R²¹; with the proviso that R¹² cannot be H when R²⁰ is C(O)R¹² or CO₂R¹²;

R¹², R¹³, R¹⁴ and R²¹ are each independently H; C₁-C₆alkyl optionally substituted with from 1 to 3 substituents independently selected from OH, —NH₂, (C₁-C₆alkyl)NH—, (C₁-C₆alkyl)(C₁-C₆alkyl)N—, C₆-C₁₀ aryl, (C₁-C₆alkyl)oxycarbonyl, and C₁-C₉ heteroaryl; perfluoro(C₁-C₆)alkyl; C₁-C₉heteroaryl optionally substituted with from 1 to 3 substituents independently selected from C₁-C₆alkyl, halo, and perfluoro(C₁-C₆)alkyl; C₆-C₁₄aryl optionally substituted with from 1 to 3 substituents independently selected from C₁-C₆alkyl, halo, and perfluoro(C₁-C₆)alkyl; C₂-C₈ heterocyclyl; or C₃-C₈cycloalkyl;

or when R²⁰ is CONR¹²R¹³, R¹² and R¹³ taken together with the N they are attached to form a 3-10 membered heterocyclyl with 1-3 hetero atoms selected from N, O and S, wherein the 3-10 membered heterocyclyl is optionally substituted with 1-3 substituents selected from straight or branched C₁-C₆alkyl optionally substituted with fluorine, C₆-C₁₄aryl, C₃-C₈ cycloalkyl, C₃-C₈ heterocyclyl, CN, ═O, NO₂, (CH₂)ₙO(C₁-C₆alkyl), (CH₂), —NH₂, (C₁-C₆alkyl)NH—(CH₂)ₙ—, and (C₁-C₆alkyl)(C₁-C₆alkyl)N—(CH₂)ₙ—;

wherein n is 0 or 1;

R¹⁴ and R²¹ are each independently C₁-C₆alkyl, C₁-C₆hydroxylalkyl-, or C₆-C₁₄aryl;

R⁵ is H; C₁-C₆alkyl; C₆-C₁₄aryl; C₃-C₈cycloalkyl; halo; C₁-C₉heteroaryl; C₁-C₆heterocyclylalkyl; C₁-C₆ perfluoroalkyl-; R¹⁵R¹⁶NC(O)—; (C₁-C₆alkoxy)carbonyl; or CO₂H;

R¹⁵ and R¹⁶ are each independently H; C₁-C₆alkyl optionally substituted with from 1 to 3 substituents independently selected from —NH₂, (C₁-C₆alkyl)NH—, (C₁-C₆alkyl)(C₁-C₆alkyl)N—, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;

or R¹⁵ and R¹⁶ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R⁶—R⁹ are each independently: (a) H; (b) C₁-C₆alkoxy-; (c) C₁-C₆alkyl-optionally substituted by C₆-C₁₄aryl-; (d) C₂-C₆alkenyl-optionally substituted by C₆-C₁₄aryl-; (e) C₂-C₆alkynyl-optionally substituted by C₆-C₁₄aryl-; (f) (C₁-C₆alkyl)amido-; (g) C₁-C₆alkylcarboxy-; (h) (C₁-C₆alkyl)carboxyamido-; (i) (C₁-C₆alkyl)SO₂—; (j) 06-C₁₄aryl-optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy-, (v) (C₁-C₆alkyl)carboxyamido-, (vi) C₁-C₆alkoxy-optionally substituted by C₁-C₆alkoxy or C₁-C₉heteroaryl, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (k) (C₆-C₁₄aryl)alkyl-O—; (l) halo; (m) C₁-C₉heteroaryl optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₆heterocyclyl-, (iii) (C₁-C₆alkyl)amido-, (iv) (C₁-C₆alkyl)carboxy, (v) (C₁-C₆alkyl)carboxyamido-, (vi) C₁-C₆alkoxy-optionally substituted by C₁-C₆alkoxy- or C₁-C₉heteroaryl-, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (n) hydroxyl; (o) C₁-C₉ heterocyclyl-optionally substituted by: (i) C₁-C₆alkyl-, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) hydroxyl, or (iv) R¹⁷R¹⁸N—; (p) C₁-C₆ perfluoroalkyl-; (q) NC—; (r) (C₁-C₆alkoxy)carbonyl-; (s) HOOC—; or (t) O₂N—;

R¹⁷ and R¹⁸ are each independently H; C₁-C₆alkyl optionally substituted with from 1 to 3 substituents independently selected from C₁-C₆alkoxy, —NH₂, (C₁-C₆alkyl)NH—, (C₁-C₆alkyl)(C₁-C₆alkyl)N—, C₆-C₁₄aryl, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;

or R¹⁷ and R¹⁸ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;

R¹⁹ is C₁-C₆alkyl or C₆-C₁₄aryl;

or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;

R¹⁰ is H; C₁-C₆alkyl optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, NO₂, and C₁-C₉ heterocyclyl such as aziridinyl, azetidinyl, pyrrolidinyl, piperidinyl, azepanyl, or piperazinyl, each C₁-C₉ heterocyclyl optionally substituted with C₁-C₆alkyl; C₂-C₁₀ alkenyl; C₆-C₁₄aryl; C₃-C₈cycloalkyl; C₁-C₆heteroaryl; or C₁-C₆heterocyclylalkyl group optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, C₁-C₆alkyl, —C(O)OH, —C(O)O(C₁-C₆alkyl), —C(O)(C₁-C₆alkyl), 4- to 7-membered monocyclic heterocycle, C₆-C₁₄aryl C₁-C₉heteroaryl, C₁-C₆heterocyclylalkyl, and C₃-C₈cycloalkyl;

or R⁵ and R¹⁰ taken together with the atoms connecting them form a fused C₅-C₈ heterocyclic ring containing 2-3 hetero atoms selected from N, O, and S, and optionally substituted with halogen, hydroxy, O—C₁-C₆ alkoxy, CN, ═O, C₁-C₆alkyl, NO₂, NH₂, NHC₁-C₆alkyl, N(C₁-C₆alkyl)₂, C(O)C₁-C₆alkyl, CO₂C₁-C₆alkyl, CONH₂, CONHC₁-C₆alkyl, or CON(C₁-C₆alkyl)₂; and

R¹¹ is H or C₁-C₆alkyl.

In one embodiment, A is oxygen.

In one embodiment, R² is H.

In one embodiment, R⁴ is H.

In one embodiment, R⁵ is H.

In one embodiment, R⁶ is C₆-C₁₄aryl, optionally independently substituted with from 1 to 3 substituents as specified in Formula 1.

In one embodiment, R⁷ is H.

In one embodiment, R⁸ is H.

In one embodiment, R²⁰ is C(O)R¹².

In one embodiment, R²⁰ is CO₂R¹².

In one embodiment, R²⁰ is CONR¹²R¹³. In an example of this embodiment, R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1 herein.

In one embodiment, R²⁰ is P(O)(OR¹²)(OR¹³).

In one embodiment, R²⁰ is P(O)R¹²(OR¹³).

In one embodiment, R¹⁰ is C₁-C₆alkyl optionally substituted as defined in formula 1.

In one embodiment, R¹⁰ is methyl.

In one embodiment, R¹¹ is H.

In one embodiment, R⁵═R⁷═R⁸═H and R¹⁰ is CH₃.

In one embodiment, R⁶ is C₆-C₁₄aryl, optionally independently substituted with from 1 to 3 substituents as specified in Formula 1, and R¹¹ is H.

In one embodiment, R², R⁴, R⁵, R⁷, R⁸, R¹¹H, R⁶ is C₆-C₁₄aryl, optionally independently substituted with from 1 to 3 substituents as specified in Formula 1, and R¹¹ is CH₃.

In one embodiment of the compound of formula 3, R¹ and R³ are each OR²⁰ and R² and R⁴ are each H. In one example of this embodiment, each R²⁰ is CO₂R¹², wherein each R¹² is C₁-C₆alkyl optionally substituted as defined in claim 1 or each R¹² is C₆-C₁₄ aryl optionally substituted as defined in formula 1. In another example, each R²⁰ is CONR¹²R¹³, wherein R¹² and R¹³ are each C₆-C₁₄ aryl or each C₁-C₆alkyl wherein each alkyl or aryl is optionally substituted as defined for formula 1, or one of R¹² and R¹³ is C₆-C₁₄ aryl and the other of R¹² and R¹³ is C₁-C₆alkyl wherein the aryl or alkyl is optionally substituted as defined for formula 1, for example with a C₁-C₆alkyloxycarbonyl, or one of R¹² and R¹³ is hydrogen and the other of R¹² and R¹³ is C₁-C₆alkyl wherein the alkyl is optionally substituted as defined for formula 1, for example with a C₁-C₆alkyloxycarbonyl, or for each CONR¹²R¹³R¹² and R¹³ taken together with the N they are attached to form a 3-10 membered heterocyclyl with 1-3 hetero atoms selected from N, O and S, wherein the 3-10 membered heterocyclyl is optionally substituted as defined for formula 1. In another example, each R²⁰ is COR¹², wherein each R¹² is C₆-C₁₄ aryl or each R¹² is C₁-C₆alkyl or each R¹² is C₂-C₈ heterocyclyl wherein the alkyl or aryl or heterocyclyl is optionally substituted as defined in formula 1. In another example, each R²⁰ is P(O)(OR¹²)(OR¹³), wherein R¹² and R¹³ are each C₁-C₆alkyl or each hydrogen.

In another embodiment of the compound of formula 3, R³ is OR²⁰ and R¹, R², and R⁴ are each H or OH, provided that no more than one of R¹, R², and R⁴ can be OH.

In one example of this embodiment, R²⁰ is COR¹², wherein R¹² is C₁-C₆alkyl or C₆-C₁₄ aryl wherein the alkyl or aryl is optionally substituted as defined in formula 1. In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl wherein each alkyl or aryl is optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen.

In another example, R²⁰ is P(O)R¹²(OR¹³), wherein R¹² is C₆-C₁₄ aryl optionally substituted as defined in formula 1 and R¹³ is hydrogen.

In another embodiment of the compound of formula 3, R¹ is OR²⁰ and R³, R², and R⁴ are each H or OH, provided that no more than one of R², R³, and R⁴ can be OH. In one example of this embodiment, R²⁰ is COR¹², wherein R¹² is C₁-C₆alkyl or C₆-C₁₄ aryl wherein the alkyl or aryl is optionally substituted as defined in formula 1. In another example of this embodiment, R²⁰ is COOR¹², wherein R¹² is C₆-C₁₄ aryl optionally substituted as defined in formula 1. In another example, R²⁰ is CONR¹²R¹³, wherein R¹² and R¹³ are each C₁-C₆alkyl or each C₆-C₁₄ aryl optionally substituted as defined in formula 1, or wherein one of R¹² and R¹³ is hydrogen and the other of R¹² and R¹³ is C₁-C₆alkyl optionally substituted as defined in formula 1. In another example, R²⁰ is P(O)(OR¹²)(OR¹³), wherein R¹² and R¹³ are each hydrogen. In another example, R²⁰ is P(O)R¹²(OR¹³), wherein R¹² is C₆-C₁₄ aryl optionally substituted as defined in formula 1 and R¹³ is hydrogen.

In another embodiment of the compound of formula 3, R² is OR²⁰ and R³, R¹, and R⁴ are each H or OH, provided that no more than one of R³, R¹, and R⁴ can be OH. In one example of this embodiment, R²⁰ is CONR¹²R¹³, wherein one of R¹² and R¹³ is hydrogen and the other of R¹² and R¹³ is C₁-C₆alkyl optionally substituted as defined in formula 1.

In another embodiment of the compound of formula 3, R¹⁰ is selected from hydrogen; (C₁-C₆)alkyl optionally substituted with di(C₁-C₆)alkylamino or with C₁-C₉ heterocyclyl such as piperazinyl which is optionally substituted with C₁-C₆alkyl;

In another embodiment of the compound of formula 3, R⁷ is selected from hydrogen, (C₁-C₆)alkoxy,

In another embodiment of the compound of formula 3, R⁵ is selected from hydrogen, (C₆-C₁₄)aryl, C₁-C₆alkyl,

In another embodiment of the compound of formula 3, R⁶ is selected from hydrogen, (C₆-C₁₄)aryl, or C₁-C₉ heterocyclyl such as (8-oxa-3-azabicyclo[3.2.1]oct-3-yl).

Illustrative compounds of formula 1 are each of the compounds 1-48 below, or a geometric isomer thereof or a pharmaceutically acceptable salt thereof:
(2Z)-2-({1-[3-(dimethylamino)propyl]-5-methoxy-1H-indol-3-yl}methylene)-3-oxo-2,3-dihydro-1-benzofuran-4,6-diyl dimorpholine-4-carboxylate;
(2Z)-2-({1-[3-(dimethylamino)propyl]-5-methoxy-1H-indol-3-yl}methylene)-3-oxo-2,3-dihydro-1-benzofuran-4,6-diylbis[methyl(phenyl)carbamate];
(2Z)-2-[(5-methoxy-2-phenyl-1H-indol-3-yl)methylene]-3-oxo-2,3-dihydro-1-benzofuran-4,6-diyl diacetate;
(2Z)-2-({1-[3-(dimethylamino)propyl]-5-methoxy-1H-indol-3-yl}methylene)-3-oxo-2,3-dihydro-1-benzofuran-4,6-diylbis(diisopropylcarbamate);
(2Z)-2-({1-[3-(dimethylamino)propyl]-5-methoxy-1H-indol-3-yl}methylene)-3-oxo-2,3-dihydro-1-benzofuran-4,6-diyldiisopropyl biscarbonate;
(2Z)-2-[(5-methoxy-2-phenyl-1H-indol-3-yl)methylene]-3-oxo-2,3-dihydro-1-benzofuran-4,6-diyl dibenzoate;
diisopropyl (2Z)-2-[(5-methoxy-2-phenyl-1H-indol-3-yl)methylene]-3-oxo-2,3-dihydro-1-benzofuran-4,6-diyl biscarbonate;
(2Z)-2-({1-[3-(dimethylamino)propyl]-5-methoxy-1H-indol-3-yl}methylene)-3-oxo-2,3-dihydro-1-benzofuran-4,6-diylbis(dimethylcarbamate);
(2Z)-2-({1-[3-(dimethylamino)propyl]-5-methoxy-1H-indol-3-yl}methylene)-3-oxo-2,3-dihydro-1-benzofuran-4,6-diyl bis(3-methylbutanoate);
(2Z)-2-[(5-methoxy-2-phenyl-1H-indol-3-yl)methylene]-3-oxo-2,3-dihydro-1-benzofuran-4,6-diyl bis(2,2-dimethylpropanoate);
(2Z)-2-[(5-methoxy-2-phenyl-1H-indol-3-yl)methylene]-3-oxo-2,3-dihydro-1-benzofuran-4,6-diyl bis(diphenylcarbamate);
(2Z)-2-({5-methoxy-2-methyl-1-[2-(4-methylpiperazin-1-yl)pethyl]-1H-indol-3-yl}methylene)-3-oxo-2,3-dihydro-1-benzofuran-4,6-diyl bis(dimethylcarbamate);
(2Z)-2-({1-[3-(dimethylamino)propyl]-5-methoxy-1H-indol-3-yl}methylene)-3-oxo-2,3-dihydro-1-benzofuran-4,6-diyl dimethyl biscarbonate;
(2Z)-2-({1-[3-(dimethylamino)propyl]-5-methoxy-1H-indol-3-yl}methylene)-3-oxo-2,3-dihydro-1-benzofuran-4,6-diyl bis(2,2-dimethylpropanoate);
(2Z)-2-({1-[3-(dimethylamino)propyl]-5-methoxy-1H-indol-3-yl}methylene)-3-oxo-2,3-dihydro-1-benzofuran-4,6-diyl diacetate;
(2Z)-2-({5-methoxy-2-methyl-1-[2-(4-methylpiperazin-1-yl)pethyl]-1H-indol-3-yl}methylene)-3-oxo-2,3-dihydro-1-benzofuran-4,6-diyl dibenzoate;
(2Z)-2-[(5-methoxy-2-phenyl-1H-indol-3-yl)methylene]-3-oxo-2,3-dihydro-1-benzofuran-6-yl benzoate;
(2Z)-2-[(5-methoxy-2-phenyl-1H-indol-3-yl)methylene]-3-oxo-2,3-dihydro-1-benzofuran-6-yl dimethylcarbamate;
(2Z)-2-[(5-methoxy-2-phenyl-1H-indol-3-yl)methylene]-3-oxo-2,3-dihydro-1-benzofuran-6-yl phenyl carbonate;
(2Z)-2-[(5-methoxy-2-phenyl-1H-indol-3-yl)methylene]-3-oxo-2,3-dihydro-1-benzofuran-6-yl diphenylcarbamate;
(2Z)-2-({1-[3-(dimethylamino)propyl]-5-methoxy-1H-indol-3-yl}methylene)-3-oxo-2,3-dihydro-1-benzofuran-4,6-diyl bis(diphenylcarbamate);
(2Z)-2-[(5-methoxy-2-phenyl-1H-indol-3-yl)methylene]-3-oxo-2,3-dihydro-1-benzofuran-4,6-diyl dimethyl biscarbonate;
(2Z)-2-[(5-methoxy-2-phenyl-1H-indol-3-yl)methylene]-3-oxo-2,3-dihydro-1-benzofuran-4,6-diyl diphenyl biscarbonate;
(2Z)-2-({1-[3-(dimethylamino)propyl]-5-methoxy-1H-indol-3-yl}methylene)-3-oxo-2,3-dihydro-1-benzofuran-4,6-diyl dibenzoate;
ethyl N-({[(2Z)-2-({1-[3-(dimethylamino)propyl]-5-methoxy-1H-indol-3-yl}methylene)-3-oxo-2,3-dihydro-1-benzofuran-6-yl]oxy}carbonyl)glycinate;
(2Z)-2-[(5-methoxy-2-phenyl-1H-indol-3-yl)methylene]-3-oxo-2,3-dihydro-1-benzofuran-6-yl hydrogen phenylphosphonate;
(2Z)-2-[(5-methoxy-2-phenyl-1H-indol-3-yl)methylene]-3-oxo-2,3-dihydro-1-benzofuran-6-yl dihydrogen phosphate;
(2Z)-2-({5-methoxy-2-methyl-1-[2-(4-methylpiperazin-1-yl)pethyl]-1H-indol-3-yl}methylene)-3-oxo-2,3-dihydro-1-benzofuran-4,6-diyl bis(diisopropylcarbamate);
(2Z)-2-({5-methoxy-2-methyl-1-[2-(4-methylpiperazin-1-yl)pethyl]-1H-indol-3-yl}methylene)-3-oxo-2,3-dihydro-1-benzofuran-4,6-diyl bis(diphenylcarbamate);
(2Z)-2-[(5-methoxy-2-phenyl-1H-indol-3-yl)methylene]-3-oxo-2,3-dihydro-1-benzofuran-4,6-diyl bis(dimethylcarbamate);
(2Z)-2-[(5-methoxy-2-phenyl-1H-indol-3-yl)methylene]-3-oxo-2,3-dihydro-1-benzofuran-4,6-diyl bis(diisopropylcarbamate);
tetraethyl (2Z)-2-[(5-methoxy-2-phenyl-1H-indol-3-yl)methylene]-3-oxo-2,3-dihydro-1-benzofuran-4,6-diyl bis(phosphate);
(2Z)-2-({1-[3-(dimethylamino)propyl]-5-methoxy-1H-indol-3-yl}methylene)-3-oxo-2,3-dihydro-1-benzofuran-4,6-diyl tetraethyl bis(phosphate);
diethyl (2Z)-2-[(5-methoxy-2-phenyl-1H-indol-3-yl)methylene]-3-oxo-2,3-dihydro-1-benzofuran-6-yl phosphate;
diethyl 2,2′-[[(2Z)-2-({1-[3-(dimethylamino)propyl]-5-methoxy-1H-indol-3-yl}methylene)-3-oxo-2,3-dihydro-1-benzofuran-4,6-diyl]bis(oxycarbonylimino)]diacetate;
(2Z)-2-({1-[3-(dimethylamino)propyl]-5-methoxy-1H-indol-3-yl}methylene)-3-oxo-2,3-dihydro-1-benzofuran-4,6-diyl bis(4-methylpiperazine-1-carboxylate);
(2Z)-2-({1-[3-(dimethylamino)propyl]-5-methoxy-1H-indol-3-yl}methylene)-3-oxo-2,3-dihydro-1-benzofuran-4,6-diyl bis(4-benzylpiperazine-1-carboxylate);
(2Z)-2-[(5-methoxy-2-phenyl-1H-indol-3-yl)methylene]-3-oxo-2,3-dihydro-1-benzofuran-4,6-diyl bis[dihydrogen (phosphate)];
(2Z)-6-hydroxy-2-({5-methoxy-2-methyl-1-[2-(4-methylpiperazin-1-yl)pethyl]-1H-indol-3-yl}methylene)-3-oxo-2,3-dihydro-1-benzofuran-4-yl dimethylcarbamate;
(2Z)-2-({1-[3-(dimethylamino)propyl]-5-methoxy-1H-indol-3-yl}methylene)-6-hydroxy-3-oxo-2,3-dihydro-1-benzofuran-4-yl dimethylcarbamate;
(2Z)-4-hydroxy-2-({5-methoxy-2-methyl-1-[2-(4-methylpiperazin-1-yl)pethyl]-1H-indol-3-yl}methylene)-3-oxo-2,3-dihydro-1-benzofuran-6-yl dimethylcarbamate;
(2Z)-2-({1-[3-(dimethylamino)propyl]-5-methoxy-1H-indol-3-yl}methylene)-4-hydroxy-3-oxo-2,3-dihydro-1-benzofuran-6-yl dimethylcarbamate;
(2Z)-2-[(1-methyl-4-phenyl-1H-pyrrolo[2,3-b]pyridin-311)methylene]-3-oxo-2,3-dihydro-1-benzofuran-4,6-diyl bis(dimethylcarbamate);
(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-4-yl dimethylcarbamate;
(2Z)-2-({5-methoxy-1-[2-(4-methylpiperazin-1-yl)pethyl]-1H-indol-3-yl}methylene)-3-oxo-2,3-dihydro-1-benzofuran-5-yl methylcarbamate;
(2Z)-2-({5-methoxy-2-methyl-1-[2-(4-methylpiperazin-1-yl)pethyl]-1H-indol-3-yl}methylene)-3-oxo-2,3-dihydro-1-benzofuran-6-ylmethyl(phenyl)carbamate;
(2Z)-2-({5-methoxy-2-methyl-1-[2-(4-methylpiperazin-1-yl)pethyl]-1H-indol-3-yl}methylene)-3-oxo-2,3-dihydro-1-benzofuran-6-yl diisopropylcarbamate; and
(2Z)-2-[(1-methyl-4-phenyl-1H-indol-3-yl)methylene]-3-oxo-2,3-dihydro-1-benzofuran-4,6-diyl bis(dimethylcarbamate).

The compounds of the invention may be made, for example, from phenolic compounds having one or more hydroxyl groups on the benzene moiety of the benzofuranone group or benzothiophenone group of formula 1. Illustrative phenolic compounds are set forth below:
4,6-dihydroxy-2-[(5-methoxy-2-phenyl-1H-indol-3-yl)methylene]-1-benzofuran-3(21-1)-one;
(2Z)-6-hydroxy-2-({5-methoxy-2-methyl-1-[2-(4-methylpiperazin-1-yl)pethyl]-1H-indol-3-yl}methylene)-1-benzofuran-3(21-1)-one;
(2Z)-4,6-dihydroxy-2-({5-methoxy-2-methyl-1-[2-(4-methylpiperazin-1-yl)pethyl]-1H-indol-3-yl}methylene)-1-benzofuran-3(21-1)-one;
(2Z)-6-hydroxy-2-[(5-methoxy-2-phenyl-1H-indol-3-yl)methylene]-1-benzofuran-3(21-1)-one;
(2Z)-2-({1-[3-(dimethylamino)propyl]-5-methoxy-1H-indol-3-yl}methylene)-4,6-dihydroxy-1-benzofuran-3(21-1)-one;
(2Z)-2-({1-[3-(dimethylamino)propyl]-5-methoxy-1H-indol-3-yl}methylene)-6-hydroxy-1-benzofuran-3(2H)-one;
(2Z)-5-hydroxy-2-({5-methoxy-2-methyl-1-[2-(4-methylpiperazin-1-yl)pethyl]-1H-indol-3-yl}methylene)-1-benzofuran-3(21-1)-one;
(2Z)-4,6-dihydroxy-2-[(1-methyl-4-phenyl-1H-pyrrolo[2,3-b]pyridin-3-yl)methylene]-1-benzofuran-3(21-1)-one;
(2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(21-1)-one; and
(2Z)-4,6-dihydroxy-2-[(1-methyl-4-phenyl-1H-indol-3-yl)methylene]-1-benzofuran-3(21-1)-one;

or geometric isomers thereof or pharmaceutically acceptable salts thereof.

In other aspects, the invention provides pharmaceutical compositions comprising compounds or pharmaceutically acceptable salts of the compounds of any of the present Formulas 1-3 and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of any of the Formulas 1-3; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In other aspects, the second compound is Avastin.

In other aspects, the invention provides a method of treating a PI3K-related disorder, comprising administering to a mammal in need thereof a compound of any of the Formulas 1-3 in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

In other aspects, the invention provides a method of treating an mTOR-related disorder, comprising administering to a mammal in need thereof a compound of any of the Formulas 1-3 in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

In other aspects, the invention provides a method of treating advanced renal cell carcinoma, comprising administering to a mammal in need thereof a compound of any of the Formulas 1-3 in an amount effective to treat advanced renal cell carcinoma.

In other aspects, the invention provides a method of treating acute lymphoblastic leukemia, comprising administering to a mammal in need thereof a compound of any of the Formulas 1-3 in an amount effective to treat acute lymphoblastic leukemia.

In other aspects, the invention provides a method of treating acute malignant melanoma, comprising administering to a mammal in need thereof a compound of any of the Formulas 1-3 in an amount effective to treat malignant melanoma.

In other aspects, the invention provides a method of treating soft-tissue or bone sarcoma, comprising administering to a mammal in need thereof a compound of any of the Formulas 1-3 in an amount effective to treat soft-tissue or bone sarcoma.

In other aspects, the invention provides a method of treating a cancer selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer comprising administering to a mammal in need thereof a composition comprising a compound of any of the Formulas 1-3; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier. in an amount effective to treat the cancer.

In other aspects, the invention provides a method of inhibiting mTOR in a subject, comprising administering to a subject in need thereof a compound of any of the Formulas 1-3 in an amount effective to inhibit mTOR.

In other aspects, the invention provides a method of inhibiting PI3K in a subject, comprising administering to a subject in need thereof a compound of any of the Formulas 1-3 in an amount effective to inhibit PI3K.

In other aspects, the invention provides a method of inhibiting mTOR and PI3K together in a subject, comprising administering to a subject in need thereof a compound of any of the Formulas 1-3 in an amount effective to inhibit mTOR and PI3K.

In other aspects, the invention provides a method of synthesizing a compound of Formula 1, comprising:

a) reacting a compound of the formula 4

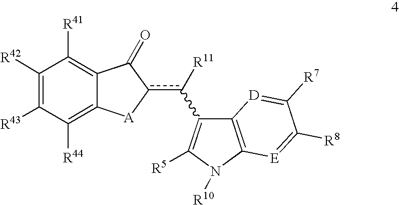

wherein A, R¹¹, R⁵, R¹¹, D, E, R⁷ and R⁸ are defined as in Formula 1, and R⁴¹, R⁴², R⁴³, and R⁴⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³ NC(O)NH—; R¹²C(O)NH—; R¹⁴OC(O)NH—; halo; or hydroxyl;

wherein R¹², R¹³, and R¹⁴ are defined as in Formula 1, and

wherein at least one of R⁴¹—R⁴⁴ is hydroxyl,

with a compound of the formula R²⁰—X, wherein R²⁰ is defined as in Formula 1 and X is a leaving group,

to form a compound of Formula 1 or a geometric isomer thereof,

b) optionally reacting with an acid to form a pharmaceutically acceptable salt of the compound of claim 1 or geometric isomer thereof.

The method in which two of R⁴¹—R⁴⁴ in formula 4 are hydroxyls and two of R¹-R⁴ in the compound of Formula 1 are OR²⁰.

The method in which two of R⁴¹-R⁴⁴ in formula 4 are hydroxyls and one of R¹-R⁴ in the compound of Formula 1 is OR²⁰.

The method in which one of R⁴¹-R⁴⁴ in formula 4 is hydroxyl and one of R¹-R⁴ in the compound of Formula 1 is OR²⁰.

A method of synthesizing a compound of Formula 1 comprising:

a) condensing a compound of the formula 5 with a compound of formula 6:

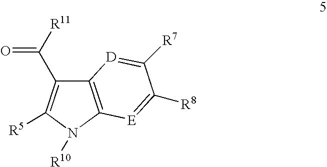

under acidic conditions, wherein

A, R¹¹, R⁵, R¹⁰, D, E, R⁷, R⁸, R¹, R², R³, and R⁴ are defined as in Formula 1,

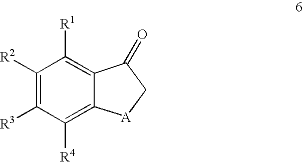

to form a compound of Formula 1 wherein represents a second carbon-to-carbon bond; and

b) optionally reducing the compound of Formula 1 wherein represents a second carbon-to-carbon bond to form a compound of Formula 1 wherein the second carbon-to-carbon bond is absent.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

The compounds within the present invention in a preferred embodiment possess double bonds connecting the fused indole to the benzofuran or benzothiophene nucleolus. These double bonds can exist as geometric isomers, and the invention includes both E and Z isomers of such double bonds. All such stable isomers are contemplated in the present invention.

An “effective amount” when used in connection a compound of the present invention of this invention is an amount effective for inhibiting mTOR or PI3K in a subject.

### DEFINITIONS

The following definitions are used in connection with the compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups, two hydroxyl groups on a single carbon atom, a hydroxyl group on a non-aromatic double bond). Such impermissible substitution patterns are well known to the skilled artisan. In each of the below groups, when a subgroup is designated with a multiple occurrence, each occurrence is selected independently. For example, in di(C₁-C₆alkyl)amino-e.g. (C₁-C₆alkyl)₂N—, the C₁-C₆alkyl groups can be the same or different.

“Acyl-” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. The carbon count includes the carbonyl carbon atom. Examples of a C₁-C₈acyl-group include HC(O)—, acetyl-, benzoyl-, p-toluoyl, nicotinoyl-, propionyl-, isobutyryl-, oxalyl-, and the like. Lower-acyl-refers to acyl groups containing one to four carbons. An acyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl—, C₆-C₁₄aryl-, C₁-C₉heteroaryl-, or C₃-C₈cycloalkyl-.

“Alkenyl-” refer to a straight or branched chain unsaturated hydrocarbon containing at least one double bond. Where E- and/or Z-isomers are possible, the term “alkenyl” is intended to include all such isomers. Examples of a C₂-C₆alkenyl-group include, but are not limited to, ethylene, propylene, 1-butylene, 2-butylene, isobutylene, sec-butylene, 1-pentene, 2-pentene, isopentene, penta-1,4-dien-1-yl, 1-hexene, 2-hexene, 3-hexene, and isohexene. An alkenyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl—, C₆-C₁₄aryl-, C₁-C₉heteroaryl-, and C₃-C₈cycloalkyl-.

“Alkoxy-” refers to the group R—O— where R is an alkyl group, as defined below. Exemplary C₁-C₆alkoxy-groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy-, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—;

“(Alkoxy)carbonyl-” refers to the group alkyl-O—C(O)—. Exemplary (C₁-C₆alkoxy)carbonyl-groups include but are not limited to methoxy, ethoxy, n-propoxy, 1-propoxy, n-butoxy and t-butoxy. An (alkoxy)carbonyl-group can be unsubstituted or substituted with one or more of the following groups: halogen, hydroxyl, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, C₁-C₆acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“Alkyl-” refers to a hydrocarbon chain that may be a straight chain or branched chain, containing the indicated number of carbon atoms, for example, a C₁-C₁₀alkyl-group may have from 1 to 10 (inclusive) carbon atoms in it. In the absence of any numerical designation, “alkyl” is a chain (straight or branched) having 1 to 6 (inclusive) carbon atoms in it. Examples of C₁-C₆alkyl-groups include, but are not limited to, methyl, ethyl, propyl, butyl, pentyl, hexyl, isopropyl, isobutyl, sec-butyl, tert-butyl, isopentyl, neopentyl, and isohexyl. An alkyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“(Alkyl)amido-” refers to a —NHC(O)— group in which the nitrogen atom of said group is attached to an alkyl group, as defined above. Representative examples of a (C₁-C₆alkyl)amido-group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an NH— group, the nitrogen atom of said group being attached to an alkyl group, as defined above. Representative examples of an (C₁-C₆alkyl)amino-group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“Alkylcarboxy-” refers to an alkyl group, defined above, attached to the parent structure through the oxygen atom of a carboxyl (C(O)—O—) functionality. Examples of (C₁-C₆alkyl)carboxy-include acetoxy, ethylcarboxy, propylcarboxy, and isopentylcarboxy.

“-Alkylene-”, “-alkenylene-”, and “-alkynylene-” refer to alkyl, alkenyl and alkynyl groups, as defined above, having two points of attachment within a chemical structure. Examples of —C1-C₆alkylene-include ethylene (—CH₂CH₂—), propylene (—CH₂CH₂CH₂—), and dimethylpropylene (—CH₂C(CH₃)₂CH₂—). Likewise, examples of —C₂-C₆alkenylene-include ethenylene (—CH═CH— and propenylene (—CH═CH—CH₂—). Examples of —C₂-C₆alkynylene-include ethynylene (—C≡C—) and propynylene (—C≡C—CH₂—).

“Alkylthio-” refers to the group R—S— where R is an alkyl group, as defined above, attached to the parent structure through a sulfur atom. Examples of C₁-C₆alkylthio-include methylthio, ethylthio, n-propylthio, i-propylthio, n-butylthio, i-butylthio, s-butylthio, t-butylthio, n-pentylthio and n-hexylthio.

“Alkynyl-” refers to a straight or branched chain unsaturated hydrocarbon containing at least one triple bond. Examples of a C₂-C₆alkynyl-group include, but are not limited to, acetylene, propyne, 1-butyne, 2-butyne, isobutyne, sec-butyne, 1-pentyne, 2-pentyne, isopentyne, penta-1,4-diyn-1-yl, 1-hexyne, 2-hexyne, 3-hexyne, and isohexyne. An alkynyl group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₆acyl-, C₆-C₁₄aryl-, C₁-C₉heteroaryl-, and C₃-C₈cycloalkyl-.

“Amido(aryl)-” refers to an aryl group, as defined below, wherein one of the aryl group's hydrogen atoms has been replaced with one or more H₂NC(O)— groups. Representative examples of an amido(C₆-C₁₄aryl)-group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with H₂N—. Representative examples of an C₁-C₆-aminoalkyl-group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃, —C(CH₃)₂(CH₂NH₂), —CH₂CH₂CH₂CH₂CH₂NH₂, and —CH₂CH₂CH(NH₂)CH₂CH₃. An aminoalkyl-group can be unsubstituted or substituted with one or two of the following groups: C₁-C₆alkoxy-, C₆-C₁₄aryl-, C₁-C₉heteroaryl-, C₃-C₈cycloalkyl-, and C₁-C₆alkyl-.

Aryl-refers to an aromatic hydrocarbon group. Examples of an C₆-C₁₄aryl-group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. An aryl group can be monocyclic or polycyclic as long as at least one ring is aromatic and the point of attachment is at an aromatic carbon atom. An aryl group can be unsubstituted or substituted with one or more of the following groups: C₁-C₆alkyl-, halogen, haloalkyl-, hydroxyl, hydroxyl(C₁-C₆alkyl)-, H₂N—, aminoalkyl-, di(C₁-C₆alkyl)amino-, HO₂O—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Aryl)alkyl-” refers to an alkyl group, as defined above, wherein one or more of the alkyl group's hydrogen atoms has been replaced with an aryl group as defined above. (C₆-C₁₄Aryl)alkyl-moieties include benzyl, benzhydryl, 1-phenylethyl, 2-phenylethyl, 3-phenylpropyl, 2-phenylpropyl, 1-naphthylmethyl, 2-naphthylmethyl and the like. An (aryl)alkyl-group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“(Aryl)amino-” refers to a radical of formula (aryl)-NH—, wherein aryl is as defined above. Examples of (C₆-C₁₄aryl)amino-radicals include, but are not limited to, phenylamino (anilido), 1-naphthylamino, 2-naphthylamino, and the like. An (aryl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, or C₃-C₈cycloalkyl-.

“(Aryl)oxy-” refers to the group Ar—O— where Ar is an aryl group, as defined above. Exemplary (C₆-C₁₄aryl)oxy-groups include but are not limited to phenyloxy, α-naphthyloxy, and β-naphthyloxy. An (aryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆-aminoalkyl-, di(C₁-C₆alkyl)amino-, HO₂O—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Cycloalkyl-” refers to a monocyclic saturated hydrocarbon ring. Representative examples of a C₃-C₈cycloalkyl-include, but are not limited to, cyclopropyl, cyclobutyl, cyclopentyl, cyclohexyl, cycloheptyl, and cyclooctyl. A cycloalkyl-can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, or C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle-containing two oxygen atoms.

“Bicyclic cycloalkyl-” refers to a bicyclic saturated hydrocarbon ring system. Representative examples of a C₆-C₁₀bicyclic cycloalkyl-include, but are not limited to, cis-1-decalinyl, trans 2-decalinyl, cis-4-perhydroindanyl, and trans-7-perhydroindanyl. A bicyclic cycloalkyl-can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl-, C₆-C₁₄aryl-, C₁-C₉heteroaryl-, or C₃-C₈cycloalkyl-, haloalkyl-, aminoalkyl-, (C₁-C₆alkyl)carboxy-, carbonylamidoalkyl-, or O₂N—. Additionally, each of any two hydrogen atoms on the same carbon atom of the bicyclic cycloalkyl-rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carbonylamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl-, C₂-C₆alkenyl, C₂-C₆alkynyl, C₆-C₁₄aryl-, C₁-C₉heteroaryl-, or C₃-C₈cycloalkyl-, attached to the parent compound by an —C₁-C₆alkylene-group as defined above. Exemplary C₁-C₆-carbonylamidoalkyl-groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₆NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl-” refers to non-aromatic carbocyclic rings with one or more carbon-to-carbon double bonds within the ring system. The “cycloalkenyl” may be a single ring or may be multi-ring. Multi-ring structures may be bridged or fused ring structures. Examples of C₃-C₁₀cycloalkenyl-groups include, but are not limited to, cyclopropenyl, cyclobutenyl, cyclopentenyl, cyclohexenyl, 4,4a-octalin-3-yl, and cyclooctenyl. A cycloalkenyl can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl-, C₆-C₁₄aryl-, C₁-C₉heteroaryl-, or C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N— Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms.

“Di(alkyl)amido-” refers to a —NC(O)— group in which the nitrogen atom of said group is attached to two alkyl groups, as defined above. Each alkyl group can be independently selected. Representative examples of a di(C₁-C₆alkyl)amido-group include, but are not limited to, —C(O)N(CH₃)₂, —C(O)N(CH₂CH₃)₂, —C(O)N(CH₃)CH₂CH₃, —C(O)N(CH₂CH₂CH₂CH₃)₂, —C(O)N(CH₂CH₃)CH₂CH₂CH₃, —C(O)N(CH₃)CH(CH₃)₂, —C(O)N(CH₂CH₃)CH₂CH(CH₃)₂, —C(O)N(CH(CH₃)CH₂CH₃)₂, —C(O)N(CH₂CH₃)C(CH₃)₃ and —C(O)N(CH₂CH₃)CH₂C(CH₃)₃.

“Di(alkyl)amino-” refers to a nitrogen atom attached to two alkyl groups, as defined above. Each alkyl group can be independently selected. Representative examples of an di(C₁-C₆alkyl)amino-group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂—N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(H)—, —N(C₁-C₆alkyl)-, —N(C₃-C₈cycloalkyl)-, —N(C₆-C₁₄aryl)-, —N(C₁-C₉heteroaryl)-, —N(C₁-C₆-aminoalkyl)-, —N(C₆-C₁₄arylamino)-, —O—, —S—, —S(O)—, or —S(O)₂—.

“Halo” or “halogen” refers to fluorine, chlorine, bromine, or iodine.

“Haloalkyl-” refers to an alkyl group, as defined above, wherein one or more of the hydrogen atoms has been replaced with —F, —Cl, —Br, or —I. Each substitution can be independently selected. Representative examples of an C₁-C₆haloalkyl-group include, but are not limited to, —CH₂F, —CCl₃, —CF₃, CH₂CF₃, —CH₂Cl, —CH₂CH₂Br, —CH₂CH₂₁, —CH₂CH₂CH₂F, —CH₂CH₂CH₂Cl, —CH₂CH₂CH₂CH₂Br, —CH₂CH₂ CH₂CH₂₁, —CH₂CH₂CH₂CH₂CH₂Br, —CH₂CH₂CH₂CH₂CH₂₁, —CH₂CH(Br)CH₃, —CH₂CH(Cl)CH₂CH₃, —CH(F)CH₂CH₃ and —C(CH₃)₂(CH₂Cl).

“Heteroaryl-” refers to 5-10-membered mono and bicyclic aromatic groups containing at least one heteroatom selected from oxygen, sulfur and nitrogen, wherein any S can optionally be oxidized, and any N can optionally be quaternized with an C₁-C₆alkyl group. Examples of monocyclic C₁-C₉heteroaryl-radicals include, but are not limited to, oxazinyl, thiazinyl, diazinyl, triazinyl, thiadiazolyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl-radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl-rings or ring systems have a minimum of 5 members. Therefore, for example, C₁heteroaryl-radicals would include but are not limited to tetrazolyl, C₂heteroaryl-radicals include but are not limited to triazolyl, thiadiazolyl, and tetrazinyl, C₉heteroaryl-radicals include but are not limited to quinolinyl and isoquinolinyl. A heteroaryl-group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆-aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Heteroaryl)alkyl-” refers to an alkyl group, as defined above, wherein one or more of the alkyl group's hydrogen atoms has been replaced with a heteroaryl-group as defined above.

Examples of (C₁-C₉heteroaryl)alkyl-moieties include 2-pyridylmethyl, 2-thiophenylethyl, 3-pyridylpropyl, 2-quinolinylmethyl, 2-indolylmethyl, and the like. A (heteroaryl)alkyl-group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₆acyl-, C₈-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“(Heteroaryl)oxy-” refers to the group Het-O— where Het is a heteroaryl-group, as defined above. Exemplary (C₁-C₉heteroaryl)oxy-groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆-aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Heteroatom” refers to a sulfur, nitrogen, or oxygen atom.

“Heterocycle” or “heterocyclyl-” refers to 3-10-membered monocyclic, fused bicyclic, and bridged bicyclic groups containing at least one heteroatom selected from oxygen, sulfur and nitrogen, wherein any S can optionally be oxidized, and any N can optionally be quaternized by a C₁-C₆alkyl group. A heterocycle may be saturated or partially saturated. Exemplary C₁-C₉heterocyclyl-groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octane, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁heterocyclyl-radicals would include but are not limited to oxaziranyl, diaziridinyl, and diazirinyl, C₂heterocyclyl-radicals include but are not limited to aziridinyl, oxiranyl, and diazetidinyl, C₉heterocyclyl-radicals include but are not limited to azecanyl, tetrahydroquinolinyl, and perhydroisoquinolinyl.

“Heterocyclyl(alkyl)-” refers to an alkyl group, as defined above, wherein one or more of the alkyl group's hydrogen atoms has been replaced with a heterocycle group as defined above. C₁-C₉Heterocyclyl (C₁-C₆alkyl)-moieties include 2-pyridylmethyl, 1-piperazinylethyl, 4-morpholinylpropyl, 6-piperazinylhexyl, and the like. A heterocyclyl(alkyl)-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₆acyl-, 4- to 7-membered monocyclic heterocycle, C₆-C₁₄aryl-, C₁-C₉heteroaryl-, or C₃-C₈cycloalkyl-.

“Hydroxylalkyl-” refers to an alkyl group, as defined above, wherein one or more of the alkyl group's hydrogen atoms has been replaced with hydroxyl groups. Examples of C₁-C₆hydroxylalkyl-moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to an alkenyl group, defined above, and substituted on one or more sp³ carbon atoms with a hydroxyl group. Examples of C₃-C₆hydroxylalkenyl-moieties include chemical groups such as CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Leaving group” refers an atom or group (charged or uncharged) that becomes detached from an atom in what is considered to be the residual or main part of the substrate in a specified reaction. For example, in the heterolytic solvolysis of benzyl bromide in acetic acid: the leaving group is bromide. In the reaction of N,N,N-trimethyl-1-phenylmethanaminium ion with methanethiolate, the leaving group is trimethylamine. In the electrophilic nitration of benzene, it is H. The term has meaning only in relation to a specified reaction. Examples of leaving groups include, for example, carboxylates (i.e. CH₃COO⁻, CF₃CO₂^{-), F}⁻, water, Cl⁻, Br, I⁻, N₃⁻, SCN⁻, trichloroacetimidate, thiopyridyl, tertiary amines (i.e. trimethylamine), phenoxides (i.e. nitrophenoxide), and sulfonates (i.e. tosylate, mesylate, triflate).

The term “monocyclic heterocycle” refers to a monocyclic 3- to 7-membered aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A monocyclic heterocycle group can be unsubstituted or substituted with one or more of the following groups: C₁-C₈acyl, C₁-C₆alkyl, heterocyclyl(C₁-C₆alkyl), (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂.

“Bicyclic heterocycle” refers to a bicyclic cycloalkyl or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a 6- to 10-membered bicyclic heterocycle group include, but are not limited to, indolinyl, indazolyl, tetrahydroquinolinyl, perhydroquinazolinyl, 5,6-dihydro-4H-1,3-oxazin-2-yl, 8-oxa-3-azabicyclo[3.2.1]octan-3-yl, 2-methyl-2,5-diazabicyclo[2.2.1]heptane-5-yl, and indazolyl. A bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Nitrogen-containing heteroaryl-” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl-radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl-radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing heteroaryl-group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆-aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Perfluoroalkyl-” refers to alkyl group, defined above, having two or more fluorine atoms. Examples of a C₁-C₆ perfluoroalkyl-group include CF₃, CH₂CF₃, CF₂CF₃ and CH(CF₃)₂.

The term “optionally substituted”, unless otherwise specified, as used herein means that at least one hydrogen atom of the optionally substituted group has been substituted with halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-.

A “subject” is a mammal, e.g., a human, mouse, rat, guinea pig, dog, cat, horse, cow, pig, or non-human primate, such as a monkey, chimpanzee, baboon or gorilla.

The compounds of the present invention may act as prodrugs whereby cleavage of one or more O—R²⁰ bonds in the compounds of the present invention provides compounds having one or more phenolic O—H groups corresponding to the one or more cleaved O—R²⁰ groups, wherein the compounds having one or more phenolic O—H groups may exhibit a PI3K and/or mTOR activity. Additionally, the compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

Accordingly, the use of the term “prodrugs” for the compounds of the present invention is not intended to exclude that the compounds of the present invention themselves may have a PI3K and/or mTOR inhibitory activity. In addition, in a compound of the invention having two O—R²⁰ groups, one of the bonds may be cleaved to provide a compound having an O—R²⁰ group and an OH group. Such a compound may owe its PI3K and/or mTOR inhibitory activity at least in part to the OH group formed, but it would still be a prodrug due to the presence of one remaining O—R²⁰ group.

Thus, the compounds of the present invention and/or the compounds formed from the compounds of the present invention by cleavage of one or more O—R²⁰ bonds are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention and/or the compounds formed from the compounds of the present invention by cleavage of one or more O—R²⁰ bonds possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention and/or the compounds formed from the compounds of the present invention by cleavage of one or more O—R²⁰ bonds exhibit an PI3 kinase inhibitory activity and therefore, can be utilized in order to inhibit abnormal cell growth in which PI3 kinases play a role. Thus, the compounds of the present invention and/or the compounds formed from the compounds of the present invention by cleavage of one or more O—R²⁰ bonds are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention and/or the compounds formed from the compounds of the present invention by cleavage of one or more O—R²⁰ bonds possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of the present invention and/or the compounds formed from the compounds of the present invention by cleavage of one or more O—R²⁰ bonds will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention may inhibit both mTOR and PI3 kinase simultaneously and, therefore, can be utilized in order to inhibit abnormal cell growth in which both mTOR and PI3 kinases simultaneously play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of any of the Formulas 1-3 will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be made into tablets, placed in a hard gelatin capsule in powder or pellet form, or in the form of a troche or lozenge. The solid carrier may contain conventional excipients such as binding agents, fillers, lubricants used to make tablets, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of any of the Formulas I-III directly in parenteral formulations. The pharmaceutical compositions are prepared by conventional techniques appropriate to the desired preparation containing appropriate amounts of the active ingredient, that is, the compound of any of the Formulas I-III according to the invention. See, for example, Remington: The Science and Practice of Pharmacy, 20th Edition. Baltimore, Md.: Lippincott Williams & Wilkins, 2000.

The dosage of the compounds of any of the Formulas 1-3 to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of any of the Formulas 1-3 or pharmaceutical composition thereof for a mammal, including man, suffering from, or likely to suffer from any condition as described herein is an amount of active ingredient from about 0.01.mg/kg to 10 mg/kg body weight. For parenteral administration, the dose may be in the range of 0.1.mg/kg to 1 mg/kg body weight for intravenous administration. For oral administration, the dose may be in the range about 0.1.mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salts thereof is an amount that is effective for inhibiting mTOR or PI3K in a subject or an amount wherein upon cleavage of one or more O—R²⁰ bonds, a compound having corresponding one or more phenolic O—H groups is formed in an amount that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

The Schemes shown in Scheme Sections A and B below, and the Preparations following the Schemes (Preparations A and B) describe the general procedures used to synthesize the precursors to the compounds of the present invention are described in Schemes 1-24 and are illustrated in the examples. These precursors are phenolic compounds having at least one hydroxyl group corresponding to R¹, R², R³, or R⁴, which hydroxyl group can be then converted to an OR²⁰ group as further described below. Reasonable variations of the described procedures, which would be evident to one skilled in the art, are intended to be within the scope of the present invention:

Scheme Section A (Schemes A-1 to A-61) below describes the preparation of 3-substituted-1H-indole compounds having a benzofuranone or benzothiophenone with at least one hydroxyl on the benzofuranone or benzothiophenone phenyl ring. Scheme Section B (Schemes B-1 to B-24) describes the preparation of 3-substituted-1H-pyrrolo[2,3-b]pyridine and 3-substituted-1H-pyrrolo[3,2-b]pyridine compounds having a benzofuranone or benzothiophenone with at least one hydroxyl on the benzofuranone or benzothiophenone phenyl ring. Scheme Section C (Schemes C-1 to C-6) below describes the preparation of the compounds of the invention from the compounds made according to Scheme Section A or Scheme Section B. Schemes A-1 to A-61: preparation of 3-substituted-1H-indole compounds having a benzofuranone or benzothiophenone with at least one hydroxyl on the benzofuranone or benzothiophenone phenyl ring.

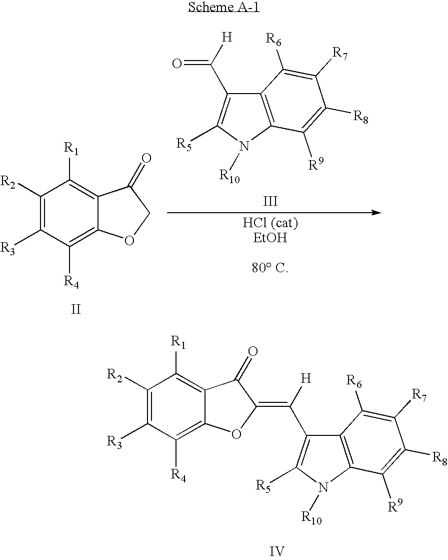

Benzofuranone molecules IV may be prepared according to Scheme A-1 by reacting benzofuranone compounds II with heteroaryl aldehydes III in alcohols such as EtOH with a catalytic amount of an acid such as HCl, AcOH, or TFA at 80° C. Benzofuranone compounds II and heteroaryl aldehydes III can be purchased commercially or prepared synthetically via standard organic chemistry protocols.

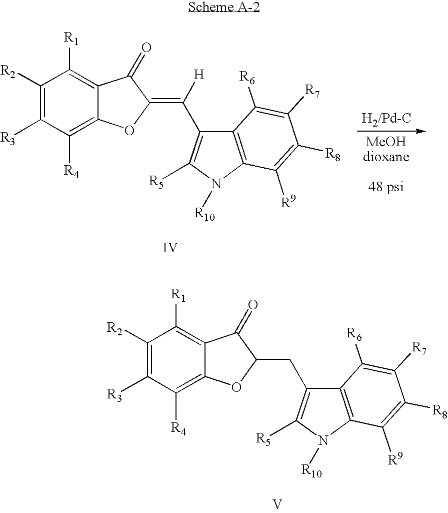

2-Methylbenzofuranone molecules V may be prepared according to Scheme A-2 by reduction of 2-methylenebenzofuranones IV with Pd/C in MeOH/dioxane under 48 psi atmosphere of hydrogen.

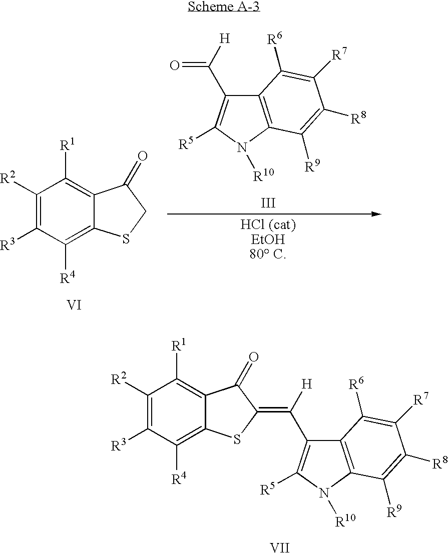

Benzothiophenone molecules VII may be prepared according to Scheme A-3 by reacting benzothiophenone VI with the heteroaryl aldehydes III in a hydrocarbon solvent such as benzene with catalytic amounts of as base such as piperidine at 80° C. Benzothiophenone VI and heteroaryl aldehydes III can be purchased commercially or prepared synthetically via standard organic chemistry protocols.

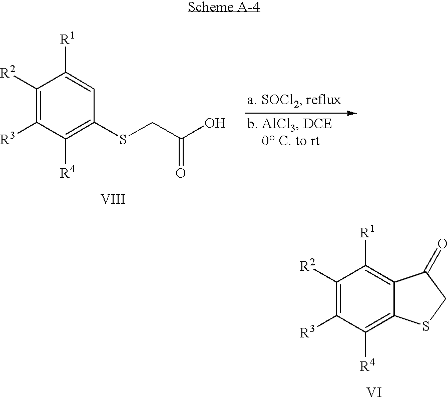

Benzothiophenones VI as described in Scheme A-4 can be obtained from the corresponding acids VIII using known literature procedures. To the acid (15.6 mmol) is added SOCl₂ (10 mL). After heating the resulting suspension to 85° C. for 1 hour, the reaction is concentrated in vacuo and placed under vacuum for 30 minutes. To the reaction is added methylene chloride (30 mL) and cooled on an ice-salt bath for 15 minutes. AlCl₃ (2.5 g) is added in portions over 20 minutes. The reaction is stirred with cooling for 15 minutes and then allowed to stir for 45 minutes at room temperature. The reaction is quenched with ice water, extracted with methylene chloride and concentrated in vacuo to afford the desired compound without further purification.

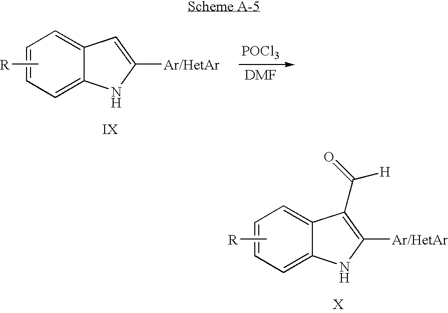

Several 3-Indole carboxaldehyde compounds as described in scheme 1 can be obtained commercially, while others can be synthesized using various synthetic methods outlined below. 3-Indole carboxaldehyde compounds as described by Scheme A-5 can be obtained from the corresponding indole via reaction with POCl₃ under standard literature conditions.

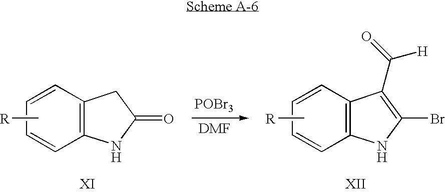

3-Indole carboxaldehyde compounds as described by Scheme A-6 can be obtained from the corresponding oxindole via reaction with POBr₃ in DMF using literature procedures described in Arch. Pharmazie, 1972, 305, 523.

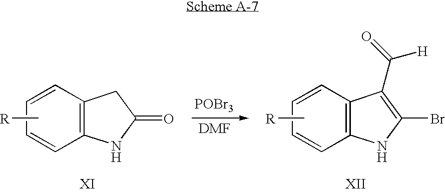

3-Indole carboxaldehyde compounds as described by Scheme A-7 can be obtained from the corresponding indole via reaction with DMF/POCl₃ under standard literature conditions and then subsequent alkylation using alkyl halides and NaH in DMF under standard literature conditions.

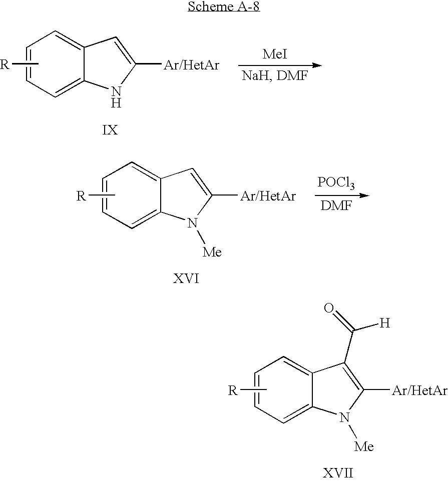

3-Indole carboxaldehyde compounds as described by Scheme A-8 can be obtained from the corresponding indole via methylation using MeI and NaH in DMF under standard literature conditions and then subsequent reaction with POCl₃ under standard literature conditions.

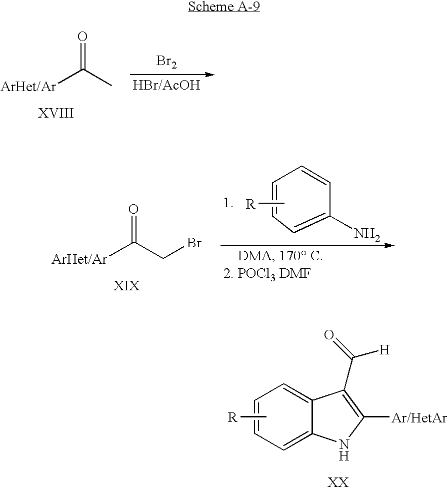

3-Indole carboxaldehyde compounds as described by Scheme A-9 can be obtained from brominating the corresponding aryl or heteroaryl acetyl using procedure described in Austr. J. Chem. 1989, 42, 1735 then reacting the resulting the a-bromo ketone with anisidine, as described in Bioorg. Med. Chem. 2002, 10, 3941, to afford the desired indole. The 3-indole carboxaldehyde derivative was then obtained via method 1.

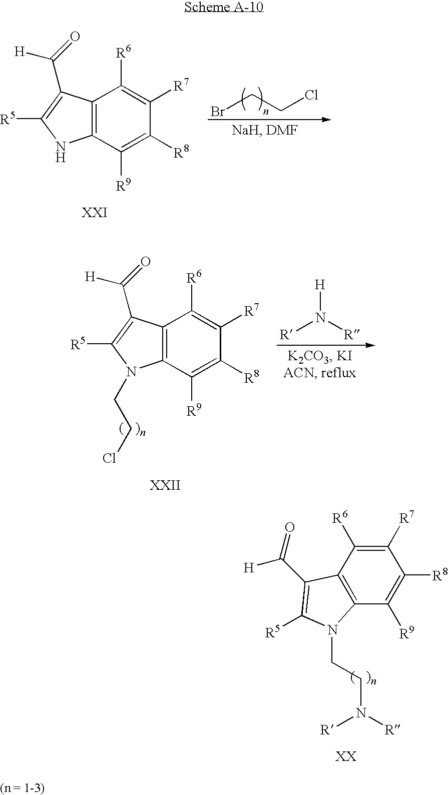

3-Indolecarboxaldehydes as described by Scheme A-10 can be obtained by alkylation of the 3-indolecarboxaldehydes XXI using the corresponding ω-bromochloroalkanes and a base like NaH in a polar solvent like DMF under standard literature conditions. The resulting alkyl chloride XXII was then reacted with the desired secondary amine using potassium carbonate and potassium iodide in ACN at 80° C. under standard literature conditions.

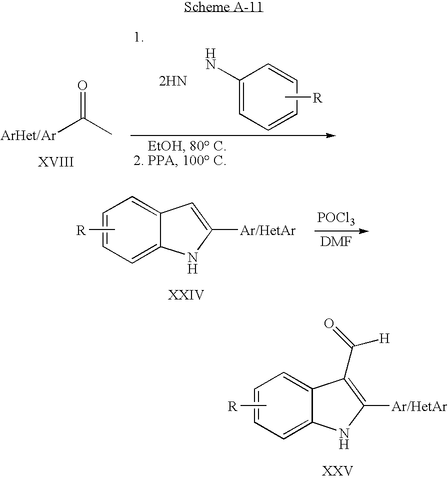

3-Indole carboxaldehyde compounds as described by Scheme A-11 can be obtained from the corresponding ketone and hydrazine under standard Fischer-indole synthesis literature conditions.

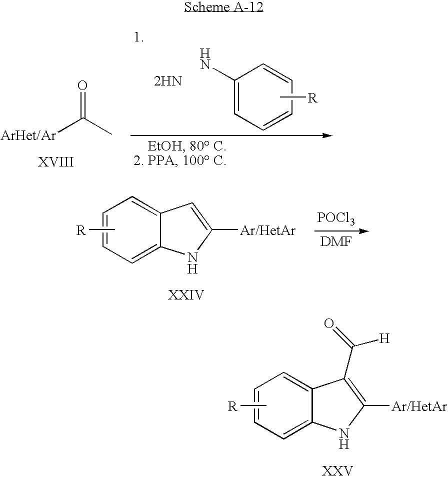

3-Indole carboxaldehyde compounds as described by Scheme A-12 can be obtained from the corresponding indole via reaction with DMF/POCl₃ under standard literature conditions and then subsequent methylation using 2 equivalents of MeI and NaH in DMF under standard literature conditions.

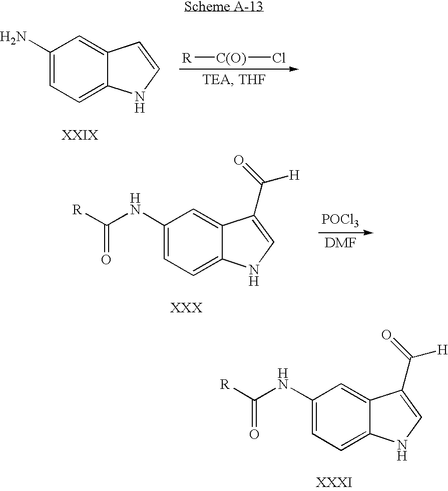

3-Indole carboxaldehyde compounds as described by Scheme A-13 can be obtained from the corresponding indole via acylation with acid chlorides in THF in the presence of TEA under standard literature conditions and then subsequent reaction with DMF/POCl₃ under standard literature conditions.

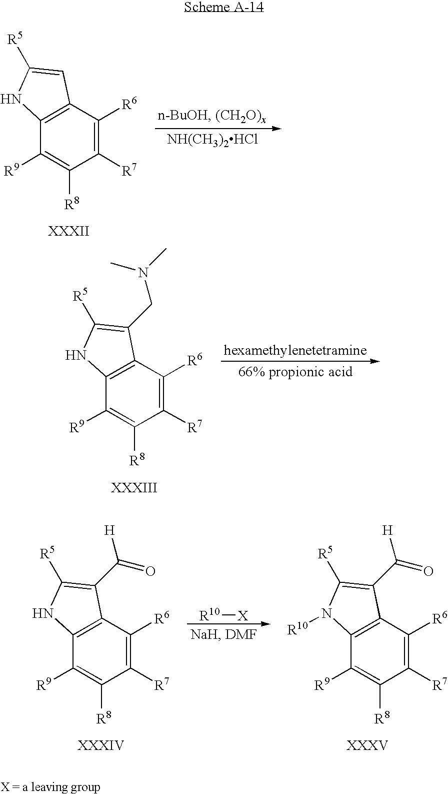

3-Indole carboxaldehyde compounds XXXV as described in Scheme A-14 can be obtained by first generating gramine from indole XXXII, paraformaldehyde, and dimethylamine, by Mannich reaction followed by hydrolysis using literature procedures described in JACS 1955, 77, 457. This was followed by alkylation using R¹⁰—X and a base like NaH in an aprotic solvent like DMF under standard literature conditions.

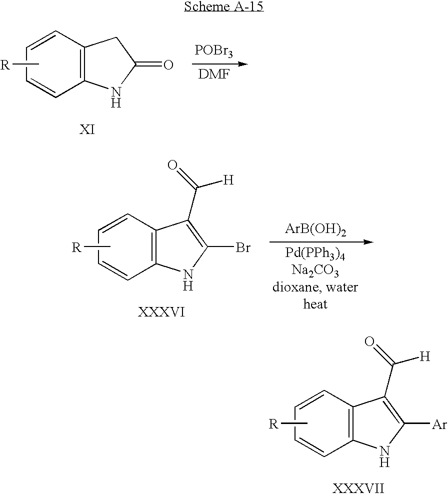

3-Indole carboxaldehyde compounds as described by Scheme A-15 can be obtained from the corresponding oxindole via reaction with POBr₃ in DMF using literature procedures described in Arch. Pharmazie, 1972, 305, 523. The bromo derivative can be further subjected to a Suzuki coupling reaction with variety of boronic acids.

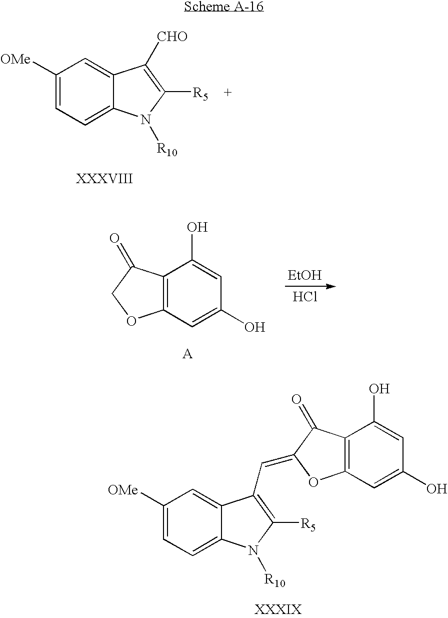

Condensation between 4,6-dihydroxy-benzofuran-3-one (A) and 5-methoxy-indole-3-carbaldehydes XXXVIII is shown in Scheme A-16.

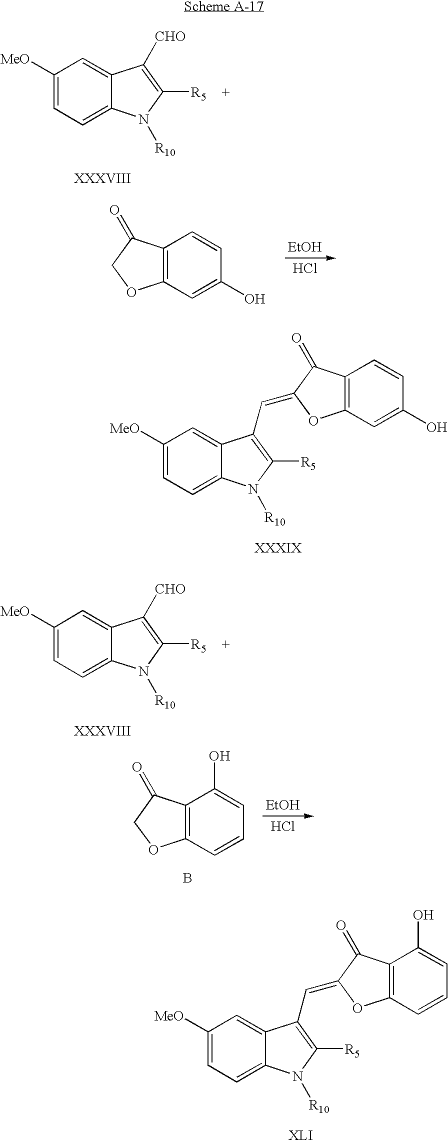

Condensation between mono-hydroxy-benzofuran-3-ones and 5-methoxy-indole-3-carbaldehydes, 6-mono-hydroxy derivatives and 4-mono-hydroxy derivatives is shown in Scheme A-17.

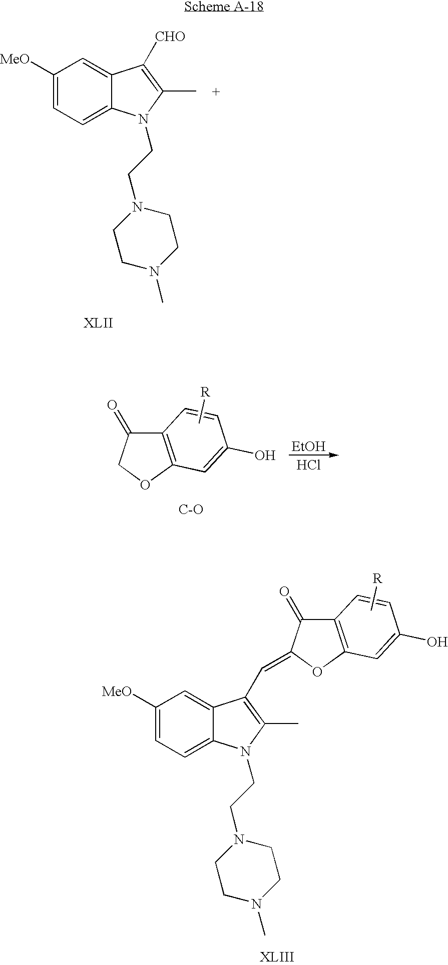

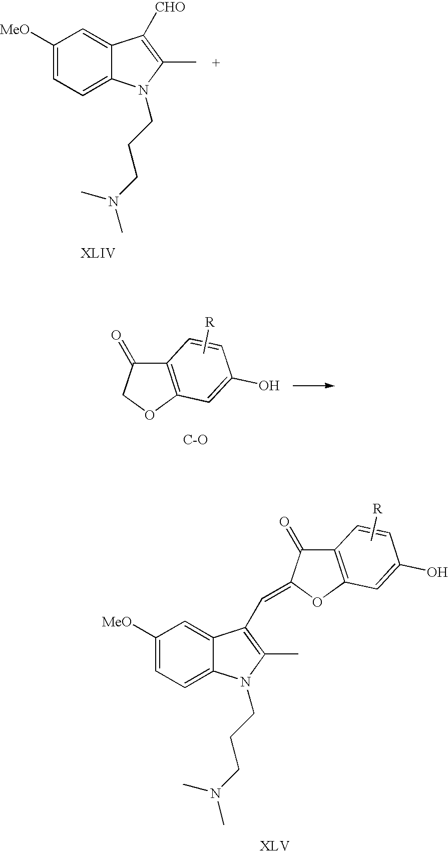

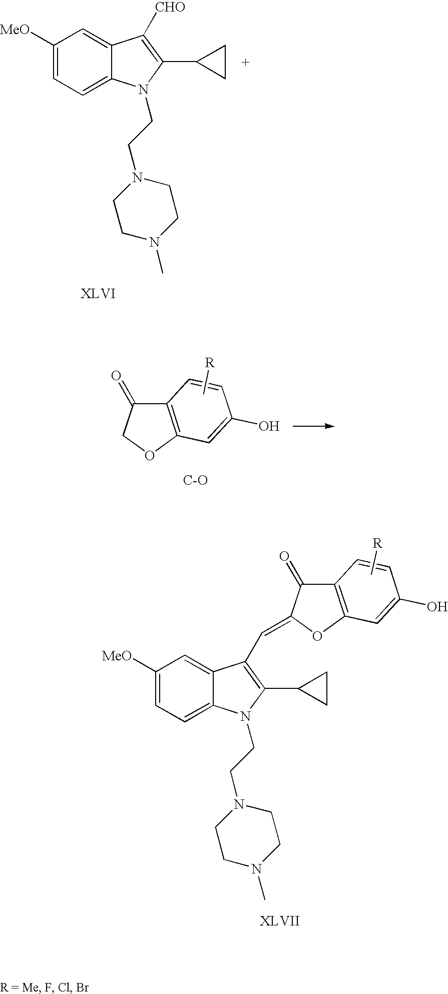

Condensation between substituted 6-hydroxy-benzofuranones and 5-methoxy-indole-3-carbaldehydes C-O is shown in Scheme A-18.

Benzofuranone compounds C—O

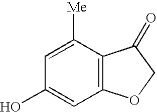

C

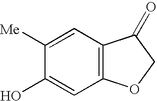

D

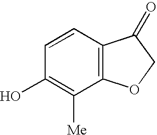

E

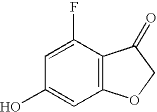

F

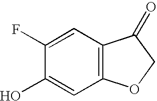

G

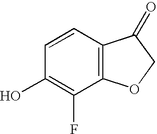

H

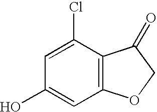

I

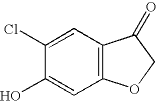

L

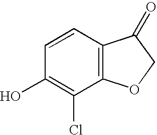

M

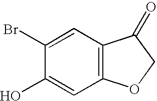

N

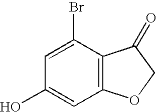

O

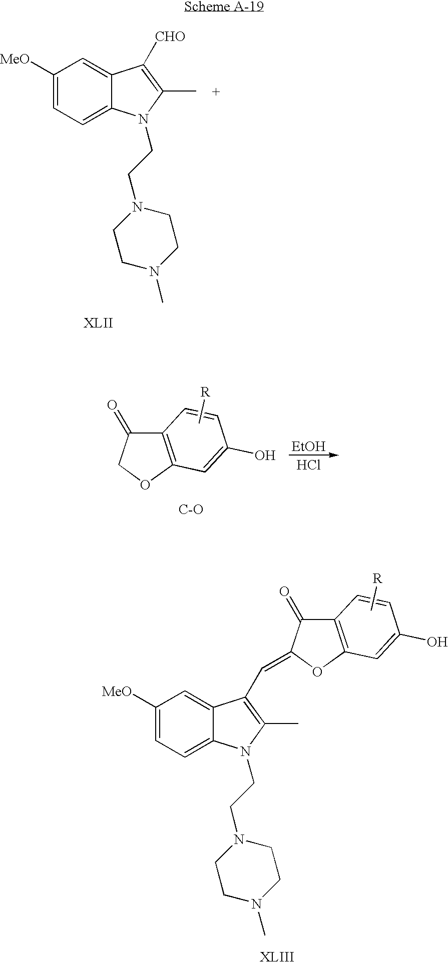

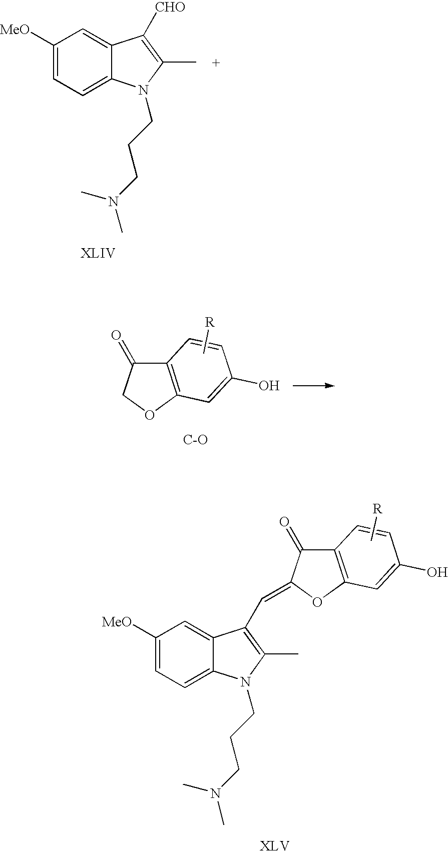

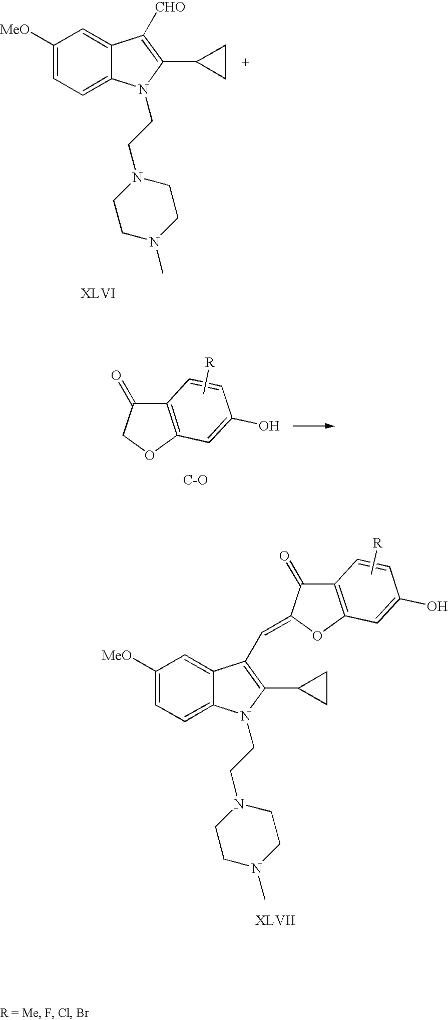

Preparation of (2Z)-2[(4-aryl-1-methyl-1H-indol-3-yl)methylene]-4,6-dihydroxy-1-benzofuran-3(2H)-one compounds (LI) is shown in Scheme A-19.

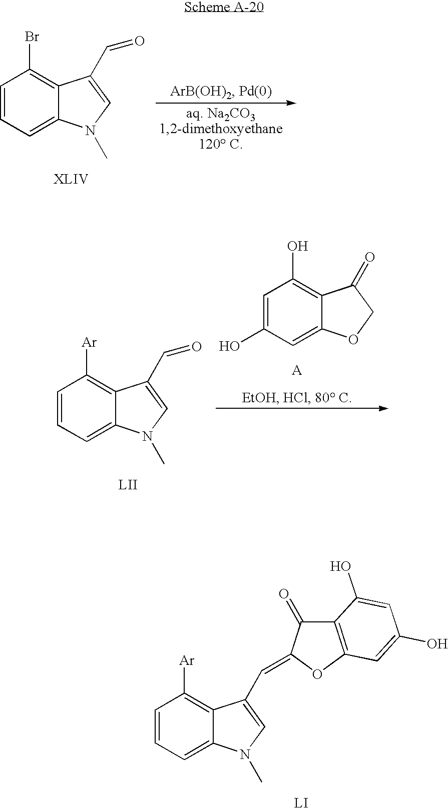

An alternative preparation of (2Z)-2[(4-aryl-1-methyl-1H-indol-3-yl)methylene]-4,6-dihydroxy-1-benzofuran-3(2H)-one (LI) is shown in Scheme A-20.

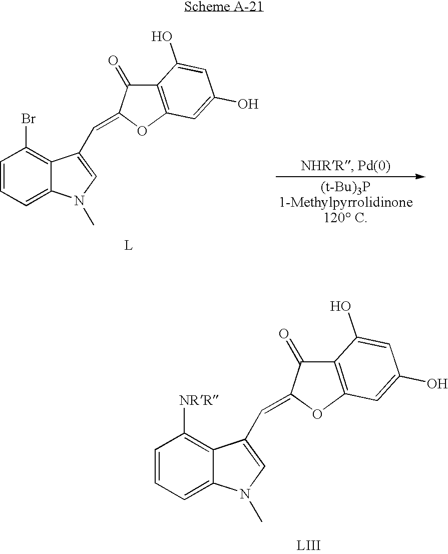

The preparation of (2Z)-2[(4-amino-1-methyl-1H-indol-3-yl)methylene]-4,6-dihydroxy-1-benzofuran-3(2H)-one (LIII) is shown in Scheme A-21.

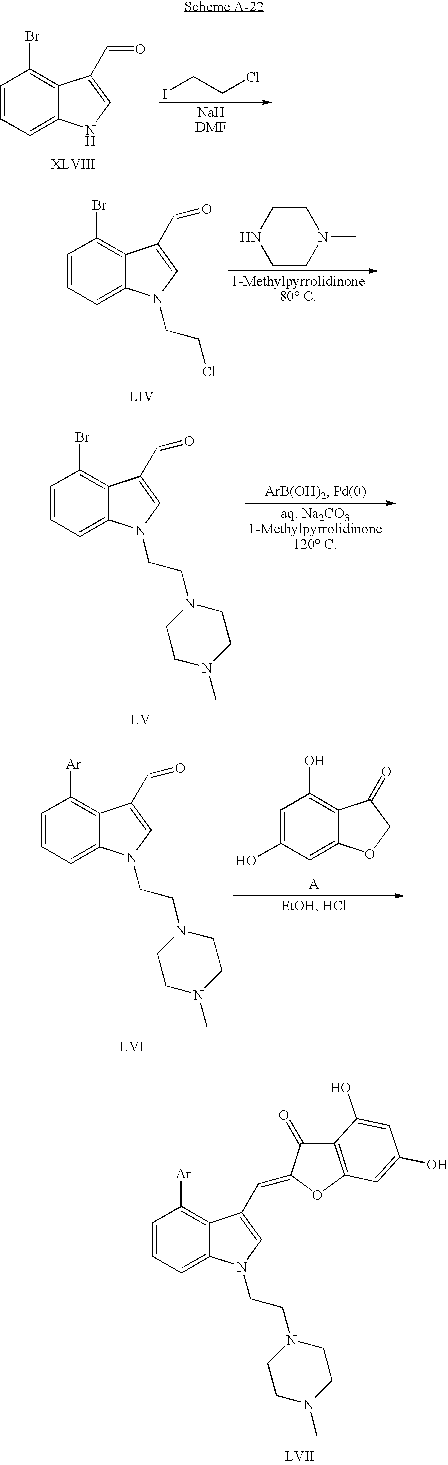

The preparation of (2Z)-2-({4-aryl-1-[2-(4-methylpiperazin-1-yl)ethyl]-1H-indol-3-yl}methylene)-4,6-dihydroxy-1-benzofuran-3(2H)-one compounds (LVII) is shown in Scheme A-22.

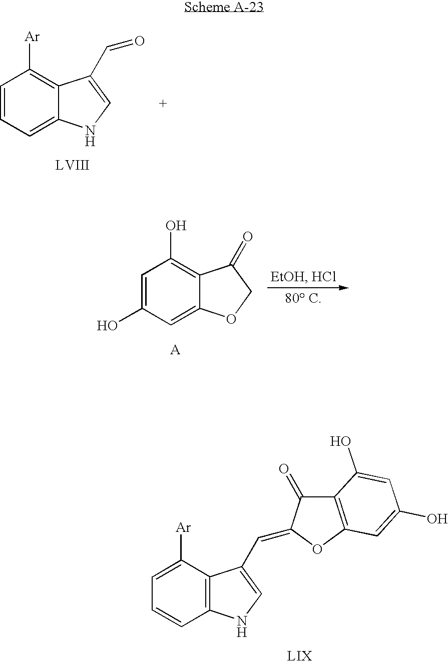

The preparation of (2Z)-2[(4-aryl-1H-indol-3-yl)methylene]-4,6-dihydroxy-1-benzofuran-3(2H)-one (LIX) is shown in Scheme A-23.

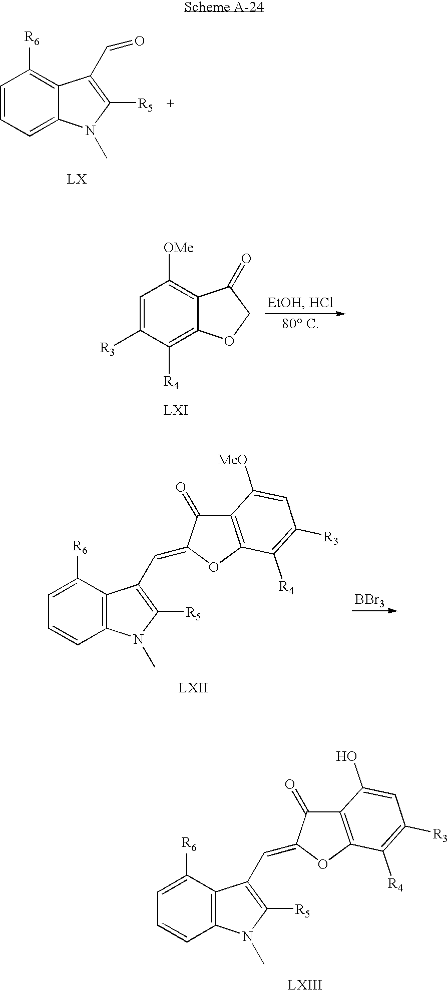

The preparation of (2Z)-2(-1H-indol-3-yl)methylene-4-methoxy-1-benzofuran-3(2H)-one (LXII) and its demethylation to (2Z)-2(-1H-indol-3-yl)methylene-4-hydroxy-1-benzofuran-3(2H)-one (LXIII) are shown in Scheme A-24.

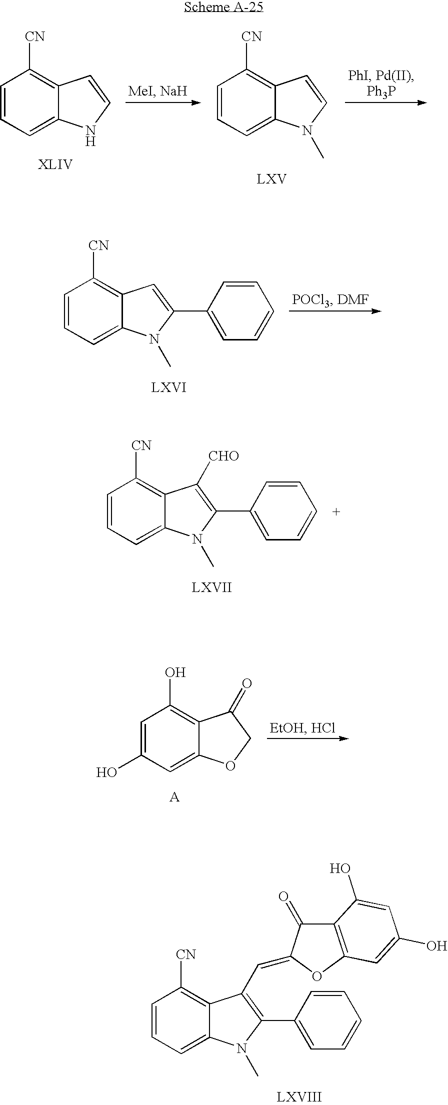

The preparation of 3-[(Z)-(4,6-dihydroxy-3-oxo-1-benzofuran-2(3H)-ylidene)methyl]-1-methyl-2-phenyl-1H-indole-4-carbonitrile (LXVIII) is shown in Scheme A-25.

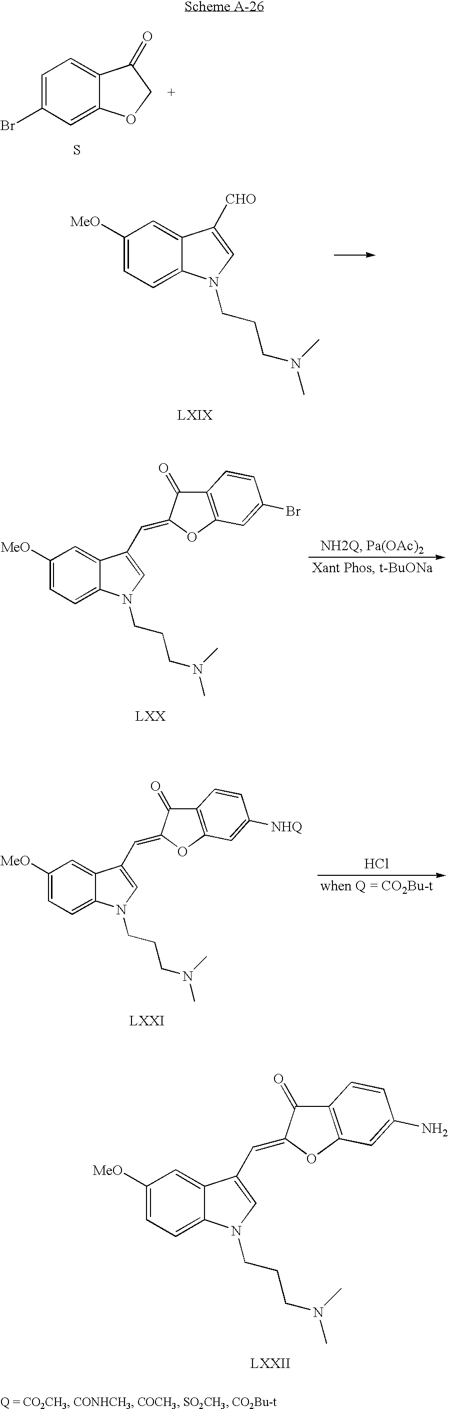

The preparation of 6-substituted (2Z)-2-({1-[3-(dimethylamino)propyl]-5-methoxy-1H-indol-3-yl}methylene)-1-benzofuran-3(2H)-one (LXXII) is shown in Scheme A-26,

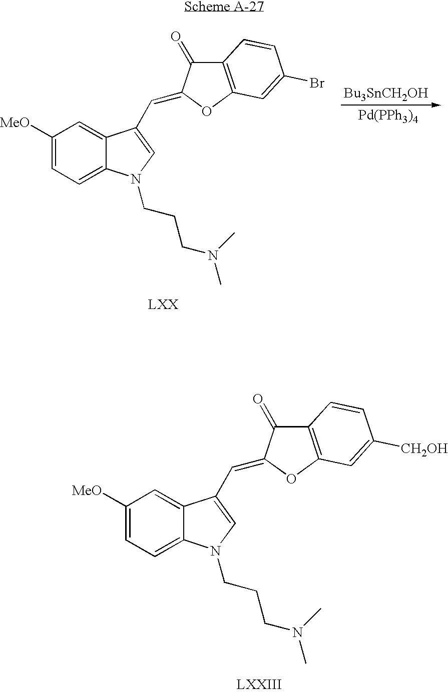

The preparation of (2Z)-2-({1-[3-(dimethylamino)propyl]-5-methoxy-1H-indol-3-yl}methylene)-6-(hydroxymethyl)-1-benzofuran-3(2H)-one (LXXIII) is shown in Scheme A-27.

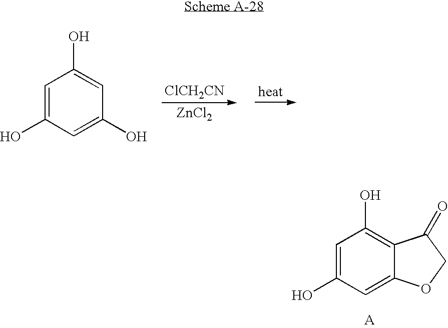

Preparation of 4,6-dihydroxybenzofuranone (Compound A) from phloroglucinol by thermal cyclization of the intermediate phenoxyacetonitrile, as shown in Scheme A-28.

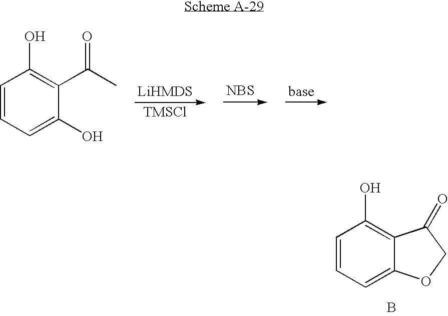

Preparation of 4-hydroxybenzofuranone (Compound B) from 1-(2,6-dihydroxyphenyl)ethanone by bromination of the enol ether followed by base-induced cyclization, as shown in Scheme A-29.

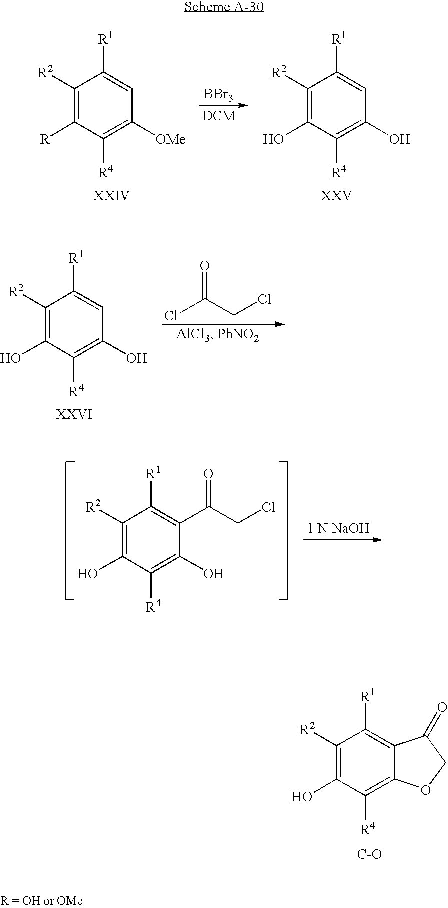

Preparation of monosubstituted 6-hydroxy benzofuranones (Compounds C-O) from anisole compounds LXXIV as shown in Scheme A-30.

| Benzofuranone | R¹ | R² | R⁴ |
|---|---|---|---|
| C | ME | H | H |
| D | H | Me | H |
| E | H | H | ME |
| F | F | H | H |
| G | H | F | H |
| H | H | H | F |
| I | CL | H | H |
| L | H | CL | H |
| M | H | H | CL |
| N | H | BR | H |
| O | Br | H | H |

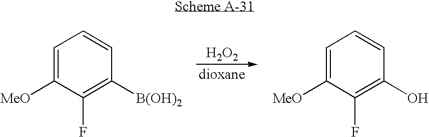

Preparation of 2-fluoro-3-methoxy-phenol is shown in Scheme A-31.

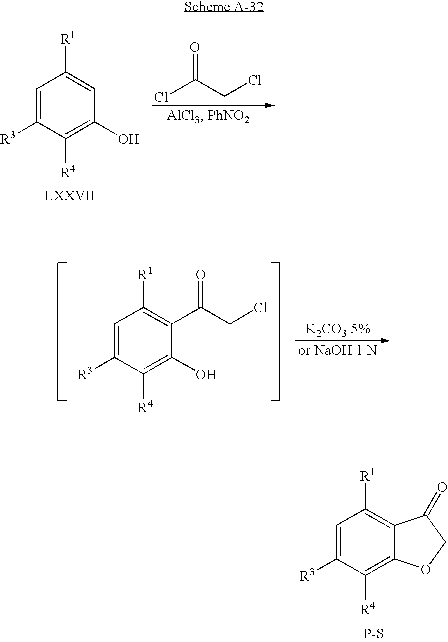

Preparation of other commercially non-available benzofuranone compounds (Compounds P-S) as shown in Scheme A-32.

| Benzofuranone | R¹ | R³ | R⁴ |
|---|---|---|---|
| P | OMe | OMe | H |
| Q | H | H | Br |
| R | OMe | OH | H |
| S | H | Br | H |

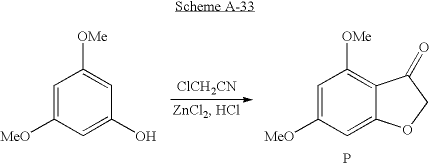

Preparation of 4,6-dimethoxybenzofuran-3(2H)-one (Compound P) as shown above in Scheme A-33 by a one-step alkylation-cyclization process.

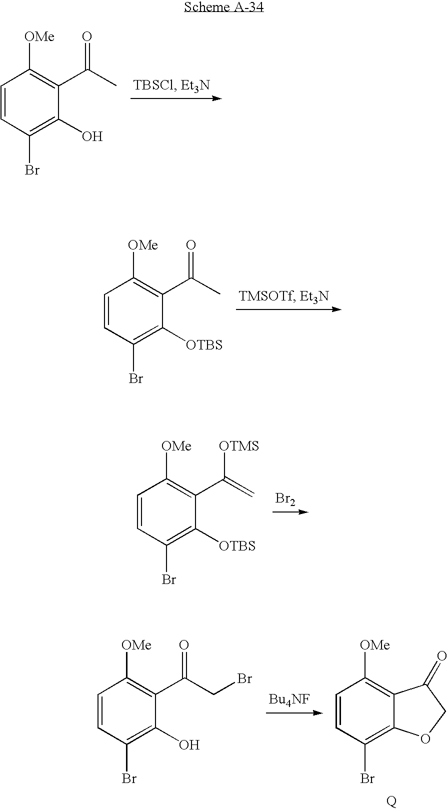

Preparation of 7-bromo-4-methoxybenzofuran-3(2H)-one (Compound Q) from 1-(3-bromo-2-hydroxy-6-methoxyphenyl)ethanone by bromination of the enol ether followed by fluoride-induced cyclization, as shown in Scheme A-34.

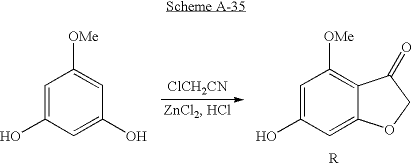

Preparation of 6-hydroxy-4-methoxybenzofuran-3(2H)-one (Compound R) as shown above in Scheme A-35 by a one-step alkylation-cyclization process.

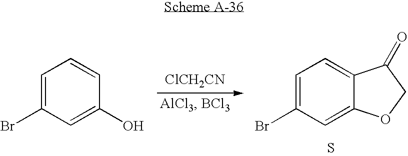

Preparation of 6-bromobenzofuran-3(2H)-one (Compound S) as shown above in Scheme A-36 by another one-step alkylation-cyclization process.

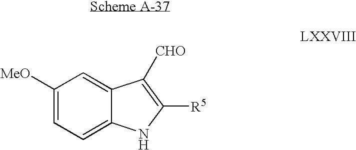

R⁵ substituent
H
a
Me
b

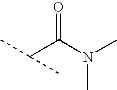

c

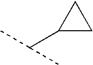

d

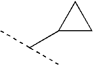

e

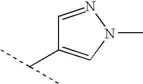

f

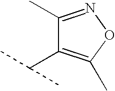

g

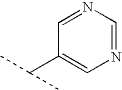

h

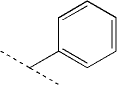

i

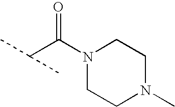

j

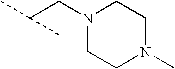

k

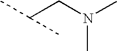

l

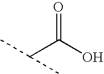

m

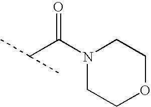

n

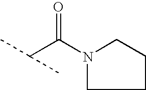

o

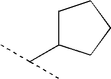

p

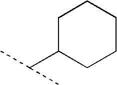

q

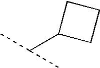

r

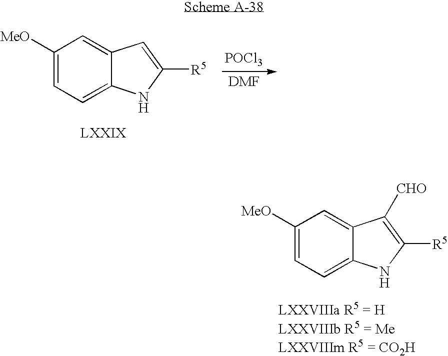

The preparation of 5-methoxy-indole-3-carbaldehyde (LXXVIIIa), 5-methoxy-2-methyl-indole-3-carbaldehyde (LXXVIIIb), and 3-formyl-5-methoxy-indole-2-carboxylic acid (LXXVIIIm) is shown in Scheme A-38.

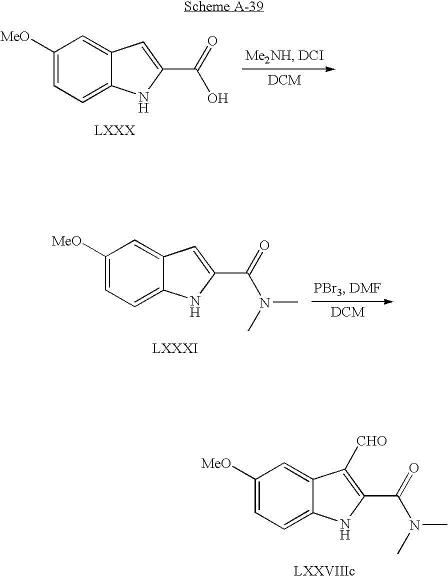

The preparation of 3-formyl-5-methoxy-indole-2-carboxylic acid dimethylamide (LXXVIIIc) is shown in Scheme A-39.

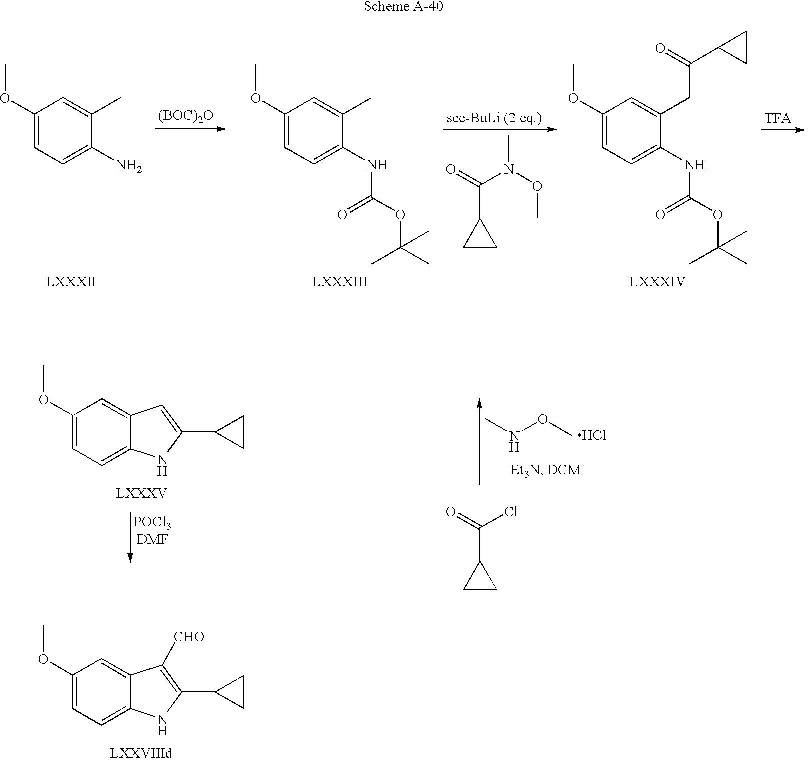

The preparation of 5-methoxy-2-cyclopropyl-indole-3-carbaldehyde (LXXVIIId) is shown in Scheme A-40.

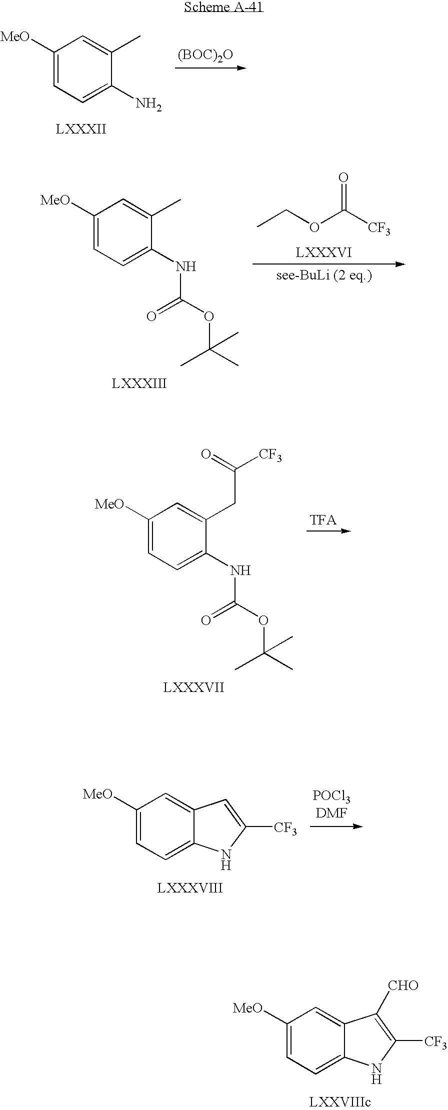

The preparation of 5-methoxy-2-trifluoromethyl-indole-3-carbaldehyde (LXXVIlle) is shown in Scheme A-41.

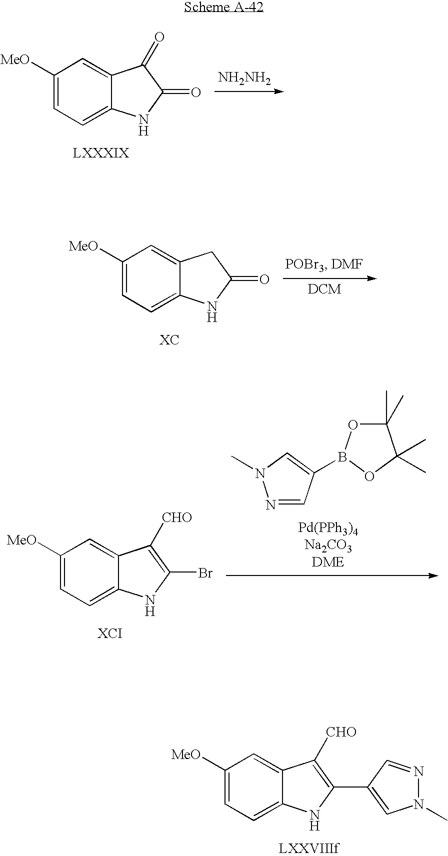

The preparation of 5-methoxy-2-(1-methyl-1H-pyrazol-4-yl)-indole-3-carbaldehyde (LXXVIIIf) is shown in Scheme A-42.

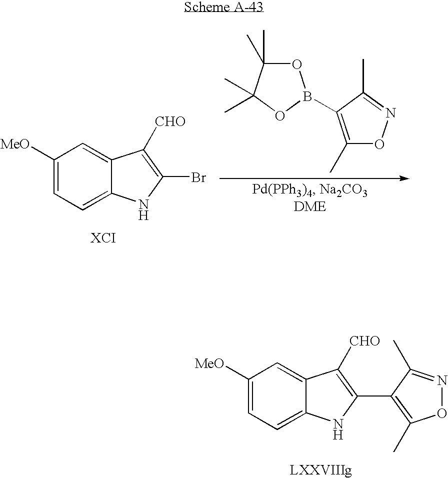

The preparation of 2-(3,5-Dimethyl-isoxazol-4-yl)-5-methoxy-indole-3-carbaldehyde (LXXVIIIg) is shown in Scheme A-43.

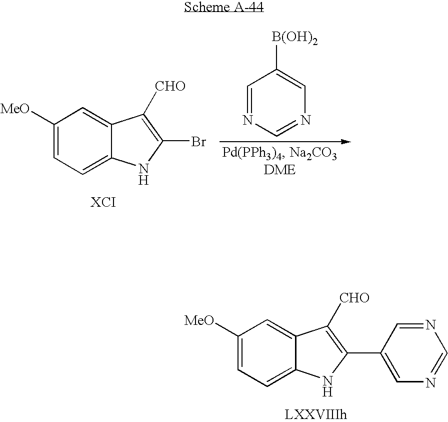

The preparation of 5-methoxy-2-pyrimidin-5-yl-indole-3-carbaldehyde (LXXVIIIh) is shown in Scheme A-44.

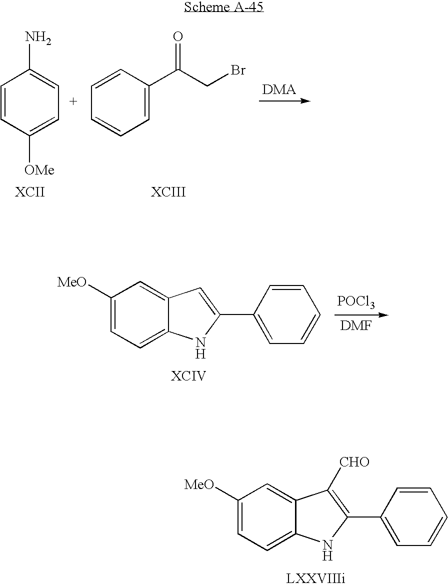

The preparation of 5-methoxy-2-phenyl-indole-3-carbaldehyde (LXXVIlli) is shown in Scheme A-45.

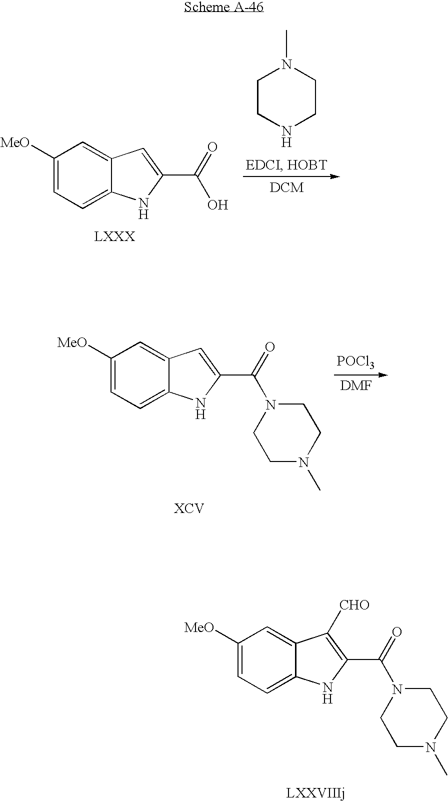

The preparation of 5-methoxy-2-(4-methyl-piperazine-1-carbonyl)-indole-3-carbaldehyde (LXXVIIIj) is shown in Scheme A-46.

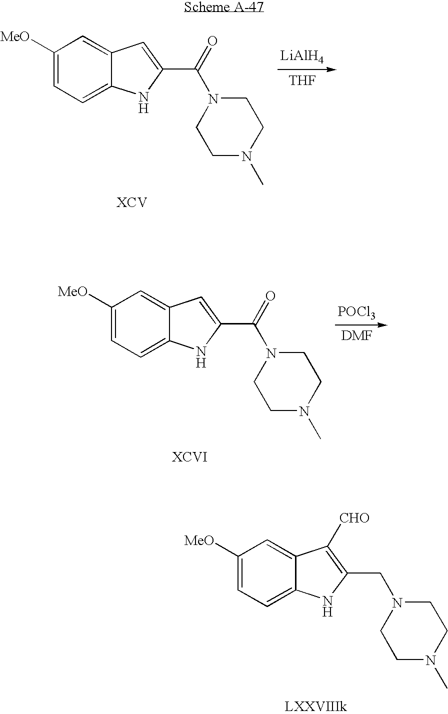

The preparation of 5-methoxy-2-(4-methyl-piperazin-1-ylmethyl)-indole-3-carbaldehyde (LXXVIIIk) is shown in Scheme A-47.

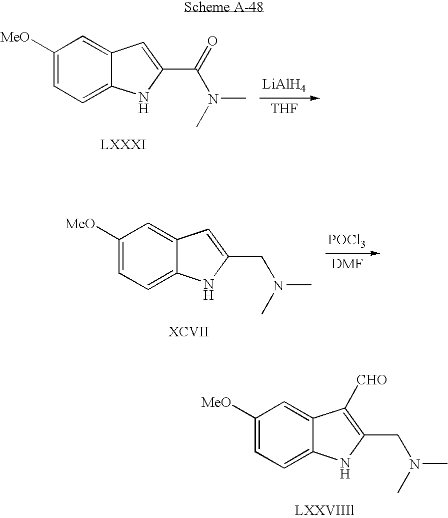

The preparation of 2-dimethylaminomethyl-5-methoxy-indole-3-carbaldehyde (LXXVIIII) is shown in Scheme A-48.

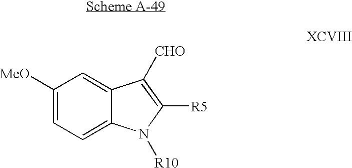

The synthesis of N-substituted 5-methoxy-indole-3-carbaldehydes (XCVIIIx-y) is summarized in Scheme A-49.

R⁵ substituent
H
a
Me
b

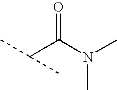

c

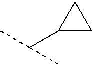

d
CF₃
e

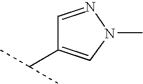

f

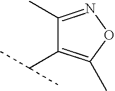

g

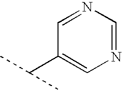

h

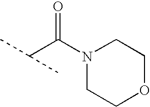

n

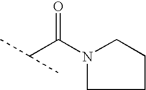

o

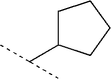

p

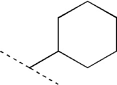

q

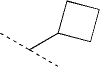

r

R¹⁰ substituent

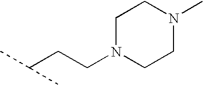

1

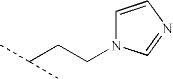

2

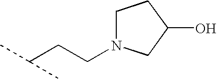

3

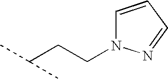

4

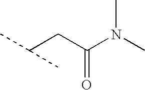

5

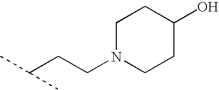

6

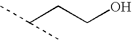

7

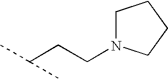

8

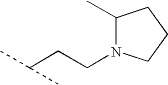

9

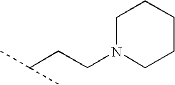

10

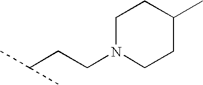

11

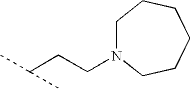

12

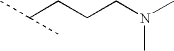

13

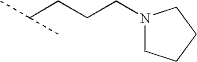

14

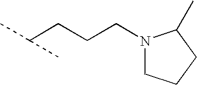

15

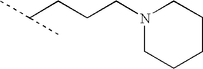

16

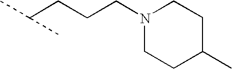

17

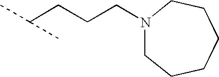

18

One route for the preparation of XCVIIIx-y is shown in Scheme A-50.

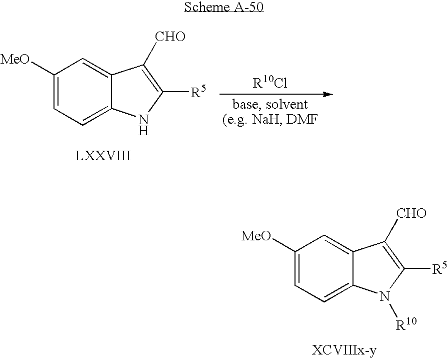

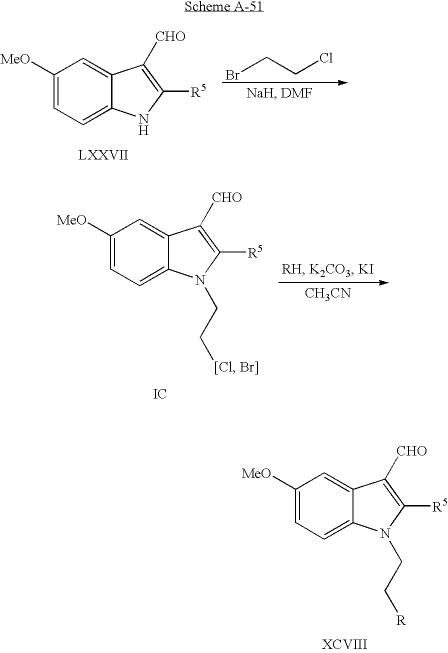

A dialkylation process was used to make the XCVIII compounds containing a heterocyclyl(ethylene) substituent as R¹⁰, as shown in Scheme A-51.

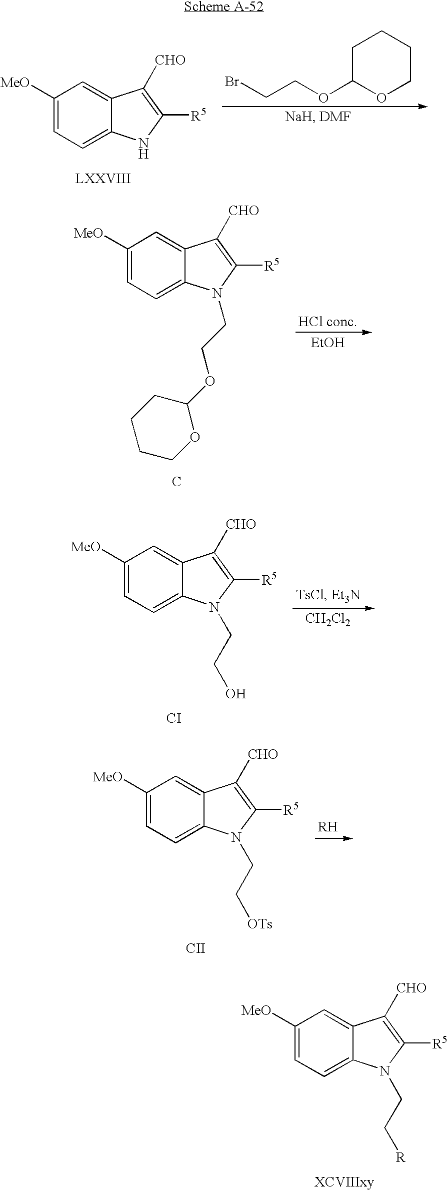

A dialkylation process was also used to make the XCVIIIxy compounds containing a heterocyclyl(ethylene) substituent as R¹⁰, via a protected 2-bromoethanol reagent, as shown in Scheme A-52.

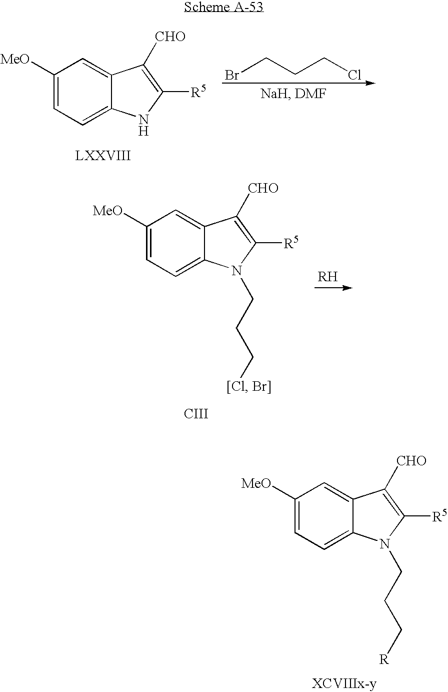

A dialkylation process was used to make the XCVIII compounds containing a heterocyclyl(propylene) substituent as R¹⁰, as shown in Scheme A-53.

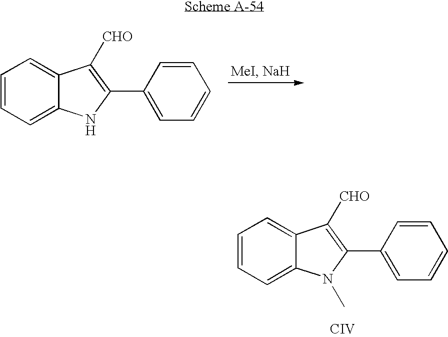

The preparation of 1-methyl-2-phenyl-1H-indole-3-carbaldehyde (CIV) is shown in Scheme A-54.

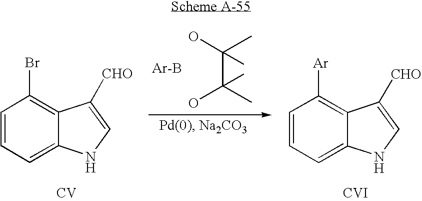

The preparation of 4-aryl-1H-indole-3-carbaldehyde (CVI) by Suzuki coupling is shown in Scheme A-55.

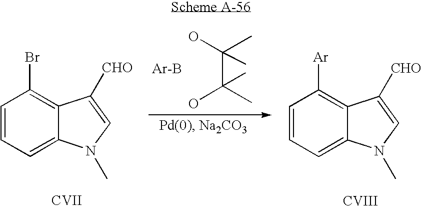

The preparation of 4-aryl-1-methyl-1H-indole-3-carbaldehyde (CVIII) by Suzuki coupling on the alkylated intermediate CVII is shown in Scheme A-56.

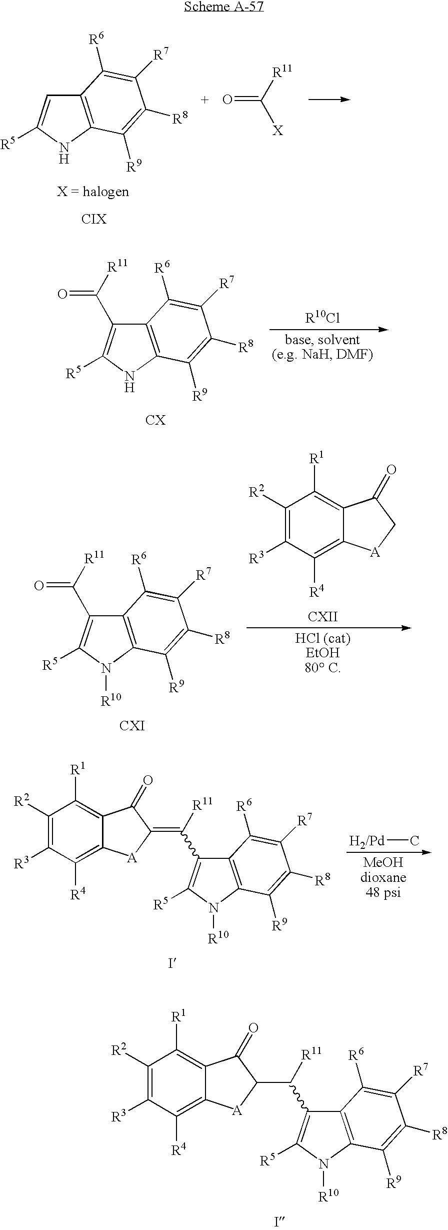

A synthesis of the 1H-indol-3-yl)methylene compounds of Formula I′ (compounds of formula 1 with a second carbon-to-carbon bond) and of the reduced indol-3-yl)methyl compounds I″ (compounds of formula 1 with absent) is shown in Scheme A-57. Acylation with R¹¹C(O)X, wherein X is halogen, or Vilsmeier-Haack formylation, of a compound of formula CIX thereby producing a compound of formula CX and optionally alkylating the compound of formula CX with R¹⁰Cl, thereby producing a compound of Formula CXI.

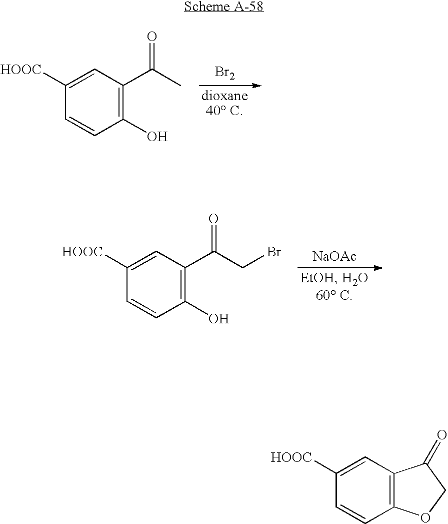

Preparation of 3-oxo-2,3-dihydrobenzofuran-5-carboxylic acid is shown above in Scheme A-58 by a two-step bromination-cyclization process.

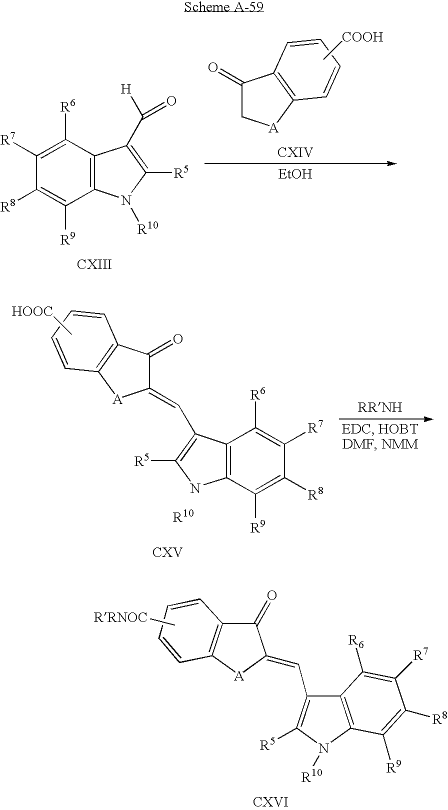

Condensation of 3-oxo-2,3-dihydrobenzofuran carboxylic acids CXIV with 1H-indole-3-carbaldehydes CXIII as shown above in Scheme A-59.

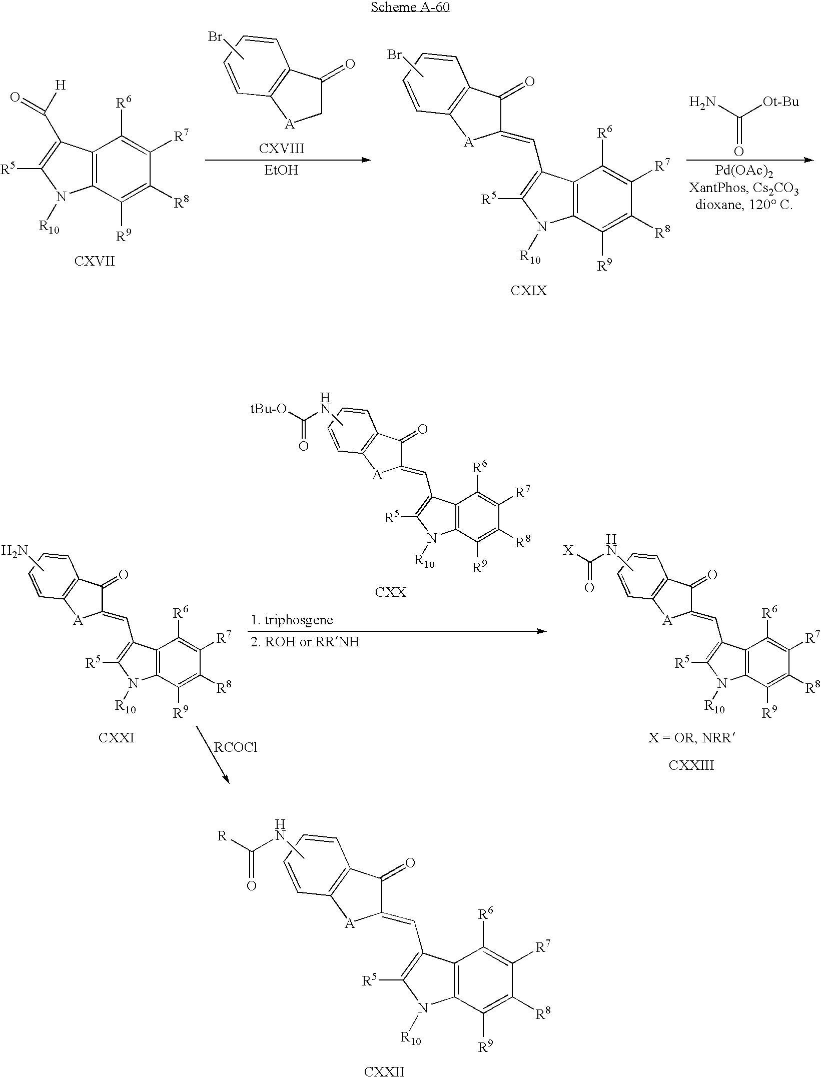

Condensation of bromo-3-oxo-2,3-dihydrobenzofuran CXVIII with 1H-indole-3-carbaldehydes CXVII as shown above in Scheme A-60.

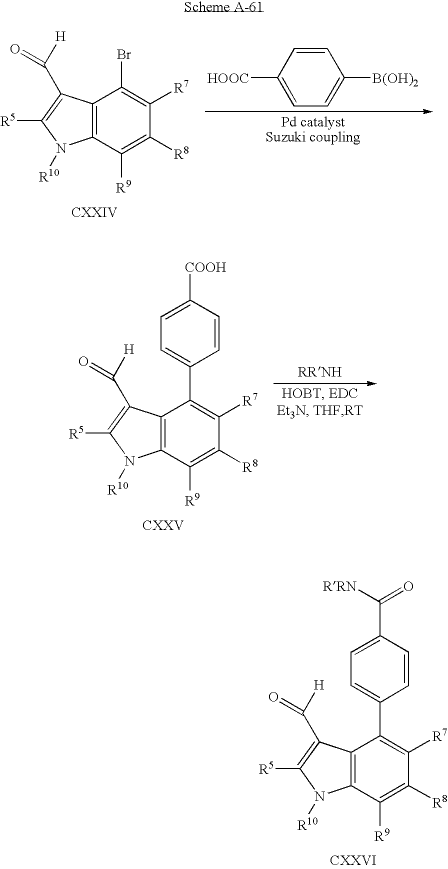

Preparation of 4-(3-formyl-1H-indol-4-yl)benzamide intermediates (CXXVI) as shown above in Scheme A-61 by Suzuki coupling on the 4-bromo-3-formyl-1H-indol-4-yl)benzamide CXXV.

Schemes B-1 to B-24 describes the: preparation of 3-substituted-1H-pyrrolo[2,3-b]pyridine and 3-substituted-1H-pyrrolo[3,2-b]pyridine compounds having a benzofuranone or benzothiophenone with at least one hydroxyl on the benzofuranone or benzothiophenone phenyl ring.

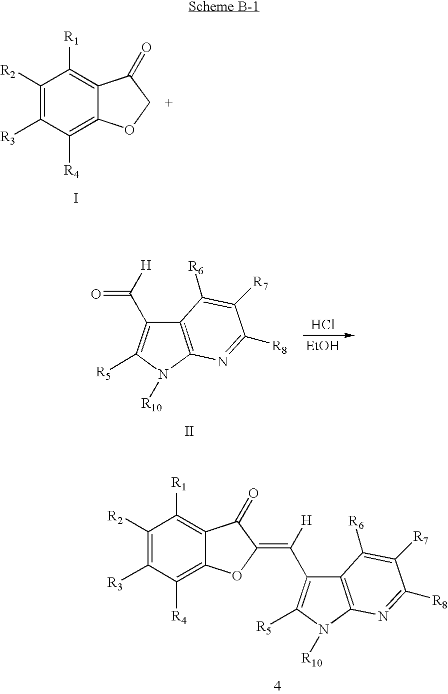

Benzofuranone molecules 4 may be prepared according to Scheme B-1 by reacting benzofuranone compounds I with heteroaryl aldehydes II in EtOH with catalytic amounts of HCl at 80 C. Benzofuranone compounds I and heteroaryl aldehydes II can be purchased commercially or prepared synthetically via standard organic chemistry protocols.

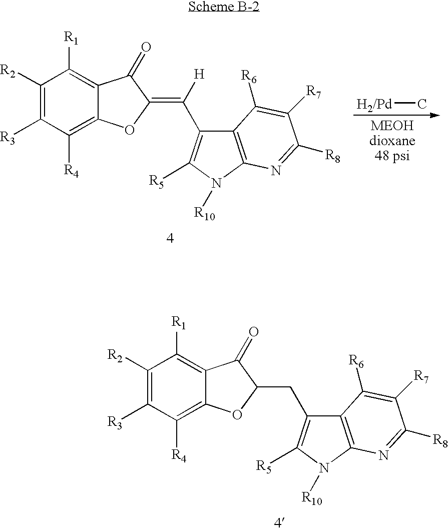

2-Methylbenzofuranone molecules 4′ may be prepared according to Scheme B-2 by reduction of 2-methylenebenzofuranones 4 with Pd/C in MeOH/dioxane under 48 psi atmosphere of hydrogen.

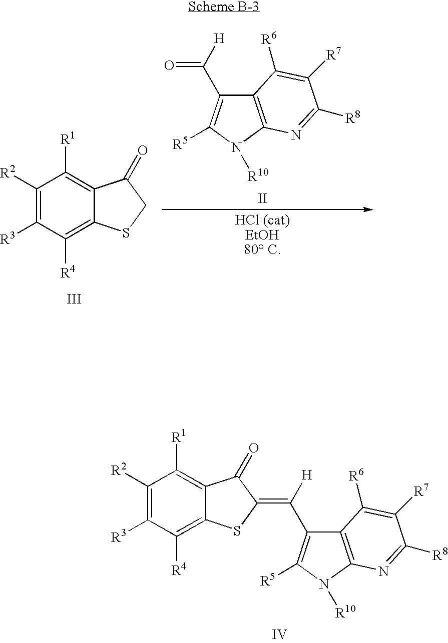

Benzothiophenone molecules IV may be prepared according to Scheme B-3 by reacting benzothiophenone III with the heteroaryl aldehydes II in benzene with catalytic amounts of piperidine at 85 C. Benzothiophenone III and heteroaryl aldehydes II can be purchased commercially or prepared synthetically via standard organic chemistry protocols.

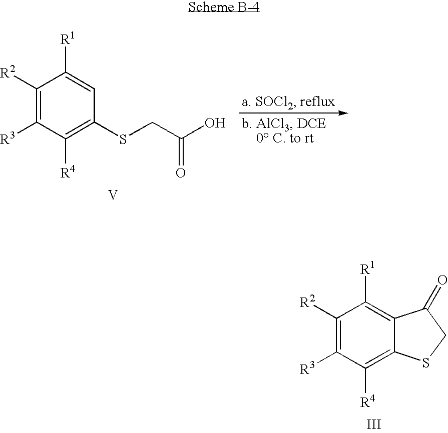

Benzothiophenones III as described in Scheme B-4 can be obtained from the corresponding acids V using known literature procedures. To the acid (15.6 mmol) is added SOCl₂ (10 mL). After heating the resulting suspension to 85° C. for 1 hour, the reaction is concentrated in vacuo and placed under vacuum for 30 minutes. To the reaction is added methylene chloride (30 mL) and cooled on an ice-salt bath for 15 minutes. AlCl₃ (2.5 g) is added in portions over 20 minutes. The reaction is stirred with cooling for 15 minutes and then allowed to stir for 45 minutes at room temperature. The reaction is quenched with ice water, extracted with methylene chloride, and concentrated in vacuo to afford the desired compound without further purification.

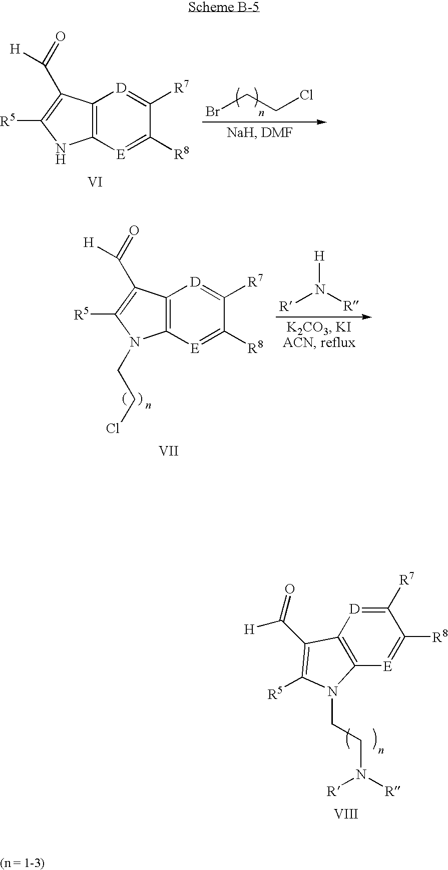

3-Indolecarboxaldehydes as described by Scheme B-5 can be obtained by alkylation of the 3-indolecarboxaldehydes VI using the corresponding co-bromochloroalkanes and NaH in DMF under standard literature conditions. The resulting alkyl chloride was then reacted with the desired secondary amine using potassium carbonate and potassium iodide in ACN at 80° C. under standard literature conditions.

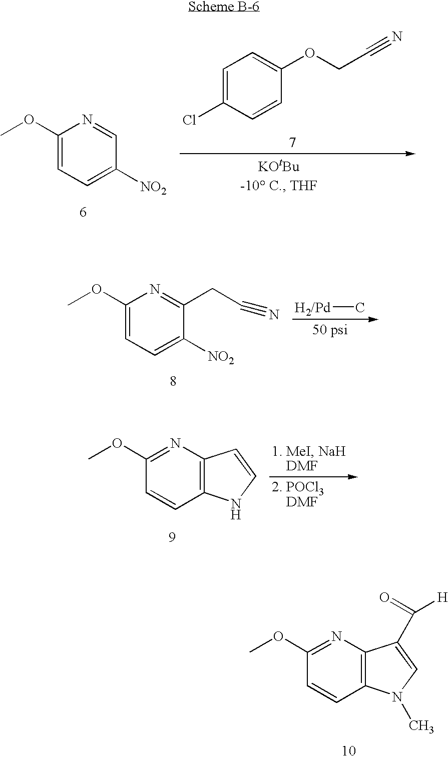

5-Methoxy-1-methyl-1H-pyrrolo[3,2-b]pyridine-3-carbaldehyde (10) as described in Scheme B-6 can be obtained by first generating 5-methoxy-1H-pyrrolo[3,2-b]pyridine from 2-methoxy-5-nitro-pyridine 6 using literature procedures described in Eur. J. Med. Chem. 2004, 39, 515. The azaindole was then converted into 5-methoxy-1-methyl-1H-pyrrolo[3,2-b]pyridine-3-carbaldehyde using Vilsmeier-Haack methods.

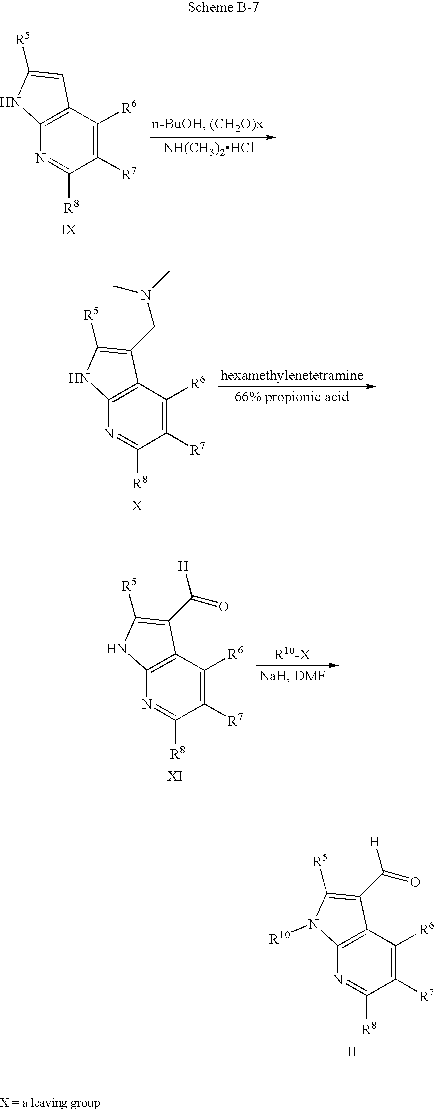

7-Aza-3-indole carboxaldehyde compounds II as described in Scheme B-7 can be obtained by first generating 7-azagramine from 7-azaindole IX, paraformaldehyde, and dimethylamine, by Mannich reaction followed by hydrolysis using literature procedures described in JACS 1955, 77, 457. This was followed by methylation using MeI and NaH in DMF under standard literature conditions.

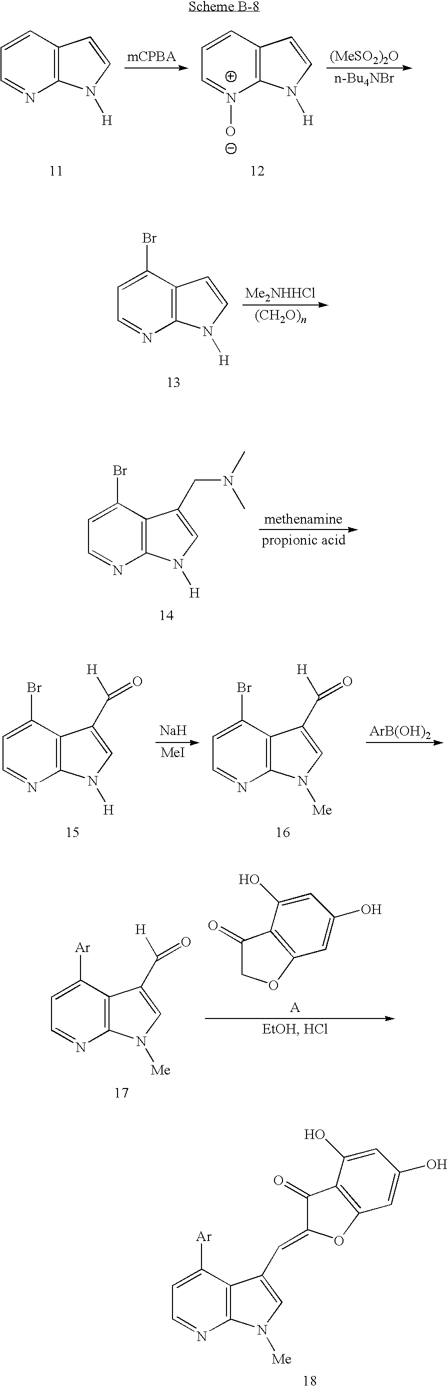

Preparation of (2Z)-2[(4-aryl-1-methyl-1H-pyrrolo[2,3-b]pyridine-3-yl)methylene]-4,6-dihydroxy-1-benzofuran-3(2H)-one (18) is shown in Scheme B-8. 4-Bromo-1H-pyrrolo[2,3-b]pyridine was prepared by a modified N-oxide rearrangement procedure. The 7-azagramine 14 was obtained from 7-azaindole 13, paraformaldehyde, and dimethylamine, by Mannich reaction followed by hydrolysis. This was followed by methylation using MeI and NaH.

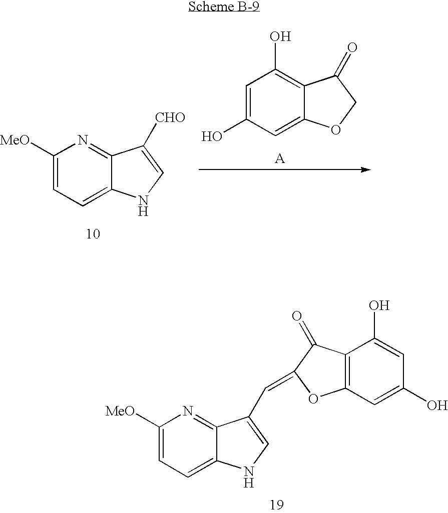

Preparation of (2Z)-4,6-dihydroxy-2-[(5-methoxy-1H-pyrrolo[3,2-b]pyridin-311)methylene]-1-benzofuran-3(2H)-one (19) is shown in Scheme B-9. 5-Methoxy-1H-pyrrolo[3,2-b]pyridine-3-carbaldehyde (10) was condensed with furanone A, which proceeded smoothly.

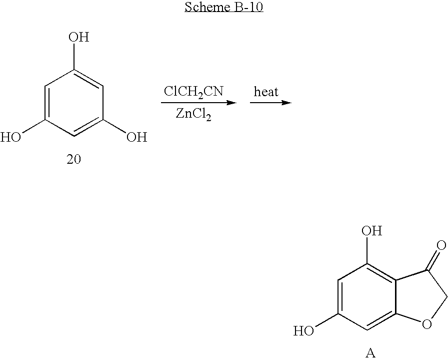

Preparation of 4,6-dihydroxybenzofuranone (Compound A) from phloroglucinol by thermal cyclization of the intermediate phenoxyacetonitrile, as shown in Scheme B-10.

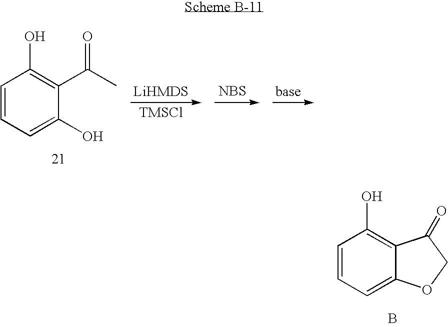

Preparation of 4-hydroxybenzofuranone (Compound B) from 1-(2,6-dihydroxyphenyl)ethanone (21) by bromination of the enol ether followed by base-induced cyclization, as shown in Scheme B-11.

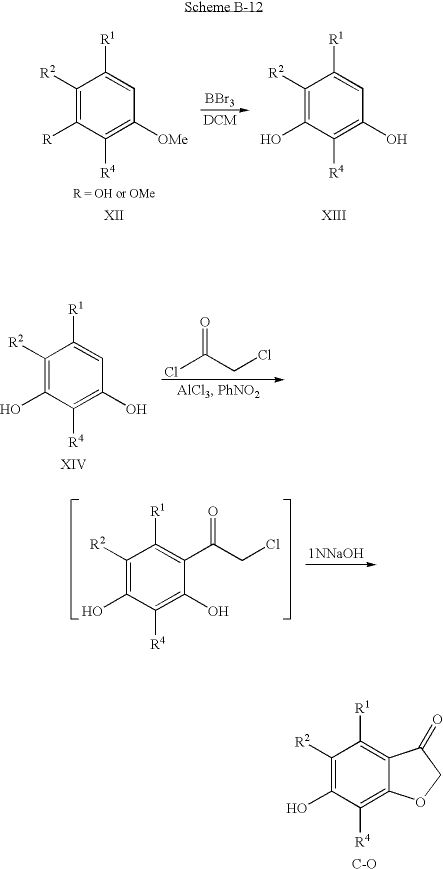

Preparation of monosubstituted 6-hydroxy benzofuranone compounds (Compounds C-O) from anisole compounds XII as shown in Scheme B-12.

| Benzofuranone | R¹ | R² | R⁴ |
|---|---|---|---|
| C | ME | H | H |
| D | H | Me | H |
| E | H | H | ME |
| F | F | H | H |
| G | H | F | H |
| H | H | H | F |
| I | CL | H | H |
| L | H | CL | H |
| M | H | H | CL |
| N | H | BR | H |
| O | Br | H | H |

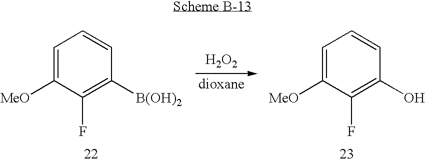

Preparation of 2-fluoro-3-methoxy-phenol (23) as shown in Scheme B-13.

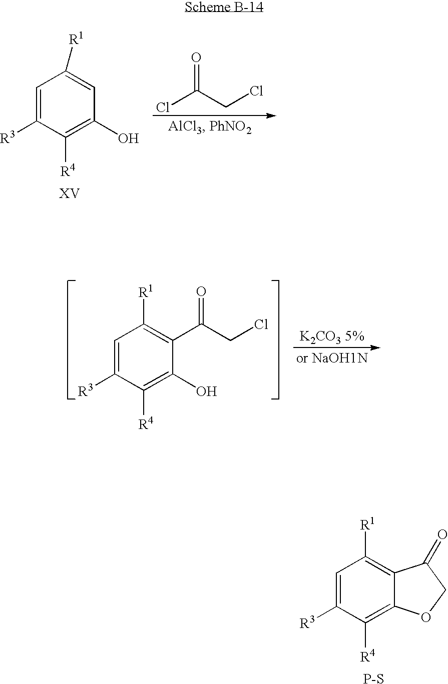

Preparation of other commercially non-available benzofuranone compounds (Compounds P-S) as shown in Scheme B-14.

| Benzofuranone | R₁ | R₃ | R₄ |
|---|---|---|---|
| P | OMe | OMe | H |
| Q | H | H | Br |
| R | OMe | OH | H |
| S | H | Br | H |

Preparation of 4,6-dimethoxybenzofuran-3(2H)-one (Compound P) as shown above in

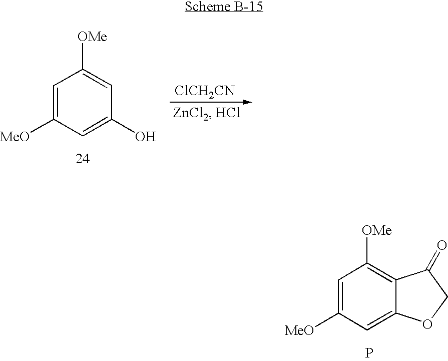

Scheme B-15 by a one-step alkylation-cyclization process.

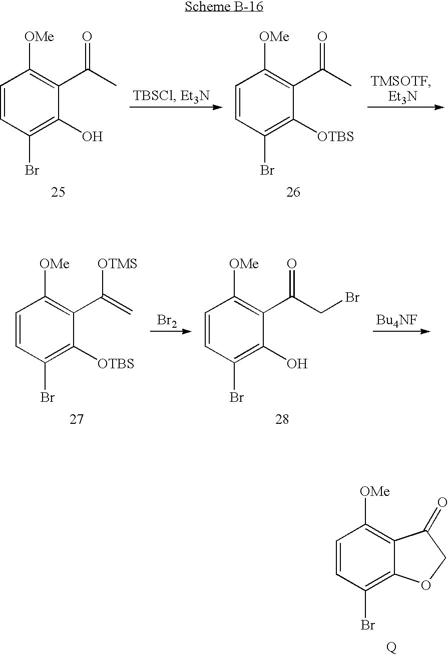

Preparation of 7-bromo-4-methoxybenzofuran-3(2H)-one (Compound Q) from 1-(3-bromo-2-hydroxy-6-methoxyphenyl)ethanone by bromination of the enol ether followed by fluoride-induced cyclization, as shown in Scheme B-16.

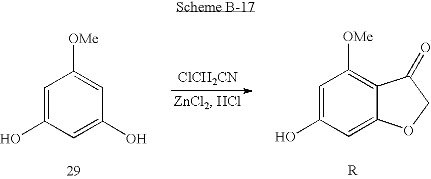

Preparation of 6-hydroxy-4-methoxybenzofuran-3(2H)-one (Compound R) as shown above in Scheme B-17 by a one-step alkylation-cyclization process.

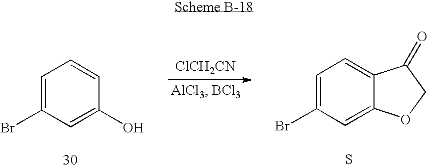

Preparation of 6-bromobenzofuran-3(2H)-one (Compound S) as shown above in Scheme B-18 by another one-step alkylation-cyclization process.

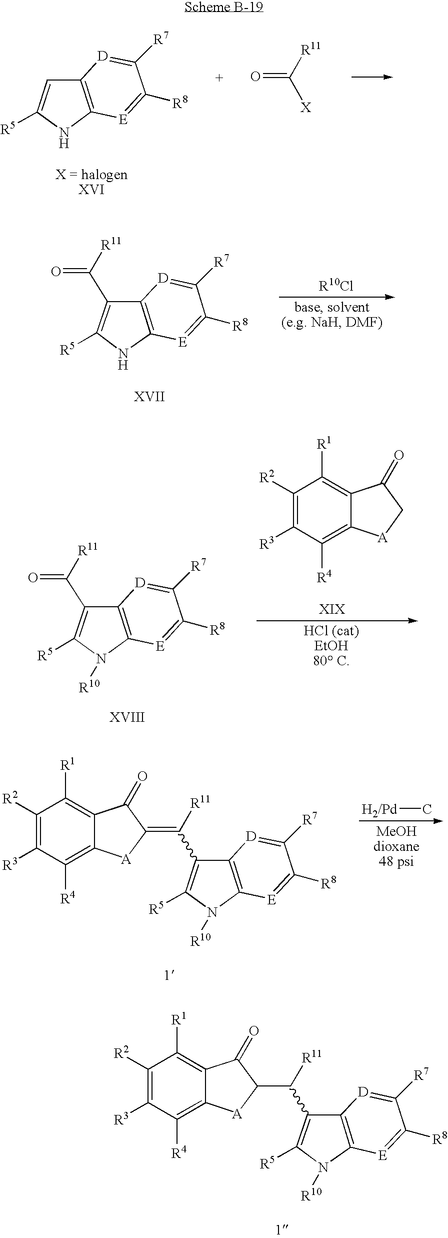

A synthesis of the 1H-pyrrolopyridin-3-yl)methylene compounds of Formula 1′ (compounds of Formula 1 with . . . a second carbon to carbon bond) and of the reduced pyrrolopyridin-3-yl)methyl compounds 1″ (compounds of Formula 1 with . . . absent) is shown in Scheme B-19.

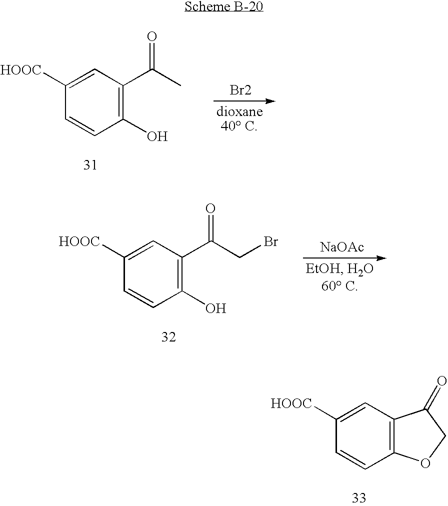

Preparation of 3-oxo-2,3-dihydrobenzofuran-5-carboxylic acid (33) as shown above in Scheme B-20 by a two-step bromination-cyclization process.

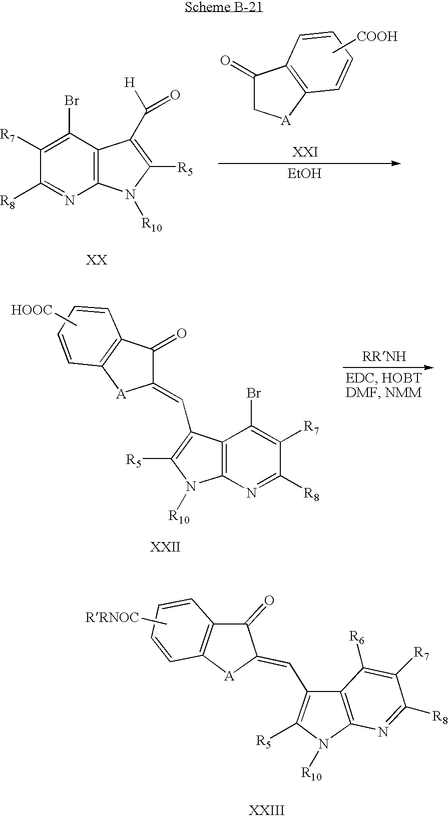

Condensation of 3-oxo-2,3-dihydrobenzofuran carboxylic acids XXI with 1H-pyrrolo[2,3-b]pyridine-3-carbaldehydes XX as shown above in Scheme B-21.

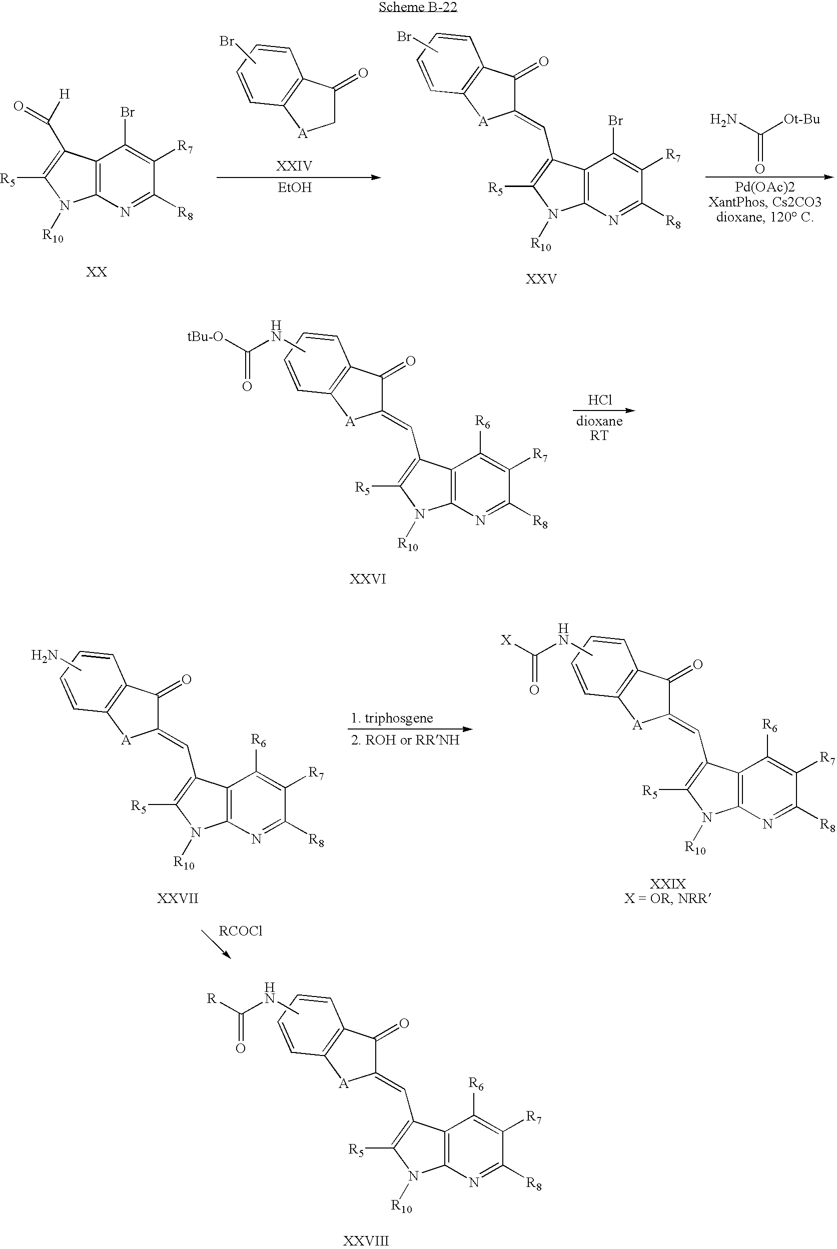

Condensation of bromo-3-oxo-2,3-dihydrobenzofuran XXIV with 1H-pyrrolo[2,3-b]pyridine-3-carbaldehydes XX as shown above in Scheme B-22.

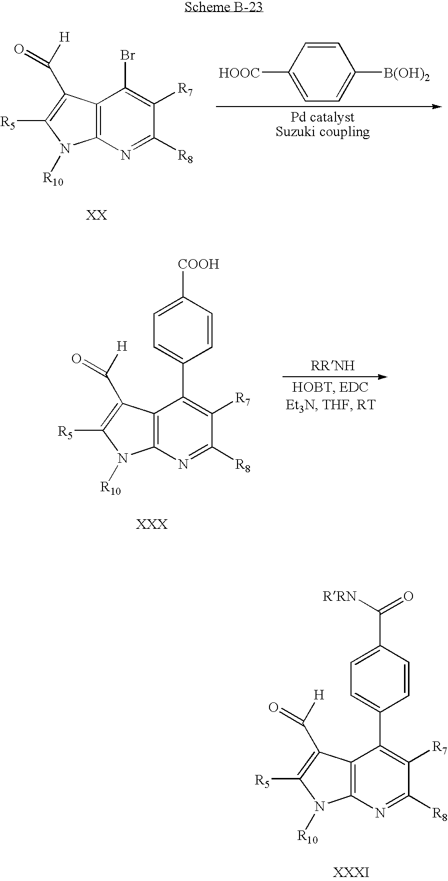

Preparation of 4-(3-formyl-1H-pyrrolo[2,3-b]pyridin-4-yl)benzamide intermediates Compound XXXI as shown above in Scheme B-23 by Suzuki coupling on the 4-bromo-3-formyl-1H-pyrrolo[2,3-b]pyridin-4-yl)benzamide XX.

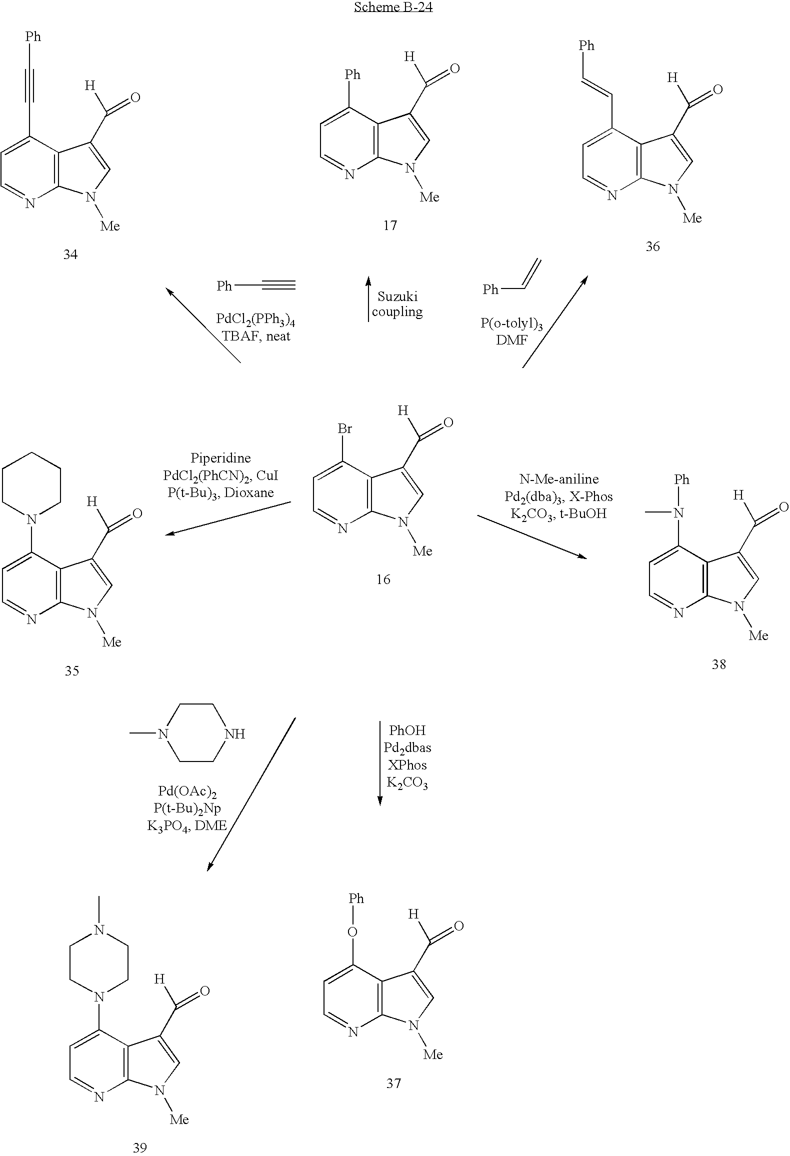

Scheme B-24 summarizes the synthesis of various 1-methyl-1-H-pyrrolo[2,3-b]pyridine-3-carbaldehyde intermediates from 4-4-romo-1-methyl-1-H-pyrrolo[2,3-b]pyridine-3-carbaldehyde 16.

Synthetic Schemes C-1 to C-6 describing the preparation of the compounds of the Invention (Prodrugs) are shown below.

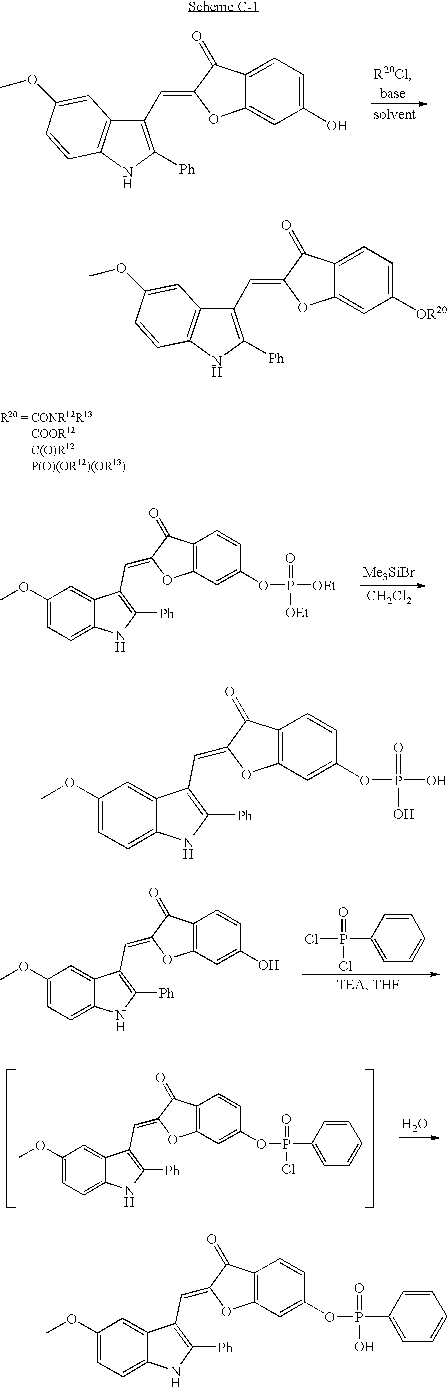

Preparation of prodrugs of (2Z)-6-hydroxy-2-[(5-methoxy-2-phenyl-1H-indol-3-yl)methylene]-1-benzofuran-3(2H)-one compounds including phosphoric acid mono-{2-[1-(5-methoxy-2-phenyl-1H-indol-3-yl)-meth-(Z)-ylidene]-3-oxo-2,3-dihydro-benzofuran-6-yl}ester and phenyl-phosphonic acid mono-{2-[1-(5-methoxy-2-phenyl-1H-indol-3-yl)meth-(Z)-ylidene]-3-oxo-2,3-dihydro-benzofuran-6-yl}ester are shown in Scheme C-1.

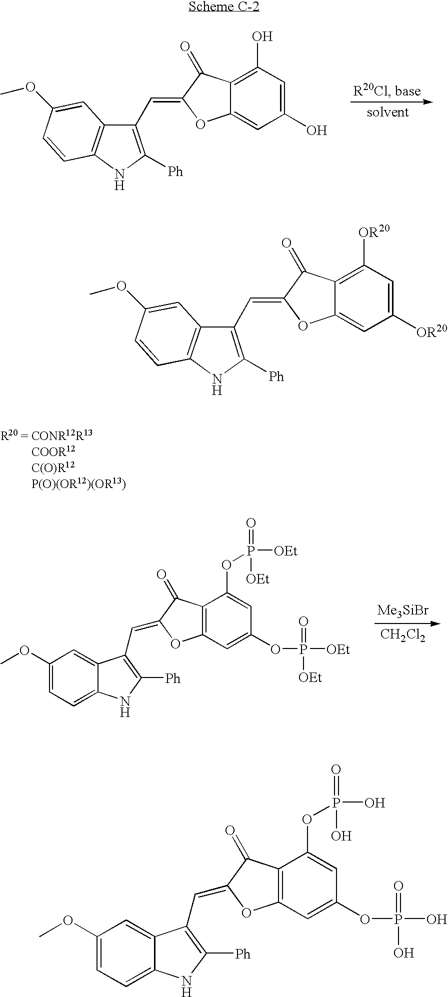

Prodrugs of 4,6-dihydroxy-2-[(5-methoxy-2-phenyl-1H-indol-3-yl)methylene]-1-benzofuran-3(2H)-one including phosphoric acid mono-{2-[1-(5-methoxy-2-phenyl-1H-indol-3-yl)-meth-(Z)-ylidene]-3-oxo-4-phosphonooxy-2,3-dihydro-benzofuran-6-yl}ester are shown in Scheme C-2.

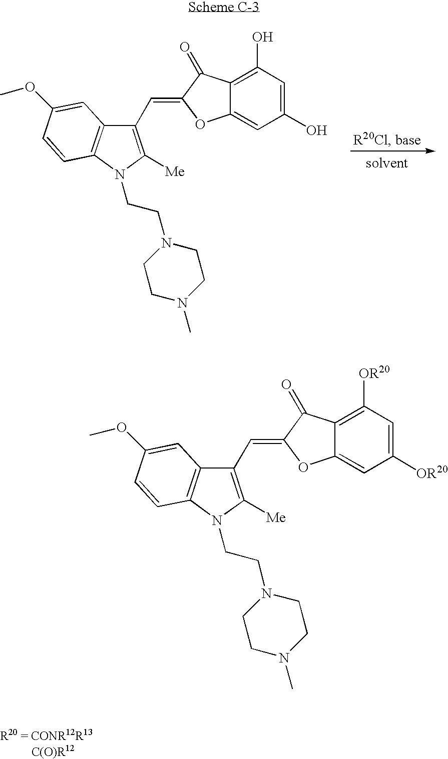

Prodrugs of 4,6-dihydroxy-2-({5-methoxy-2-methyl-1-[2-(4-methylpiperazin-1-yl)pethyl]-1H-indol-3-yl}methylene)-1-benzofuran-3(2H)-one are shown in Scheme C-3.

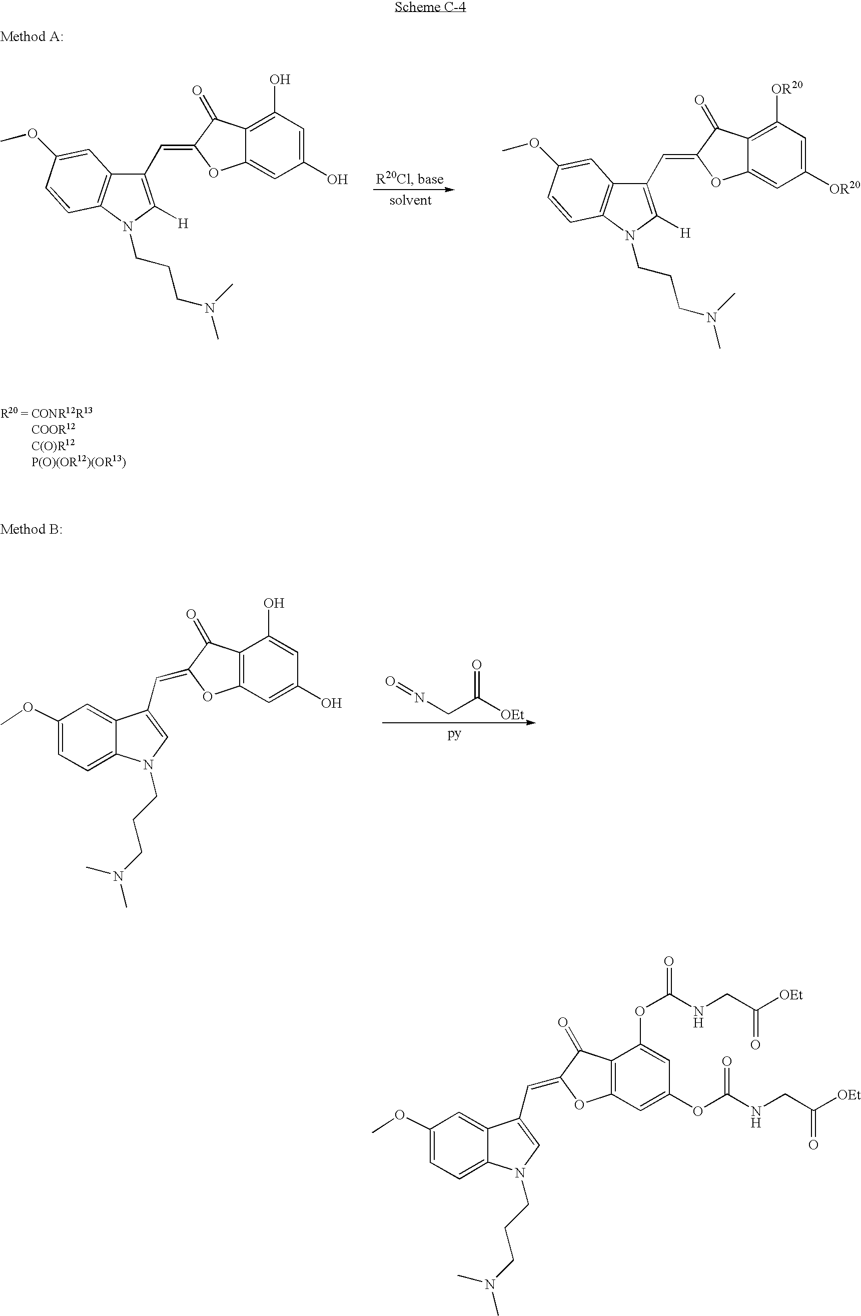

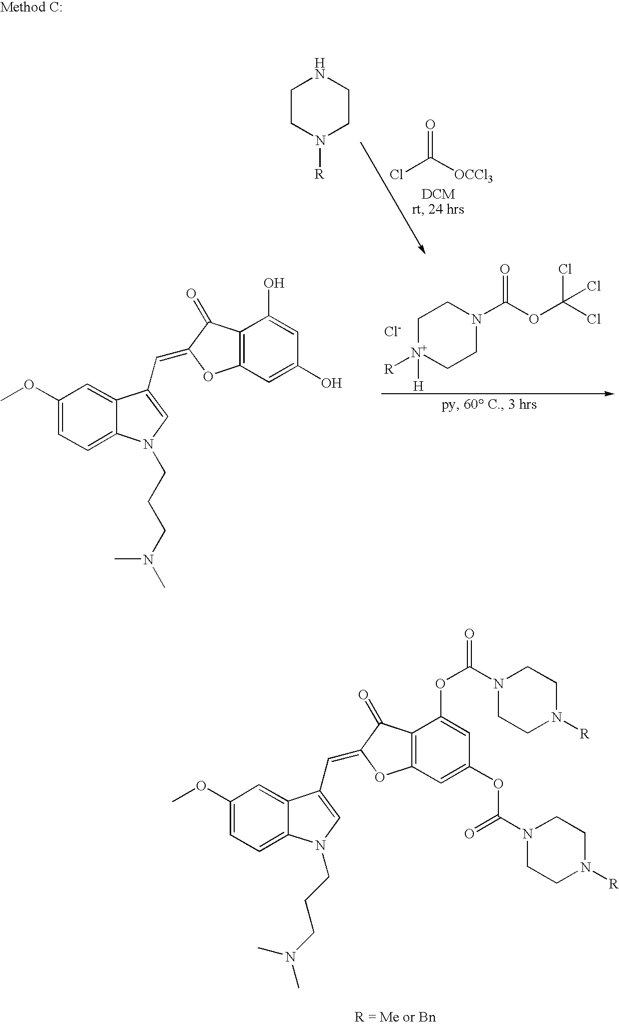

Prodrugs of (2Z)-2-({1-[3-(dimethylamino)propyl]-5-methoxy-1H-indol-3-yl}methylene)-4,6-dihydroxy-1-benzofuran-3(2H)-one are shown in Scheme C-4.

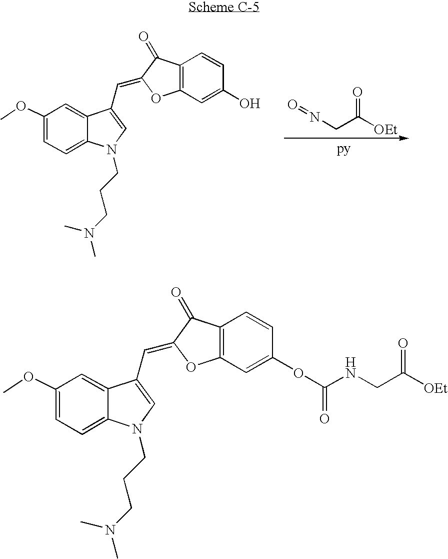

Prodrug of (2Z)-2-({1-[3-(dimethylamino)propyl]-5-methoxy-1H-indol-3-yl}methylene)-6-hydroxy-1-benzofuran-3(2H)-one is shown in Scheme C-5.

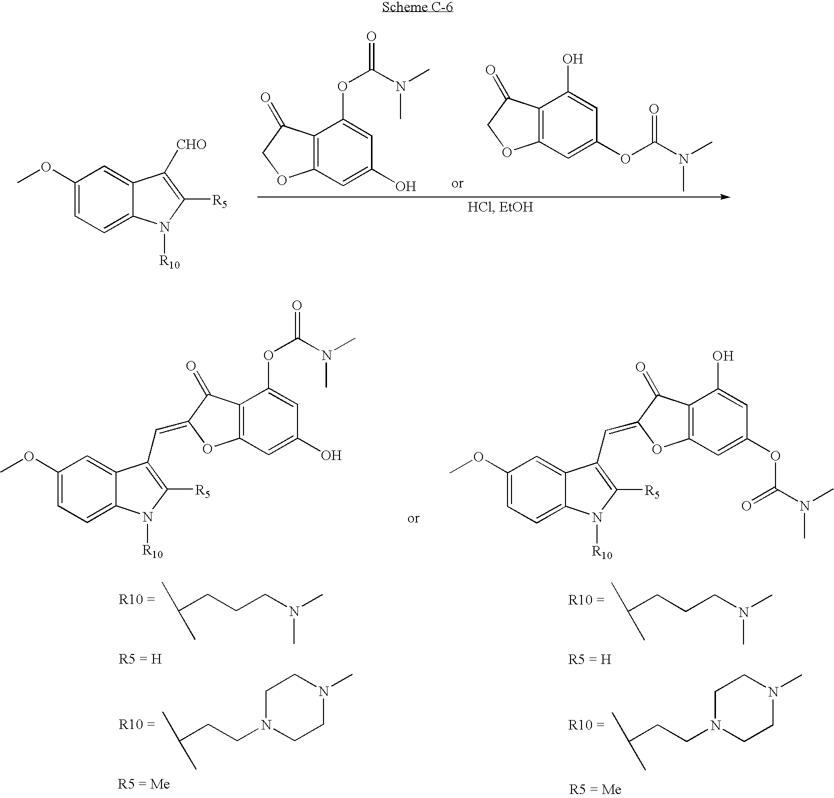

Mono-Prodrugs of (2Z)-4,6-dihydroxy-2-({5-methoxy-2-methyl-1-[2-(4-methylpiperazin-1-yl)pethyl]-1H-indol-3-yl}methylene)-1-benzofuran-3(2H)-one and (2Z)-2-({1-[3-(dimethylamino)propyl]-5-methoxy-1H-indol-3-yl}methylene)-4,6-dihydroxy-1-benzofuran-3(2H)-one are shown in Scheme C-6.

Schemes for preparing benzofuranone intermediates: dimethyl-carbamic acid 6-hydroxy-3-oxo-2,3-dihydro-benzofuran-4-yl ester and dimethyl-carbamic acid 4-hydroxy-3-oxo-2,3-dihydro-benzofuran-6-yl ester:

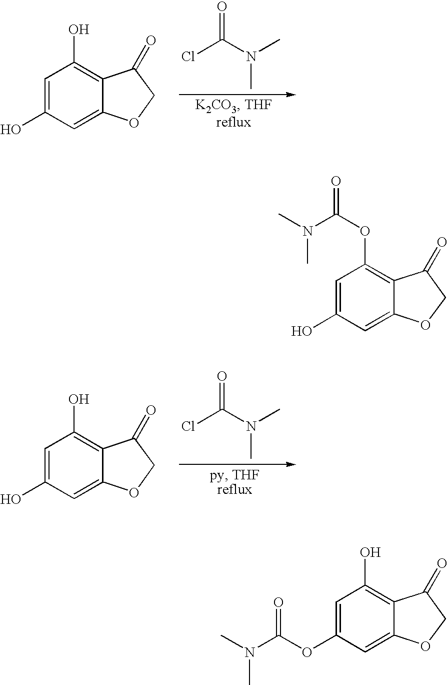

Scheme for the preparation of the compounds of the invention:

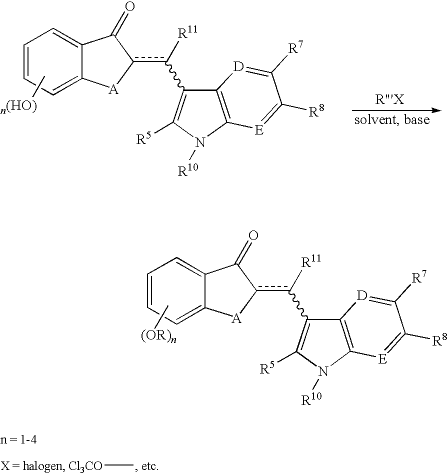

One of skill in the art will recognize that Schemes A-1 to C-6 can be adapted to produce the other compounds of Formulas 1-3 and pharmaceutically acceptable salts of compounds of Formulas 1-3 according to the present invention.

### EXAMPLES

The following abbreviations are used herein and have the indicated definitions: ACN is acetonitrile, AcOH is acetic acid. ATP is adenosine triphosphate. Biotage Initiator™ 60 is a 60-position sample microwave synthesizer. Initiator™ is a registered trademark of Biotage AB, Uppsala, Sweden. BOC is t-butoxycarbonyl. Celite™ is flux-calcined diatomaceous earth. Celite™ is a registered trademark of World Minerals Inc. CHAPS is (3-[(3-cholamidopropyl)dimethylammonio]-1-propanesulfonic acid, DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation. EDCl is 3′-dimethylaminopropyl)carbodiimide or water-soluble carbodiimide, EDTA is ethylenediaminetetraacetic acid, ESI stands for Electrospray Ionization, EtOAc is ethyl acetate, and EtOH is ethanol. HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, MS is mass spectrometry, and NEt₃ is triethylamine. Ni(Ra) is Raney™ nickel, a sponge-metal catalyst produced when a block of nickel-aluminum alloy is treated with concentrated sodium hydroxide. Raney™ is a registered trademark of W. R. Grace and Company. NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is trichloroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

### Synthetic Methods

### A-I. Synthesis of Benzofuranone Intermediates

### Preparation of 4,6-dihydroxybenzofuranone (Compound A)

To a solution of phloroglucinol (2 g, 16 mmol, 1 eq.) in ethyl ether (20 mL), ClCH₂CN (10 mL), ZnCl₂ (0.2 g, 1.6 mmol, 0.1 eq.) and 10% HCl/Et₂O (15 mL) were added. The mixture was stirred at room temperature overnight. The yellow precipitate (imine hydrochloride) was filtered off and washed three times with ethyl ether. Then, it was dissolved in 25 mL of water and heated at 100° C. overnight. The red solid was filtered off, washed three times with water, and dried to give pure 4,6-dihydroxy-benzofuran-3-one. Yield: 70%. MS (m/z): 167.2 (MH+).

### Preparation of 4-hydroxybenzofuranone (Compound B)

LiHMDS (1M solution in THF, 3.1 mL, 3.1 mmol, 3.6 eq.) was slowly added to a solution of 2′,6′-dihydroxyacetophenone (131 mg, 0.86 mmol, 1 eq.) in anhydrous THF (4.5 mL) under argon atmosphere at −78° C. After 30 minutes, TMSCl (0.65 mL, 5.16 mmol, 6 eq.) was added and the resulting mixture was stirred for 4 hours. Then NBS (171 mg, 0.95 mmol, 1.1 eq.) was slowly added and the solution was stirred for 1 hour at −78° C. and for 10 minutes at rt. 1M NaOH (2 mL) was added and the resulting solution was stirred until complete disappearance of the starting material. The reaction was quenched by adding 1M HCl until pH 4. The aqueous layer was extracted with EtOAc and the collected organic extracts were washed with brine, dried on anhydrous Na₂SO₄ and evaporated under reduced pressure. The oily crude mixture was purified by silica gel column chromatography (eluent: EtOAc/petroleum ether 15:85). The title compound was obtained as a pale yellow solid. Yield: 46%. MS (m/z): 151.5 (MH+).

### Preparation of monosubstituted 6-hydroxy benzofuranones (Compounds C-O) Preparation of 2-fluoro-3-methoxy-phenol

Hydrogen peroxide (35% in water, 5 mL) was added to a solution of 2-fluoro-3-methoxyphenylboronic acid (500 mg, 2.94 mmol) in dioxane (5 mL). The reaction mixture was stirred at 100° C. for 2.5 hours and then allowed to cool to rt. Water was added and the aqueous layer was extracted with methylene chloride. The combined organic layers were dried on Na₂SO₄ and evaporated affording the title compound as dark oil. Yield: 71%. MS (m/z): 143.1 (MH⁺).

General Procedure for the Demethylation with BBr₃

To a solution of the methoxy-derivative (8.7 mmol) in methylene chloride (40 mL), cooled to −78° C., BBr₃ (1 M in methylene chloride, 4 eq. for each methoxy group) was added in drops. The reaction was stirred overnight allowing to the cooling bath to expire. The mixture was cooled again to −78° C. and quenched by addition of water in drops. The aqueous layer was extracted with EtOAc. The combined organic layers were dried on Na₂SO₄ and evaporated. The residue was triturated with EtOAc to give crude resorcinol that was used for the following reaction without further purification. This procedure was used to obtain the following compounds:

### 2-Fluoro-benzene-1,3-diol

Yield: 93%. MS (m/z): 129.1 (MH⁺).

### 5-Fluoro-benzene-1,3-diol

Yield: 97%. MS (m/z): 129.2 (MH⁺).

### 5-Chloro-benzene-1,3-diol

Yield: 87%. MS (m/z): 145.4 (MH⁺).

General Procedure for the Preparation of 6-hydroxybenzofuranones

Chloroacetyl chloride (0.33 mL, 4.15 mmol, 1.2 eq.) was added to a suspension of AlCl₃ (2.3 g, 17.3 mmol, 5 eq.) in nitrobenzene (6 mL), cooled to 0° C. The selected resorcinol (3.46 mmol, 1 eq.) was dissolved in nitrobenzene (6 mL) and added at 0° C. to the reaction mixture. The reaction was stirred at room temperature overnight, then poured into ice and extracted with EtOAc. The organic layer was extracted with 1 N NaOH; the separated aqueous layer was acidified with HCl and extracted with EtOAc. The combined organic layers were dried on Na₂SO₄ and evaporated. The crude mixture was triturated with Acute or methylene chloride to give pure benzofuranones. This procedure was used to obtain the following compounds:

### 6-Hydroxy-4-methyl-benzofuran-3-one (C)

Yield: 17%. MS (m/z): 165.1 (MH⁺).

### 6-Hydroxy-5-methyl-benzofuran-3-one (D)

Yield: 69%. MS (m/z): 165.1 (MH⁺).

### 6-Hydroxy-7-methyl-benzofuran-3-one (E)

Yield: 22%. MS (m/z): 165.2 (MH⁺).

### 4-Fluoro-6-hydroxy-benzofuran-3-one (F)

Yield: 27%. MS (m/z): 169.1 (MK⁺)

### 5-Fluoro-6-hydroxy-benzofuran-3-one (G)

Yield: 28%. MS (m/z): 169.1 (MH⁺).

### 7-Fluoro-6-hydroxy-benzofuran-3-one (H)

Yield: 29%. MS (m/z): 169.2 (MH⁺).

4-Chloro-6-hydroxy-benzofuran-3-one (I)

Yield: 9%. MS (m/z): 185.1 (MH⁺).

### 5-Chloro-6-hydroxy-benzofuran-3-one (L)

Yield: 38%. MS (m/z): 185.1 (MH⁺).

### 7-Chloro-6-hydroxy-benzofuran-3-one (M)

Yield: 30%. MS (m/z): 185.3 (MH⁺).

### 5-Bromo-6-hydroxy-benzofuran-3-one (N)

Yield: 51%. MS (m/z): 228.9 (MH⁺).

### 4-Bromo-6-hydroxy-benzofuran-3-one (O)

Yield: 20%. MS (m/z): 229.0 (MH⁺).

### Preparation of 4,6-dimethoxybenzofuran-3(2H)-one (Compound P)

To a mixture of 3,5-dimethoxyphenol (47.1 g, 306 mmol), 2-chloroacetonitrile (23.07 g, 306 mmol) and zinc chloride (22.90 g, 168 mmol) in ether (450 mL) was bubbled thru Hydrochloric acid gas over 2 hours. An oil separates, this mixture was allowed to stir overnight. The ether was decanted from the now solidified oil, the solid rinsed with fresh ether and the ether decanted. To the solid was added 400 mL of water and the mixture boiled for 1 hour, cooled to room temperature, filtered, washed with water. The solid was mixed with 50 grams of sodium acetate and 400 mL ethanol and the mixture refluxed for 5 hours and cooled. The solid was collected and washed with ethanol. The solid was washed with dichloromethane. The washes were evaporated and the solid isolated with ethyl acetate to give 4,6-dimethoxybenzofuran-3(2H)-one (7.85 g, 40.4 mmol, 13.23% yield).

### Preparation of 7-bromo-4-methoxybenzofuran-3(2H)-one (Compound Q)

To a solution of 1-(3-bromo-2-hydroxy-6-methoxyphenyl)ethanone (6.49 g, 26.5 mmol) in triethylamine (17 mL) and dichloromethane (120 mL) was added TBSCl (4.29 g, 28.5 mmol). This solution was stirred overnight. Reaction mixture was evaporated in-vacuo and treated with 150 mL water, stirred 1 hour, extracted with ether (3×75 mL). The combined ether extracts were combined, washed with 2N hydrochloric acid, water, dried over sodium sulfate, filtered, evaporated and the resulting semi-solid 1-[3-bromo-2-(tert-butyldimethylsilyloxy)-6-methoxyphenyl]ethanone (9.35 g, 26.0 mmol, 98% yield), used as is in the next step.

To a solution of 1-(3-bromo-2-(tert-butyldimethylsilyloxy)-6-methoxyphenyl)ethanone (9.35 g, 26.0 mmol) in TEA (17 mL) and dichloromethane (120 mL) was added TMSOTf (5.64 mL, 31.2 mmol), cooled with an ice bath. This solution was stirred overnight and allowed to warm to room temperature. Chloroform was added, 120 mL, and the mixture extracted with brine (2×150 mL). The organic layer was dried over sodium sulfate, filtered and evaporated to give a dark brown semi-solid, placed under high-vacuum to remove volatiles, 1-[3-bromo-2-(tert-butyldimethylsilyloxy)-6-methoxyphenyl]vinyloxytrimethylsilane (12.18 g, 26.0 mmol, 100% yield), assumed to be 92% pure, used as is for the next step.

To a solution of 1-[3-bromo-2-(tert-butyldimethylsilyloxy)-6-methoxyphenyl]vinyloxytrimethylsilane (12.18 g, 26.0 mmol) in carbon tetrachloride (120 mL), (some dark oil does not dissolve) cooled in an ice-bath, was added bromine (1.512 mL, 29.3 mmol) in 25 mL carbon tetrachloride in drops over 15 minutes. This was stirred at ice bath temp for 30 minutes then the ice bath was removed and the reaction allowed to warm to room temperature. Reaction mixture was treated with 200 mL water, layers separated. Aqueous extracted with concentrated hydrochloric acid (2×50 mL). Combined organic layers washed with aqueous Na₂S₂O₃, dried over sodium sulfate, filtered thru a little Magnesol™, evaporated to give an orange oil, 11.38 g, 2-bromo-1-[3-bromo-2-(tert-butyldimethylsilyloxy)-6-methoxyphenyl]ethanone, used as is in the next step.

To a solution of 2-bromo-1-[3-bromo-2-(tert-butyldimethylsilyloxy)-6-methoxyphenyl]ethanone (11.38 g, 26.0 mmol) in tetrahydrofuran (100 mL), cooled in an ice-bath, was added tetrabutylammonium fluoride (29 ml, 29.0 mmol) (1M in tetrahydrofuran). This was stirred at ice bath temp for 10 minutes then the ice bath was removed and the reaction allowed to warm to room temperature, stirred for 30 minutes. Reaction mixture was quenched with 30 mL saturated ammonium chloride solution. The tetrahydrofuran was removed in-vacuo; water and ether were added. The aqueous layer was extracted with ether (2×25 mL). Combined ether layers washed with water, brine, dried over sodium sulfate, filtered and evaporated to give a yellow residue, purified by chromatography using a hexane-ethyl acetate gradient the product peak was collected, evaporated and the solid isolated with 1:1 hexanes-ethyl acetate, washed with fresh solvent and dried to give a pale yellow solid, 7-bromo-4-methoxybenzofuran-3(2H)-one (587 mg, 9.30% yield).

### Preparation of 6-hydroxy-4-methoxybenzofuran-3(2H)-one (Compound R)

A mixture of 5-methoxybenzene-1,3-diol (10.05 g, 71.7 mmol), 2-chloroacetonitrile (5.41 g, 71.7 mmol), zinc chloride (5.38 g, 39.4 mmol) and ether (100 ml) was stirred in a 500 mL 3N Morton flask. Dry hydrogen chloride gas was bubbled through, solids dissolved and were replaced by a dark oil. After an hour of bubbling hydrochloric acid gas thru the mixture the oil became a salmon-colored solid. Hydrochloric acid gas is bubbled through for an additional hour. The mixture was stirred overnight. The mixture was filtered, and the flask rinsed with ether and this ether was used as a wash. Any solids remaining in the flask are left there. The solids were transferred back to the flask and treated with 100 mL of 2N hydrochloric acid and the mixture stirred and brought to reflux. All solids dissolved after heating for a while some solid precipitates. Heated for 2 hours and cooled, the salmon colored solid collected and washed well with water and dried, 9.73 g. A one gram portion of this was purified by chromatography using a hexane-ethyl acetate gradient; the product peak was collected, evaporated to give a yellow solid, 180 mg, MS (m/z) 181.2 (MH⁺), used as is for the next step.

### Preparation of 6-bromo-1-benzofuran-3(2H)-one (Compound S)

To a stirred solution of boron trichloride in methylene chloride (1.0 M, 6 mL, 6.0 mmol) at 0° C. was added a mixture of 3-bromophenol (870 mg, 5 mmol) in 2 mL of methylene chloride followed by chloroacetonitrile (0.38 mL, 6 mmol) and aluminum chloride (334 mg, 2.5 mmol). The mixture was stirred at room temperature for 20 hours. Then, ice and hydrochloric acid (2N, 4 mL, 8 mmol) were added and the mixture was stirred for 30 minutes. The mixture was extracted with methylene chloride (×3) and the organic layer was washed with saturated sodium chloride solution, dried over magnesium sulfate, and concentrated. The residue was purified by chromatography over silica, eluting with hexanes to 5% ethyl acetate in hexanes. The desired 1-(4-bromo-2-hydroxyphenyl)-2-chloroethanone was obtained as a mixture with the starting material 3-bromophenol, and was used without further purification. MS (m/z): 246.9 (MH−).

The crude product in the previous step was dissolved in 20 mL of acetonitrile and 3 mL of triethylamine was added. The mixture was stirred at room temperature for 40 minutes, and concentrated. The residue was purified by chromatography over silica, eluting with hexanes to 2% ethyl acetate in hexanes. The desired 6-bromo-1-benzofuran-3(2H)-one was obtained as a yellow solid (350 mg). MS (m/z): 213.0 (MH⁺).

### A-II. Synthesis of Indole-3-carbaldehyde Intermediates

### Preparation of 5-methoxy-2-phenyl-1H-indole-3-carbaldehyde

POCl₃ (2.05 mL, 22 mmol, 1.1 eq) was added to DMF (7.74 mL, 5 eq) at 0° C. Let stir 30 minutes. The Vilsmeier-Haack reagent was added to a stirring solution of 2-phenyl-5-methoxyindole (4.47 g, 20 mmol, 1 eq) in DMF (15 mL) at 5° C. Stirred in ice water bath 30 minutes, then let reaction warm to ambient temperature. The reaction was poured onto ice and basified to pH 10 with 5N aqueous NaOH solution. The reaction was heated to boiling then allowed to cool and acidified to pH 4 with 2N aqueous HCl solution. The resulting precipitate was filtered to isolate title compound as a solid dried in vacuo.

### Preparation of 5-Methoxy-2-methyl-1-(2-(4-methylpiperazin-1-yl)ethyl)-1H-indole-3-carbaldehyde

### Step 1)

### 5-methoxy-2-methyl-1H-indole-3-carbaldehyde

POCl₃ (2.05 mL, 22 mmol, 1.1 eq) was added to DMF (7.74 mL, 5 eq) at 0° C. Let stir 30 minutes. The Vilsmeier-Haack reagent was added to a stirring solution of 2-methyl-5-methoxyindole (3.22 g, 20 mmol, 1 eq) in DMF (15 mL) at 5° C. Stirred on ice water bath 30 minutes, then let reaction warm to ambient temperature. The reaction was poured onto ice and Basified to pH 10 with 5N aqueous NaOH solution. The mixture was heated to boiling and the allowed to cool. The mixture was acidified to pH 4 with 2N aqueous HCl solution and the resulting precipitate formed filtered to isolate the title compound as a solid.

### Step 2

### 1-(2-chloroethyl)-5-methoxy-2-methyl-1H-indole-3-carbaldehyde

To 5-methoxy-2-methyl-1H-indole-3-carbaldehyde (1.0 g, 5.7 mmol) in DMF (100 mL) cooled to 0 C was added NaH (0.46 g of 60% dispersion in mineral oil, 11.4 mmol, 2 eq.). The resulting suspension was stirred for 15 minutes followed by addition of 1-bromo-2-chloro-ethane (2.4 mL, 29 mmol, 5 eq.). The ice was removed and the mixture stirred overnight at room temperature. The reaction was quenched with the addition of water (50 mL), extracted with EtOAc (100 mL), washed with water (50 mL) and brine (50 mL) and dried (Na₂SO₄) and concentrated in vacuo. Silica gel chromatography (5:5 Hex:EtOAc) afforded 0.28 g of the title compound as a white solid.

### Step 3

### 5-Methoxy-2-methyl-1-(2-(4-methylpiperazin-1-yl)ethyl)-1H-indole-3-carbaldehyde

To 1-(2-chloroethyl)-5-methoxy-2-methyl-1H-indole-3-carbaldehyde (60 mg, 0.24 mmol) in acetonitrile (5 mL) was added K₂CO₃ (165 mg, 1.2 mmol, 5 eq.), KI (99 mg, 0.6 mmol, 2.5 eq.), and N-Methyl piperazine (86 μL, 0.95 mmol, 4 eq.). The resulting suspension was heated to 90 C and stirred for 48 hrs. To the reaction mixture was added water (10 mL) and EtOAc (10 mL). The layers were separate and the aqueous layer washed with EtOAc (20 mL). Combination of the organic layers followed by drying (Na₂SO₄) and concentration in vacuo afforded the crude product used directly in the next reaction.

### Preparation of 4-bromo-1-methyl-H-indole-3-carbaldehyde

A mixture of 3 g (13.38 mmol) of 4-bromo-3-formylindole, and 482.9 mg (20.1 mmol) of sodium hydride was stirred in N,N-dimethylformamide (30 mL) at 0° C. until no more gas evolved. Then 1.25 mL (20.1 mmol) of methyl iodide was added into the mixture, and let it warm up to room temperature overnight. To the mixture was added a solution of ethyl acetate and ether (1:1). The organic layer was washed five times with brine, dried over sodium sulfate and evaporated to give a pink solid 2.8 g (88% yield). MS (m/z) 238.1 (MH⁺).

### Preparation of 4-(4-isopropoxy-phenyl)-1-methyl-1H-indole-3-carboxylaldehyde

A mixture of 300 mg (1.26 mmol) of 4-bromo-1-methyl-H-indole-3-carbaldehyde, 340.2 mg (1.89 mmol) of isopropoxyphenylboronic acid, 145.6 mg (0.126 mmol) of tetrakis (triphenylphosphine)palladium(0), and saturated aqueous sodium carbonate (1 mL), was placed in a microwave vial. To the mixture was added 3 mL of 1,2-dimethoxyethane. The sealed tube was heated by microwave for twenty minutes at 120° C. After cooling, the mixture was filtered through Celite™ and washed with ethyl acetate. After the solvent was evaporated, the residue was purified by column chromatography (70% ethyl acetate in hexane) to give 283 mg of 4-(4-isopropoxy-phenyl)-1-methyl-1H-indole-3-carboxylaldehyde as a light brown solid (77% yield). MS (m/z) 294.4 (MH⁺).

### Preparation of 4-bromo-1-(2-chloroethyl)-1H-indole-3-carbaldehyde

A mixture of 5 g (22.23 mmol) of 4-bromo-3-formylindole (Frontier), and 1.6 g (66.69 mmol) of sodium hydride was stirred in N,N-dimethylformamide (60 mL) at 0° C. until no more gas evolved. Then, 4.1 mL (44.46 mmol) of 1-chloro-2-iodoethane was added into the mixture, and let it warm up to room temperature overnight. To the mixture was added a solution of ethyl acetate. The organic layer was washed five times with brine, dried over sodium sulfate and evaporated to give a off white solid. The solid was purified by column chromatography to give 2.4 g of 4-bromo-1-(2-chloroethyl)-1H-indole-3-carbaldehyde (38% yield). MS (m/z) 287.55 (MH⁺).

### Preparation of 4-bromo-1-[2-(4-methylpiperizin-1-yl)ethyl]-1H-indole-3-carbaldehyde

A mixture of 2 g (7.0 mmol) of 4-bromo-1-(2-chloroethyl)-1H-indole-3-carbaldehyde, 3.1 mL (28 mmol) of 1-methylpiperazin, 2.1 g (14.0 mmol) of sodium iodide and 2.39 g (7.0 mmol) of tetrabutylammonium iodide was stirred in 20 mL of 1-methylpyrrolidinone at 80° C. for two hours. After cooling the mixture to room temperature, 30 mL of water was added and made basic with saturated potassium carbonate. The solution was extracted three times with methylene chloride, dried over sodium sulfate, and evaporated. The product was purified by column chromatography (20% methanol:methylene chloride) to give 1.6 g of 4-bromo-1-[2-(4-methylpiperizin-1-yl)ethyl]-1H-indole-3-carbaldehyde as a yellow oil (67% yield). MS (m/z) 351.25 (MH⁺).

### Preparation of 3-formyl-1-methyl-2-phenyl-1H-indole-4-carbonitrile

### Step 1

4-Cyanoindole (5.0 g, 35.2 mmol) was dissolved in 70 mL DMF and cooled to 0° C. 60% sodium hydride (2.1 g, 52.8 mmol) was added in portions and let react for 30 minutes. Iodomethane (4.4 mL, 70.4 mmol) was added and let warm to room temperature. The reaction was then quenched with cold water and extracted with ethyl acetate 3 times. The organics were washed with brine, dried over magnesium sulfate and concentrated in vacuo. The residue was filtered and dried to afford 1-methyl-1H-indole-4-carbonitrile (5.2 g, 33.3 mmol, 95% yield).

### Step 2

In a 25 mL round bottom flask was combined 1-methyl-1H-indole-4-carbonitrile (0.41 g, 2.6 mmol), triphenylphosphine (14 mg, 0.052 mmol), palladium II acetate (30 mg, 0.13 mmol), cesium acetate (1.04 g, 5.2 mmol), iodobenzene (0.35 mL, 3.12 mmol) in 1.5 mL N,N-dimethylacetamide. The reaction mixture was heated to 125° C. for 24 hours. The black mixture was diluted with dichloromethane, filtered through Celite™, concentrated and purified on a 40 g ISCO silica column using 20% ethyl acetate:hexane gradient. Combined desired fractions, concentrated in vacuo to afford 0.21 g (0.90 mmol, 35% yield) of 1-methyl-2-phenyl-1H-indole-4-carbonitrile. MS (m/z) 233.4 (MH⁺).

### Step 3

In an oven-dried 3 neck round bottom flask equipped with N2 and thermocouple was charged DMF (0.31 mL, 3.96 mmol) and was cooled to 0° C. POCl₃ (0.092 mL, 0.99 mmol) was added by drops, while keeping the temperature below 5° C. 1-Methyl-2-phenyl-1H-indole-4-carbonitrile (0.21 g, 0.9 mmol) was dissolved in 3 mL DMF and added by drops to the reaction mixture. This was heated to 35 C for 2 hours. The reaction was cooled to room temp, then quenched with ice. Solids formed which were filtered and dried in vacuo to afford 0.153 g (0.588 mmol, 66% yield) of 3-formyl-1-methyl-2-phenyl-1H-indole-4-carbonitrile. MS (ESI): MS (m/z) 261.3 (MH⁺).

### Synthesis of 5-methoxy-indole-3-carbaldehydes

### Preparation of 5-methoxy-indole-3-carbaldehyde, 5-methoxy-2-methyl-indole-3-carbaldehyde, and 3-formyl-5-methoxy-indole-2-carboxylic acid

POCl₃ (1.6 mL, 17 mmol, 1.1 eq.) was added to DMF (6 mL) at 0° C. and the solution was stirred for 30 minutes. This mixture was added to a stirring solution of the selected 5-methoxy-indole (15.5 mmol, 1 eq.) in DMF (11.5 mL) at 0° C. The resulting mixture was stirred at 0° C. for 30 minutes, then allowed to warm to room temperature. The reaction was poured into ice, basified to pH 10 with 5 N NaOH, warmed to room temperature, refluxed for 5 minutes and allowed to cool to rt. Finally, it was acidified to pH 4 with 2 N HCl and the resulting precipitate was filtered and washed with water until pH 7. The solid product was dried under vacuum.

### 5-Methoxy-indole-3-carbaldehyde

Yield: 85%. MS (m/z): 176.2 (MH⁺).

5-Methoxy-2-methyl-indole-3-carbaldehyde

Yield: 94%. MS (m/z): 190.2 (MH⁺).

### 3-Formyl-5-methoxy-indole-2-carboxylic Acid

Yield: 98%. MS (m/z): 220.3 (MH⁺).

### Preparation of 3-formyl-5-methoxy-indole-2-carboxylic Acid Dimethylamide

CDI (0.55 g, 3.4 mmol, 1.3 eq.) was added to a solution of 5-methoxy-indole-2-carboxylic acid (0.5 g, 2.6 mmol, 1.0 eq.) in methylene chloride (10 mL) at 0° C. The reaction mixture was stirred for 30 minutes, then dimethylamine (3 mL of 28% solution in THF, ˜10 eq.) was added. The reaction mixture was stirred at room temperature in a sealed tube overnight, then water was added. The aqueous layer was separated and extracted with methylene chloride. The combined organic layers were washed with saturated NaHCO₃ and brine, dried on Na₂SO₄ and evaporated to give 5-methoxy-indole-2-carboxylic acid dimethylamide. Yield: 75%. MS (m/z): 219.3 (MH⁺).

Phosphorus tribromide (155 mg, 0.57 mmol, 2.5 eq.) was added by drops to a solution of dry DMF (39 mg, 0.68 mmol, 3 eq.) in dry methylene chloride (1 mL) at 0° C. The mixture was stirred at 0° C. for 1 hour and a pale yellow suspension formed. A solution of 5-methoxy-indole-2-carboxylic acid dimethylamide (50 mg, 0.23 mmol) in dry methylene chloride (1 mL) was added and the resulting mixture was refluxed for 3 hours. The reaction mixture was poured into ice and neutralized with NaHCO₃. The aqueous layer was separated and extracted with methylene chloride. The combined organic layers were dried on Na₂SO₄. Evaporation of the solvent afforded the crude product that was purified by silica gel column chromatography (eluent: CHCl₃/MeOH 98:2). Yield: 44%. MS (m/z): 247.3 (MH⁺).

### Preparation of 5-methoxy-2-cyclopropyl-indole-3-carbaldehyde

A solution of 4-methoxy-2-methylaniline (10 g, 72.9 mmol, 1 eq.) and tert-butyl dicarbonate (18.3 g, 84.8 mmol, 1.2 eq.) in THF (90 mL) was refluxed for 2 hours. After cooling, the reaction mixture was evaporated under reduced pressure and the residue was dissolved in EtOAc. The organic layer was washed with a saturated NH₄Cl and brine, dried on Na₂SO₄ and evaporated to give crude N-(tert-butoxycarbonyl)-4-methoxy-2-methylaniline that was used without further purification. Yield: quant. MS (m/z): 238.9 (MH⁺).

Et₃N (3.3 mL) was added to a solution of MeNH(OMe).HCl (1.2 g, 12.4 mmol, 1 eq.) in methylene chloride (35 mL). The solution was stirred at room temperature for 30 minutes, then the reaction was cooled to 0° C. and cyclopropanecarbonylchloride (1 g, 12.4 mmol, 1 eq.) was added. After 5 hours, the reaction mixture was diluted with methylene chloride, washed with 1 N HCl and saturated NaHCO₃. The organic layer was dried on Na₂SO₄ and evaporated to give crude N-methoxy-N-methylcyclopropanecarboxamide, which was utilized in the next step without further purification. Yield: 94%.

A solution of N-(tert-Butoxycarbonyl)-4-methoxy-2-methylaniline (2.7 g, 11.6 mmol) in THF (34 mL) was cooled to −78° C. under N2 and sec-BuLi (1.3 M in cyclohexane, 17.9 mL, 23.2 mmol) was added slowly keeping the temperature below −40° C. After 15 minutes, a solution of N-methoxy-N-methylcyclopropanecarboxamide (1.5 g, 11.6 mmol) in THF (34 mL), was added by drops. The reaction mixture was stirred for 1 hour, then the cooling bath was removed and the mixture was stirred for additional 1 hour. The reaction was poured into a mixture of Et₂O and 1 N HCl. The organic layer was separated, washed with water, dried on Na₂SO₄ and evaporated under reduced pressure to give crude t-butyl-2-(2-cyclopropyl-2-oxoethyl)-4-methoxyphenyl carbamate. The desired compound was purified by flash chromatography. Yield: 61%. MS (m/z): 306.3 (MH⁺).

A solution of t-butyl-2-(cyclopropyl-2-oxopropyl)-4-methoxyphenylcarbamate (1.5 g, 4.9 mmol) and trifluoroacetic acid (5 mL) in methylene chloride (25 mL) was stirred for 4 hours. Water was added and the organic layer separated, dried on Na₂SO₄ and evaporated to give 5-methoxy-2-cyclopropyl-indole. Yield: 69%.

For the formylation step, the same procedure described for 5-methoxy-indole-3-carbaldehyde and 5-methoxy-2-methyl-indole-3-carbaldehyde was used. Yield: 95%. MS (m/z): 216.2 (MH⁺).

### Preparation of 5-methoxy-2-trifluoromethyl-indole-3-carbaldehyde

A solution of N-(tert-butoxycarbonyl)-4-methoxy-2-methylaniline (2.6 g, 11 mmol) in THF (34 mL) was cooled to −78° C. and sec-BuLi (1.4 M in cyclohexane, 17.1 mL, 24 mmol, 2.2 eq.) was slowly added, keeping the temperature below −40° C. After 15 minutes, a solution of ethyl trifluoroacetate (1.56 mL, 13.1 mmol, 1.2 eq) in THF (34 mL) was by drops added. The cooling bath was removed and the mixture was stirred for 3 hours. The reaction was poured into a mixture of Et₂O and 1 N HCl. The organic layer was separated, washed with water, dried on Na₂SO₄ and evaporated under reduced pressure to give crude tert-butyl 2-(3,3,3-trifluoro-2-oxopropyl)-4-methoxyphenylcarbamate that was used in the following step without further purification. Yield: 92%.

A solution of tert-butyl 2-(3,3,3-trifluoro-2-oxopropyl)-4-methoxyphenylcarbamate (1.34 g, 4.9 mmol) and trifluoroacetic acid (5 mL) in methylene chloride (25 mL) was stirred for 24 hours. Water was added and the organic layer was separated, dried on Na₂SO₄ and evaporated to give 2-trifluoromethyl-5-methoxy-indole. Yield: 70%.

For the formylation step, the classical Vilsmeier-Haack procedure with POCl₃ was used performing the reaction at 50° C. A mixture of indole-3-carboxaldehyde and indole-4-carboxaldehyde formed. The title compound was isolated by trituration with Et₂O. Both the isomers were characterized:

### 2-(Trifluoromethyl)-5-methoxy-indole-3-carbaldehyde

MS (m/z): 244.3 (MH⁺).

### 2-(Trifluoromethyl)-5-methoxy-indole-4-carbaldehyde

MS (m/z): 244.3 (MH⁺).

### Preparation of 5-methoxy-2-(1-methyl-1H-pyrazol-4-yl)-indole-3-carbaldehyde

5-Methoxy isatin (0.2 g, 1.1 mmol, 1 eq.) was dissolved in hydrazine hydrate (1.2 mL, 38 mmol, 34 eq.) and refluxed for 15 minutes. The reaction mixture was poured into cold water and extracted with EtOAc. The combined organic extracts were dried on Na₂SO₄. The solvent was evaporated to afford crude 5-methoxy-1,3-dihydro-indol-2-one that was purified by silica gel column chromatography (eluent: hexane/EtOAc from 10:0 to 6:4). Yield: 27%. MS (m/z): 164.2 (MH⁺).

Phosphorous oxybromide (0.35 mL, 3.1 mmol, 2.5 eq.) was added drop wise to a solution of DMF (0.3 mL, 3.7 mmol, 3 eq.) in dry methylene chloride at 0° C. The mixture was stirred at 0° C. for 30 minutes, then a solution of 5-methoxy-1,3-dihydro-indol-2-one (0.2 g, 1.2 mmol, 1 eq.) in dry methylene chloride (2 mL) was added and the mixture was refluxed for 3 hours. The solution was neutralized with solid NaHCO₃ and extracted with methylene chloride. The organic layer was dried on Na₂SO₄ and evaporated under reduced pressure. The crude mixture was purified by silica gel column chromatography (eluent: hexane/AcOEt 6:4 to 4:6) to give pure 2-bromo-5-methoxy-indole-3-carbaldehyde. Yield: 45%. MS (m/z): 254.1 (MH⁺).

A stirred solution of 2-bromo-5-methoxy-indole-3-carbaldehyde (2.0 g, 7.9 mmol, 1 eq.) in DME (2 mL) was deoxygenated by bubbling argon for 10 minutes at rt. Pd(PPh₃)₄ (0.9 g, 0.8 mmol, 0.1 eq.) was added followed by a solution of 1-methyl-4-(4,4,5,5-tetramethyl-[1,3,2]dioxaborolan-2-yl)-1H-pyrazole (2.4 g, 11.63 mmol, 1.48 eq.) in ethanol (2.5 mL). 2M Na₂CO₃ (33 mL, 8.5 eq.) was also deoxygenated with argon and added. The resulting mixture was heated at 78° C. for 18 hours. The reaction mixture was cooled to room temperature, quenched with water, and extracted with methylene chloride. Organic layer was dried on anhydrous Na₂SO₄ and evaporated under reduced pressure to give the crude product 1f. Yield: 89%. MS (m/z): 256.1 (MH⁺).

### 2-(3,5-Dimethyl-isoxazol-4-yl)-5-methoxy-indole-3-carbaldehyde

The compound was obtained with the same Suzuki coupling described with 1f, (3,5-dimethyl-4-(4,4,5,5-tetramethyl-[1,3,2]dioxaborolan-2-yl)-isoxazole was used as boronic reagent).

The crude product was purified by silica gel column chromatography (eluent: AcOEt/hexane 1:1). Yield: 57%. MS (m/z): 271.3 (MH⁺).

### Preparation of 5-methoxy-2-pyrimidin-5-yl-indole-3-carbaldehyde

To a stirred solution of Pd(PPh₃)₄ (0.818 g, 0.7 mmol, 0.1 eq.) in propanol (5 mL), deoxygenated 2M Na₂CO₃ (4.2 mL, 8.5 mmol, 1.2 eq.) was added and the resulting mixture was stirred for 10 minutes at room temperature under argon atmosphere. 2-Bromo-5-methoxy-indole-3-carbaldehyde (1.80 g, 7.08 mmol, 1 eq.) and 5-pyrimidinyl boronic acid (1.05 g, 8.5 mmol, 1.2 eq.) in 1-propanol (20 mL) were added and the reaction mixture was stirred for 10 minutes. The temperature was slowly raised to 80° C. and the reaction was stirred overnight. The reaction mass was cooled to room temperature, quenched with water and extracted with EtOAc. The organic layer was washed with 5% NaHCO₃ solution, brine, and dried on anhydrous Na₂SO₄. Evaporation of the solvent afforded a crude mixture that was purified by silica gel column chromatography (eluent: CHCl₃/MeOH 100:0 to 95:5). Yield: 50%. MS (m/z): 254.1 (MH⁺).

### Preparation of 5-methoxy-2-phenyl-indole-3-carbaldehyde

A solution of p-anisidine (3 g, 24 mmol, 1 eq.) and 2-bromoacetophenone (4.8 g, 24 mmol, 1 eq.) in DMA (5 mL) was heated at 170° C. with microwave irradiation for 1 hour. The reaction mixture was diluted with methylene chloride and washed with 2 N HCl. The organic layer was dried on Na₂SO₄ and evaporated. The crude mixture was filtered on a pad of silica gel (methylene chloride as eluent) and the obtained product was triturated with Et₂O. 5-Methoxy-2-phenylindole was obtained as a white solid. Yield: 40%. MS (m/z): 224.3 (MH⁺).

For the formylation step, the same procedure described for 5-methoxy-indole-3-carbaldehyde and 5-methoxy-2-methyl-indole-3-carbaldehyde was used.

### Preparation of 5-methoxy-2-(4-methyl-piperazine-1-carbonyl)-indole-3-carbaldehyde

To a stirred solution of 5-methoxy-indole-2-carboxylic acid (0.3 g, 1.56 mmol, 1.0 eq.) in methylene chloride (10 mL) at 0° C., EDCI (0.36 g, 1.88 mmol, 1.2 eq.) and HOBT (0.23 g, 1.72 mmol, 1.1 eq.) were added. The mixture was stirred for 30 minutes, then N-methyl-piperazine (0.18 g, 1.88 mmol, 1.2 eq.) was added. The reaction was stirred at room temperature overnight, water was added, and organic layer was separated. The organic layer was washed with saturated NaHCO₃ and brine, dried on Na₂SO₄ and evaporated to give (5-methoxy-indol-2-yl)-(4-methyl-piperazin-1-yl)-methanone. Yield: 70%. MS (m/z): 274.4 (MH⁺).

Classical Vilsmeier-Haack conditions were used on (5-methoxy-indol-2-yl)-(4-methyl-piperazin-1-yl)-methanone. Yield: 63%. MS (m/z): 302.2 (MH⁺).

### Preparation of 5-methoxy-2-(4-methyl-piperazin-1-ylmethyl)-indole-3-carbaldehyde

To a suspension of LiAlH₄ (0.15 g, 3.7 mmol, 3.7 eq.) in THF (10 mL), (5-methoxy-indol-2-yl)-(4-methyl-piperazin-1-yl)-methanone (0.50 g, 1.0 mmol) was added at 5° C. The resulting mixture was stirred for 3 hours, then it was quenched with saturated ammonium chloride solution and filtered. The filtrate was extracted with EtOAc. The organic layer was dried on Na₂SO₄ and evaporated. The crude product was purified by silica gel column chromatography (eluent: CHCl₃/MeOH 98:2). Yield: 85%. MS (m/z): 260.1 (MH⁺).

A solution of POCl₃ (1.18 g, 7.7 mmol, 5 eq.) in DMF (0.56 g, 7.7 mmol, 5 eq.) was stirred for 30 minutes at 0° C. 5-methoxy-2-(4-methyl-piperazin-1-ylmethyl)-indole (0.40 g, 1.5 mmol, 1 eq.) was added at 0° C. and the resulting mixture was stirred for 6 hours at room temperature. The reaction was quenched with ice, basified with NaOH to pH 9, and extracted with methylene chloride. The organic layer was dried on Na₂SO₄ and evaporated to give crude 5-methoxy-2-(4-methyl-piperazin-1-ylmethyl)-indole-3-carbaldehyde 1k. Yield: 95%. MS (m/z): 288.2 (MH⁺).

### Preparation of 2-dimethylaminomethyl-5-methoxy-indole-3-carbaldehyde

To a suspension of LiAlH₄ (1.03 g, 27.4 mmol, 10 eq.) in THF (20 mL), 5-methoxy-indole-2-carboxylic acid dimethylamide (0.60 g, 2.7 mmol, 1 eq.) was added at room temperature. The resulting mixture was stirred for 1 hour, then it was quenched with saturated ammonium chloride solution and filtered. The filtrate was extracted with EtOAc. The organic layer was dried on Na₂SO₄ and evaporated to give (5-methoxy-indol-2-ylmethyl)-dimethyl-amine. Yield: 90%. MS (m/z): 205.2 (MH⁺).

A solution of POCl₃ (0.93 g, 5.9 mmol, 5.9 eq.) in DMF (0.28 g, 4.9 mmol, 4.9 eq.) was stirred for 30 minutes at 0° C. To this solution, (5-methoxy-indol-2-ylmethyl)-dimethyl-amine (0.20 g, 1.0 mmol, 1 eq.) was added at 0° C. and the resulting mixture was stirred at room temperature overnight. The reaction was quenched with ice, basified with NaOH to pH 9, and extracted with methylene chloride. The organic layer was dried on Na₂SO₄ and evaporated to give the crude product that was purified by silica gel column chromatography (eluent: CHCl₃/MeOH 98:2). Yield: 95%. MS (m/z): 233.1 (MH⁺).

### Preparation of 5-methoxy-2-(morpholine-4-carbonyl)-indole-3-carbaldehyde

5-Methoxy-2-(morpholine-1-carbonyl)-indole-3-carbaldehyde is synthesized analogously to 1j, using morpholine instead of 1-methylpiperazine. Yield: 76%. MS (m/z): 289.1 (MH⁺).

### Preparation of 5-methoxy-2-(pyrrolidine-1-carbonyl)-indole-3-carbaldehyde

5-Methoxy-2-(pyrrolidine-1-carbonyl)-indole-3-carbaldehyde is synthesized analogously to 1j, using pyrrolidine instead of 1-methylpiperazine. Yield: 74%. MS (m/z): 273.1 (MH⁺).

### Preparation of 2-cyclopentyl-5-methoxy-indole-3-carbaldehyde

2-Cyclopentyl-5-methoxy-indole-3-carbaldehyde is synthesized analogously to 1d, using of cyclopentanecarbonyl chloride instead of cyclopropanecarbonyl chloride. Yield: 87%. MS (m/z): 244.3 (MH⁺).

### Preparation of 2-cyclohexyl-5-methoxy-indole-3-carbaldehyde

2-Cyclohexyl-5-methoxy-indole-3-carbaldehyde is synthesized analogously to 1d, using of cyclohexanecarbonyl chloride instead of cyclopropanecarbonyl chloride. Yield: 93%. MS (m/z): 258.3 (MH⁺).

### Preparation of 2-cyclobutyl-5-methoxy-indole-3-carbaldehyde

2-Cyclobutyl-5-methoxy-indole-3-carbaldehyde is synthesized analogously to 1d, using of cyclobutanecarbonyl chloride instead of cyclopropanecarbonyl chloride. Yield: 67%. MS (m/z): 230.3 (MH⁺).

### Synthesis of N-substituted 5-methoxy-indole-3-carbaldehydes. For the preparation of 5-methoxy-indole-3-carbaldehydes, four general routes (A-D) were used

General Procedure for the Alkylation with 1-(2-chloro-ethyl)-imidazole (Compounds with y=2)

To a solution of the selected 5-methoxy-indole-3-carbaldehyde 1× (5.7 mmol, 1 eq.) in acetonitrile (20 mL), K₂CO₃ (3.9 g, 28.5 mmol, 5 eq.), KI (2.3 g, 14 mmol, 2.5 eq.) and 1-(2-chloro-ethyl)-imidazole (3.0 g, 22.8 mmol, 4 eq.) were added. The resulting suspension was stirred at 90° C. for 24 hours, and then water was added. The aqueous layer was separated and extracted with EtOAc. The combined organic layers were dried on Na₂SO₄ and evaporated. The crude products were further purified as described below. According to this procedure, the following compounds were obtained.

### 1-(2-Imidazol-1-yl-ethyl)-5-methoxy-indole-3-carbaldehyde

Purified by silica gel column chromatography (eluent: CHCl₃/MeOH 95:5). Yield: 40%. MS (m/z): 270.3 (MH⁺).

### 3-Formyl-1-(2-imidazol-1-yl-ethyl)-5-methoxy-1H-indole-2-carboxylic acid dimethylamide

Purified by silica gel column chromatography (eluent: CHCl₃/MeOH 97:3). Yield: 72%. MS (m/z): 341.2 (MH⁺).

General Procedure for the Alkylation with 2-chloro-N,N-dimethyl-acetamide (Compounds with y=5) 60% NaH in mineral oil (2.0 g, 50 mmol, 2.2 eq.) was pre-washed with hexane and suspended in dry DMF (4 mL) under nitrogen. The suspension was cooled with an ice bath and a solution of the selected 5-methoxy-indole-3-carbaldehyde 1× (22 mmol, 1 eq.) in dry DMF (8 mL) was added by drops over 15 minutes. The cooling bath was removed and the mixture was stirred for 30 minutes. The reaction mixture was cooled again and a solution of 2-chloro-N,N-dimethyl-acetamide (5.9 g, 44 mmol, 2 eq.) in dry DMF (8 mL) was added by drops over 10 minutes. The reaction mixture was stirred according to the conditions indicated below. The solvent was evaporated and the residue was partitioned between EtOAc and water. The combined organic layers were washed with water and brine and dried on Na₂SO₄. Evaporation of the solvent afforded a crude mixture that was purified by silica gel column chromatography. According to this procedure, the following compounds were obtained.

### 2-(3-Formyl-5-methoxy-indol-1-yl)-N,N-dimethyl-acetamide

Reaction conditions: room temperature for 18 hours. Purified by silica gel column chromatography (eluent: gradient from CHCl₃ to CHCl₃/MeOH 95:5). Yield: 44%. MS (m/z): 261.1 (MH⁺).

### 2-(3-Formyl-5-methoxy-2-methyl-indol-1-yl)-N,N-dimethyl-acetamide

Reaction conditions: room temperature for 18 hours. Purified by silica gel column chromatography (eluent: gradient from CHCl₃ to CHCl₃/MeOH 95:5). Yield: 82%. MS (m/z): 275.1 (MH⁺).

### 1-Dimethylcarbamoylmethyl-3-formyl-5-methoxy-indole-2-carboxylic acid dimethylamide

Reaction conditions: MW heating (250 W, 20 minutes, 80° C.). Purified by silica gel column chromatography (eluent: gradient from CHCl₃/MeOH 10:0 to 9:1). Yield: 59%. MS (m/z): 332.4 (MH⁺).

### 2-(2-Cyclopropyl-3-formyl-5-methoxy-indole-1-yl)-N,N-dimethyl-acetamide

Reaction conditions: 60° C. for 48 hours. Purified by silica gel column chromatography (eluent: gradient from petroleum ether/EtOAc 1:1 to EtOAc). Yield: 24%. MS (m/z): 301.2 (MH⁺).

### 2-(2-Trifluoromethyl-3-formyl-5-methoxy-indole-1-yl-N,N-dimethyl-acetamide

Reaction conditions: 60° C. for 48 hours. Purified by silica gel column chromatography (eluent: gradient from petroleum ether/AcOEt 5:5 to 0:10). Yield: 58%. MS (m/z): 329.3 (MH⁺).

### 2-[3-Formyl-5-methoxy-2-(1-methyl-1H-pyrazol-4-yl)-indol-1-yl]-N,N-dimethyl-acetamide

Reaction conditions: room temperature for 24 hours. Purified by silica gel column chromatography (eluent: CHCl₃). Yield: 60%. MS (m/z): 341.1 (MH⁺).

General Procedure for the Alkylation with 1-bromo-2-chloroethane

NH (60% dispersion in mineral oil, 1.2 g, 29.2 mmol, 2 eq.) was added to a solution of the selected 5-methoxy-indole-3-carbaldehyde 1× (14.6 mmol, 1 eq.) in DMF (250 mL), cooled to 0° C. The resulting suspension was stirred for 15 minutes, and then 1-bromo-2-chloro-ethane (6.1 mL, 73 mmol, 5 eq.) was added. The ice was removed and the mixture was stirred under the condition indicated below. The reaction was quenched with the addition of water and extracted with EtOAc. The organic layer was washed with water and brine, dried on Na₂SO₄ and evaporated to give a crude mixture that was purified by silica gel column chromatography. According to this procedure, the following compounds were obtained.

### 1-(2-Chloro-ethyl)-5-methoxy-indole-3-carbaldehyde

Reaction conditions: room temperature for 12 hours. Purified by silica gel column chromatography (eluent: CHCl₃). Yield: 56%. MS (m/z): 238.3 (MH⁺).

### 1-(2-Chloro-ethyl)-5-methoxy-2-methyl-indole-3-carbaldehyde

Reaction conditions: 90° C. for 4 days, fresh 1-bromo-2-chloro-ethane (2.5 eq.) added every 12 hours. Purified by silica gel column chromatography (eluent: gradient from hexane:AcOEt 7:3 to hexane/EtOAc 1:1). Yield: 61%. MS (m/z): 252.2 (MH⁺).

### 1-(2-Chloro-ethyl)-3-formyl-5-methoxy-indole-2-carboxylic acid dimethyl amide

Reaction conditions: room temperature for 48 hours. Purified by silica gel column chromatography (eluent: MeOH/CHCl₃ 0.75:99.25). Yield: 60%. MS (m/z): 309.1 (MH⁺).

### 1-(2-Chloro-ethyl)-2-cyclopropyl-5-methoxy-indole-3-carbaldehyde

Reaction conditions: 90° C. for 4 days, fresh 1-bromo-2-chloro-ethane (2.5 eq.) added every 12 hours. Purified by silica gel column chromatography (eluent: methylene chloride/MeOH 98:2). Yield: 13%. MS (m/z): 278.2 (MH⁺).

### 1-(2-Chloro-ethyl)-5-methoxy-2-(morpholine-4-carbonyl)-indole-3-carbaldehyde

Reaction conditions: room temperature for 12 hours. Purified by silica gel column chromatography (eluent: MeOH/CHCl₃ 1:99). Yield: 70%. MS (m/z): 351.2 (MH⁺).

### 1-(2-Chloro-ethyl)-5-methoxy-2-(pyrrolidine-4-carbonyl)-indole-3-carbaldehyde

Reaction conditions: room temperature for 12 hours. Purified by silica gel column chromatography (eluent: MeOH/CHCl₃ 1:99). Yield: 70%. MS (m/z): 335.2 (MH⁺). *Yields were calculated assuming the product as only chloro derivative (the bromo derivative is usually <30%).

### General Procedure for the Nucleophilic Displacement

To a solution of the selected 1-(2-chloro-ethyl)-5-methoxy-indole-3-carbaldehyde 3× (8.6 mmol, 1 eq.) in acetonitrile (180 mL), K₂CO₃ (5.94 g, 43.0 mmol, 5 eq.), KI (3.57 g, 21.5 mmol, 2.5 eq.) and the nucleophile (34.4 mmol, 4 eq.) were added. The resulting suspension was stirred at 90° C. for 48 hours, then water and EtOAc were added. The layers were separated and the aqueous layer was extracted with EtOAc. Combination of the organic layers, followed by drying on Na₂SO₄ and evaporation, afforded the crude product. According to this procedure, the following compounds were obtained.

### 5-Methoxy-1-[2-(4-methyl-piperazin-1-yl)-ethyl]-indole-3-carbaldehyde

Nucleophile: N-methyl-piperazine. Purified by silica gel column chromatography (eluent: CHCl₃/MeOH 98:2). Yield: 51%. MS (m/z): 302.4 (MH⁺).

### 1-[2-(3-Hydroxy-pyrrolidin-1-yl)-ethyl]-5-methoxy-indole-3-carbaldehyde

Nucleophile: pyrrolidin-3-ol. Purified by silica gel column chromatography (eluent: CHCl₃/MeOH 95:5). Yield: 66%. MS (m/z): 289.2 (MH⁺).

### 1-[2-(4-Hydroxy-piperidin-1-yl)-ethyl]-5-methoxy-indole-3-carbaldehyde

Nucleophile: piperidin-4-ol. Purified by silica gel column chromatography (eluent: CHCl₃/MeOH 95:5). Yield: 55%. MS (m/z): 303.4 (MH⁺).

### 5-Methoxy-2-methyl-1-[2-(4-methyl-piperazin-1-yl)-ethyl]-indole-3-carbaldehyde

Nucleophile: N-methyl-piperazine. Purified by silica gel column chromatography (eluent: CH₂Cl₂/MeOH 98:2⁺0.5% NH₃ aq.). Yield: 40%. MS (m/z): 316.2 (MH⁺).

### 1-[2-(4-Hydroxy-piperidin-1-yl)-ethyl]-5-methoxy-2-methyl-indole-3-carbaldehyde

Nucleophile: piperidin-4-ol. Purified by silica gel column chromatography (eluent: gradient from CHCl₃ to CHCl₃/MeOH 95:5). Yield: 47%. MS (m/z): 317.2 (MH⁺).

### 5-Methoxy-2-methyl-1-(2-pyrrolidin-1-yl-ethyl)-indole-3-carbaldehyde

Nucleophile: pyrrolidine. Purified by silica gel column chromatography (eluent: CHCl₃/MeOH 98:2). Yield: 35%. MS (m/z): 287.1 (MH⁺).

### 5-Methoxy-2-methyl-1-(2-piperidin-1-yl-ethyl)-indole-3-carbaldehyde Nucleophile: piperidine. Purified by silica gel column chromatography (eluent: CHCl₃/MeOH 98:2). Yield: 50%. MS (m/z): 301.1 (MH⁺).

### 3-Formyl-5-methoxy-1-[2-(4-methyl-piperazin-1-yl)-ethyl]-indole-2-carboxylic acid dimethylamide

Nucleophile: N-methyl-piperazine. Purified by silica gel column chromatography (eluent: CHCl₃/MeOH 95:5). Yield: 62%. MS (m/z): 373.2 (MH⁺).

### 3-Formyl-1-[2-(3-hydroxy-pyrrolidin-1-yl)-ethyl]-5-methoxy-indole-2-carboxylic dimethylamide

Nucleophile: pyrrolidin-3-ol. Purified by silica gel column chromatography (eluent: CHCl₃/MeOH 95:5). Yield: 86%. MS (m/z): 360.1 (MH⁺).

### 3-Formyl-1-[2-(4-hydroxy-piperidin-1-yl)-ethyl]-5-methoxy-indole-2-carboxylic Acid Dimethylamide

Nucleophile: piperidin-4-ol. Purified by silica gel column chromatography (eluent: CHCl₃/MeOH 95:5). Yield: 69%. MS (m/z): 374.2 (MH⁺).

### 2-Cyclopropyl-5-methoxy-1-(2-(4-methylpiperazin-1-yl)ethyl)-indole-3-carbaldehyde

Nucleophile: N-methyl-piperazine. Purified by silica gel column chromatography (eluent: methylene chloride/MeOH 9:1). Yield: 28%. MS (m/z): 342.5 (MH⁺).

### 5-Methoxy-1-[2-(4-methyl-piperazin-1-yl)-ethyl]-2-(morpholine-4-carbonyl)-indole-3-carbaldehyde

Nucleophile: N-methyl piperazine. Purified by silica gel column chromatography (eluent: CHCl₃/MeOH 94:6). Yield: 45%. MS (m/z): 415.3 (MH⁺).

### 5-Methoxy-1-[2-(4-methyl-piperazin-1-yl)-ethyl]-2-(pyrrolidine-4-carbonyl)-indole-3-carbaldehyde

Nucleophile: N-methyl piperazine. Purified by silica gel column chromatography (eluent: CHCl₃/MeOH 94:6). Yield: 67%. MS (m/z): 399.4 (MH⁺).

General Procedure for the Alkylation with 2-(2-bromo-ethoxy)-tetrahydro-pyran

NaH (1.76 g of 60% dispersion in mineral oil, 44 mmol, 2 eq.) was pre-washed with hexane and suspended in dry DMF (4 mL) under nitrogen. The suspension was cooled with an ice bath and a solution of the selected 5-methoxy-indole-3-carbaldehyde 1× (22 mmol, 1 eq.) in dry DMF (8 mL) was added by drops over 15 minutes. The cooling bath was removed and the mixture was stirred for 30 minutes. The reaction mixture was cooled again and a solution of 2-(2-bromo-ethoxy)-tetrahydro-pyran (6.0 g, 28.6 mmol, 1.3 eq.) in dry DMF (8 mL) was added by drops over 10 minutes. The reaction mixture was stirred according to the conditions indicated below. Then, the solvent was evaporated and the residue was partitioned between EtOAc and water. The combined organic layers were washed with water and brine and dried on Na₂SO₄. Evaporation of the solvent afforded a crude mixture that was purified by silica gel column chromatography. According to this procedure, the following compounds were obtained.

### 5-Methoxy-2-methyl-1-[2-(tetrahydro-pyran-2-yloxy)-ethyl]indole-3-carbaldehyde

Reaction conditions: room temperature for 18 hours. Purified by silica gel column chromatography (eluent: gradient from CHCl₃ to CHCl₃/MeOH 95:5). Yield: 39%. MS (m/z): 318.2 (MH⁺).

### 2-Cyclopropyl-5-methoxy-1-[2-(tetrahydro-pyran-2-yloxy)-ethyl]indole-3-carbaldehyde

Reaction conditions: 60° C. for 48 hours. The crude product was used without further purification. Yield: 76%. MS (m/z): 344.1 (WE).

### 2-(Trifluoromethyl)-5-methoxy-1-(2-(tetrahydro-2H-pyran-2-yloxy)ethyl)-indole carbaldehyde

Reaction conditions: 60° C. for 48 hours. The crude product was used without further purification. MS (m/z): 372.2 (WE).

### 5-Methoxy-2-(1-methyl-1H-pyrazol-4-yl)-1-[2-(tetrahydro-pyran-2-yloxy)-ethyl]-indole-3-carbaldehyde

Reaction conditions: room temperature for 2 days. Purified by silica gel column chromatography (eluent: CHCl₃/MeOH 98:2). Yield: 49%. MS (m/z): 384.2 (MH⁺).

### 2-(3,5-Dimethyl-isoxazol-4-yl)-5-methoxy-1-[2-(tetrahydro-pyran-2-yloxy)-ethyl]indole-3-carbaldehyde

Reaction conditions: room temperature for 2 days. Purified by silica gel column chromatography (eluent: CHCl₃/MeOH 98:2). Yield: 51%. MS (m/z): 399.2 (MH⁺).

### 5-Methoxy-2-pyrimidin-5-yl-1-[2-(tetrahydro-pyran-2-yloxy)-ethyl]-indole-3-carbaldehyde

Reaction conditions: room temperature for 18 hours. The crude product was directly used for the following reaction. Yield: 87%. MS (m/z): 382.3 (MH⁺).

### 2-Cyclopentyl-5-methoxy-1-[2-(tetrahydro-pyran-2-yloxy)-ethyl]indole-3-carbaldehyde

Reaction conditions: 60° C. for 48 hours. The crude product was used without further purification. Yield: 76%. MS (m/z): 372.4 (WE).

### 2-Cyclohexyl-5-methoxy-1-[2-(tetrahydro-pyran-2-yloxy)-ethyl]indole-3-carbaldehyde

Reaction conditions: room temperature for 18 hours. The crude product was directly used for the following reaction. Yield: 87%. MS (m/z): 386.5 (MH⁺).

### 2-Cyclobutyl-5-methoxy-1-[2-(tetrahydro-pyran-2-yloxy)-ethyl]-indole-3-carbaldehyde

Reaction conditions: room temperature for 18 hours. The crude product was directly used for the following reaction. Yield: 87%. MS (m/z): 358.0 (MH⁺).

### General Procedure for the Cleavage of the THP Group

To a solution of the selected 4× (1.5 mmol) in EtOH (10 mL), conc. HCl (0.5 mL) was added. The resulting suspension was stirred for 2 hours, and then water and AcOEt were added. The layers were separated and the aqueous layer was extracted with AcOEt. Combination of the organic layers, followed by drying on Na₂SO₄ and evaporation, afforded the crude product that was further purified as described below. According to this procedure, the following compounds were obtained.

### 1-(2-Hydroxy-ethyl)-5-methoxy-2-methyl-indole-3-carbaldehyde

Purified by trituration with Et₂O. Yield: 85%. MS (m/z): 234.2 (MH⁺).

### 2-Cyclopropyl-1-(2-hydroxy-ethyl)-5-methoxy-indole-3-carbaldehyde

Purified by triturated with Et₂O and silica gel column chromatography (eluent: hexane/EtOAc 1:1). Yield: 45%. MS (m/z): 260.1 (MH⁺).

### 2-(Trifluoromethyl)-1-(2-hydroxyethyl)-5-methoxy-indole-3-carbaldehyde

Purified by silica gel column chromatography (eluent: petroleum ether/AcOEt 8:2). Yield: 37%. MS (m/z): 288.1 (MH⁺).

### 1-(2-Hydroxy-ethyl)-5-methoxy-2-(1-methyl-1H-pyrazol-4-yl)-indole-3-carbaldehyde

Purified by trituration with Et₂O. Yield: 75%. MS (m/z): 300.2 (MH⁺).

### 2-(3,5-Dimethyl-isoxazol-4-yl)-1-(2-hydroxy-ethyl)-5-methoxy-indole-3-carbaldehyde

Purified by trituration with Et₂O. Yield: 85%. MS (m/z): 315.3 (MH⁺).

### 1-(2-Hydroxy-ethyl)-5-methoxy-2-pyrimidin-5-yl-indole-3-carbaldehyde

The crude product was used without further purification. Yield: 89%. MS (m/z): 298.2 (MH⁺).

### 2-Cyclopentyl-1-(2-hydroxy-ethyl)-5-methoxy-indole-3-carbaldehyde

Purified by silica gel column chromatography (eluent: petroleum ether/AcOEt 7:3). Yield (two steps from 1p): 48%. MS (m/z): 288.3 (MH⁺).

### 2-Cyclohexyl-1-(2-hydroxy-ethyl)-5-methoxy-indole-3-carbaldehyde

Purified by silica gel column chromatography (eluent: petroleum ether/AcOEt 7:3). Yield (two steps from 1q): 54%. MS (m/z): 302.4 (WE).

### 2-Cyclobutyl-1-(2-hydroxy-ethyl)-5-methoxy-indole-3-carbaldehyde

Purified by silica gel column chromatography (eluent: petroleum ether/AcOEt 7:3). Yield (two steps from 1r): 42%. MS (m/z): 274.3 (WE).

### General Procedure for the Preparation of the Intermediate Tosyl Esters

To a solution of the selected ester (1.12 mmol, 1 eq.) in dry methylene chloride (10 mL), Et₃N (0.24 mL, 1.7 mmol, 1.5 eq.) and DMAP (catalytic amount) were added at 0° C. After 10 minutes, TsCl (229 mg, 1.2 mmol, 1.07 eq.) was slowly added. The solution was stirred at room temperature overnight, and then the reaction mixture was diluted with methylene chloride and washed with water. The organic layer was dried on Na₂SO₄ and evaporated to give the crude product that was purified as indicated below. According to this procedure, the following compounds were obtained.

### Toluene-4-sulfonic acid 2-(3-formyl-5-methoxy-2-methyl-indol-1-yl)-ethyl ester

Purified by trituration with Et₂O. Yield: 85%. MS (m/z): 388.2 (MH⁺).

### Toluene-4-sulfonic acid 2-(2-cyclopropyl-3-formyl-5-methoxy-indol-1-yl)-ethyl ester

Purified by trituration with Et₂O. Yield: 66%. MS (m/z): 414.3 (MH⁺).

### Toluene-4-sulfonic acid 2-(3-formyl-5-methoxy-2-trifluoromethyl-indol-1-yl)-ethyl ester

The crude product was used without further purification. Yield: 92%. MS (m/z): 442.5 (WE).

### Toluene-4-sulfonic acid 2-[3-formyl-5-methoxy-2-(1-methyl-1H-pyrazol-4-yl)-indol-1-yl]-ethyl Ester

Purified by silica gel column chromatography (eluent: CHCl₃/CH₃OH 98:2). Yield: 57%. MS (m/z): 454.2 (WE).

### Toluene-4-sulfonic acid 2-[2-(3,5-dimethyl-isoxazol-4-yl)-3-formyl-5-methoxy-indol-1-yl]-ethyl Ester

Purified by silica gel column chromatography (eluent: EtOAc/hexane 1:4). Yield: 53%. MS (m/z): 469.3 (WE).

### Toluene-4-sulfonic acid 2-(3-formyl-5-methoxy-2-pyrimidin-5-yl-indol-1-yl)-ethyl ester

Purified by silica gel column chromatography (eluent: MeOH/CHCl₃ 0.5:99.5). Yield: 74%. MS (m/z): 452.2 (MH⁺).

### Toluene-4-sulfonic acid 2-(2-cyclopentyl-3-formyl-5-methoxy-indol-1-yl)-ethyl ester

The crude product was used without further purification. MS (m/z): 442.5 (MH⁺).

### Toluene-4-sulfonic acid 2-(2-cyclohexyl-3-formyl-5-methoxy-indol-1-yl)-ethyl ester

The crude product was used without further purification. MS (m/z): 456.1 (MH⁺).

### Toluene-4-sulfonic acid 2-(2-cyclobutyl-3-formyl-5-methoxy-indol-1-yl)-ethyl ester

The crude product was used without further purification. MS (m/z): 428.4 (MH⁺).

### General Procedures (A-D) for the Nucleophilic Displacement of the Tosylate Compounds

### Procedure A

To a solution of the tosylate (0.74 mmol, 1 eq.) in acetonitrile (15 mL), K₂CO₃ (510 mg, 3.7 mmol, 5 eq.), KI (307 mg, 1.85 mmol, 2.5 eq.) and the selected nucleophile (2.96 mmol, 4 eq.) were added. The resulting suspension was stirred at 90° C. for 48 hours, and then water and EtOAc were added. The layers were separated and the aqueous layer was extracted with EtOAc. Combination of the organic layers, followed by drying on Na₂SO₄ and evaporation, afforded the crude product that was purified as described below. According to this procedure, the following compounds were obtained.

### 2-Cyclopropyl-1-(2-(3-hydroxypyrrolidin-1-ypethyl)-5-methoxy-indole-3-carbaldehyde

Nucleophile: pyrrolidin-3-ol. Purified by silica gel column chromatography (eluent: methylene chloride/MeOH 9:1). Yield: 54%. MS (m/z): 329.1 (MH⁺).

### 2-cyclopropyl-1-[2-(4-hydroxy-piperidin-1-yl)-ethyl]-5-methoxy-indole-3-carbaldehyde

Nucleophile: piperidin-4-ol. Purified by silica gel column chromatography (eluent: methylene chloride/MeOH 9:1). Yield: 46%. MS (m/z): 343.5 (MH⁺).

### 2-Trifluoromethyl-5-methoxy-1-(2-(4-methyl piperazin-1-yl)ethyl)-indole-3-carbaldehyde

Nucleophile: N-methyl-piperazine. Purified by silica gel column chromatography (eluent: petroleum ether/AcOEt 2:8, then methylene chloride/MeOH 9:1). Yield: 32%. MS (m/z): 370.2 (MH⁺).

### Procedure B

Tosylate (2.06 mmol, 1 eq.) was dissolved in DMF (8 mL) and the selected nucleophile (8.26 mmol, 4 eq.) was added. The resulting solution was heated at 100° C. by microwave irradiation for 20 minutes. DMF was evaporated and the residue was purified as described below. According to this procedure, the following compounds were obtained.

### 1-(2-Imidazol-1-yl-ethyl)-5-methoxy-2-methyl-indole-3-carbaldehyde

Nucleophile: imidazole. Purified by silica gel column chromatography (eluent: CHCl₃/MeOH 98:2). Yield: 70%. MS (m/z): 284.1 (MH⁺).

### 1-[2-(3-Hydroxyl-pyrrolidin-1-yl)-ethyl]-5-methoxy-2-methyl-indole-3-carbaldehyde

Nucleophile: pyrrolidin-3-ol. Purified by silica gel column chromatography (eluent: CHCl₃/MeOH 98:2). Yield: 62%. MS (m/z): 303.2 (MH⁺).

### 5-Methoxy-2-methyl-1-[2-(2-methyl-pyrrolidin-1-yl)-ethyl]-indole-3-carbaldehyde

Nucleophile: 2-methylpyrrolidine. Purified by silica gel column chromatography (eluent: CHCl₃/MeOH 98:2). Yield: 52%. MS (m/z): 301.3 (MH⁺).

### 5-Methoxy-2-methyl-1-[2-(4-methyl-piperidin-1-yl)-ethyl]-indole-3-carbaldehyde

Nucleophile: 4-methyl piperidine. Purified by silica gel column chromatography (eluent: CHCl₃/MeOH 98:2). Yield: 52%. MS (m/z): 315.2 (MH⁺).

### 1-(2-Azepan-1-yl-ethyl)-5-methoxy-2-methyl-indole-3-carbaldehyde

Nucleophile: azepane. Purified by silica gel column chromatography (eluent: CHCl₃/MeOH 98:2). Yield: 58%. MS (m/z): 315.2 (MH⁺).

### 5-Methoxy-1-[2-(4-methyl-piperazin-1-yl)-ethyl]-2-(1-methyl-1H-pyrazol-4-yl)-indole-3-carbaldehyde

Nucleophile: N-methyl-piperazine. Purified by silica gel column chromatography (eluent: CHCl₃/MeOH 99:1 to 97:3). Yield: 40%. MS (m/z): 382.4 (MH⁺).

2-(3,5-Dimethyl-isoxazol-4-yl)-5-methoxy-1-[2-(4-methyl-piperazin-1-yl)-ethyl]-indole-3-carbaldehyde

Nucleophile: N-methyl-piperazine. Purified by silica gel column chromatography (eluent: CHCl₃/MeOH 98:2). Yield: 49%. MS (m/z): 397.2 (MH⁺).

### 5-Methoxy-1-[2-(4-methyl-piperazin-1-yl)-ethyl]-2-pyrimidin-5-yl-indole-3-carbaldehyde

Nucleophile: N-methyl-piperazine. Purified by silica gel column chromatography (eluent: CHCl₃/MeOH 98:2). Yield: 63%. MS (m/z): 380.3 (MH⁺).

### Procedure C

NaH (60% dispersion in mineral oil, 1.2 g, 0.56 mmol, 1.1 eq.) was added to a solution of the selected nucleophile (0.51 mmol, 1 eq.) in DMF (10 mL) cooled to 0° C. The resulting suspension was stirred for 45 minutes, and then tosylate 6× (0.87 mmol, 1.7 eq.) was added. The ice bath was removed and the mixture was heated at 50° C. overnight. After cooling to room temperature, the reaction was partitioned between water and EtOAc. The organic layer was washed with water and brine, dried on Na₂SO₄ and evaporated under reduced pressure. The crude mixture was purified as described below. According to this procedure, the following compounds were obtained.

### 2-Cyclopropyl-1-(2-imidazol-1-yl-ethyl)-5-methoxy-indole-3-carbaldehyde

Nucleophile: imidazole. Purified by silica gel column chromatography (eluent: petroleum ether/AcOEt 4:6, then methylene chloride/MeOH 95:5). Yield: 34%. MS (m/z): 310.4 (MH⁺). ¹H NMR (300 MHz, CDCl₃): 10.38 (so, 1H); 7.90 (bs, 1H); 7.22-7.13 (m, 2H); 7.03-6.92 (m, 2H); 6.46 (s, 1H); 4.64 (t, 2H); 4.39 (t, 2H); 3.91 (s, 3H); 1.89-1.53 (bs, 1H); 1.11-1.03 (m, 2H); 0.77-0.70 (m, 2H).

### 2-Cyclopropyl-5-methoxy-1-(2-pyrazol-1-yl-ethyl)-indole-3-carbaldehyde

Nucleophile: pyrazole. Purified by silica gel column chromatography (eluent: petroleum ether/AcOEt 3:7). Yield: 74%. MS (m/z): 310.3 (MH⁺).

### 5-Methoxy-1-(2-pyrazol-1-yl-ethyl)-2-trifluoromethyl-indole-3-carbaldehyde

Nucleophile: pyrazole. Purified by silica gel column chromatography (eluent: petroleum ether/EtOAc 3:7). Yield: 19%. MS (m/z): 338.3 (MH⁺).

### 2-Cyclopentyl-5-1-[2-(4-methyl piperazin-1-yl)ethyl]-indole-3-carbaldehyde

Nucleophile: N-methyl piperazine. Purified by silica gel column chromatography (eluent: petroleum ether/EtOAc 2:8). Yield: 38%. MS (m/z): 370.3 (MH⁺).

### 2-Cyclohexyl-5-1-[2-(4-methyl piperazin-1-yl)pethyl]-indole-3-carbaldehyde

Nucleophile: N-methyl piperazine. Purified by silica gel column chromatography (eluent: dichloromethane/MeOH 20:1). Yield: 86%. MS (m/z): 384.3 (MH⁺).

### 2-Cyclobutyl-5-1-[2-(4-methylpiperazin-1-yl)ethyl]-indole-3-carbaldehyde

Nucleophile: N-methyl piperazine. Purified by silica gel column chromatography (eluent: dichloromethane/MeOH 95:5). Yield: 78%. MS (m/z): 356.3 (MH⁺).

General Procedure for the Alkylation with 1-bromo-3-chloro-propane

To a solution of the selected 5-methoxy-indole-3-carbaldehyde 1× (24.6 mmol) in DMF (90 mL), cooled to 0° C., NaH (60% dispersion in mineral oil, 1.97 g, 49.3 mmol, 2 eq.) was added. The resulting suspension was stirred for 15 minutes, and then 1-bromo-3-chloro-propane (12.2 mL, 123.1 mmol, 5 eq.) was added. The ice was removed and the reaction mixture was allowed to stir overnight at room temperature. The reaction was quenched with the addition of water and extracted with AcOEt. The organic layer was washed with brine, dried on Na₂SO₄ and evaporated to give a crude mixture that was further purified as described below. According to this procedure, the following compounds were obtained.

### 1-(3-Chloro-propyl)-5-methoxy-indole-3-carbaldehyde

Purified by silica gel column chromatography (eluent: gradient from hexane/EtOAc 7:3 to hexane/EtOAc 1:1). Yield: 86%. MS (m/z): 252.1 (MH⁺).

### 1-(3-Chloro-propyl)-5-methoxy-2-methyl-indole-3-carbaldehyde

Purified by silica gel column chromatography (eluent: CHCl₃/MeOH 99.8:0.2). Yield: 78%. MS (m/z): 266.1 (MH⁺).

### 1-(3-Chloro-propyl)-3-formyl-5-methoxy-indole-2-carboxylic acid dimethyl amide

Purified by silica gel column chromatography (eluent: CHCl₃/MeOH 99:1). Yield: 53%. MS (m/z): 323.2 (MH⁺).

### 1-(3-Chloro-propyl)-2-cyclopropyl-5-methoxy-indole-3-carbaldehyde

Purified by silica gel column chromatography (eluent: petroleum ether/AcOEt 7:3). Yield: 57%. MS (m/z): 292.3 (MH⁺). *Yields were calculated assuming the product as only chloro derivative.

### General Procedures (A, B) for the Nucleophilic Displacement (Preparation of Carbaldehyde Compounds

### Procedure A

To a solution of 7× (21.24 mmol, 1 eq.) in acetonitrile (350 mL), K₂CO₃ (14.66 g, 106.2 mmol, 5 eq.), KI (8.82 g, 53.1 mmol, 2.5 eq.) and dimethylamine (2M in THF, 42.5 mL, 85 mmol, 4 eq.) were added. The resulting suspension was heated to 90° C. for 24 hours. The reaction mixture was allowed to cool to room temperature and filtered. The recovered solid was washed with AcOEt. To the filtrate water was added, the layers were separated and the aqueous layer was extracted with AcOEt. Combination of the organic layers, followed by drying on Na₂SO₄ and evaporation, afforded a crude mixture that was further purified as described below. According to this procedure, the following compounds were obtained.

### 1-(3-Dimethylamino-propyl)-5-methoxy-indole-3-carbaldehyde

Purified by silica gel column chromatography (eluent: CH₂Cl₂/MeOH 98:2⁺0.5% NH₃ aq.). Yield: 71%. MS (m/z): 261.1 (MH⁺).

### 1-(3-Dimethylamino-propyl)-5-methoxy-2-methyl-indole-3-carbaldehyde

Purified by silica gel column chromatography (eluent: CHCl₃/MeOH 95:5). Yield: 83%. MS (m/z): 275.4 (MH⁺).

### 1-(3-Dimethylamino-propyl)-3-formyl-5-methoxy-indole-2-carboxylic acid dimethylamide

Purified by silica gel column chromatography (eluent: CHCl₃/MeOH 96:4). Yield: 73%. MS (m/z): 332.2 (MH⁺).

### 2-Cyclopropyl-1-(3-dimethylamino-propyl)-5-methoxy-indole-3-carbaldehyde

Purified by silica gel column chromatography (eluent: CH₂Cl₂/MeOH 99:1+0.5% NH₃ aq.). Yield: 80%. MS (m/z): 301.1 (MH⁺).

### Procedure B

1-(3-Chloro-propyl)-5-methoxy-2-methyl-indole-3-carbaldehyde (0.50 g, 1.879 mmol, 1 eq.) and the selected nucleophile (16.91 mmol, 9 eq.) were heated at 80° C. by microwave irradiation for 15 minutes. Excess nucleophile was evaporated and the crude mixture was further purified as indicated below. According to this procedure, the following compounds were obtained.

### 5-Methoxy-2-methyl-1-(3-pyrrolidin-1-yl-propyl)-indole-3-carbaldehyde

Nucleophile: pyrrolidine. Purified by silica gel column chromatography (eluent: CHCl₃/MeOH 97:3). Yield: 33%. MS (m/z): 301.3 (MH⁺).

### 5-Methoxy-2-methyl-1-[3-(2-methyl-pyrrolidin-1-yl)-propyl]-indole-3-carbaldehyde

Nucleophile: 2-methylpyrrolidine. Purified by silica gel column chromatography (eluent: CHCl₃/MeOH 98:2). Yield: 71%. MS (m/z): 315.3 (MH⁺).

### 5-Methoxy-2-methyl-1-(3-piperidin-1-yl-propyl)-indole-3-carbaldehyde

Nucleophile: piperidine. Purified by silica gel column chromatography (eluent: CHCl₃/MeOH 96:4). Yield: 70%. MS (m/z): 315.2 (MH⁺).

### 5-Methoxy-2methyl-1-[3-(4-methylpiperidin-1-yl)-propyl]-indole-3-carbaldehyde

Nucleophile: 4-methyl piperidine. Purified by silica gel column chromatography (eluent: CHCl₃/MeOH 96:4). Yield: 89%. MS (m/z): 329.1 (MH⁺).

### 1-(3-Azepan-1-yl-propyl)-5-methoxy-2-methyl-indole-3-carbaldehyde

Nucleophile: azepane. Purified by silica gel column chromatography (eluent: CHCl₃/MeOH 96:4). Yield: 64%. MS (m/z): 329.1 (MH⁺).

### Synthesis of Other indole-3-carbaldehydes

### Preparation of 1-methyl-2-phenyl-1H-indole-3-carbaldehyde

To a solution of 2-phenyl-1H-indole-3-carbaldehyde (7.41 g, 33.5 mmol) in DMF (50 ml) cooled to 0° C. was added in portions, sodium hydride (2.68 g, 67.0 mmol). After stirring for 30 minutes, iodomethane (4.18 ml, 67.0 mmol) was added and the reaction stirred for 30 minutes, then allowed to warm to room temperature and stirred overnight. Water (150 mL) was added and the resulting solid was filtered, washed well with water and air dried to give a light green solid 1-methyl-2-phenyl-1H-indole-3-carbaldehyde (5.30 g, 22.53 mmol, 67.3% yield), MS (m/z) 236.3 (MH⁺).

### Preparation of 4-(4-methoxyphenyl)-1H-indole-3-carbaldehyde

To a mixture of 4-bromo-1H-indole-3-carbaldehyde (112 mg, 0.5 mmol), Tetrakis(triphenylphosphine)palladium(0) (57.8 mg, 0.050 mmol), 2-(4-methoxyphenyl)-4,4,5,5-tetramethyl-1,3,2-dioxaborolane (129 mg, 0.550 mmol) and dimethoxyethane (3.0 mL) in a 2-5 mL-microwave tube was added 0.75 mL of 2M sodium carbonate (1.5 mmol). This was capped and heated in the microwave for 1 hour at 110° C. Work-up by quenching into 20 mL water, mixture extracted with ethyl acetate (2×10 mL) the ethyl acetate layer evaporated to give a gum which was dissolved dichloromethane passed through a short pad of silica-gel, the product was removed from the silica gel eluting with 1:1 hexane/ethyl acetate then evaporated to give 4-(4-methoxyphenyl)-1H-indole-3-carbaldehyde (130 mg, 0.517 mmol, 103% yield). Used as is for the next step.

### Preparation of 4-(4-methoxyphenyl)-1-methyl-1H-indole-3-carbaldehyde

To a mixture of 4-bromo-1-methyl-1H-indole-3-carbaldehyde (238 mg, 1.0 mmol), Tetrakis(triphenylphosphine)palladium(0) (116 mg, 0.100 mmol), 2-(4-methoxyphenyl)-4,4,5,5-tetramethyl-1,3,2-dioxaborolane (258 mg, 1.100 mmol) and dimethoxyethane (3.0 mL) in a 2-5 mL-microwave tube was added 1.125 mL of 2M sodium carbonate (1.5 mmol). This was capped and heated in the microwave for 1 hour at 120° C. Work-up by quenching into 20 mL water, mixture extracted with ethyl acetate (2×10 mL) the ethyl acetate layer evaporated to give a gum which was dissolved dichloromethane, loaded onto 2 grams of silica gel and purified by chromatography on the ISCO Companion™ using a hexane/ethyl acetate gradient on a 40 gram column, combined cuts containing product were then evaporated to give a gum. After standing overnight, the gum showed some crystals. This was treated with 6:1 hexanes/ethyl acetate, the off white solid was collected on a sintered glass funnel, washed with fresh solvent and air dried to give 4-(4-methoxyphenyl)-1-methyl-1H-indole-3-carbaldehyde (132 mg, 0.498 mmol, 49.8% yield), MS (m/z): 266.1 (MH⁺).

### Condensation of indole-3-carbaldehydes and Benzofuranones or Benzothiopheneones 2-(1H-indol-3-ylmethylene)-1-benzofuran-3(2H)-on

To benzofuranone (15.6 mmol, 0.9 eq) and 3-indole aldehyde (17.3 mmol, 1 eq) in EtOH (2 mL) was added a catalytic amount of HCl (12 N). The resulting mixture was stirred for 120 minutes at 80° C. and allowed to cool to room temperature. The solution was concentrated in a Speed-Vac and the resulting residue purified via preparative HPLC conditions to afford the title compound. LCMS RT=2.40 MS=260.1.

### 4,6-dihydroxy-2-[(5-methoxy-2-phenyl-1H-indol-3-yl)methylene]-1-benzofuran-3(2H)-one

To the 4,6-dihydroxy-benzofuran-3-one (125 mgs, 0.75 mmol, 1 eq) and desired 5-methoxy-2-phenyl-1H-indole-3-carbaldehyde (188 mgs, 0.75 mmol, 1 eq) in EtOH (3 mL) was added a catalytic amount of HCl (12 N). The resulting mixture was stirred for 180 minutes at 80° C. and allowed to cool to room temperature. The suspension was filtered. The red solid was dried in a Speed-Vac and purified via preparative HPLC to afford the title compound. LCMS RT=2.19 MS=398.1

### 4,6-dihydroxy-2-({5-methoxy-2-methyl-1-[2-(4-methyl piperazin-1-yl)ethyl]-1H-indol-3-yl}methylene)-1-benzofuran-3(2H)-one

To 1-(2-chloroethyl)-5-methoxy-2-methyl-1H-indole-3-carbaldehyde (crude product taken directly from previous reaction) in EtOH (3 mL) was added the desired 4,6-dihydroxy-benzofuran-3-one (70 mgs) and HCl (12N, 8 drops). The reaction mixture was heated to 90 C and stirred for 2.5 hrs—LCMS indicated no remaining benzofuranone and product formation. The reaction was allowed to cool. Concentration of the solution in a Speed-Vac and purification via preparative HPLC afforded the title compound. LCMS RT=1.89 MS=464.2.

### 4,6-dihydroxy-2-[(5-methoxy-1H-indol-3-yl)methyl]-1-benzofuran-3(2H)-one

4,6-dihydroxy-2-((5-methoxy-1H-indol-3-yl)methylene)benzofuran-3(2H)-one (0.09 mmol) synthesized as in Preparation 1 in 10 mL MeOH and 2 mL dioxane was hydrogenated under 48 psi H2 atmosphere for 24 hrs. The reaction was filtered and concentrated in a Speed-Vac. The resulting residue purified via preparative HPLC conditions to afford the title compound. LCMS RT=1.75 MS=324.1.

### The preparation of (2Z)-2-{[4-(4-Fluorophenyl)-1-methyl-1H-indol-3-yl]methylene}-4,6-dihydroxy-1-benzofuran-3(2H)-one

### Step 1

### Preparation of 2-[(4-bromo-1-methyl-1H-indol-3-yl)methylene]-4,6-dihydroxy-1-benzofuran-3(2H)-one

A mixture of 2 g (12.04 mmol) of 4,6-dihydroxycoumaranone, 3.15 g (13.24 mmol) of 4-bromo-1-methyl-H-indole-3-carbaldehyde, 2.5 mL of conc. HCl, and 47.5 mL of absolute ethanol was stirred at 80° C. overnight. After cooling, the precipitate was filtered and washed with 10% methanol in methylene chloride. The solid was dried under house vacuum to give 3.8 g of yellow solid (82% yield). MS (m/z) 386.2 (MH⁺).

### Step 2

A mixture of 120 mg (0.31 mmol) of 2-[(4-bromo-1-methyl-1H-indol-3-yl)methylene]-4,6-dihydroxy-1-benzofuran-3(2H)-one, 86.5 mg (0.62 mmol) of 4-fluorophenyl boronic acid, 53.7 mg (0.047 mmol) of tetrakis(triphenylphosphine)palladium(0), and saturated aqueous sodium carbonate (1 mL), was placed in a microwave vial. To the mixture were added 3 mL of 1-methyl-2-pyrrolidinone and 1,2-dimethoxyethane (1:3). The sealed tube was heated by microwave for twenty minutes at 120° C. After cooling, the mixture was filtered through Celite™ and washed with 12% methanol in methylene chloride. After the solvent was evaporated, the residue was purified by column chromatography (10% methanol in ethyl acetate) to give 55 mg of a yellow solid (44° A) yield). MS (m/z) 402.2 (MH⁺).

### Preparation of (2Z)-4,6-dihydroxy-2-{[4-(4-isopropoxyphenyl)-1-methyl-1H-indol-3-yl]methylene}-1-benzofuran-3(2H)-one

A mixture of 100 mg (0.66 mmol) of 4,6-dihydroxycoumaranone) 158 mg (0.66 mmol) of 4-(4-isopropoxy-phenyl)-1-methyl-1H-indole-3-carboxylaldehyde, 0.25 mL of conc. HCl, and 4.75 mL of absolute ethanol was stirred at 80° C. overnight. After cooling, the reddish mixture was evaporated and purified by reverse phase HPLC to give 103.5 mg of (2Z)-4,6-dihydroxy-2-{[4-(4-isopropoxyphenyl)-1-methyl-1H-indol-3-yl]methylene}-1-benzofuran-3(2H)-one as a yellow solid (77% yield). MS (m/z) 442.2 (MH⁺).

### Preparation of (2Z)-4,6-dimethoxy-2-[(1-methyl-2-phenyl-1H-indol-3-yl)methylene]benzofuran-3(2H)-one

To a mixture of 1-methyl-2-phenyl-1H-indole-3-carbaldehyde (471 mg, 2.002 mmol), 4,6-dimethoxybenzofuran-3(2H)-one (389 mg, 2.002 mmol) and ethanol (30 mL) was added 2 drops of concentrated hydrochloric acid. All solids dissolve to give a deep maroon solution, which slowly lightens and precipitates a solid, while heated by an oil bath at 80° C. Stirred overnight. Reaction mixture cooled and the solid collected washed with ethanol and air dried to give an orange brown solid, (2Z)-4,6-dimethoxy-2-[(1-methyl-2-phenyl-1H-indol-3-yl)methylene]benzofuran-3(2H)-one (699 mg, 1.699 mmol, 85% yield), mp 257-8. MS (m/z) 414.2 (MH⁺).

### (2Z)-4-Hydroxy-2-[(1-methyl-4-phenyl-1H-indol-3-yl)methylene]-1-benzofuran-3(2H)-one

A mixture of 1-methyl-4-phenyl-1H-indole-3-carbaldehyde, 4,6-dihydroxycoumaranone, ethanol, and conc. HCl was heated. After heating, the precipitate was filtered and washed with ethanol to yield (2Z)-4-Hydroxy-2-[(1-methyl-4-phenyl-1H-indol-3-yl)methylene]-1-benzofuran-3(2H)-one, MS (m/z) 368.3 (MH⁺).

### Condensation between 4,6-dihydroxy-benzofuran-3-one (Compound A) and 5-methoxy-indole-3-carbaldehydes

To a solution of the selected 5-methoxy-indole-3-carbaldehyde compounds (4 mmol, 1 eq.) and 4,6-dihydroxy-benzofuran-3-one A (664 mg, 4 mmol, 1 eq.) in EtOH (16 mL), a catalytic amount of 12 N HCl was added. The resulting mixture was stirred at 85° C. until disappearance of the starting materials and then allowed to cool to room temperature. The formed solid was recovered by filtration, washed with ethyl ether and dried under vacuum. In some cases, as needed, after cooling the reaction mixture to room temperature, excess hexane could be added, the mixture stirred for 30 minutes, the formed solid removed by filtration and the solvents evaporated, with the residue being purified by preparative HPLC.

Preparation of 3-substituted-1H-pyrrolo[2,3-b]pyridine and 3-substituted-1H-pyrrolo[3,2-b]pyridine compounds. The following methods outline the synthesis of the phenolic compounds having at least one hydroxyl group corresponding to R¹, R², R³, or R⁴, which hydroxyl group can be then converted to an OR²⁰ group to give the compounds of the invention as further described below.

### Synthesis of Benzofuranone Intermediates

The preparation of 4,6-dihydroxybenzofuranone, 4-hydroxybenzofuranone, monosubstituted 6-hydroxy benzofuranones, 2-fluoro-3-methoxy-phenol; the General procedure for the demethylation with BBr₃ to obtain 2-Fluoro-benzene-1,3-diol, 5-Fluoro-benzene-1,3-diol, 5-Chloro-benzene-1,3-diol; the General procedure for the preparation of 6-hydroxybenzofuranones to obtain 6-Hydroxy-4-methyl-benzofuran-3-one, 6-Hydroxy-5-methyl-benzofuran-3-one, 6-Hydroxy-7-methyl-benzofuran-3-one, 4-Fluoro-6-hydroxy-benzofuran-3-one, 5-Fluoro-6-hydroxy-benzofuran-3-one, 7-Fluoro-6-hydroxy-benzofuran-3-one, 4-Chloro-6-hydroxy-benzofuran-3-one, 5-Chloro-6-hydroxy-benzofuran-3-one, 7-Chloro-6-hydroxy-benzofuran-3-one, 5-Bromo-6-hydroxy-benzofuran-3-one, 4-Bromo-6-hydroxy-benzofuran-3-one; and the preparation of 6-hydroxy-4-methoxybenzofuran-3(2H)-one can be performed similarly to what is described in Section A-I hereinabove.

### Synthesis of Pyrrolopyridine-3-carbaldehyde Intermediates

### Preparation of 5-Methoxy-1H-pyrrolo[3,2-b]pyridine-3-carbaldehyde

2-Methoxy-5-nitro-pyridine (4 g, 25.6 mmol) and 4-chlorophenoxyacetonitrile (4.8 g, 28.5 mmol) were dissolved in THF (58 mL). The resulting solution was slowly added to a solution of t-BuOK (6.3 g, 56.3 mmol) in THF dry (60 mL) at −10° C. The reaction mixture was stirred for 3 hours at −10° C., and then water was added. The aqueous layer was extracted with EtOAc. The combined organic layers were dried on Na₂SO₄ and evaporated to give a crude that was purified by silica gel column chromatography (eluent: petroleum ether/EtOAc 8:2) to give (6-methoxy-3-nitro-pyridin-2-yl)-acetonitrile (Yield: 50%. MS (m/z): 194.1 (MH⁺)).

To a solution of (6-methoxy-3-nitro-pyridin-2-yl)-acetonitrile (1 g, 5.18 mmol) in EtOH (30 mL), 10% Pd/C was added. The mixture was hydrogenated at 45 psi at room temperature overnight. The catalyst was filtered off and the solvent was evaporated. The crude product was purified by silica gel column chromatography (eluent: petroleum ether/EtOAc 8:2) to give 5-methoxy-1H-pyrrolo[3,2-b]pyridine (Yield: 64%. MS (m/z): 149 (MH⁺).

To a solution of 5-methoxy-1H-pyrrolo[3,2-b]pyridine (498 mg, 3.36 mmol) in 33% acetic acid (5.2 mL), hexamethylenetetramine (714 mg, 5.05 mmol) was added. The reaction mixture was refluxed for 4 hours. After cooling, the reaction was extracted with EtOAc. The combined organic layers were dried on Na₂SO₄ and evaporated to give a crude that was purified twice by silica gel column chromatography (eluent: methylene chloride/MeOH 95:5) to give 5-methoxy-1H-pyrrolo[3,2-b]pyridine-3-carbaldehyde (Yield: 27%. MS (m/z): 177.17 (MH⁺)).

### Preparation of 1-methyl-4-phenyl-1H-pyrrolo[2,3-b]pyridine-3-carbaldehyde

A solution of 70% mCPBA (11.54 g, 66.87 mmol) in ethyl acetate (25 mL) was added by drops to a solution of 7-azaindole (5 g, 42.3 mmol) in ethyl acetate (40 mL) at 0° C. with a good stirrer. After addition was completed, the mixture was stirred at room temperature for 1 to 2 hours until no starting material left. The mixture was cooled, filtered, and washed with ethyl acetate to give a solid. It was dissolved in 50 mL of water and treated with 30% K₂CO₃ solution (˜16 mL) to pH 9.5-10.5 to give a precipitate. It was stirred at room temperature for 1 hour, cooled, filtered, and washed with a small amount of cold water to give 2.484 g of 1H-pyrrolo[2,3-b]pyridine 7-oxide as a white crystal (43.8% yield). MS (m/z): 135.1 (MH⁺).

A solution of methanesulfonic anhydride (6.066 g, 34.82 mmol) and acetonitrile (11.7 mL) was added by drops to a solution of 1H-pyrrolo[2,3-b]pyridine 7-oxide (2.333 g, 17.41 mmol), tetramethyl ammonium bromide (4.023 g, 26.12 mmol) in DMF (11.7 mL) at 0° C. After stirring at 0° C. for 45 minutes, additional DMF (11.7 mL) was added in drops to the thick mixture at 0° C., and then stirred at room temperature overnight. To the mixture was added ice water (35 mL), followed by 10 N NaOH (˜4.66 mL) to pH 7. After stirring at the room temperature, a precipitate formed. It was filtered and washed with water to give 1.891 g of 4-bromo-1H-pyrrolo[2,3-b]pyridine as a pale peach solid (55% yield). MS (m/z): 197 (MH⁺). NMR (DMSO-d₆) showed 6-9% impurity which is likely to be the 4,6-dibromo compound based on LC/MS analysis.

A mixture of 4-bromo-1H-pyrrolo[2,3-b]pyridine (197 mg, 1 mmol), dimethylamine hydrochloride (88 mg, 1.079 mmol), and paraformaldehyde (33 mg, 1.1 mmol) in n-butanol (2 mL) was heated at 120° C. for 1.25 hours. After removal of the solvent, the residue was treated with ice water and three drops of concentrated HCl. After washing with ether, the aqueous layer was basified with saturated K₂CO₃ solution and extracted with methylene chloride. The organic layer was dried over sodium sulfate, filtered, and the solvent dried to give 106 mg of 1-(4-bromo-1H-pyrrolo[2,3-b]pyridin-3-yl)-N,N-dimethylmethanamine as a light pink solid (42%). MS (m/z): 254.2 (MH⁺).

A solution of 1-(4-bromo-1H-pyrrolo[2,3-b]pyridin-3-yl)-N,N-dimethylmethanamine (341 mg, 1.34 mmol) and hexamethylenetetramine (190 mg, 1.34 mmol) in 66% propionic acid was added by drops to a refluxing solution of hexamethylenetetramine (190 mg, 1.34 mmol) in 66% propionic acid (0.8 mL) at 120° C. The reaction mixture was heated for 2-4 hours, and monitored by MS. It was cooled, treated with water (4 mL), and filtered to give 238 mg of 4-bromo-1H-pyrrolo[2,3-b]pyridine-3-carbaldehyde as a beige solid (79%). MS (m/z): 225.0 (MH⁺).

Sodium hydride (60%, 27.4 mg, 0.686 mmol) was added in portions to a suspension of 4-bromo-1H-pyrrolo[2,3-b]pyridine-3-carbaldehyde (128.6 mg, 0.572 mmol) in 5 mL of DMF and 1 mL of THF at 0° C. After stirring at 0° C. for 20 minutes, methyl iodide (39.2 μL, 0.6292 mmol) was added by drops into the mixture and warmed up to room temperature for 2.5 hours. The solvents were evaporated and the residue was treated with methylene chloride, filtered, and dried. This was further treated with hexane. The mixture was filtered again and washed with hexane to give a beige solid, which was recrystallized from chloroform and hexane to yield 102 mg of 4-bromo-1-methyl-1H-pyrrolo[2,3-b]pyridine-3-carbaldehyde as crystals (74%). MS (ESI): m/z 239 (M+H).

A mixture of 4-bromo-1-methyl-1H-pyrrolo[2,3-b]pyridine-3-carbaldehyde (38 mg, 0.159 mmol), phenyl boronic acid (38.8 mg, 0.318 mmol), and tetrakis(triphenylphosphine)palladium (0) (27.6 mg, 0.0238 mmol) in saturated sodium carbonate (0.37 mL) and 1,2-dimethoxylethane (1.4 mL) was heated at 120° C. in microwave for 20 minutes. It was filtered through a pad of silica gel and washed with 5% MeOH in ethyl acetate. After the solvent was evaporated, acetonitrile was added to the residue, and filtered to remove a bright yellow solid. The filtrate was concentrated to yield 51.4 mg of 1-methyl-4-phenyl-1H-pyrrolo[2,3-b]pyridine-3-carbaldehyde as white crystals (Ar=phenyl, 76%). MS (ESI): m/z 237(M+H).

### Synthesis of 1-methyl-4-phenylethynyl-1H-pyrrolo[2, 3-b]pyridine-3-carbaldehyde (34)

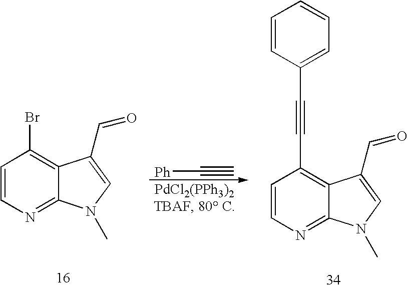

4-Bromo-1-methyl-1-H-pyrrolo[2,3-b]pyridine-3-carbaldehyde (0.10 g, 0.42 mmol) was combined with phenylacetylene (0.051 g, 0.5 mmol), bis(triphenylphosphine) palladium (II) chloride (8.8 mg, 0.126 mmol) and tetrabutylammonium fluoride (0.33 g, 1.26 mmol) and heated to 80° C. overnight. The thick black solution was diluted with water and extracted with ethyl acetate. The organic layer was washed with brine, dried over MgSO₄, concentrated and purified via silica gel (50% EtOAc: Hex gradient) to produce 79 mg (72%) 1-methyl-4-phenylethynyl-1H-pyrrolo[2,3-b]pyridine-3-carbaldehyde as an off-white solid. Reference: JOC, 2006 (71) 379.

### Synthesis of 1-methyl-4-piperidin-1-yl-1-H-pyrrolo[2,3-b]pyridine-3-carbaldehyde (35)

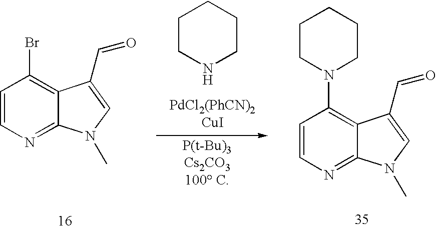

To a mixture of 4-bromo-1-methyl-1-H-pyrrolo[2,3-b]pyridine-3-carbaldehyde (0.06 g, 0.25 mmol) in dioxane (5 mL) was added piperidine (0.12 mL, 1.25 mmol), bis(benzonitrile)dichloro palladium (1.4 mg, 0.0038 mmol), copper(I) iodide (1.4 mg, 0.0075 mmol), tri-tert-butyl phosphine (2.3 mg, 0.011 mmol) and cesium carbonate (0.16 g, 0.5 mmol) and was heated to 100° C. overnight. The reaction was concentrated and purified on silica using a 50% EtOAc/Hex gradient to produce 0.035 g (54%) of 1-methyl-4-piperidin-1-yl-1-H-pyrrolo[2,3-b]pyridine-3-carbaldehyde as an off white solid. Reference: Synlett, 2001 (5) p. 609.

### Synthesis of 1-methyl-4-styryl-1H-pyrrolo[2,3-b]pyridine-3-carbaldehyde (36)

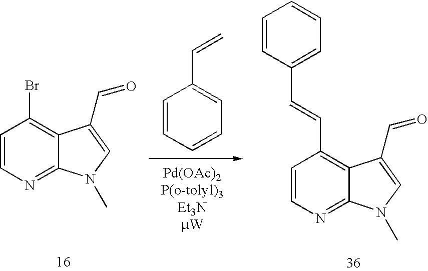

To a mixture of 4-bromo-1-methyl-1-H-pyrrolo[2,3-b]pyridine-3-carbaldehyde (0.08 g, 0.33 mmol) in DMF (2 mL) in a 2-5 mL Biotage microwave vial was added palladium acetate (6 mg, 0.027 mmol), tri-o-tolylphosphine (23.4 mg, 0.077 mmol), triethyl amine (0.19 mL, 1.34 mmol) and styrene (0.077 mL, 0.67 mmol). It was irradiated at 160° C. for 45 minutes (Biotage Initiator™ 60). The solution was stripped to dryness and purified on silica gel (50% EtOAc/Hex gradient) to give 0.045 g (51%) 1-methyl-4-styryl-1H-pyrrolo[2,3-b]pyridine-3-carbaldehyde. Ref: Synlett, 2001 (5) p. 609.

### Synthesis of 1-methyl-4-phenoxy-1H-pyrrolo[2,3-b]pyridine-3-carbaldehyde (37)

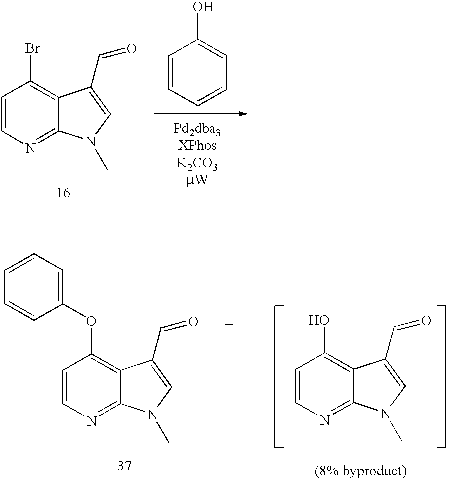

To a mixture of 4-bromo-1-methyl-1-H-pyrrolo[2,3-b]pyridine-3-carbaldehyde (0.2 g, 0.84 mmol) in toluene (2 mL) was added phenol (0.12 g, 1.25 mmol), tris(dibenzylideneacetone)dipalladium (0.04 g, 0.042 mmol), 2-(dicyclohexylphosphino)-2′,4′,6′-triisopropyl-1,1′-biphenyl (0.04 g, 0.084 mmol) (X-Phos), potassium carbonate (0.26 g, 1.85 mmol) and degassed in a 2-5 mL microwave tube. The mixture was irradiated to 130° C. for 3 hours (Biotage Initiator™ 60), cooled, filtered through a Whatman 45 micron filter and concentrated. Purification on silica gel using a 50% EtOAc/Hex gradient afforded 0.095 g (45%) of 1-methyl-4-phenoxy-1H-pyrrolo[2,3-b]pyridine-3-carbaldehyde as a white solid. Ref: Synthesis, 2006 (4) p. 629.

### Synthesis of 4-hydroxy-1-methyl-1-H-pyrrolo[2,3-b]pyridine-3-carbaldehyde

As described in the synthesis of 1-methyl-4-phenoxy-1H-pyrrolo[2,3-b]pyridine-3-carbaldehyde, 0.017 g (8%) of 4-hydroxy-1-methyl-1-H-pyrrolo[2,3-b]pyridine-3-carbaldehyde was isolated as a minor by-product.

### 1-Methyl-4[methyl(phenyl)amino]-1-H-indole-3-carbaldehyde (38)

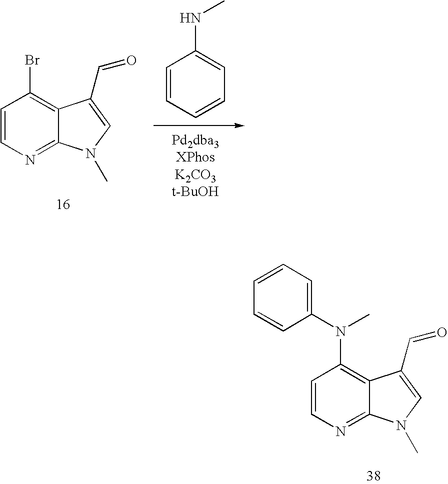

To a mixture of 4-bromo-1-methyl-1-H-pyrrolo[2,3-b]pyridine-3-carbaldehyde (0.05 g, 0.21 mmol) in t-butanol (1 mL) was added N-methyl aniline (0.026 g, 0.24 mmol), tris(dibenzylideneacetone)dipalladium (0.01 g, 0.042 mmol), 2-(dicyclohexylphosphino)-2′,4′,6′-triisopropyl-1,1′-biphenyl (0.011 g, 0.023 mmol) (X-Phos), potassium carbonate (0.064 g, 0.46 mmol) and degassed in a pressure tube. The mixture was heated at 100° C. for 20 hours, cooled, diluted with 20 mL of methylene chloride, filtered through Celite™ and concentrated. Purification on a preparative LC a 50% EtOAc/Hex gradient afforded 0.033 g (29%) of 1-methyl-4[methyl(phenyl)amino]-1-H-indole-3-carbaldehyde as a pale yellow solid. Ref: Synthesis, 2006 (4) p. 629.

### Synthesis of 1-methyl-2-phenyl-1H-pyrrolo[2,3-b]pyridine (40)

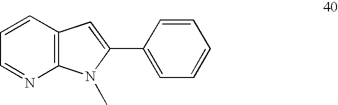

A mixture of 1-methyl-1H-pyrrolo[2,3-b]pyridine (0.919 g, 6.96 mmol), palladium acetate (7.8 mg, 0.035 mmol), triphenylphosphine (36.5 mg, 0.139 mmol), phenyl iodide (0.935 mL, 8.352 mmol), cesium acetate (2.645 g, 13.78 mmol) in dimethyl acetamide (0.92 mL) was heated at 125° C. for 14.5 hours. It was filtered through a pad of silica gel and washed with ethyl acetate. After the solvents were removed, the residue was purified by preparative TLC (developed by 40% ethyl acetate in hexane) to yield 0.767 g (53%) of 1-methyl-2-phenyl-1H-pyrrolo[2,3-b]pyridine as a light yellow oil: MS (ESI) m/z 209.2 (M+H)⁺.

### Synthesis of N,N-dimethyl-1-(1-methyl-2-phenyl-1H-pyrrolo[2,3-b]pyridin-3-yl)methanamine (41)

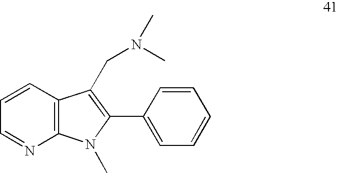

A mixture of 1-methyl-2-phenyl-1H-pyrrolo[2,3-b]pyridine (320 mg, 1.536 mmol), dimethylamine hydrochloride (135 mg, 1.658 mmol) and paraldehyde (50.6 mg, 1.69 mmol) in n-butanol (3 mL) was heated at 120° C. for 1.5 hours. After removal of the solvent, the residue was treated with ice, 3 drops of concentrated HCl, and ether. The aqueous layer was separated and treated with potassium carbonate, followed by treatment with methylene chloride. The organic layer was dried to yield 0.3315 g (81%) of the title compound as a yellow oil: MS (ESI) m/z 266.3 (M+H)⁺.

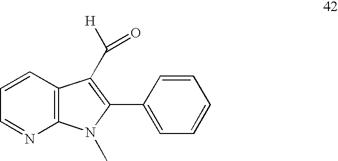

### Synthesis of 1-methyl-2-phenyl-1H-pyrrolo[2,3-b]pyridine-3-carbaldehyde (42)

A solution of N,N-dimethyl-1-(1-methyl-2-phenyl-1H-pyrrolo[2,3-b]pyridin-3-yl)methanamine (295 mg, 1.11 mmol), hexamethylenetetramine (156 mg, 1.11 mmol) and 66% propionic acid (1.2 mL) was added in drops to a refluxing solution of hexamethylenetetramine (156 mg, 1.11 mmol) and 66% propionic acid (0.7 mL). After refluxing for 27 hours, it was treated with ice water and methylene chloride. The organic layer was purified by chromatography on a silica gel column and eluted with 40% ethyl acetate in hexane. The fractions were collected and dried to give 0.158 g (60%) of the title compound as a white solid: MS (ESI) m/z 237.2 (M+H)⁺.

### B-III. Condensation of pyrrolopyridine-3-carbaldehydes and Benzofurans

The condensation of pyrrolopyridine-3-carbaldehydes was performed analogously to what was described in Section A-III above. Below are shown some representative examples of the condensation.

### 4,6-dihydroxy-2-(1H-pyrrolo[2,3-b]pyridin-3-yl methylene)-1-benzofuran-3(2H)-one

To 1H-pyrrolo[2,3-b]pyridin-3-carbaldehyde (0.158 g) in EtOH (3 mL) was added 4,6-dihydroxy-benzofuran-3-one (70 mgs) and HCl (12N, 8 drops). The reaction mixture was heated to 90° C. and stirred for 2.5 hrs—LCMS indicated no remaining benzofuranone and product formation. The reaction was allowed to cool. Concentration of the solution in a Speed-Vac and purification via preparative HPLC afforded the title compound. LCMS retention time=1.78 MS=295.1.

Using the procedure above Compounds 1-10 were also prepared. In some cases the reaction suspension was filtered and the solid recrystallized, if necessary, from EtOH. Otherwise the reaction was concentrated via Speed-Vac and purified via preparative HPLC to afford the desired compounds. Compound and analytical data are show in Table I below.

### Preparation of (2Z)-4,6-dihydroxy-2-[(1-methyl-4-phenyl-1H-pyrrolo[2,3-b]pyridin-3-yl)methylene]-1-benzofuran-3(2H)-one

A mixture of 1-methyl-4-phenyl-1H-pyrrolo[2,3-b]pyridine-3-carbaldehyde (Ar=phenyl, 18 mg, 0.076 mmol), 4,6-dihydroxycoumaranone (12.7 mg, 0.076 mmol), ethanol (0.36 mL), and conc. HCl (0.061 mL) was heated at 80° C. After it dissolved, a precipitate formed. After heating for 3 hours, the precipitate was filtered and washed with ethanol to yield 19.8 mg of (2Z)-4,6-dihydroxy-2-[(1-methyl-4-phenyl-1H-pyrrolo[2,3-b]pyridin-3-yl)methylene]-1-benzofuran-3(2H)-one as a yellow solid (Ar=phenyl, 67%). MS (m/z): 385.2 (MH⁺). ¹H NMR (400 MHz, DMSO-d₆) δ ppm 3.99 (s, 3H), 6.03 (s, 1H), 6.22 (s, 1H), 6.29 (s, 1H), 7.11 (d, J=5.1 Hz, 1H), 7.55 (m, 5H), 8.37 (s, 1H), 8.40 (d, J=5.1 Hz, 1H).

### Preparation of (2Z)-4,6-dihydroxy-2-[(5-methoxy-1H-pyrrolo[3,2-b]pyridin-3-yl)methylene]-1-benzofuran-3(2H)-one

To a solution of 5-methoxy-1H-pyrrolo[3,2-b]pyridine-3-carbaldehyde and 4,6-dihydroxy-benzofuran-3-one A (664 mg, 4 mmol, 1 eq.) in EtOH (16 mL), a catalytic amount of 12 N HCl was added. The resulting mixture was stirred at 85° C. until disappearance of the starting materials and then allowed to cool to room temperature. The formed solid was recovered by filtration, washed with ethyl ether and dried under vacuum. The product was obtained by filtration. Yield: 62%. MS (m/z): 325.19 (MH⁺).

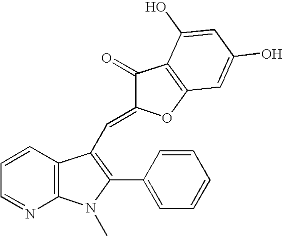

### Synthesis of (2Z)-4,6-dihydroxy-2-[(1-methyl-2-phenyl-1H-pyrrolo[2,3-b]pyridin-3-yl)methylene]-1-benzofuran-3(2H)-one

A mixture of 1-methyl-2-phenyl-1H-pyrrolo[2,3-b]pyridine-3-carbaldehyde (70 mg, 0.296 mmol), 4-hydroxy-1-benzofuran-3(2H)-one (49 mg, 0.296 mmol), ethanol (2.18 mL) and conc. HCl (0.235 mL) was heated to 80° C. After heating 3 hours, the formed precipitate was filtered and washed with ethanol to yield 94 mg (82%) of the title compound as a pale yellow solid: MS (ESI) m/z 385.2 (M+H)⁺. ¹H NMR (400 MHz, DMSO-d₆) δ ppm 3.73 (s, 3H), 6.10 (s, 1H), 6.27 (s, 1H), 6.47 (s, 1H), 7.36 (ds, J=8.1, 4.6 Hz, 1H), 7.63 (m, 5H), 8.43 (dd, J=4.2, 1.8 Hz, 1H), 8.80 (dd, J=9, 2.5 Hz, 1H), 10.81 (bd, 2H).

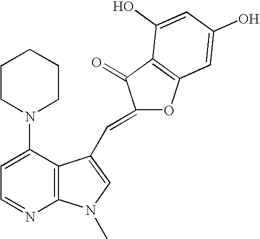

### (2Z)-4,6-Dihydroxy-2-[(1-methyl-4-piperidin-1-yl-1H-pyrrolo[2,3-b]pyridin-3-yl)methylene]-1-benzofuran-3(2H)-one

¹H NMR (400 MHz, DMSO-d₆) δ ppm 1.66 (m, 2H), 1.80 (m, 4H), 3.23 (m, 4H), 3.96 (s, 3H), 6.11 (s, 1H), 6.26 (s, 1H), 6.92 (d, J=6.3 Hz, 1H), 6.96 (s, 1H), 8.17 (s, 1H), 8.25 (d, J=6.3 Hz, 1H).

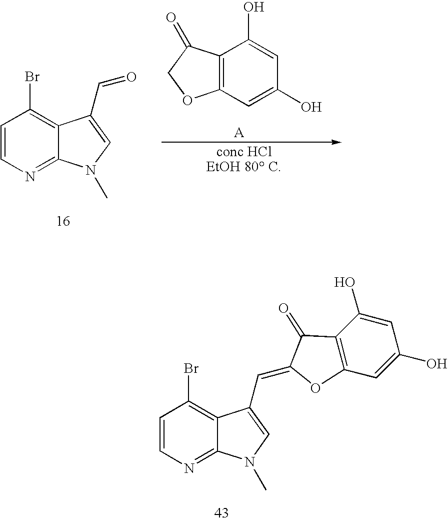

### Synthesis of 2-(4-bromo-1-methyl-1-H-pyrrolo[2,3-b ]pyridin-3-ylmethylene)-4,6-dihydroxy-benzofuran-3-one (43)

A mixture of 4-bromo-1-methyl-1-H-pyrrolo[2,3-b]pyridine-3-carbaldehyde (1.0 g, 4.2 mmol), 4-hydroxy-1-benzofuran-3(2H)-one (0.69 g, 4.2 mmol), ethanol (50 mL) and conc. HCl (0.25 mL) was heated to 80° C. After heating 6 hours, the formed precipitate was filtered and rinsed with ethanol to yield 0.66 g (41%) of a deep orange solid.

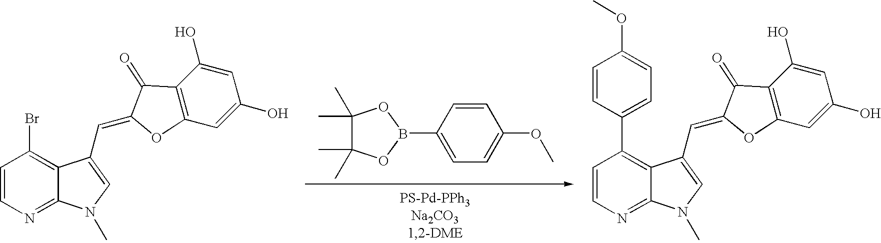

### Synthesis of (2Z)-4,6-dihydroxy-2-{[4-(4-methoxyphenyl)-1-methyl-1H-pyrrolo[2,3-b]pyridin-3-yl]methylene}-1-benzofuran-3(2H)-one

A mixture of 2-(4-bromo-1-methyl-1-H-pyrrolo[2,3-b]pyridin-3-ylmethylene)-4,6-dihydroxy-benzofuran-3-one (0.08 g, 0.21 mmol), 4-methoxyphenylboronic acid pinacol ester (0.1 g, 0.413 mmol), polymer supported palladium triphenylphosphine catalyst (Biotage, 0.11 mmol/g, 19 mg, 0.0021 mmol), in saturated sodium carbonate (0.5 mL) and 1,2-dimethoxyethane (2 mL) was heated to 120° C. in the microwave (Biotage Initiator 60) for 45 minutes. The slurry was filtered through a Whatman 45 micron filter, rinsed with methanol and concentrated. It was then purified on HPLC to afford 33 mg (38%) of mustard colored solids.

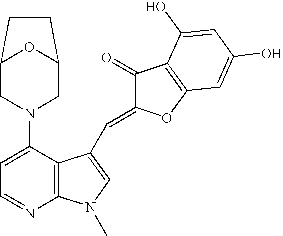

### Synthesis of (2Z)-4,6-dihydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one

A mixture of 4-(8-oxa-3-azabicyclo[3.2.1]octan-3-yl)-1-methyl-1H-pyrrolo[2,3-b]pyridine-3-carbaldehyde (37 mg, 0.136 mmol), 4,6-dihydroxy-1-benzofuran-3(2H)-one (23 mg, 0.139 mmol), ethanol (1 mL) and conc. HCl (0.11 mL) was heated to 80° C. After heating 21 hours, the formed precipitate was filtered and washed with ethanol to yield 42 mg (74%) of the title compound as an orange solid: MS (ESI) m/z 420.3 (M+H)⁺. ¹H NMR (400 MHz, DMSO-d₆) δ ppm 1.89 (bs, 2H), 2.25 (d, J=7.1, 2H), 3.15 (d, J=11.5, 2H), 3.47 (d, J=11.5, 2H), 4.41 (bs, 2H), 6.08 (d, J=1.8, 2H), 6.25 (d, J=1.8, 2H), 6.98 (s, 1H), 7.01 (d, J=6.5 Hz, 1H), 8.17 (s, 1H), 8.22 (d, J=6.5 Hz, 1H), 10.9 (bd, 2H).

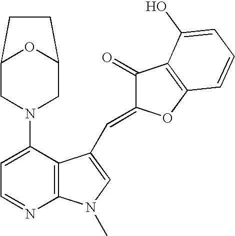

### Synthesis of (2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one

A mixture of 4-(8-oxa-3-azabicyclo[3.2.1]octan-3-yl)-1-methyl-1H-pyrrolo[2,3-b]pyridine-3-carbaldehyde (37 mg, 0.136 mmol), 4-hydroxy-1-benzofuran-3(2H)-one (20.9 mg, 0.139 mmol), ethanol (1 mL) and conc. HCl (0.11 mL) was heated to 80° C. After heating 21 hours, the formed precipitate was filtered and washed with ethanol to yield 48 mg (88%) of the title compound as a yellow solid: MS (ESI) m/z 404.3 (M+H)⁺. ¹H NMR (400 MHz, DMSO-d₆) δ ppm 1.92 (bs, 2H), 2.25 (d, J=7.1, 2H), 3.17 (d, J=11.4, 2H), 3.46 (d, J=11.4, 2H), 4.42 (bs, 2H), 6.65 (d, J=8.3, 1H), 6.85 (d, J=8.0, 2H), 7.0 (d, J=6 Hz, 1H), 7.14 (s, 1H), 7.54 (t, J=8.3 Hz, 1H), 8.23 (d, J=6.3 Hz, 1H), 8.25 (s, 1H), 11.1 (bd, 1H).

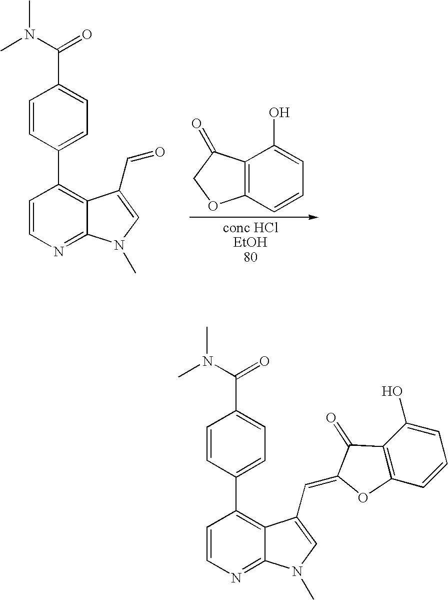

### 4-{3-[(Z)-(4-hydroxy-3-oxo-1-benzofuran-2(3H)-ylidene)methyl]-1-methyl-1H-pyrrolo[2,3-b]pyridin-4-yl}-N,N-dimethyl benzamide

A mixture of 4-(3-formyl-1-methyl-1-H-pyrrolo[2,3-b]pyridin-4-yl)-N,N-dimethyl-benzamide (0.78 g, 0.256 mmol),), 4-hydroxy-1-benzofuran-3(2H)-one (0.038 g, 0.256 mmol), ethanol (5 mL), and conc. HCl (0.025 mL) was heated to 80° C. After heating 6 hours, the formed precipitate was filtered and rinsed with ethanol to yield 0.078 g (69%) of an orange solid. HRMS (ESI) m/e calcd for C26H21N3O4 439.1605, found 439.1603 (M+H)⁺1; ¹H NMR (400 MHz, DMSO-d₆) δ ppm 3.10 (d, J=10.8 Hz, 1H), 4.01 (s, 3H), 6.37 (s, 1H), 6.57 (d, J=8.4 Hz, 1H), 6.83 (d, J=8.4 Hz, 1H), 7.18 (d, J=4.2 Hz, 1H), 7.48 (t, J=8.4 Hz, 1H), 7.61 (s, 4H), 8.43-8.45 (m, 2H), 11.87 (broad, 1H).

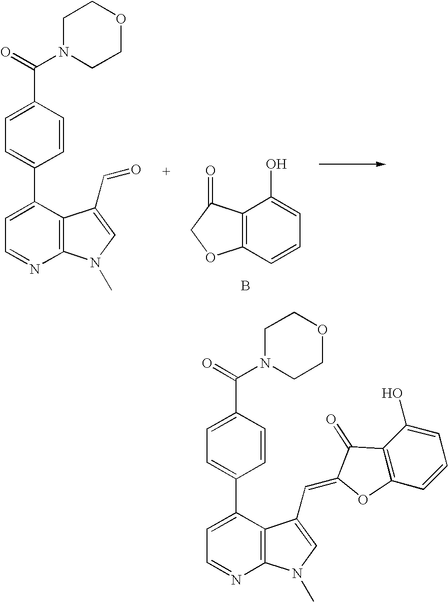

### (Z)-4-Hydroxy-24(1-methyl-4-(4-(morpholine-4-carbonyl)phenyl)-1H-pyrrolo[2,3-b]pyridin-3-yl)methylene)benzofuran-3(2H)-one

1-Methyl-4-(4-(morpholine-4-carbonyl)phenyl)-1-H-pyrrolo[2,3-b]pyridine-3 carbaldehyde (94.5 mg, 0.27 mmol) and 4-hydroxybenzofuran-3(2H)-one (42.6 mg, 0.285 mmol) were stirred in absolute EtOH (2.2 mL), followed by addition of 0.22 mL of concentrated HCl. It was heated at 80° C. After 4.5 hours, the reaction mixture was cooled in ice bath and filtered. The solid was washed with 4 mL of cold absolute EtOH, dried in vacuum, gave 99.0 mg (76%) of the title product, as a pale yellow solid. HRMS (ESI) m/e calcd for C28H23N3O5 481.16385, found 482.17066 (M+H)⁺1; ¹H NMR (400 MHz, DMSO-d₆) δ ppm 2.50 (d, J=2.0 Hz, 4H), 3.70 (bs, 4H), 4.00 (s, 3H), 6.28 (s, 1H), 6.58 (d, J=2.0 Hz, 1H), 6.82 (d, J=2.0 Hz, 1H), 7.17 (d, J=2.0 Hz, 1H), 7.45 (t, J=2.0 Hz, 1H), 7.59 (s, 3H), 8.33 (s, 1H), 8.42 (d, J=2.0 Hz, 1H), 10.89 (bs, 1H).

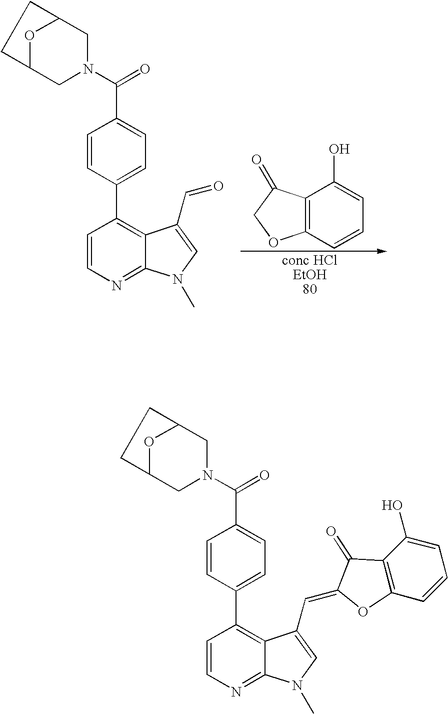

### (2Z)-4-hydroxy-2-({1-methyl-4-[4-(8-oxa-3-azabicyclo[3.2.1]oct-3-ylcarbonyl)phenyl]-1H-pyrrolo[2,3-b]pyridin-3-yl}methylidene)-1-benzofuran-3(2H)-one

A mixture of 1-methyl-4-[4-(8-oxa-3-azabicyclo[3.2.1]octane-3-carbonyl)-phenyl]-1H-pyrrolo[2,3-b]pyridine-3-carbaldehyde (0.093 g, 0.25 mmol), 4-hydroxy-1-benzofuran-3(2H)-one (0.037 g, 0.25 mmol), ethanol (5 mL) and conc. HCl (0.025 mL) was heated to 80° C. After heating 6 hours, the formed precipitate was filtered and rinsed with ethanol to yield 0.089 g (70%) of a yellow solid. HRMS (ESI) m/e calcd for C₃₀H₂₅N₃O₅ 508.1867, found 508.1864 (M+H)⁺¹; ¹H NMR (400 MHz, DMSO-d₆) δ ppm 1.65-1.9 (m, 4H), 3.04 (d, J=12.1 Hz, 1H), 3.51 (d, J=12.1 Hz, 1H), 4.00 (s, 3H), 4.03-4.3 (m, assume 2H, overlapping with water), 4.42 (s, 2H), 6.26 (s, 1H); 6.60 (d, J=8.2 Hz, 1H), 6.82 (d, J=8.2 Hz, 1H), 7.17 (d, J=5.8 Hz, 1H), 7.43 (t, J=8.2 Hz, 1H), 7.57 (m, 4H), 8.41 (m, 2H), 11.1 (broad, 1H).

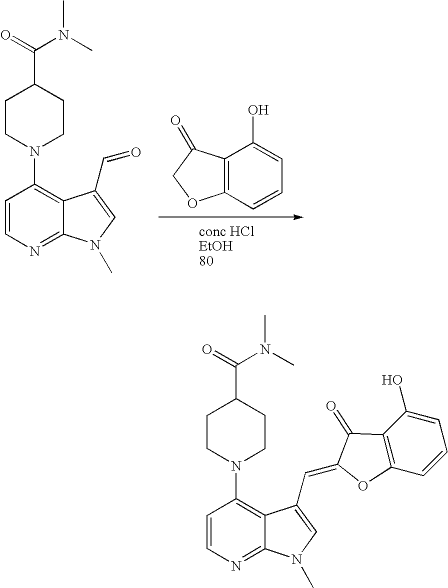

### 1-{3-[(Z)-(4-hydroxy-3-oxo-1-benzofuran-2(3H)-ylidene)methyl]-1-methyl-1H-pyrrolo[2,3-b]pyridin-4-yl}-N,N-dimethyl piperidine-4-carboxamide

A mixture of 1-(3-formyl-1-methyl-1H-pyrrolo[2,3-b]pyridin-4-yl)-piperidine-4-carboxylic acid dimethylamide (0.072 g, 0.23 mmol),), 4-hydroxy-1-benzofuran-3(2H)-one (0.034 g, 0.23 mmol), ethanol (5 mL) and conc. HCl (0.025 mL) was heated to 80° C. After heating 6 hours, the solution was cooled and concentrated to half volume. The solids were filtered and rinsed with acetonitrile to yield 0.036 g (35%) of a yellow solid. HRMS (ESI) m/e calcd for C₂₅H₂₆N₄O₄ 447.2027, found 447.2032 (M+H)⁺¹; ¹H NMR (400 MHz, DMSO-d₆) δ ppm 1.83 (d, J=12.4 Hz, 2H), 1.94 (q, J=12.4 Hz, 2H), 2.86 (s, 3H), 3.06 (s, 3H), 2.82-3.07 (m, assume 3H buried), 3.71 (d, J=12.4 Hz, 2H), 3.96 (s, 3H), 6.65 (d, J=8.5 Hz, 1H); 6.86 (d, J=7.6 Hz, 1H), 6.94 (d, J=7.6 Hz, 1H), 7.08 (s, 1H), 7.53 (t, J=8.5 Hz, 1H), 8.26 (d, J=7.6 Hz, 2H), 11.02 (broad, 1H).

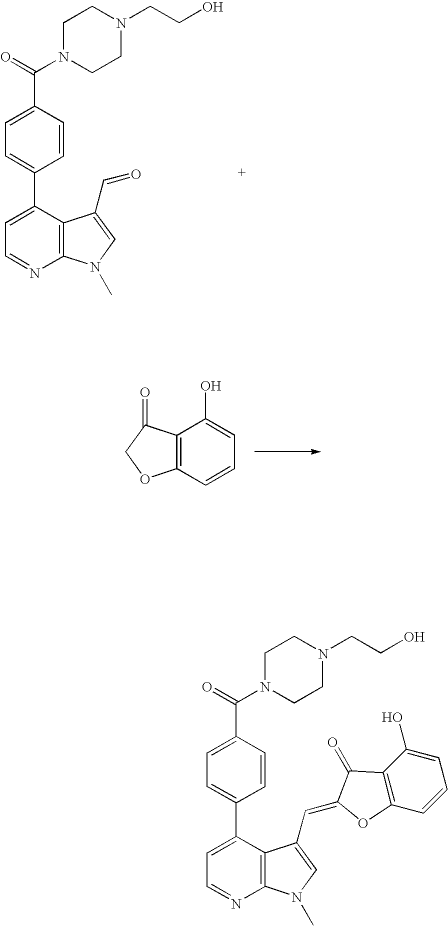

### (Z)-4-hydroxy-2-((4(4-(4-(4-(2-hydroxyethyl)piperazine-1-carbonyl)phenyl)-1-methyl-1H-pyrrolo[2,3-b]pyridin-3-yl)methylene)benzofuran-3(2H)-one

4-(4-(4-(2-Hydroxyethyl)piperazine-1-carbonyl)phenyl)-1-methyl-1H-pyrrolo[2,3-b]pyridine-3-carbaldehyde (160 mg, 0.408 mmol) and 4-hydroxybenzofuran-3(2H)-one (64.2 mg., 0.428 mmol) were stirred in absolute EtOH (3.2 mL), followed by addition of 0.34 mL of concentrated HCl. It was heated at 80° C. After 5.2 hours, the reaction mixture was cooled in ice bath and filtered. The solid washed with 4 mL of cold absolute EtOH, dried in vacuum, gave 154.0 mg (72%) of the title product, as a pale yellow solid. MS (ESI) m/e calcd for C₃₀H₂₈N₄O₅524.2, found 525.2 (M+H)⁺¹. ¹H NMR (400 MHz, DMSO-d₆) δ ppm 3.24 (bd, J=3.0 Hz, 2H), 3.62-3.65 (bd, J=3.0 Hz, 2H), 3.81-3.83 (m, 4H), 3.86 (m, 4H), 6.24 (s, 1H), 6.63 (d, J=2.0 Hz, 1H), 6.83 (d, J=2.0 Hz, 1H), 7.20 (d, J=2.0 Hz, 1H), 7.50 (t, J=2.0 Hz, 1H), 7.61-7.67 (m, 3H), 8.42 (s, 1H), 8.45 (d, J=1.0 Hz, 1H), 10.98 (bs, 1H).

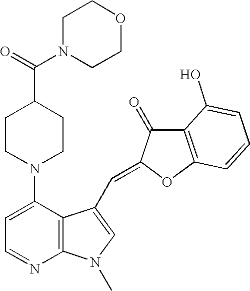

### (Z)-4-hydroxy-2-((1-methyl-4-(4-(morpholine-4-carbonyl)piperidin-1-yl)-1H-pyrrolo[2,3-b]pyridin-3-yl)methylene)benzofuran-3(2H)-one

To a mixture of 1-methyl-4-(4-(morpholine-4-carbonyl)piperidin-1-yl)-1H-pyrrolo[2,3-b]pyridine-3-carbaldehyde (89 mg, 0.25 mmole-4-hydroxybenzofuran-3(2H)-one (38 mg, 0.25 mmol) and EtOH (5 ml) was added 3 drops of concentrated hydrochloric acid. This was heated to 80° C. and stirred overnight. The reaction mixture was cooled and the orange solid collected by filtration, washed with ethanol, washed with ether, air dried and vacuum dried to give (Z)-4-hydroxy-2-((1-methyl-4-(4-(morpholine-4-carbonyl)piperidin-1-yl)-1H-pyrrolo[2,3-b]pyridin-3-yl)methylene)benzofuran-3(2H)-one (82 mg, 67% yield) mp 358-61 (dec). Mol Ion: M+H 489.2; ¹H NMR (400 MHz, DMSO-d₆) δ ppm 1.76-1.87 (m, 2H), 1.87-2.01 (m, 2H), 2.86-2.96 (m, 1H), 2.96-3.07 (m, 2H), 3.46-3.71 (m, 10H), 3.95 (s, 3H), 6.63 (d, J=8.3 Hz, 1H), 6.86 (d, J=8.1 Hz, 1H), 6.92 (d, J=5.9 Hz, 1H), 7.11 (s, 1H) 7.58 (t, J=8.2 Hz, 1H) 8.25 (d, J=5.9 Hz, 1H) 8.26 (s, 1H) 11.01 (obs, 1H).

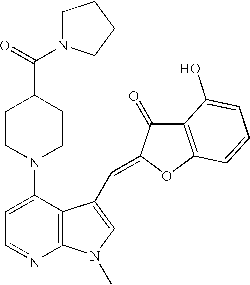

### (Z)-4-hydroxy-2-((1-methyl-4-(4-(pyrrolidine-1-carbonyl)piperidin-1-yl)-1H-pyrrolo[2,3-b]pyridin-3-yl)methylene)benzofuran-3(2H)-one

To a mixture of 1-methyl-4-(4-(pyrrolidine-1-carbonyl)piperidin-1-yl)-1H-pyrrolo[2,3-b]pyridine-3-carbaldehyde (55 mg, 0.162 mmole-4-hydroxybenzofuran-3(2H)-one (24 mg, 0.162 mmol) and EtOH (3 ml) was added 2 drops of concentrated hydrochloric acid. This was heated to 80° C. and stirred overnight. The reaction mixture was cooled and the yellow solid collected by filtration, washed with ethanol, washed with ether, air dried and vacuum dried to give (Z)-4-hydroxy-2-((1-methyl-4-(4-(pyrrolidine-1-carbonyl)piperidin-1-yl)-1H-pyrrolo[2,3-b]pyridin-3-yl)methylene)benzofuran-3(2H)-one (36 mg, 46% yield) mp 191-207 (dec). Mol Ion: M+H 473.2; ¹H NMR (400 MHz, DMSO-d₆) δ ppm 1.74-2.02 (m, 8H), 2.63-2.73 (m, 1H), 2.87-2.98 (m, 2H), 3.30 (t, J=6.9 Hz, 2H), 3.51 (t, J=6.9 Hz, 2H), 3.58-3.67 (m, 2H), 3.94 (s, 3H), 6.62 (d, J=8.2 Hz, 1H), 6.86 (d, J=8.1 Hz, 1H), 6.89 (d, J=5.9 Hz, 1H), 7.16 (s, 1H) 7.52 (t, J=8.2 Hz, 1H) 8.23 (d, J=5.7 Hz, 1H) 8.26 (s, 1H) 10.97 (obs, 1H).

Using the procedure of any of the preparations of Preparations Sections A-III or B-III, the following phenolic compounds were prepared. The compounds shown below could be formed as the (Z) isomer or as a mixture of (Z) and (E) isomers.
4,6-dihydroxy-2-[(5-methoxy-2-phenyl-1H-indol-3-yl)methylene]-1-benzofuran-3(21-1)-one;
(2Z)-6-hydroxy-2-({5-methoxy-2-methyl-1-[2-(4-methylpiperazin-1-yl)pethyl]-1H-indol-3-yl}methylene)-1-benzofuran-3(21-1)-one;
(2Z)-4,6-dihydroxy-2-({5-methoxy-2-methyl-1-[2-(4-methylpiperazin-1-yl)pethyl]-1H-indol-3-yl}methylene)-1-benzofuran-3(21-1)-one;
(2Z)-6-hydroxy-2-[(5-methoxy-2-phenyl-1H-indol-3-yl)methylene]-1-benzofuran-3(21-1)-one;
(2Z)-2-({1-[3-(dimethylamino)propyl]-5-methoxy-1H-indol-3-yl}methylene)-4,6-dihydroxy-1-benzofuran-3(21-1)-one;
(2Z)-2-({1-[3-(dimethylamino)propyl]-5-methoxy-1H-indol-3-yl}methylene)-6-hydroxy-1-benzofuran-3(21-1)-one;
(2Z)-5-hydroxy-2-({5-methoxy-2-methyl-1-[2-(4-methylpiperazin-1-yl)pethyl]-1H-indol-3-yl}methylene)-1-benzofuran-3(21-1)-one;
(2Z)-4,6-dihydroxy-2-[(1-methyl-4-phenyl-1H-pyrrolo[2,3-b]pyridin-3-yl)methylene]-1-benzofuran-3(2H)-one;
(2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(21-1)-one; and
(2Z)-4,6-dihydroxy-2-[(1-methyl-4-phenyl-1H-indol-3-yl)methylene]-1-benzofuran-3(21-1)-one.

### Experimental Procedures for the Benzofuranone Prodrugs

To a solution of a phenolic compound prepared as described by any of the schemes in Sections A-III or B-III (0.35 mmol) in the selected solvent (10 mL), the base, and the selected electrophile (R²⁰Cl) were added at 0° C. The amounts of both reagents are specified in the following tables, for the different reactions. The cooling bath was removed and the solution was stirred at room temperature for the time indicated in the tables. The reaction mixture was then submitted to one of the following work-up procedures:

A) solvent was evaporated and the residue was diluted with water and extracted with methylene chloride;

B) the reaction was diluted with water and extracted with methylene chloride;

C) the reaction was diluted with 1 N HCl and extracted with methylene chloride;

D) methylene chloride was added and the organic phase was washed with 5% citric acid;

E) methylene chloride was added and the organic phase was washed with 1 N HCl and then with 0.5 N NaHCO₃;

F) methylene chloride was added and the organic phase was washed with 5% citric acid and then with 0.5 N NaHCO₃.

Work-up, purification methods, and yields of the final products are reported in Tables I-IV. The procedures below illustrate methods of preparation of representative compounds.

| | |
|---|---|
| 1 | (2Z)-2-({1-[3-(dimethylamino)propyl]-5-methoxy-1H-indol-3- |
| | yl}methylene)-3-oxo-2,3-dihydro-1-benzofuran-4,6-diyl |
| | dimorpholine-4-carboxylate |
| 2 | (2Z)-2-({1-[3-(dimethylamino)propyl]-5-methoxy-1H-indol-3- |
| | yl}methylene)-3-oxo-2,3-dihydro-1-benzofuran-4,6-diyl |
| | bis[methyl(phenyl)carbamate] |
| 3 | (2Z)-2-[(5-methoxy-2-phenyl-1H-indol-3-yl)methylene]-3-oxo-2,3- |
| | dihydro-1-benzofuran-4,6-diyl diacetate |
| 4 | (2Z)-2-({1-[3-(dimethylamino)propyl]-5-methoxy-1H-indol-3- |
| | yl}methylene)-3-oxo-2,3-dihydro-1-benzofuran-4,6-diyl |
| | bis(diisopropylcarbamate) |
| 5 | (2Z)-2-({1-[3-(dimethylamino)propyl]-5-methoxy-1H-indol-3- |
| | yl}methylene)-3-oxo-2,3-dihydro-1-benzofuran-4,6-diyl diisopropyl |
| | biscarbonate |
| 6 | (2Z)-2-[(5-methoxy-2-phenyl-1H-indol-3-yl)methylene]-3-oxo-2,3- |
| | dihydro-1-benzofuran-4,6-diyl dibenzoate |
| 7 | diisopropyl (2Z)-2-[(5-methoxy-2-phenyl-1H-indol-3-yl)methylene]- |
| | 3-oxo-2,3-dihydro-1-benzofuran-4,6-diyl biscarbonate |
| 8 | (2Z)-2-({1-[3-(dimethylamino)propyl]-5-methoxy-1H-indol-3- |
| | yl}methylene)-3-oxo-2,3-dihydro-1-benzofuran-4,6-diyl |
| | bis(dimethylcarbamate) |
| 9 | (2Z)-2-({1-[3-(dimethylamino)propyl]-5-methoxy-1H-indol-3- |
| | yl}methylene)-3-oxo-2,3-dihydro-1-benzofuran-4,6-diyl bis(3- |
| | methylbutanoate) |
| 10 | (2Z)-2-[(5-methoxy-2-phenyl-1H-indol-3-yl)methylene]-3-oxo-2,3- |
| | dihydro-1-benzofuran-4,6-diyl bis(2,2-dimethylpropanoate) |
| 11 | (2Z)-2-[(5-methoxy-2-phenyl-1H-indol-3-yl)methylene]-3-oxo-2,3- |
| | dihydro-1-benzofuran-4,6-diyl bis(diphenylcarbamate) |
| 12 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-4,6-diyl |
| | bis(dimethylcarbamate) |
| 13 | (2Z)-2-({1-[3-(dimethylamino)propyl]-5-methoxy-1H-indol-3- |
| | yl}methylene)-3-oxo-2,3-dihydro-1-benzofuran-4,6-diyl dimethyl |
| | biscarbonate |
| 14 | (2Z)-2-({1-[3-(dimethylamino)propyl]-5-methoxy-1H-indol-3- |
| | yl}methylene)-3-oxo-2,3-dihydro-1-benzofuran-4,6-diyl bis(2,2- |
| | dimethylpropanoate) |
| 15 | (2Z)-2-({1-[3-(dimethylamino)propyl]-5-methoxy-1H-indol-3- |
| | yl}methylene)-3-oxo-2,3-dihydro-1-benzofuran-4,6-diyl diacetate |
| 16 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-4,6-diyl |
| | dibenzoate |
| 17 | (2Z)-2-[(5-methoxy-2-phenyl-1H-indol-3-yl)methylene]-3-oxo-2,3- |
| | dihydro-1-benzofuran-6-yl benzoate |
| 18 | (2Z)-2-[(5-methoxy-2-phenyl-1H-indol-3-yl)methylene]-3-oxo-2,3- |
| | dihydro-1-benzofuran-6-yl dimethylcarbamate |
| 19 | (2Z)-2-[(5-methoxy-2-phenyl-1H-indol-3-yl)methylene]-3-oxo-2,3- |
| | dihydro-1-benzofuran-6-yl phenyl carbonate |
| 20 | (2Z)-2-[(5-methoxy-2-phenyl-1H-indol-3-yl)methylene]-3-oxo-2,3- |
| | dihydro-1-benzofuran-6-yl diphenylcarbamate |
| 21 | (2Z)-2-({1-[3-(dimethylamino)propyl]-5-methoxy-1H-indol-3- |
| | yl}methylene)-3-oxo-2,3-dihydro-1-benzofuran-4,6-diyl |
| | bis(diphenylcarbamate) |
| 22 | (2Z)-2-[(5-methoxy-2-phenyl-1H-indol-3-yl)methylene]-3-oxo-2,3- |
| | dihydro-1-benzofuran-4,6-diyl dimethyl biscarbonate |
| 23 | (2Z)-2-[(5-methoxy-2-phenyl-1H-indol-3-yl)methylene]-3-oxo-2,3- |
| | dihydro-1-benzofuran-4,6-diyl diphenyl biscarbonate |
| 24 | (2Z)-2-({1-[3-(dimethylamino)propyl]-5-methoxy-1H-indol-3- |
| | yl}methylene)-3-oxo-2,3-dihydro-1-benzofuran-4,6-diyl dibenzoate |
| 25 | ethyl N-({[(2Z)-2-({1-[3-(dimethylamino)propyl]-5-methoxy-1H- |
| | indol-3-yl}methylene)-3-oxo-2,3-dihydro-1-benzofuran-6- |
| | yl]oxy}carbonyl)glycinate |
| 26 | (2Z)-2-[(5-methoxy-2-phenyl-1H-indol-3-yl)methylene]-3-oxo-2,3- |
| | dihydro-1-benzofuran-6-yl hydrogen phenylphosphonate |
| 27 | (2Z)-2-[(5-methoxy-2-phenyl-1H-indol-3-yl)methylene]-3-oxo-2,3- |
| | dihydro-1-benzofuran-6-yl dihydrogen phosphate |
| 28 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-4,6-diyl |
| | bis(diisopropylcarbamate) |
| 29 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-4,6-diyl |
| | bis(diphenylcarbamate) |
| 30 | (2Z)-2-[(5-methoxy-2-phenyl-1H-indol-3-yl)methylene]-3-oxo-2,3- |
| | dihydro-1-benzofuran-4,6-diyl bis(dimethylcarbamate) |
| 31 | (2Z)-2-[(5-methoxy-2-phenyl-1H-indol-3-yl)methylene]-3-oxo-2,3- |
| | dihydro-1-benzofuran-4,6-diyl bis(diisopropylcarbamate) |
| 32 | tetraethyl (2Z)-2-[(5-methoxy-2-phenyl-1H-indol-3-yl)methylene]- |
| | 3-oxo-2,3-dihydro-1-benzofuran-4,6-diyl bis(phosphate) |
| 33 | (2Z)-2-({1-[3-(dimethylamino)propyl]-5-methoxy-1H-indol-3- |
| | yl}methylene)-3-oxo-2,3-dihydro-1-benzofuran-4,6-diyl tetraethyl |
| | bis(phosphate) |
| 34 | diethyl (2Z)-2-[(5-methoxy-2-phenyl-1H-indol-3-yl)methylene]-3- |
| | oxo-2,3-dihydro-1-benzofuran-6-yl phosphate |
| 35 | diethyl 2,2′-[[(2Z)-2-({1-[3-(dimethylamino)propyl]-5-methoxy-1H- |
| | indol-3-yl}methylene)-3-oxo-2,3-dihydro-1-benzofuran-4,6- |
| | diyl]bis(oxycarbonylimino)]diacetate |
| 36 | (2Z)-2-({1-[3-(dimethylamino)propyl]-5-methoxy-1H-indol-3- |
| | yl}methylene)-3-oxo-2,3-dihydro-1-benzofuran-4,6-diyl bis(4- |
| | methylpiperazine-1-carboxylate) |
| 37 | (2Z)-2-({1-[3-(dimethylamino)propyl]-5-methoxy-1H-indol-3- |
| | yl}methylene)-3-oxo-2,3-dihydro-1-benzofuran-4,6-diyl bis(4- |
| | benzylpiperazine-1-carboxylate) |
| 38 | (2Z)-2-[(5-methoxy-2-phenyl-1H-indol-3-yl)methylene]-3-oxo-2,3- |
| | dihydro-1-benzofuran-4,6-diyl bis[dihydrogen (phosphate)] |
| 39 | (2Z)-6-hydroxy-2-({5-methoxy-2-methyl-1-[2-(4-methylpiperazin-1- |
| | yl)ethyl]-1H-indol-3-yl}methylene)-3-oxo-2,3-dihydro-1- |
| | benzofuran-4-yldimethylcarbamate |
| 40 | (2Z)-2-({1-[3-(dimethylamino)propyl]-5-methoxy-1H-indol-3- |
| | yl}methylene)-6-hydroxy-3-oxo-2,3-dihydro-1-benzofuran-4-yl |
| | dimethylcarbamate |
| 41 | (2Z)-4-hydroxy-2-({5-methoxy-2-methyl-1-[2-(4-methylpiperazin-1- |
| | yl)ethyl]-1H-indol-3-yl}methylene)-3-oxo-2,3-dihydro-1- |
| | benzofuran-6-yldimethylcarbamate |
| 42 | (2Z)-2-({1-[3-(dimethylamino)propyl]-5-methoxy-1H-indol-3- |
| | yl}methylene)-4-hydroxy-3-oxo-2,3-dihydro-1-benzofuran-6-yl |
| | dimethylcarbamate |
| 43 | (2Z)-2-[(1-methyl-4-phenyl-1H-pyrrolo[2,3-b]pyridin-3- |
| | yl)methylene]-3-oxo-2,3-dihydro-1-benzofuran-4,6-diyl |
| | bis(dimethylcarbamate) |
| 44 | (2Z)-2-{[1-methyl-4-(8-oxa-3-azabicyclo[3.2.1]oct-3-yl)-1H- |
| | pyrrolo[2,3-b]pyridin-3-yl]methylene}-3-oxo-2,3-dihydro- |
| | 1-benzofuran-4-yl dimethylcarbamate |
| 45 | (2Z)-2-({5-methoxy-1-[2-(4-methylpiperazin-1-yl)ethyl]-1H-indol-3- |
| | yl}methylene)-3-oxo-2,3-dihydro-1-benzofuran-5-yl |
| | methylcarbamate; |
| 46 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-6-yl |
| | methyl(phenyl)carbamate; |
| 47 | (2Z)-2-({5-methoxy-2-methyl-1-[2-(4-methylpiperazin-1-yl)ethyl]- |
| | 1H-indol-3-yl}methylene)-3-oxo-2,3-dihydro-1-benzofuran-6-yl |
| | diisopropylcarbamate |
| 48 | (2Z)-2-[(1-methyl-4-phenyl-1H-indol-3-yl)methylene]-3-oxo-2,3- |
| | dihydro-1-benzofuran-4,6-diyl bis(dimethylcarbamate). |

Additional Procedures:

Free phosphoric and phosphonic acids were prepared from the corresponding esters, as shown in Examples 26, 27, 38. Additional prodrugs were prepared via different procedures, as shown in Examples 25, 35, 36, 37.

### Prodrugs of (2Z)-6-hydroxy-2-[(5-methoxy-2-phenyl-1H-indol-3-yl)methylene]-1-benzofuran-3(2H)-one

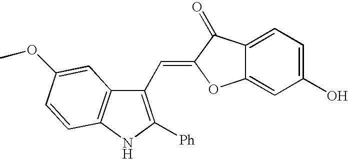

Following the procedure above and Scheme C-I, compounds in Table I were prepared. In Tables I-IV below, the symbols A-F stand for the following work-up procedures:

A) solvent was evaporated and the residue was diluted with water and extracted with methylene chloride;

B) the reaction was diluted with water and extracted with methylene chloride;

C) the reaction was diluted with 1 N HCl and extracted with methylene chloride;

D) methylene chloride was added and the organic phase was washed with 5% citric acid;

E) methylene chloride was added and the organic phase was washed with 1 N HCl and then with 0.5 N NaHCO₃;

F) methylene chloride was added and the organic phase was washed with 5% citric acid and then with 0.5 N NaHCO₃.

TABLE I

| | | | | | | | | |
|---|---|---|---|---|---|---|---|---|
| Ex. | | Eq. of | | | | | | Yield |
| # | R²⁰ Cl | R²⁰ Cl | Base | Solvent | time | workup | Purification | % |

Esters

| | | | | | | | | |
|---|---|---|---|---|---|---|---|---|
| 17 | 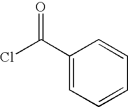 | 2.6 | Py (4 eq.) | methylene chloride | 36 hours | B | Trituration with Et₂O | 65 |

Carbamates

| | | | | | | | | |
|---|---|---|---|---|---|---|---|---|
| 18 | 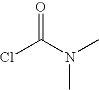 | 1.5 | / | Py | 12 hours | A | Trituration with Et₂O | 57 |
| 20 | 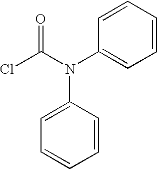 | 3 | Py (2 mL) | methylene chloride | 3.5 days | B | Silica gel column chromatography (gradient from methylene chloride to methylene chloride/AcOEt 10:1) | 43 |

Carbonates

| | | | | | | | | |
|---|---|---|---|---|---|---|---|---|
| 19 | 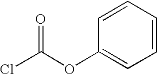 | 1.5 | / | Py | 12 hours | C | Trituration with Et₂O | 57 |

Phosphate

| | | | | | | | | |
|---|---|---|---|---|---|---|---|---|
| 34 | 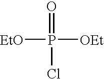 | 1.5 | Et₃N (2.5 eq.) | THF | 12 hours | B | Preparative HPLC | 43 |

### Example 17

### (2Z)-2-[(5-Methoxy-2-phenyl-1H-indol-3-yl)methylene]-3-oxo-2,3-dihydro-1-benzofuran-6-yl benzoate

MS (m/z): 488.0 (MH⁺).

### Example 18

### (2Z)-2-[(5-Methoxy-2-phenyl-1H-indol-3-yl)methylene]-3-oxo-2,3-dihydro-1-benzofuran-6-yl dimethylcarbamate

MS (m/z): 455.2 (MH⁺).

### Example 20

### (2Z)-2-[(5-Methoxy-2-phenyl-1H-indol-3-yl)methylene]-3-oxo-2,3-dihydro-1-benzofuran-6-yl diphenylcarbamate

MS (m/z): 579.2 (MH⁺).

### Example 19

### (2Z)-2-[(5-Methoxy-2-phenyl-1H-indol-3-yl)methylene]-3-oxo-2,3-dihydro-1-benzofuran-6-yl phenyl carbonate

MS (m/z): 504.2 (MH⁺).

### Example 34

### Diethyl (2Z)-2-[(5-methoxy-2-phenyl-1H-indol-3-yl)methylene]-3-oxo-2,3-dihydro-1-benzofuran-6-yl phosphate

MS (m/z): 520.3 (MH⁺).

### Example 27

### (2Z)-2-[(5-Methoxy-2-phenyl-1H-indol-3-yl)methylene]-3-oxo-2,3-dihydro-1-benzofuran-6-yl dihydrogen phosphate

To a solution of diethyl (2Z)-2-[(5-methoxy-2-phenyl-1H-indol-3-yl)methylene]-3-oxo-2,3-dihydro-1-benzofuran-6-yl phosphate (Example 34; 155 mg, 0.299 mmol, 1 eq.) in dry methylene chloride (15 mL), Me₃SiBr (788 μL, 5.973 mmol, 20 eq.) was added. The resulting mixture was refluxed for 3 days, then NaHCO₃ (602 mg, 7.17 mmol, 24 eq.) and MeOH were added. The resulting mixture was stirred at room temperature for 1 hour, and the solvents were evaporated. The crude material was purified by preparative HPLC, affording the pure title compound. Yield: 15%. MS (m/z): 464.0 (MH⁺).

### Example 26

### Preparation of (2Z)-2-[(5-methoxy-2-phenyl-1H-indol-3-yl)methylene]-3-oxo-2,3-dihydro-1-benzofuran-6-yl hydrogen phenylphosphonate

To a solution of (2Z)-6-hydroxy-2-[(5-methoxy-2-phenyl-1H-indol-3-yl)methylene]-1-benzofuran-3(2H)-one (100 mg, 0.261 mmol, 1 eq.) and triethylamine (91 μL, 0.653 mmol, 2.5 eq.) in THF (10 mL), phenylphosphoryl dichloride (113 μL, 0.783 mmol, 1.5 eq.) was added dropwise at 0° C. The resulting mixture was stirred at room temperature overnight. Water was added and the reaction mixture was allowed to stir for 2 hours. 1 N HCl was added and the solvents were evaporated. The crude product was purified by preparative HPLC affording the pure title compound. Yield: 26%. MS (m/z): 524.1 (MH⁺).

### Prodrugs of 4,6-dihydroxy-2-[(5-methoxy-2-phenyl-1H-indol-3-yl)methylene]-1-benzofuran-3(2H)-one

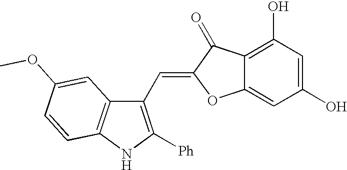

Following the general procedure above and Scheme C-II, compounds in Table II were prepared.

TABLE II

| Ex. | | Eq. of | | | | | | Yield |
|---|---|---|---|---|---|---|---|---|
| # | R²⁰ Cl | R²⁰ Cl | Base | Solvent | time | Work-up | Purification | % |
| 11 | 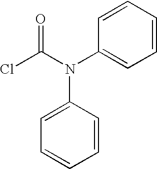 | 5 | Py (2.5 mL) | THF | 3 days | D | Trituration with i-Pr₂O | 83 |
| 30 | 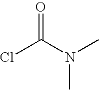 | 3 | Py (3 eq.) | THF | 3 days | D | Trituration with methylene chloride, i- Pr₂O and Et₂O | 47 |
| 31 | 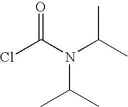 | 10 | Py (5 mL) | THF | 9 days | D | Trituration with i-Pr₂O | 60 |
| 7 | 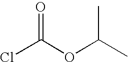 | 2.5 | Py (5 eq.) | THF | 12 hours | D | Trituration with Et₂O and hexane | 39 |
| 22 | 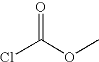 | 2.5 | Et₃N (3 eq.) | THF | 1.5 hours | D | Trituration with MeOH | 39 |
| 23 | 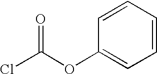 | 2.5 | Py (4 eq.) | methylene chloride | 36 hours | C | Trituration with Et₂O, then with MeOH | 8 |
| 3 | 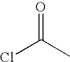 | 2.5 | Py (5 eq.) | THF | 3 hours | D | Trituration with Et₂O | 77 |
| 6 | 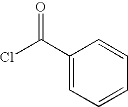 | 2.5 | Py (5 eq.) | THF | 4 days | D | Trituration with methylene chloride and Et₂O | 46 |
| 10 | 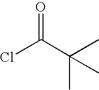 | 5 | Py (0.5 mL) | THF | 3 days | D | Trituration with methylene chloride and hexane | 75 |
| 32 | 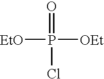 | 3 | Et₃N (5 eq.) | THF | 12 hours | B | Preparative HPLC | 20 |

### Example 11

### (2Z)-2-[(5-Methoxy-2-phenyl-1H-indol-3-yl)methylene]-3-oxo-2,3-dihydro-1-benzofuran-4,6-diyl bis(diphenylcarbamate)

MS (m/z): 790.3 (MH⁺).

### Example 30

### (2Z)-2-[(5-Methoxy-2-phenyl-1H-indol-3-yl)methylene]-3-oxo-2,3-dihydro-1-benzofuran-4,6-diyl bis(dimethylcarbamate)

MS (m/z): 542.1 (MH⁺).

### Example 31

### (2Z)-2-[(5-Methoxy-2-phenyl-1H-indol-3-yl)methylene]-3-oxo-2,3-dihydro-1-benzofuran-4,6-diyl bis(diisopropylcarbamate)

MS (m/z): 654.4 (MH⁺).

### Example 7

### Diisopropyl (2Z)-2-[(5-methoxy-2-phenyl-1H-indol-3-yl)methylene]-3-oxo-2,3-dihydro-1-benzofuran-4,6-diyl biscarbonate

MS (m/z): 572.1 (MH⁺).

### Example 22

### (2Z)-2-[(5-Methoxy-2-phenyl-1H-indol-3-yl)methylene]-3-oxo-2,3-dihydro-1-benzofuran-4,6-diyl dimethyl biscarbonate

MS (m/z): 516.0 (MH⁺).

### Example 23

### (2Z)-2-[(5-Methoxy-2-phenyl-1H-indol-3-yl)methylene]-3-oxo-2,3-dihydro-1-benzofuran-4,6-diyl diphenyl biscarbonate

MS (m/z): 640.2 (MH⁺).

### Example 3

### (2Z)-2-[(5-Methoxy-2-phenyl-1H-indol-3-yl)methylene]-3-oxo-2,3-dihydro-1-benzofuran-4,6-diyl diacetate

MS (m/z): 484.1 (MH⁺).

### Example 6

### (2Z)-2-[(5-Methoxy-2-phenyl-1H-indol-3-yl)methylene]-3-oxo-2,3-dihydro-1-benzofuran-4,6-diyl dibenzoate

MS (m/z): 608.1 (MH⁺).

### Example 10

### (2Z)-2-[(5-Methoxy-2-phenyl-1H-indol-3-yl)methylene]-3-oxo-2,3-dihydro-1-benzofuran-4,6-diyl bis(2,2-dimethylpropanoate)

MS (m/z): 568.3 (MH⁺).

### Example 32

### Tetraethyl (2Z)-2-[(5-methoxy-2-phenyl-1H-indol-3-yl)methylene]-3-oxo-2,3-dihydro-1-benzofuran-4,6-diyl bis(phosphate)

MS (m/z): 672.2 (MH⁺).

### Example 38

### Preparation of (2Z)-2-[(5-methoxy-2-phenyl-1H-indol-3-yl)methylene]-3-oxo-2,3-dihydro-1-benzofuran-4,6-diyl bis[dihydrogen (phosphate)]

The title compound was prepared from tetraethyl (2Z)-2-[(5-methoxy-2-phenyl-1H-indol-3-yl)methylene]-3-oxo-2,3-dihydro-1-benzofuran-4,6-diyl bis(phosphate), according to the procedure described for Example 27. Yield: 19%. MS (m/z): 560.0 (MH⁺). Prodrugs of (2Z)-4,6-dihydroxy-2-({5-methoxy-2-methyl-1-[2-(4-methylpiperazin-1-yl)pethyl]-1H— indol-3-yl}methylene)-1-benzofuran-3(2H)-one

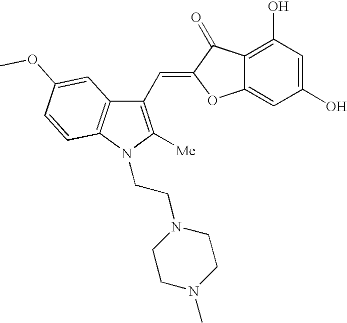

Following the general procedure above and Scheme C-III, compounds in Table III were prepared.

TABLE III

| Ex. | | Eq. of | | | | | | Yield |
|---|---|---|---|---|---|---|---|---|
| # | R²⁰ Cl | R²⁰ Cl | Base | Solvent | time | Work-up | Purification | % |
| 12 | 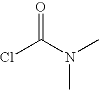 | 2.5 | Py (4 mL) | THF | 3 days | D | Preparative HPLC | 24 |
| 28 | 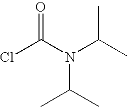 | 2.5 | / | Py | 24 hours at RT, 10 hours at 70° C. | C | Trituration with methylene chloride, I- Pr₂O and Et₂O | 64 |
| 29 | 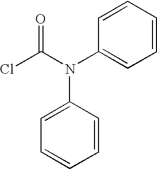 | 2.5 | / | Py | 12 hours | E | Trituration with methylene chloride and Et₂O | 26 |
| 16 | 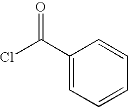 | 5.5 | / | THF/Py 9:1 | 4 days | C | Trituration with methylene chloride and I- Pr₂O | 35 |

### Example 12

### (2Z)-2-({5-Methoxy-2-methyl-1-[2-(4-methyl piperazin-1-yl)pethyl]-1H-indol-3-yl}methylene)-3-oxo-2,3-dihydro-1-benzofuran-4,6-diyl bis(dimethylcarbamate)

MS (m/z): 606.4 (MH⁺).

### Example 28

### (2Z)-2-({5-Methoxy-2-methyl-1-[2-(4-methyl piperazin-1-yl)pethyl]-1H-indol-3-yl}methylene)-3-oxo-2,3-dihydro-1-benzofuran-4,6-diyl bis(diisopropylcarbamate)

MS (m/z): 718.6 (MH⁺).

### Example 29

### (2Z)-2-({5-Methoxy-2-methyl-1-[2-(4-methyl piperazin-1-yl)pethyl]-1H-indol-3-yl}methylene)-3-oxo-2,3-dihydro-1-benzofuran-4,6-diyl bis(di phenylcarbamate)

MS (m/z): 854.7 (MH⁺).

### Example 16

### (2Z)-2-({5-Methoxy-2-methyl-1-[2-(4-methyl piperazin-1-yl)pethyl]-1H-indol-3-yl}methylene)-3-oxo-2,3-dihydro-1-benzofuran-4,6-diyl dibenzoate

MS (m/z): 672.3 (MH⁺).

### Prodrugs of (2Z)-2-({1-[3-(dimethylamino)propyl]-5-methoxy-1H-indol-3-yl}methylene)-4,6-dihydroxy-1-benzofuran-3(2H)-one

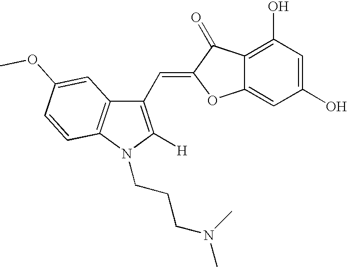

Following the general procedure above and Scheme C-IV (Methods A-C), compounds in Table IV were prepared.

TABLE IV

| Ex. | | Eq. of | | | Reaction | | | Yield |
|---|---|---|---|---|---|---|---|---|
| # | R²⁰ Cl | R²⁰ Cl | Base | Solvent | time | Work-up | Purification | % |
| 1 | 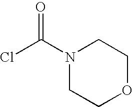 | 2.5 | / | Py | 5 hours | E | Trituration with Et₂O | 56 |
| 2 | 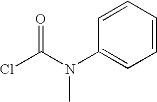 | 2.5 | / | Py | 2 hours | F | Trituration with methylene chloride and i- Pr₂O | 37 |
| 4 | 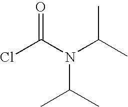 | 10 | / | Py | 5 days | F | Trituration with methylene chloride and i- Pr₂O | 49 |
| 8 | 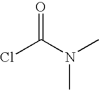 | 2.5 | / | Py | 12 hours | F | Preparative HPLC | 20 |
| 21 | 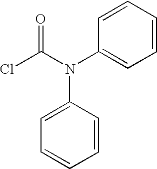 | 2.6 | / | Py | 2 hours | D | Trituration with Et₂O | 53 |
| 5 | 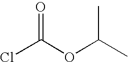 | 2.75 | Py (12 eq.) | THF | 24 hours | C | Trituration with Et₂O | 41 |
| 13 | 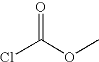 | 3.75 | Py (6 eq.) | CH₃CN | 24 hours | C | Preparative HPLC | 14 |
| 9 | 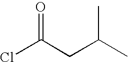 | 20 | / | THF/Py 5:1 | 7 days | C | Trituration with i-Pr₂O | 68 |
| 14 | 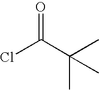 | 5 | Py (12 eq.) | THF | 24 hours | C | Preparative HPLC | 18 |
| 15 | 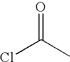 | 2.5 | Py (6 eq.) | THF | 3 hours | C | Trituration with Et₂O | 29 |
| 24 | 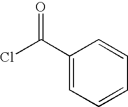 | 2.6 | / | Py | 2 hours | D | Trituration with Et₂O | 50 |
| 33 | 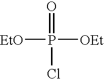 | 3 | Et₃N (5 eq.) | THF | 12 hours | B | Preparative HPLC | 20 |

### Example 1

### (2Z)-2-({1-[3-(Dimethylamino)propyl]-5-methoxy-1H-indol-3-yl}methylene)-3-oxo-2,3-dihydro-1-benzofuran-4,6-diyl dimorpholine-4-carboxylate

MS (m/z): 635.2 (MH⁺).

### Example 2

### (2Z)-2-({1-[3-(Dimethylamino)propyl]-5-methoxy-1H-indol-3-yl}methylene)-3-oxo-2,3-dihydro-1-benzofuran-4,6-diyl bis[methyl(phenyl)carbamate]

MS (m/z): 675.4 (MH⁺).

### Example 4

### (2Z)-2-({1-[3-(Dimethylamino)propyl]-5-methoxy-1H-indol-3-yl}methylene)-3-oxo-2,3-dihydro-1-benzofuran-4,6-diyl bis(diisopropylcarbamate)

MS (m/z): 663.4 (MH⁺).

### Example 8

### (2Z)-2-({1-[3-(Dimethylamino)propyl]-5-methoxy-1H-indol-3-yl}methylene)-3-oxo-2,3-dihydro-1-benzofuran-4,6-diyl bis(dimethylcarbamate)

MS (m/z): 551.2 (MH⁺).

### Example 21

### (2Z)-2-({1-[3-(Dimethylamino)propyl]-5-methoxy-1H-indol-3-yl}methylene)-3-oxo-2,3-dihydro-1-benzofuran-4,6-diyl bis(diphenylcarbamate)

MS (m/z): 799.5 (MH⁺).

### Example 5

### (2Z)-2-({1-[3-(Dimethylamino)propyl]-5-methoxy-1H-indol-3-yl}methylene)-3-oxo-2,3-dihydro-1-benzofuran-4,6-diyl diisopropyl biscarbonate

MS (m/z): 581.3 (MH⁺).

### Example 13

### (2Z)-2-({1-[3-(Dimethylamino)propyl]-5-methoxy-1H-indol-3-yl}methylene)-3-oxo-2,3-dihydro-1-benzofuran-4,6-diyl dimethyl biscarbonate

MS (m/z): 525.2 (MH⁺).

### Example 9

### (2Z)-2-({1-[3-(Dimethylamino)propyl]-5-methoxy-1H-indol-3-yl}methylene)-3-oxo-2,3-dihydro-1-benzofuran-4,6-diyl bis(3-methylbutanoate)

MS (m/z): 577.3 (MH⁺).

### Example 14

### (2Z)-2-({1-[3-(Dimethylamino)propyl]-5-methoxy-1H-indol-3-yl}methylene)-3-oxo-2,3-dihydro-1-benzofuran-4,6-diyl bis(2,2-dimethyl propanoate)

MS (m/z): 577.3 (MH⁺).

### Example 15

### (2Z)-2-({1-[3-(Dimethylamino)propyl]-5-methoxy-1H-indol-3-yl}methylene)-3-oxo-2,3-dihydro-1-benzofuran-4,6-diyl diacetate

MS (m/z): 493.2 (MH⁺).

### Example 24

### (2Z)-2-({1-[3-(Dimethylamino)propyl]-5-methoxy-1H-indol-3-yl}methylene)-3-oxo-2,3-dihydro-1-benzofuran-4,6-diyl dibenzoate

MS (m/z): 617.3 (MH⁺).

### Example 33

### (2Z)-2-({1-[3-(Dimethylamino)propyl]-5-methoxy-1H-indol-3-yl}methylene)-3-oxo-2,3-dihydro-1-benzofuran-4,6-diyl tetraethyl bis(phosphate)

MS (m/z): 681.5 (MH⁺).

### Example 35

### Preparation of diethyl 2,2∝-[[(2Z)-2-({1-[3-(dimethylamino)propyl]-5-methoxy-1H-indol-3-yl}methylene)-3-oxo-2,3-dihydro-1-benzofuran-4,6-diyl]bis(oxycarbonylimino)]diacetate

To a solution of (2Z)-2-({1-[3-(dimethylamino)propyl]-5-methoxy-1H-indol-3-yl}methylene)-4,6-dihydroxy-1-benzofuran-3(2H)-one (150 mg, 0.34 mmol, 1 eq.) in dry pyridine (8 mL), ethyl isocyanatoacetate (97 μL, 0.85 mmol, 2.5 eq.) was added. The resulting solution was stirred at 50° C. for 5 days adding fresh electrophile (60 μL, 1.5 eq.) every 24 hours. Et₂O was added and the formed precipitate was filtered and purified by preparative HPLC. Yield: 56%. MS (m/z): 667.6 (MH⁺).

### Example 36

### Preparation of (2Z)-2-({1-[3-(dimethylamino)propyl]-5-methoxy-1H-indol-3-yl}methylene)-3-oxo-2,3-dihydro-1-benzofuran-4,6-diyl bis(4-methylpiperazine-1- carboxylate)

To a solution of 1-methyl-piperazine (100 μL, 0.9 mmol, 3 eq.) in dry methylene chloride (18 mL) cooled to 0° C., trichloro-acetyl chloride (217 μL, 1.8 mmol, 6 eq.) was added dropwise under nitrogen and the mixture was allowed to stir at room temperature for 24 hours. The solvent was evaporated to obtain crude 1-methyl-4-trichloromethoxycarbonyl-piperazin-1-ium chloride reagent that was used in the next step without further purification.

1-Methyl-4-trichloromethoxycarbonyl-piperazin-1-ium chloride, from the above preparation, and (2Z)-2-({1-[3-(dimethylamino)propyl]-5-methoxy-1H-indol-3-yl}methylene)-4,6-dihydroxy-1-benzofuran-3(2H)-one (132 mg, 0.30 mmol, 1 eq.) were dissolved in pyridine (9 mL), and the mixture was stirred for 3 hours at 60° C. Solvent (methylene chloride, 30 mL) was added, then the organic layer was washed with 0.5 N NaHCO₃ (3×25 mL), dried on Na₂SO₄ and evaporated. The crude mixture was triturated with Et₂O, to afford the pure title compound. Yield: 77%. MS (m/z): 661.3 (MH⁺).

### Example 37

### Preparation of (2Z)-2-({1-[3-(dimethylamino)propyl]-5-methoxy-1H-indol-3-yl}methylene)-3-oxo-2,3-dihydro-1-benzofuran-4,6-diyl bis(4-benzyl piperazine-1-carboxylate)

A solution of 1-benzyl-piperazine (151.2 μL, 0.87 mmol, 3 eq.) was used to prepare 1-benzyl-4-trichloromethoxycarbonyl-piperazin-1-ium chloride, following the procedure used in Example 36.

Reaction of the crude reagent, 1-benzyl-4-trichloromethoxycarbonyl-piperazin-1-ium chloride and (2Z)-2-({1-[3-(dimethylamino)propyl]-5-methoxy-1H-indol-3-yl}methylene)-4,6-dihydroxy-1-benzofuran-3(2H)-one, as in Example 36, provided a crude mixture, which was triturated with Et₂O, to afford the pure title compound. Yield: 48%. MS (m/z): 813.4 (MH⁺).

### Prodrug of (2Z)-2-({1-[3-(dimethylamino)propyl]-5-methoxy-1H-indol-3-yl}methylene)-6-hydroxy-1-benzofuran-3(2H)-one

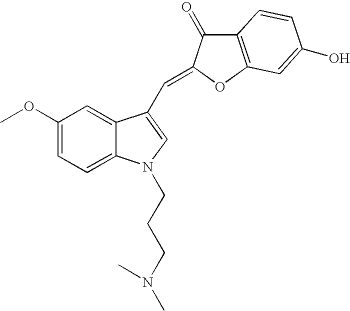

### Example 25

### Preparation of ethyl N-({[(2Z)-2-({1-[3-(dimethylamino)propyl]-5-methoxy-1H-indol-3-yl}methylene)-3-oxo-2,3-dihydro-1-benzofuran-6-yl]oxy}carbonyl)glycinate

Following the procedure of Example 35, and Scheme C-V, (2Z)-2-({1-[3-(dimethylamino)propyl]-5-methoxy-1H-indol-3-yl}methylene)-6-hydroxy-1-benzofuran-3(2H)-one (300 mg, 0.7 mmol, 1 eq.) was dissolved in dry pyridine (10 mL), ethyl isocyanatoacetate (104 μL, 0.91 mmol, 1.3 eq.) was added and the solution was stirred at 50° C. for 3 days adding fresh electrophile (104 μL, 1.3 eq.) every 24 hours. Et₂O was added and a precipitate formed. The crude title compound was purified by trituration of the solid with methylene chloride and Et₂O. Yield: 72%. MS (m/z): 522.4 (MH⁺).

### Mono-Prodrugs of (2Z)-4,6-dihydroxy-2-({5-methoxy-2-methyl-1-[2-(4-methyl piperazin-1-yl)pethyl]-1H-indol-3-yl}methylene)-1-benzofuran-3(2H)-one and (2Z)-2-({1-[3-(dimethylamino)propyl]-5-methoxy-1H-indol-3-yl}methylene)-4,6-dihydroxy-1-benzofuran-3(2H)-one

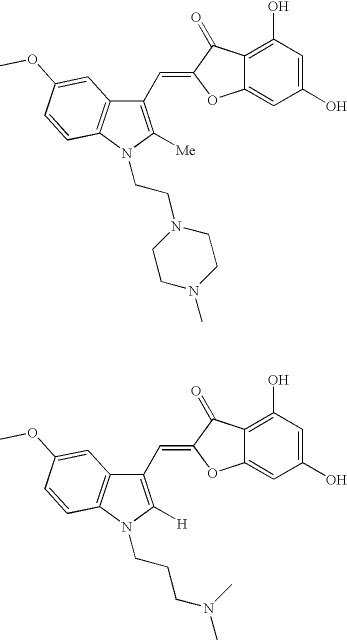

### Step 1. Preparation of Benzofuranone Intermediates

### Preparation of Dimethyl-carbamic acid 6-hydroxy-3-oxo-2,3-dihydro-benzofuran-4-yl Ester

To a solution of 4,6-dihydroxybenzofuranone (500 mg, 3 mmol, 1 eq.) and K₂OC₃ (416 mg, 3 mmol, 1 eq.) in THF (40 mL), dimethylcarbamoyl chloride (277 μL, 3 mmol, 1 eq.) was added. The reaction mixture was refluxed for 36 hours, then water was added. The mixture was acidified with 2 N HCl and extracted with AcOEt. The combined organic layers were dried on Na₂SO₄ and evaporated. The crude mixture was purified by silica gel column chromatography (eluent: gradient from methylene chloride to methylene chloride/MeOH 30:1) affording the pure title compound. Yield: 17%. MS (m/z): 238.2 (MH⁺).

### Preparation of Dimethyl-carbamic Acid 4-hydroxy-3-oxo-2,3-dihydro-benzofuran-6-yl Ester

To a solution of 4,6-dihydroxybenzofuranone (1 g, 6.02 mmol, 1 eq.) and pyridine (4.9 mL, 60.2 mmol, 10 eq.) in THF (100 mL), dimethylcarbamoyl chloride (0.55 mL, 6.02 mmol, 1 eq.) was added. The reaction mixture was refluxed for 36 hours, then water was added and the aqueous phase was extracted with AcOEt. The combined organic layers were dried on Na₂SO₄ and evaporated. The crude mixture was purified by silica gel column chromatography (eluent: gradient from hexane/AcOEt 8:2 to hexane/AcOEt 1:1), to afford the pure title compound. Yield: 7%. MS (m/z): 238.2 (MH⁺).

### Step 2. Preparation of Indole 3-Carbaldehyde Intermediates

The intermediates were prepared as described above in Scheme Section A, Section A-II.

### Step 3. General Procedure for the Condensation of Benzofuranone Intermediates with Indole 3-Carbaldehyde Intermediates to Prepare mono-Prodrug/mono-Hydroxy Analogs

To a solution of the selected 5-methoxy-indole-3-carbaldehyde (4 mmol, 1 eq.) and the mono-OH/mono-dimethylcarbamate benzofuranone (4 mmol, 1 eq.) in EtOH (16 mL), a catalytic amount of 12 N HCl was added. The resulting mixture was stirred at 85° C. for 6 hours and then allowed to cool to room temperature. The formed solid was recovered by filtration, washed with ethyl ether, and dried under vacuum. In some cases, further purification was necessary as indicated in the table.

According to this procedure and Scheme C-VI, the compounds in Table V were obtained:

TABLE V

| Ex. | | | | | Yield |
|---|---|---|---|---|---|
| # | R₁₀ | R₅ | Benzofuranone | Purification | (%) |
| 39 | 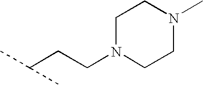 | Me | 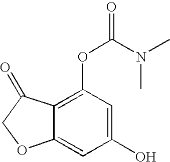 | Filtration and washing with Et₂O | 86 |
| 40 | 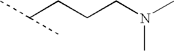 | H | 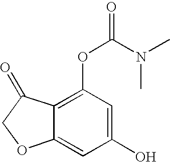 | Preparative HPLC | 34 |
| 41 | 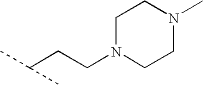 | Me | 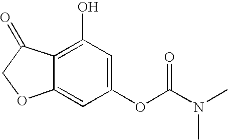 | Filtration and washing with Et₂O | 28 |
| 42 | 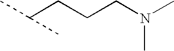 | H | 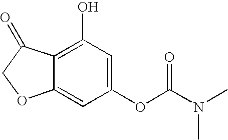 | Preparative HPLC | 19 |

### Example 39

### (2Z)-6-Hydroxy-2-({5-methoxy-2-methyl-1-[2-(4-methylpiperazin-1-yl)ethyl]-1H-indol-3-yl}methylene)-3-oxo-2,3-dihydro-1-benzofuran-4-yl dimethylcarbamate

MS (m/z): 535.17 (MH⁺).

### Example 40

### (2Z)-2-({1-[3-(Dimethylamino)propyl]-5-methoxy-1H-indol-3-yl}methylene)-6-hydroxy-3-oxo-2,3-dihydro-1-benzofuran-4-yl dimethylcarbamate

MS (m/z): 480.09 (MH⁺).

### Example 41

### (2Z)-4-Hydroxy-2-({5-methoxy-2-methyl-1-[2-(4-methyl piperazin-1-yl)ethyl]-1H-indol-3-yl}methylene)-3-oxo-2,3-dihydro-1-benzofuran-6-yl dimethylcarbamate

MS (m/z): 535.07 (MH⁺).

### Example 42

### (2Z)-2-({1-[3-(Dimethylamino)propyl]-5-methoxy-1H-indol-3-yl}methylene)-4-hydroxy-3-oxo-2,3-dihydro-1-benzofuran-6-yl dimethylcarbamate

MS (m/z): 480.09 (MH⁺).

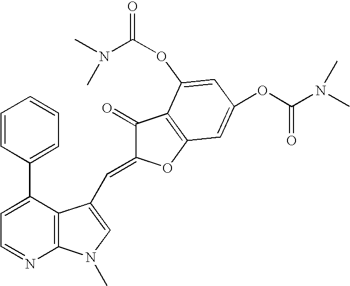

### (2Z)-2-[(1-methyl-4-phenyl-1H-pyrrolo[2,3-b]pyridin-3-yl)methylene]-3-oxo-2,3-dihydro-1-benzofuran-4,6-diyl bis(dimethylcarbamate)

To a solution of (2Z)-4,6-dihydroxy-2-[(1-methyl-4-phenyl-1H-pyrrolo[2,3-b]pyridin-3-yl)methylene]-1-benzofuran-3(2H)-one (38.4 mg, 0.1 mmol) in pyridine (0.38 mL) was added dimethylcarbamyl chloride (0.0185 mL, 0.2 mmol) at room temperature. After heating at 50° C. for 7 hours, the solution was cooled, treated with ice water. The precipitated was filtered, washed with water, and dried to give 41 mg (78%) of the title compound as a yellow solid. MS (ESI) m/z 527.3; HRMS (ESI) m/e calcd for C29H26N4O6+H⁺, 527.19251; found (ESI, [M+H]⁺ Calc'd), 527.1925.

### Example 44

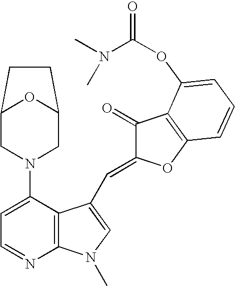

### (2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-4-yl dimethylcarbamate

To a solution of (2Z)-4-hydroxy-2-{[1-methyl-4-(8-oxa-3-azabicyclo[3.2.1]oct-3-yl)-1H-pyrrolo[2,3-b]pyridin-3-yl]methylene}-1-benzofuran-3(2H)-one (40.3 mg, 0.1 mmol) in pyridine (0.38 mL) was added dimethylcarbamyl chloride (0.0185 mL, 0.2 mmol) at room temperature. After heating at 50° C. for 5 hours, the solution was cooled, treated with ice water. The precipitated was filtered, washed with water, and dried to give 34 mg (71%) of the title compound as a yellow solid. MS (ESI) m/z 475.3; HRMS (ESI) m/e calcd for C26H26N4O5+H⁺, 475.19760; found (ESI-FTMS, [M+H]¹⁺), 475.19771.

### Example 45

### (2Z)-2-({5-Methoxy-1-[2-(4-methylpiperazin-1-yl)ethyl]-1H-indol-3-yl}methylene)-3-oxo-2,3-dihydro-1-benzofuran-5-yl methylcarbamate

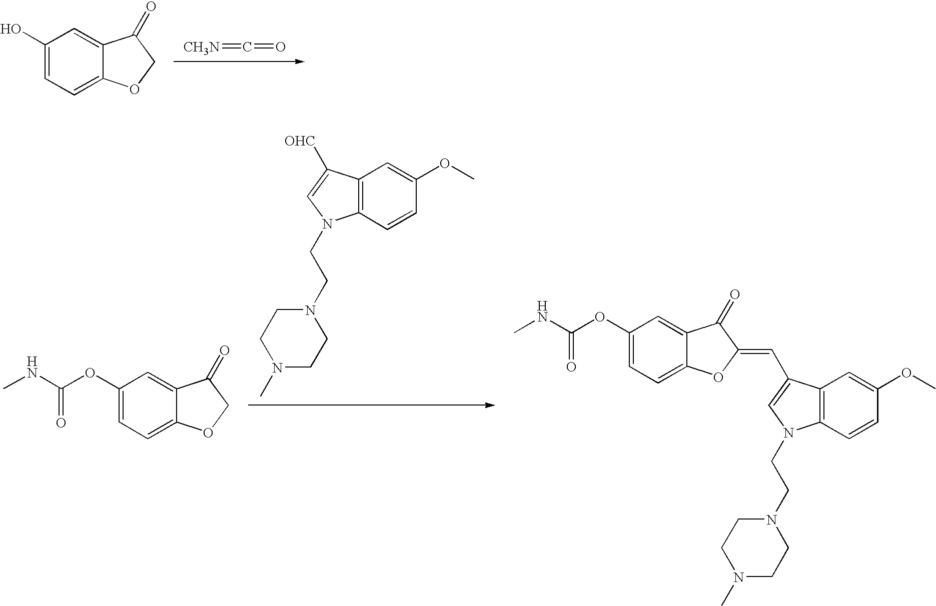

### Step A. 3-Oxo-2,3-dihydro-1-benzofuran-5-yl methylcarbamate

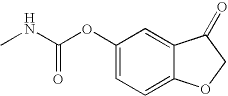

To a solution of 5-hydroxy-1-benzofuran-3(2H)-one (300 mg, 2.0 mmol) in 5 mL of tetrahydrofuran is added methyl isocyanate (1 M in toluene, 6 mL, 6 mmol) followed by 0.5 mL of triethylamine. The mixture is stirred at room temperature for 18 hrs and concentrated. The residue is chromatographed over silica gel, eluting with a gradient of 20% ethyl acetate in hexanes to 50% ethyl acetate in hexanes to give 84 mg (20%) of 3-oxo-2,3-dihydro-1-benzofuran-5-yl methylcarbamate as a white solid. MS: m/z 208.1 (M+H).

Preparation of methyl isocyanate: To a suspension of sodium azide (450 mg, 6.9 mmol) in 6.5 mL of toluene at 0 PC is added acetyl chloride (500 mg, 6.3 mmol). The mixture is refluxed with dry ice-acetone condenser cooling under nitrogen for 6 hrs, and cooled to room temperature. The supernatant is decanted, and used as 1.0 M methyl isocyanate solution in toluene.

### Step B. (2Z)-2-({5-Methoxy-1-[2-(4-methylpiperazin-1-yl)ethyl]-1H-indol-3-yl}methylene)-3-oxo-2,3-dihydro-1-benzofuran-5-yl methylcarbamate

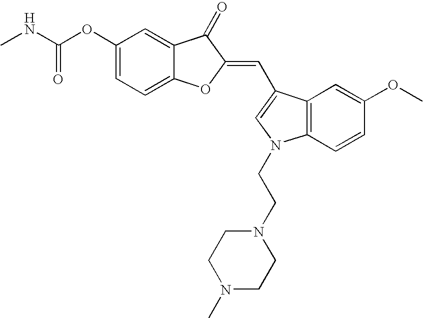

To a solution of 3-oxo-2,3-dihydro-1-benzofuran-5-yl methylcarbamate (32 mg, 0.15 mmol) and 5-methoxy-1-[2-(4-methylpiperazin-1-yl)ethyl]-1H-indole-3-carbaldehyde (45 mg, 0.15 mmol) in 5 mL of ethanol is added five drops of concentrated hydrochloric acid. The mixture is stirred at room temperature for 18 hours. The solids formed are collected by filtration, washed with 10% methanol in ethyl acetate, and dried to give 16 mg (69%) of (2Z)-2-({5-methoxy-1-[2-(4-methylpiperazin-1-yl)ethyl]-1H-indol-3-yl}methylene)-3-oxo-2,3-dihydro-1-benzofuran-5-yl methylcarbamate dihydrochloride. MS: m/z 491.2 (M+H).

### Example 46

### (2Z)-2-({5-Methoxy-2-methyl-1-[2-(4-methylpiperazin-1-yl)ethyl]-1H-indol-3-yl}methylene)-3-oxo-2,3-dihydro-1-benzofuran-6-yl methyl(phenyl)carbamate

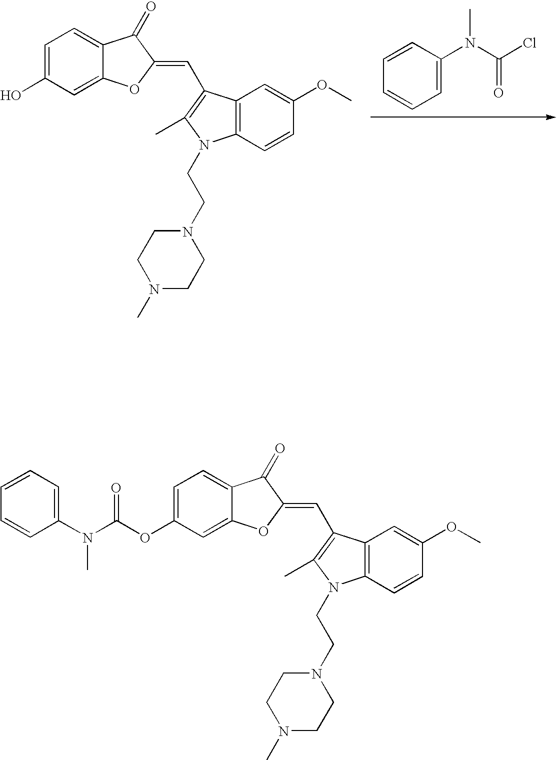

To a solution of (2Z)-6-hydroxy-2-({5-methoxy-2-methyl-1-[2-(4-methylpiperazin-1-yl)ethyl]-1H-indol-3-yl}methylene)-1-benzofuran-3(2H)-one prepared in accordance with the general procedure for the condensation between 4,6-dihydroxy-benzofuran-3-one (Compound A) and 5-methoxy-indole-3-carbaldehydes described herein, (60 mg, 0.134 mmol) in 2 mL of tetrahydrofuran and 0.5 mL of pyridine is added N-methyl-N-phenylcarbamoyl chloride (85 mg, 0.5 mmol). The mixture is stirred at room temperature for 18 hr and concentrated. HPLC purification provided 41 mg (38%) of ((2Z)-2-({5-Methoxy-2-methyl-1-[2-(4-methylpiperazin-1-yl)ethyl]-1H-indol-3-yl}methylene)-3-oxo-2,3-dihydro-1-benzofuran-6-ylmethyl(phenyl)carbamate ditrifluoroacetate. MS: m/z 581.4 (M+H).

### Example 47

### (2Z)-2-({5-Methoxy-2-methyl-1-[2-(4-methyl piperazin-1-yl)ethyl]-1H-indol-3-yl}methylene)-3-oxo-2,3-dihydro-1-benzofuran-6-yl diisopropylcarbamate

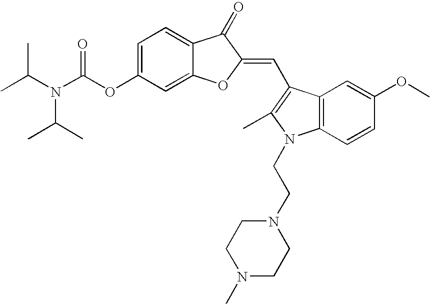

Using the same procedure described above, starting from (2Z)-6-hydroxy-2-({5-methoxy-2-methyl-1-[2-(4-methylpiperazin-1-yl)pethyl]-1H-indol-3-yl}methylene)-1-benzofuran-3(2H)-one, prepared in accordance with the general procedure for the condensation between 4,6-dihydroxy-benzofuran-3-one (Compound A) and 5-methoxy-indole-3-carbaldehydes described herein, (60 mg, 0.134 mmol) and diisopropylcarbamoyl chloride (82 mg, 0.5 mmol), 49 mg (46%) of (2Z)-2-({5-methoxy-2-methyl-1-[2-(4-methylpiperazin-1-yl)ethyl]-1H-indol-3-yl}methylene)-3-oxo-2,3-dihydro-1-benzofuran-6-yl diisopropylcarbamate ditrifluoroacetate was obtained as a red solid. MS: m/z 575.4 (M+H).

### Example 48

### (2Z)-2-[(1-methyl-4-phenyl-1H-indol-3-yl)methylene]-3-oxo-2,3-dihydro-1-benzofuran-4,6-diyl bis(dimethylcarbamate)

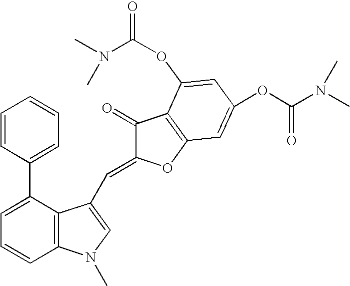

To a solution of (2Z)-4,6-dihydroxy-2-[(1-methyl-4-phenyl-1H-indol-3-yl)methylene]-1-benzofuran-3(2H)-one (25 mg, 0.065 mmol) in pyridine (0.25 mL) was added dimethylcarbamyl chloride (0.012 mL, 0.13 mmol) at room temperature. After heating at 50° C. for 6 hours, the solution was cooled, treated with ice water. The precipitated was filtered, washed with water, and dried to give 31.4 mg (92%) of the title compound as an orange solid. MS (ESI) m/z 526.3; HRMS (ESI) m/e calcd for C30H27N3O6+H⁺, 526.19726; found (ESI, [M+H]⁺ Calc'd), 526.1973.

Profiling and stability data for representative compounds of the invention (prodrugs) 1-48 and precursor phenolic compounds are shown in Tables VIa, VIb, VIc, and VId below. The precursor phenolic compounds include compounds that can be converted into the compounds of the invention by protecting one or more phenolic OH groups in each case. For such phenolic compounds, the corresponding compounds of the invention are shown in parenthesis in each case in Table VIb—the number in parenthesis corresponds to the same number for each compound of the invention in Table VIa below.

TABLE VIa
Profiling and microsomal stability data for compounds of the invention
(prodrugs) 1-48

| | | | | Microsome Stability | | |
|---|---|---|---|---|---|---|
| | | PAMPA | CACO2 | (15 min) | | |
| | Solubility | Pex10e−6 | Pex10e−6 | T ½ (min) Phase I | | |
| | ug/mL | cm/s pH | cm/s a − b/ | | nude | |
| Ex. # | pH 7.4 | 7.4 | b − a | human | mouse | rat |
| 1 | >100 | 1.4 | | | | >30.0 |
| 2 | 0 | 0 | 0.04/0.08 | 24 | >30.0 | >30.0 |
| 3 | 0 | 0 | | 12 | 9 | >30.0 |
| 4 | 0 | 0 | 0.16/0.06 | 6 | 21 | >30.0 |
| 5 | 3 | 0 | | 3 | 3 | <1.0 |
| 6 | 0 | 0.12 | | | | Poor |
| | | | | | | MS |
| 7 | 0 | 0 | | 5 | >30.0 | 3 |
| 8 | 28 | 4.53 | 4.82/7.22 | 15 | 23 | >30.0 |
| 9 | 1 | 0 | | 6 | 6 | 4 |
| 10 | 2 | 0 | | 19 | Poor | 12 |
| | | | | | MS | |
| 11 | 1 | 1.14 | | 23 | Poor | >30.0 |
| | | | | | MS | |
| 12 | 40 | 0.78 | 1.98/3.36 | 9 | 23 | 23 |
| 13 | 43 | 0.19 | | | | Poor |
| | | | | | | MS |
| 14 | 1 | 0 | | 21 | 2 | 3 |
| 15 | 49 | 0.24 | | | | Poor |
| | | | | | | MS |
| 16 | 0 | 0 | | >30.0 | Poor | 10 |
| | | | | | MS | |
| 17 | 4 | 0 | | 4 | >30.0 | 5 |
| 18 | 0 | Undetected | | 13 | >30.0 | 28 |
| 19 | 0 | 0 | | 28 | >30.0 | >30.0 |
| 20 | 0 | Undetected | | >30.0 | >30.0 | >30.0 |
| 21 | 0 | 0 | | Poor | >30.0 | Poor |
| | | | | MS | | MS |
| 22 | 0 | 0 | | 3 | 4 | Poor |
| | | | | | | MS |
| 23 | 1 | 0 | | 6 | | 8 |
| 24 | 0 | 0 | | 22 | Poor | 12 |
| | | | | | MS | |
| 25 | >100 | 0 | | | | Poor |
| | | | | | | MS |
| 26 | 11 | 0 | | | | >30.0 |
| 27 | 65 | 0 | | | | 19 |
| 28 | 0 | 0.05 | | 8 | 24 | Poor |
| | | | | | | MS |
| 29 | 5 | 0 | | | | Poor |
| | | | | | | MS |
| 30 | 0 | 0 | 0.19/0.04 | <1.0 | >30.0 | 19 |
| 31 | 0 | 0 | | 7 | >30.0 | 9 |
| 32 | 0 | 0 | | 2 | Poor | 7 |
| | | | | | MS | |
| 33 | 59 | 0.25 | | 4 | 15 | 12 |
| 34 | 0 | 0 | | 26 | >30.0 | 17 |
| 35 | 51 | 0 | | | | Poor |
| | | | | | | MS |
| 36 | 36 | 0.23 | | | | Poor |
| | | | | | | MS |
| 37 | 0 | 0 | | | | Poor |
| | | | | | | MS |
| 38 | >100 | 0.01 | | | | Poor |
| | | | | | | MS |
| 39 | >100 | 0 | | | | 27 |
| 40 | no | | | | | |
| | profiling | | | | | |
| | data | | | | | |
| 41 | no | | | | | |
| | profiling | | | | | |
| | data | | | | | |
| 42 | no | | | | | |
| | profiling | | | | | |
| | data | | | | | |
| 43 | 0 | Undetected | 2.2/1 | 11 | 12 | |
| 44 | 0 | Undetected | | 8 | 4 | 3 |
| 45 | 63 | 0.43 | | | | 9 |
| 46 | 0 | Undetected | | | | 10 |
| 47 | 0 | Undetected | | | | 16 |
| 48 | 0 | Undetected | 0.5/0.5 | 25 | 14 | |

TABLE VIb
Plasma and buffer stability data for compounds of the invention (prodrugs) 1-48

| | Plasma Stability | | | | | | | | |
|---|---|---|---|---|---|---|---|---|---|
| | (37° C.) | | | | | | | | |
| | % Remaining | | Buffer Stability (37° C.) | | | | | | |
| | (3-3.5 hours) | | % Remaining (at 16-23 hours) | | | | | | |
| Ex. # | nude mouse | rat | pH 1 | pH 4.5 | pH 6.6 | pH 7.4 | pH 9 | SGF | SIF |
| 1 | | | | | | | | | |
| 2 | 10 | 22 | 100 | 100 | 100 | 100 | 100 | 100 | 100 |
| 3 | 34 | 42 | 2 | 64 | 0 | 0 | 0 | 66 | 0 |
| 4 | 64 | 65 | 100 | 100 | | 100 | 100 | 100 | 100 |
| 5 | 16 | 0 | 95 | 82 | 63 | 14 | 0 | 94 | 0 |
| 6 | | | | | | | | | |
| 7 | 22 | 0 | 94 | 91 | 59 | 16 | 0 | 93 | 0 |
| 8 | 7 | 50 | 100 | 100 | 100 | 100 | 91 | 100 | 100 |
| 9 | 8 | 0 | 73 | 66 | 46 | 0 | 0 | 77 | 0 |
| 10 | 0 | 6 | 86 | 92 | 72 | 44 | 0 | 95 | 0 |
| 11 | 85 | 95 | 99 | 103 | 100 | 100 | 89 | 100 | 98 |
| 12 | 32 | 71 | 100 | 100 | 100 | 100 | 90 | 100 | 100 |
| 13 | | | | | | | | | |
| 14 | | | 89 | 98 | 79 | 20 | 0 | 94 | 0 |
| 15 | | | | | | | | | |
| 16 | | | 96 | 73 | 75 | 4 | 0 | 99 | 64 |
| 17 | | 2 | 95 | 93 | 95 | 32 | 0 | 96 | 0 |
| 18 | | 0 | 99 | 100 | 100 | 100 | 99 | 100 | 101 |
| 19 | | 10 | 104 | 74 | 60 | 54 | 0 | 106 | 35 |
| 20 | | 91 | 100 | 100 | 100 | 100 | 95 | 100 | 100 |
| 21 | | 107 | 118 | 103 | 91 | 98 | 87 | 99 | 45 |
| 22 | | 0 | 80 | 37 | 42 | 0 | 0 | 87 | 0 |
| 23 | | 0 | 0 | 0 | 0 | 0 | 0 | 0 | 0 |
| 24 | | 6 | 86 | 71 | 50 | 5 | 0 | 104 | 0 |
| 25 | | | | | | | | | |
| 26 | | | | | | | | | |
| 27 | | | | | | | | | |
| 28 | | | | | | | | | |
| 29 | | | | | | | | | |
| 30 | | | | | | | | | |
| 31 | | | | | | | | | |
| 32 | | | | | | | | | |
| 33 | | | | | | | | | |
| 34 | | | | | | | | | |
| 35 | | | | | | | | | |
| 36 | | | | | | | | | |
| 37 | | | | | | | | | |
| 38 | | | | | | | | | |
| 39 | | | | | | | | | |
| 40 | | | | | | | | | |
| 41 | | | | | | | | | |
| 42 | | | | | | | | | |
| 43 | 52 | 54 | 100 | 100 | 100 | 100 | 100 | 100 | 100 |
| 44 | 64 | 51 | 102 | 100 | 100 | 100 | 100 | 102 | 100 |
| 45 | | | | | | | | | |
| 46 | 19 | 22 | 101 | 99 | 98 | 97 | 96 | 98 | 100 |
| 47 | 82 | 92 | 97 | 100 | 95 | 100 | 96 | 98 | 94 |
| 48 | 80 | >100 | 100 | 100 | 100 | 100 | 100 | 100 | 100 |

TABLE VIc
Profiling and microsomal stability data for phenolic compounds precursors to
compounds of the invention

| | | | | Microsome Stability | | |
|---|---|---|---|---|---|---|
| | Solubility | PAMPA | CACO2 | (15 min) T ½ (min) Phase I | | |
| | ug/mL | Pex10e−6 cm/s | Pex10e−6 cm/s | | nude | |
| Precursor Phenolic Compounds | pH 7.4 | pH 7.4 | a − b/b − a | human | mouse | rat |
| (4,6-dihydroxy-2-[(5-methoxy-2- | 0 | 0 | 1.25/6.65 | 24/5 | 29/5 | >30/5 |
| phenyl-1H-indol-3-yl)methylene]- | | | | | | |
| 1-benzofuran-3(2H)-one) | | | | | | |
| (3, 6, 7, 10, 11, 22, 23, 30, 31 32, | | | | | | |
| 38) | | | | | | |
| ((2Z)-6-hydroxy-2-({5-methoxy-2- | 68 | 0.01 | | 6/5 | 26/20 | 12/8 |
| methyl-1-[2-(4-methylpiperazin-1- | | | | | | |
| yl)ethyl]-1H-indol-3-yl}methylene)- | | | | | | |
| 1-benzofuran-3(2H)-one) | | | | | | |
| (46, 47) | | | | | | |
| ((2Z)-4,6-dihydroxy-2-({5- | 32 | 0 | | 11/17 | >30/25 | 21/6 |
| methoxy-2-methyl-1-[2-(4- | | | | | | |
| methylpiperazin-1-yl)ethyl]-1H- | | | | | | |
| indol-3-yl}methylene)-1- | | | | | | |
| benzofuran-3(2H)-one) | | | | | | |
| (12, 16, 28, 29, 39, 41) | | | | | | |
| ((2Z)-6-hydroxy-2-[(5-methoxy-2- | 0 | 0.18 | 0.03/0.11 | 15/3 | 19/4 | 18/4 |
| phenyl-1H-indol-3-yl)methylene]- | | | | | | |
| 1-benzofuran-3(2H)-one) | | | | | | |
| (17, 18, 19, 20, 26, 27, 34) | | | | | | |
| ((2Z)-2-({1-[3- | 33 | 0 | 3.9/27 | >30/28 | 15/— | 6/— |
| (dimethylamino)propyl]-5- | | | | | | |
| methoxy-1H-indol-3- | | | | | | |
| yl}methylene)-4,6-dihydroxy-1- | | | | | | |
| benzofuran-3(2H)-one) | | | | | | |
| (1, 2, 4, 5, 8, 9, 13, 14, 15, 21, 24, | | | | | | |
| 33, 35, 36, 37, 40, 42) | | | | | | |
| ((2Z)-2-({1-[3- | 53 | 0 | | | | 12/— |
| (dimethylamino)propyl]-5- | | | | | | |
| methoxy-1H-indol-3- | | | | | | |
| yl}methylene)-6-hydroxy-1- | | | | | | |
| benzofuran-3(2H)-one) | | | | | | |
| (25) | | | | | | |
| ((2Z)-4,6-dihydroxy-2-[(1-methyl- | 1 | 0.09 | 66.3/58.1 | >30/6 | 30/2 | >30/— |
| 4-phenyl-1H-pyrrolo[2,3-b]pyridin- | | | | | | |
| 3-yl)methylene]-1-benzofuran- | | | | | | |
| 3(2H)-one) | | | | | | |
| (43) | | | | | | |
| ((2Z)-5-hydroxy-2-({5-methoxy-2- | no | | | | | |
| methyl-1-[2-(4-methylpiperazin-1- | profiling | | | | | |
| yl)ethyl]-1H-indol-3-yl}methylene)- | data | | | | | |
| 1-benzofuran-3(2H)-one) | | | | | | |
| (45) | | | | | | |
| ((2Z)-4-hydroxy-2-{[1-methyl-4-(8- | 0 | 0.79 | 5.5/8.1 | 28/3 | 13/— | 2/— |
| oxa-3-azabicyclo[3.2.1]oct-3-yl)- | | | | | | |
| 1H-pyrrolo[2,3-b]pyridin-3- | | | | | | |
| yl]methylene}-1-benzofuran-3(2H)- | | | | | | |
| one) | | | | | | |
| (44) | | | | | | |
| ((2Z)-4,6-dihydroxy-2-[(1-methyl- | 0 | 0.04 | | | | 21/— |
| 4-phenyl-1H-indol-3- | | | | | | |
| yl)methylene]-1-benzofuran-3(2H)- | | | | | | |
| one) | | | | | | |
| (48) | | | | | | |

TABLE VId
Plasma and buffer stability data for phenolic compounds precursors to
compounds of the invention

| | Plasma Stability | | | | | | | | |
|---|---|---|---|---|---|---|---|---|---|
| | (37° C.) % | | | | | | | | |
| | Remaining | | Buffer Stability (37° C.) | | | | | | |
| Precursor Phenolic | (3-3.5 hours) | | % Remaining (at 16-23 hours) | | | | | | |
| Compounds | nude mouse | rat | pH 1 | pH 4.5 | pH 6.6 | pH 7.4 | pH 9 | SGF | SIF |
| (4,6-dihydroxy-2-[(5-methoxy- | | | 97 | 96 | 83 | 68 | 72 | 95 | 77 |
| 2-phenyl-1H-indol-3- | | | | | | | | | |
| yl)methylene]-1-benzofuran- | | | | | | | | | |
| 3(2H)-one) | | | | | | | | | |
| (3, 6, 7, 10, 11, 22, 23, 30, 31 | | | | | | | | | |
| 32, 38) | | | | | | | | | |
| ((2Z)-6-hydroxy-2-({5- | | | | | | | | | |
| methoxy-2-methyl-1-[2-(4- | | | | | | | | | |
| methylpiperazin-1-yl)ethyl]- | | | | | | | | | |
| 1H-indol-3-yl}methylene)-1- | | | | | | | | | |
| benzofuran-3(2H)-one) | | | | | | | | | |
| (46, 47) | | | | | | | | | |
| ((2Z)-4,6-dihydroxy-2-({5- | | | 99 | 78 | 63 | 66 | 79 | 99 | 63 |
| methoxy-2-methyl-1-[2-(4- | | | | | | | | | |
| methylpiperazin-1-yl)ethyl]- | | | | | | | | | |
| 1H-indol-3-yl}methylene)-1- | | | | | | | | | |
| benzofuran-3(2H)-one) | | | | | | | | | |
| (12, 16, 28, 29, 39, 41) | | | | | | | | | |
| ((2Z)-6-hydroxy-2-[(5- | 83 | 111 | 97 | 98 | 98 | 98 | 99 | 93 | 97 |
| methoxy-2-phenyl-1H-indol-3- | | | | | | | | | |
| yl)methylene]-1-benzofuran- | | | | | | | | | |
| 3(2H)-one) | | | | | | | | | |
| (17, 18, 19, 20, 26, 27, 34) | | | | | | | | | |
| ((2Z)-2-({1-[3- | 78 | 77 | 99 | 93 | 83 | 82 | 94 | 95 | 57 |
| (dimethylamino)propyl]-5- | | | | | | | | | |
| methoxy-1H-indol-3- | | | | | | | | | |
| yl}methylene)-4,6-dihydroxy- | | | | | | | | | |
| 1-benzofuran-3(2H)-one) | | | | | | | | | |
| (1, 2, 4, 5, 8, 9, 13, 14, 15, | | | | | | | | | |
| 21, 24, 33, 35, 36, 37, 40, 42) | | | | | | | | | |
| ((2Z)-2-({1-[3- | | | | | | | | | |
| (dimethylamino)propyl]-5- | | | | | | | | | |
| methoxy-1H-indol-3- | | | | | | | | | |
| yl}methylene)-6-hydroxy-1- | | | | | | | | | |
| benzofuran-3(2H)-one) | | | | | | | | | |
| (25) | | | | | | | | | |
| ((2Z)-4,6-dihydroxy-2-[(1- | | | | | | | | | |
| methyl-4-phenyl-1H- | | | | | | | | | |
| pyrrolo[2,3-b]pyridin-3- | | | | | | | | | |
| yl)methylene]-1-benzofuran- | | | | | | | | | |
| 3(2H)-one) | | | | | | | | | |
| (43) | | | | | | | | | |
| ((2Z)-5-hydroxy-2-({5- | | | | | | | | | |
| methoxy-2-methyl-1-[2-(4- | | | | | | | | | |
| methylpiperazin-1-yl)ethyl]- | | | | | | | | | |
| 1H-indol-3-yl}methylene)-1- | | | | | | | | | |
| benzofuran-3(2H)-one) | | | | | | | | | |
| (45) | | | | | | | | | |
| ((2Z)-4-hydroxy-2-{[1-methyl- | | | | | | | | | |
| 4-(8-oxa-3- | | | | | | | | | |
| azabicyclo[3.2.1]oct-3-yl)-1H- | | | | | | | | | |
| pyrrolo[2,3-b]pyridin-3- | | | | | | | | | |
| yl]methylene}-1-benzofuran- | | | | | | | | | |
| 3(2H)-one) | | | | | | | | | |
| (44) | | | | | | | | | |
| ((2Z)-4,6-dihydroxy-2-[(1- | | | | | | | | | |
| methyl-4-phenyl-1H-indol-3- | | | | | | | | | |
| yl)methylene]-1-benzofuran- | | | | | | | | | |
| 3(2H)-one) | | | | | | | | | |
| (48) | | | | | | | | | |

*Phase I metabolism. in presence of NADPH; Phase II in presence of NADPH + UDPGA

Tables VIIa and VIIb below show in vivo blood, tumor levels for compounds of the invention (prodrugs) & precursor phenolic compounds. The precursor phenolic compounds include compounds that can be converted into the compounds of the invention by protecting one or more phenolic OH groups in each case. For such phenolic compounds, the corresponding compounds of the invention are shown in parenthesis in each case—the number in parenthesis corresponds to the same number for each compound of the invention in the Table.

TABLE VIIa

| | 50 mg, kg po; 0.5, 2 hours (or 0.5, 2, 6, 10, 24 h) plasma & tumor | | | | | | | |
|---|---|---|---|---|---|---|---|---|
| | concentration of prodrug and metabolites (ng, ml) | | | | | | | |
| | | | blood | tumor | blood 4- | tumor 4- | blood 6- | tumor 6- |
| | | | precursor | precursor | OH, 6- | OH, 6- | OH, 4- | OH, 4- |
| | plasma | tumor | phenolic | phenolic | OR mono | OR mono | OR mono | OR mono |
| Ex. # | prodrug | prodrug | cpd | cmpd | prodrug | prodrug | prodrug | prodrug |
| 2 | 486, 757 | | 10, 6 | | | | | |
| 4 | 411, 435 | | 78, 87 | | | | | |
| 8 | 3122, | 2170, | bql at all | 3, 1, bql. | 1515, | 348, 660, | 577, 566, | 209, 364, |
| | 3876, | 4820, | time | | 1247, | 174, 84, | 408, 88, | 165, 66, |
| | 2170, | 1482, | points | | 616, 203, | bql | bql | bql |
| | 497, 3 | 556, bql | | | bql | | | |
| 11 | bql, bql | | | | | | | |
| 12 | 2444, | 344, | 44, 48, | 18, 18, | 623, 895, | 128, 393, | 668, 870, | 101, 304, |
| | 3871, | 1278, | 39, 9, bql | bql | 499, 104, | 211, 22, | 549, 118, | 184, 10, |
| | 2027, | 629, 69, | | | bql | 21 | bql | bql |
| | 443, 1 | bql | | | | | | |
| 28 | 1141, | | | | | | | |
| | 940, | | | | | | | |
| | 1106 | | | | | | | |
| 30 | 29, 65, | | | | | | | |
| | 20 | | | | | | | |
| 34 | 6, 2, bql | | | | | | | |

bql = below quantification limit

TABLE VIIb

| | | blood |
|---|---|---|
| | | precursor |
| | plasma | phenolic cmpd |
| | prodrug | bql = below |
| | Dosed @ 50 mg, | quantification |
| Precursor phenolic Compounds | kg po | limit |
| (4,6-dihydroxy-2-[(5-methoxy-2-phenyl-1H-indol-3- | bql, bql* | |
| yl)methylene]-1-benzofuran-3(2H)-one) | | |
| ((2Z)-4,6-dihydroxy-2-({5-methoxy-2-methyl-1-[2- | | |
| (4-methylpiperazin-1-yl)ethyl]-1H-indol-3- | | |
| yl}methylene)-1-benzofuran-3(2H)-one) | | |
| ((2Z)-6-hydroxy-2-[(5-methoxy-2-phenyl-1H-indol- | bql, bql | |
| 3-yl)methylene]-1-benzofuran-3(2H)-one) | | |
| ((2Z)-2-({1-[3-(dimethylamino)propyl]-5-methoxy- | 23, 7* | *1 & 4 h conc. |
| 1H-indol-3-yl}methylene)-4,6-dihydroxy-1- | | |
| benzofuran-3(2H)-one) | | |

### Biological Evaluation —PI3K-alpha Fluorescence Polarization Assay Protocol

The reaction buffer was 20 mM HEPES pH7.5, 2 mM MgCl₂, 0.05% CHAPS, and 0.01% βME (added fresh). The substrate solution was 40 μM PIP2 (diC8, Echelon, Salt Lake City Utah cat #P-4508, 1 mM in water) and 50 μM ATP in the reaction buffer. Nunc 384-well black polypropylene fluorescent plates were used for PI3K assays. The assay is run by putting 9.5 μl of freshly diluted enzyme in the reaction buffer per well, adding 0.5 μl of diluted drug or DMSO, and mixing. Then 10 μl of the substrate solution is added to each well to start the reaction. A final concentration of 20 μM PIP2 and 25 μM ATP in the reaction was used. Reactions were allowed to proceed for 30-60 minutes at room temperature. After 30-60 minutes, 20 μl of a solution of 10 nM TAMRA detector (Red detector probe-Echelon) and 2.5 μM of GST-murineGRP (1.5 mg/ml in 17% glycerol) was added per well to stop the reaction. The resulting solution was mixed well and allowed to stand for 90-110 minutes before reading plate. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds. It is important to keep Red probe solutions dark. This procedure is adapted from Echelon Biosciences Inc procedure for their PI3-Kinase fluorescence polarization activity Assay kit Product number K-1100.

### mTOR Enzyme Assay

The routine human TOR assays with purified enzyme were performed in 96-well plates by DELFIA format as follows. Enzymes were first diluted in kinase assay buffer (10 mM HEPES (pH 7.4), 50 mM NaCl, 50 mM p-glycerophosphate, 10 mM MnCl₂, 0.5 mM DTT, 0.25 μM microcystin LR, and 100 μg/mL BSA). To each well, 12 μL of the diluted enzyme were mixed briefly with 0.5 test inhibitor or the control vehicle dimethylsulfoxide (DMSO). The kinase reaction was initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K was performed at room temperature using a monoclonal anti-P(T389)-p70S6K antibody (1A5, Cell Signaling) labeled with Europium-N-1-ITC (Eu) (10.4 Eu per antibody, PerkinElmer). The DELFIA Assay buffer and Enhancement solution were purchased from Perkin Elmer. 45 μL of the terminated kinase reaction mixture was transferred to a MaxiSorp plate (Nunc) containing 55 μL PBS. The His6-S6K was allowed to attach for 2 hours after which the wells were aspirated and washed once with PBS. 100 μL of DELFIA Assay buffer with 40 ng/mL Eu—P(T389)—S6K antibody was added. The antibody binding was continued for 1 hour with gentle agitation. The wells were then aspirated and washed 4 times with PBS containing 0.05% Tween-20 (PBST). 100 μl of DELFIA Enhancement solution was added to each well and the plates were read in a PerkinElmer Victor model plate reader. Data obtained were used to calculate enzymatic activity and enzyme inhibition by potential inhibitors

### In Vitro Cell Growth Assay

Cell lines used were human adenocarcinoma (LoVo), pancreatic (PC3), prostate (LNCap), breast (MDA468, MCF7), colon (HCT116), renal (HTB44 A498), and ovarian (OVCAR3) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (μM).

Tables VIII and IX show the results of the described biological assays for representative compounds of the invention (prodrugs) (Table VIII) & precursor phenolic compounds (Table IX). The precursor phenolic compounds include compounds that can be converted into the compounds of the invention by protecting one or more phenolic OH groups in each case. For such phenolic compounds, the corresponding compounds of the invention are shown in parenthesis in each case—the number in parenthesis corresponds to the same number for each compound of the invention in Table VIII below.

TABLE VIII

| | PI3K alpha | mTOR |
|---|---|---|
| | Mean IC₅₀ | Mean IC₅₀ |
| Ex. # | (nM) | (nM) |
| 1 | >10000 | >4000 |
| 2 | >10000 | |
| 3 | 2 | |
| 4 | >10000 | |
| 5 | >9750 | |
| 6 | 472 | |
| 7 | 896 | |
| 8 | >10000 | |
| 9 | >10000 | |
| 10 | 2778 | |
| 11 | >5050 | |
| 12 | >10000 | |
| 13 | 313 | |
| 14 | 9750 | |
| 15 | 82 | |
| 16 | 8530 | |
| 17 | 157 | |
| 18 | 6353 | |
| 19 | 181 | |
| 20 | >10000 | |
| 21 | >10000 | |
| 22 | 13 | |
| 23 | 10 | |
| 24 | >10000 | |
| 25 | 86 | 610 |
| 26 | 108 | 2200 |
| 27 | 123 | 2450 |
| 28 | 9500 | |
| 29 | 1000 | |
| 30 | 2224 | |
| 31 | 270 | 510 |
| 32 | 11000 | |
| 33 | >10000 | |
| 34 | 5674 | |
| 35 | 28 | |
| 36 | >10000 | 1075 |
| 37 | >10000 | 1650 |
| 38 | 890 | 985 |
| 39 | 842 | >4000 |
| 40 | 5879 | >20000 |
| 41 | 1128 | 13000 |
| 42 | 2799 | >20000 |
| 43 | >10000 | >4000 |
| 44 | 2488 | 31 |
| 45 | 630 | >4000 |
| 46 | >10000 | >4000 |
| 47 | >10000 | >4000 |
| 48 | >10000 | 3150 |

Compounds 1-48 are expected to have potent enzyme activity when the prodrug groups are hydrolyzed partially or fully under assay conditions to provide the corresponding precursor phenolic compounds. The PI3Ka and mTOR kinase enzyme data values of the precursor phenolic compounds are shown in the table below:

TABLE IX

| | PI3K | |
|---|---|---|
| | alpha | mTOR |
| | Mean IC₅₀ | Mean IC₅₀ |
| Precursor phenolic compounds | (nM) | (nM) |
| ((4,6-dihydroxy-2-[(5-methoxy-2-phenyl-1H- | <3.0 | 80 |
| indol-3-yl)methylene]-1-benzofuran-3(2H)- | | |
| one)) | | |
| ((2Z)-2-({1-[3-(dimethylamino)propyl]-5- | 20.5 | 930 |
| methoxy-1H-indol-3-yl}methylene)-4,6- | | |
| dihydroxy-1-benzofuran-3(2H)-one) | | |
| ((2Z)-2-({1-[3-(dimethylamino)propyl]-5- | 91.5 | 365 |
| methoxy-1H-indol-3-yl}methylene)-6-hydroxy- | | |
| 1-benzofuran-3(2H)-one) | | |
| ((2Z)-6-hydroxy-2-({5-methoxy-2-methyl-1-[2- | 12.2 | 325 |
| (4-methylpiperazin-1-yl)ethyl]-1H-indol-3- | | |
| yl}methylene)-1-benzofuran-3(2H)-one) | | |
| ((2Z)-4,6-dihydroxy-2-({5-methoxy-2-methyl-1- | 2.0 | 22.5 |
| [2-(4-methylpiperazin-1-yl)ethyl]-1H-indol-3- | | |
| yl}methylene)-1-benzofuran-3(2H)-one) | | |
| ((2Z)-4,6-dihydroxy-2-[(1-methyl-4-phenyl-1H- | 62.8 | 0.5 |
| pyrrolo[2,3-b]pyridin-3-yl)methylene]-1- | | |
| benzofuran-3(2H)-one) | | |
| ((2Z)-6-hydroxy-2-[(5-methoxy-2-phenyl-1H- | 1.3 | 92 |
| indol-3-yl)methylene]-1-benzofuran-3(2H)- | | |
| one) | | |
| ((2Z)-4-hydroxy-2-{[1-methyl-4-(8-oxa-3- | 104.0 | 0.4 |
| azabicyclo[3.2.1]oct-3-yl)-1H-pyrrolo[2,3- | | |
| b]pyridin-3-yl]methylene}-1-benzofuran-3(2H)- | | |
| one) | | |
| ((2Z)-5-hydroxy-2-({5-methoxy-2-methyl-1-[2- | 272.5 | 1050 |
| (4-methylpiperazin-1-yl)ethyl]-1H-indol-3- | | |
| yl}methylene)-1-benzofuran-3(2H)-one) | | |
| ((2Z)-4,6-dihydroxy-2-[(1-methyl-4-phenyl-1H- | 340.0 | 59 |
| indol-3-yl)methylene]-1-benzofuran-3(2H)- | | |
| one) | | |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of Formula 1: 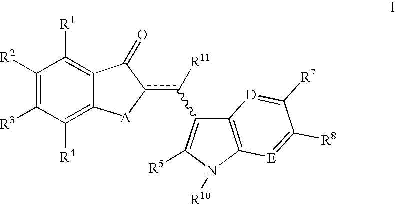
or a geometric isomer thereof, or a pharmaceutically acceptable salt thereof, wherein:
A is oxygen or sulfur;
. . . represents an optional second carbon-to-carbon bond;
D is C—R⁶ or N;
E is C—R⁹ or N;
R¹, R², R³, and R⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; OR²⁰; or hydroxyl;
wherein at least one of R¹-R⁴ is OR²⁰, wherein each R²⁹ is independently selected from C(O)R¹², CO₂R¹², CONR¹²R¹³, P(O)(OR¹²)(OR¹³), P(O)R¹²(OR¹³), C(R¹²R¹³)OR¹⁴, and C(R¹²R¹³)NR¹⁴R²¹;
with the proviso that R¹² cannot be H when R²⁹ is C(O)R¹² or CO₂R¹²;
R¹², R¹³, R¹⁴ and R²¹ are each independently H; C₁-C₆alkyl optionally substituted with from 1 to 3 substituents independently selected from OH, —NH₂, (C₁-C₆alkyl)NH—, (C₁-C₆alkyl)(C₁-C₆alkyl)N—, C₆-C₁₀ aryl, (C₁-C₆alkyl)oxycarbonyl, and C₁-C₉ heteroaryl; perfluoro(C₁-C₆)alkyl; C₁-C₉heteroaryl optionally substituted with from 1 to 3 substituents independently selected from C₁-C₆alkyl, halo, and perfluoro(C₁-C₆)alkyl; C₈-C₁₄aryl optionally substituted with from 1 to 3 substituents independently selected from C₁-C₆alkyl, halo, and perfluoro(C₁-C₈)alkyl; C₂-C₈ heterocyclyl; or C₃-C₈cycloalkyl;
or when R²⁹ is CONR¹²R¹³, R¹² and R¹³ taken together with the N they are attached to form a 3-10 membered heterocyclyl with 1-3 hetero atoms selected from N, O and S, wherein the 3-10 membered heterocyclyl is optionally substituted with 1-3 substituents selected from straight or branched C₁-C₆alkyl optionally substituted with fluorine, C₆-C₁₄aryl, C₃-C₈ cycloalkyl, C₃-C₈ heterocyclyl, CN, ═O, NO₂, (CH₂)ₙ—, O(C₁-C₆alkyl), (CH₂)ₙ—, —NH₂, (C₁-C₆alkyl)NH—(CH₂)ₙ—, and (C₁-C₆alkyl)(C₁-C₆alkyl)N—(CH₂)ₙ—;
wherein n is 0 or 1;
R⁵ is H; C₁-C₆alkyl; C₆-C₁₄aryl; C₃-C₈cycloalkyl; halo; C₁-C₉heteroaryl; C₁-C₆heterocyclylalkyl; C₁-C₆ perfluoroalkyl-; R¹⁵R¹⁶NC(O)—; (C₁-C₆alkoxy)carbonyl; or CO₂H;
R¹⁵ and R¹⁶ are each independently H; C₁-C₆alkyl optionally substituted with from 1 to 3 substituents independently selected from —NH₂, (C₁-C₆alkyl)NH—, (C₁-C₆alkyl)(C₁-C₆alkyl)N—, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
or R¹⁵ and R¹⁶ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;
R⁶—R⁹ are each independently: (a) H; (b) C₁-C₆alkoxy-; (c) C₁-C₆alkyl-optionally substituted by C₆-C₁₄aryl-; (d) C₂-C₆alkenyl-optionally substituted by C₆-C₁₄aryl-; (e) C₂-C₆alkynyl-optionally substituted by C₆-C₁₄aryl-; (f) (C₁-C₆alkyl)amido-; (g) C₁-C₆alkylcarboxy-; (h) (C₁-C₆alkyl)carboxyamido-; (i) (C₁-C₆alkyl)SO₂—; (j) C₆-C₁₄aryl-optionally substituted with from 1 to 3 substituents independently selected from: (i) C₁-C₆acyl-, (ii) C₁-C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C₁-C₉heterocyclyl-, (iii) (C₁-C₆alkyl)amido-, (iv) (C₁-C₆alkyl)carboxy-, (v) (C₁-C₆alkyl)carboxyamido-, (vi) C₁-C₆alkoxy-optionally substituted by C₁-C₆alkoxy or C₁-C₉heteroaryl, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (k) (C₆-C₁₄aryl)alkyl-O—; (l) halo; (m) O₁—C₉heteroaryl optionally substituted with from 1 to 3 substituents independently selected from: (i) O₁—C₈acyl-, (ii) O₁—C₆alkyl-, which is optionally substituted with from 1 to 3 substituents independently selected from: A) H₂N—, B) (C₁-C₆alkyl)NH—, C) (C₁-C₆alkyl)(C₁-C₆alkyl)N—, and D) C1-C₉heterocyclyl-, (iii) (C₁-C₆alkyl)amido-, (iv) (C₁-C₆alkyl)carboxy, (v) (C₁-C₆alkyl)carboxyamido-, (vi) C₁-C₆alkoxy-optionally substituted by C₁-C₆alkoxy- or C₁-C₉heteroaryl-, (vii) (C₁-C₆alkoxy)carbonyl-, (viii) (C₆-C₁₄aryl)oxy-, (ix) C₃-C₈cycloalkyl-, (x) halo, (xi) C₁-C₆haloalkyl-, (xii) C₁-C₉heterocyclyl-optionally substituted by C₁-C₆alkyl- or C₁-C₆hydroxylalkyl-, (xiii) hydroxyl, (xiv) C₁-C₆hydroxylalkyl-, (xv) C₁-C₆ perfluoroalkyl-, (xvi) C₁-C₆ perfluoroalkyl-O—, (xvii) R¹⁷R¹⁸N—, (xviii) NC—, (xix) HOOC—, (xx) R¹⁷R¹⁸NC(O)—, (xxi) R¹⁷C(O)NH—, (xxii) R¹⁷R¹⁸NS(O)₂— (xxiii) R¹⁷R¹⁸NC(O)NH—, (xxiv) R¹⁹OC(O)NH—, (xxv) (C₁-C₆alkyl)S(O)₂NH—, (xxvi) R¹⁹S(O)₂—, (xxvii) —C(═N—(OR¹⁷))—(NR¹⁷R¹⁸), and (xxviii) O₂N—; (n) hydroxyl; (o) C₁-C₉ heterocyclyl-optionally substituted by: (i) C₁-C₆alkyl-, which is optionally substituted with from 1 to 3 substituents independently selected from: A) H₂N—, B) (C₁-C₆alkyl)NH—, and C) (C₁-C₆alkyl)(C₁-C₆alkyl)N—, (ii) R¹⁷R¹⁸NC(O)—, (iii) hydroxyl, or (iv) R¹⁷R¹⁸N—; (p) C₁-C₆ perfluoroalkyl-; (q) NC—; (r) (C₁-C₆alkoxy)carbonyl-; (s) HOOC—; or (t) O₂N—;
R¹⁷ and R¹⁸ are each independently H; C₁-C₆alkyl optionally substituted with from 1 to 3 substituents independently selected from C₁-C₆alkoxy, —NH₂, (C₁-C₆alkyl)NH—, (C₁-C₆alkyl)(C₁-C₆alkyl)N—, C₆-C₁₄aryl, and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
or R¹⁷ and R¹⁸ when taken together with the nitrogen to which they are attached can form a 3- to 7-membered nitrogen containing heterocycle wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;
R¹⁹ is C₁-C₆alkyl or C₆-C₁₄aryl;
or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;
R¹⁹ is H; C₁-C₆alkyl optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), O₆—C₁₄aryl, C₁-C₉heteroaryl, C₃-C₈cycloalkyl, C₁-C₆haloalkyl-, C₁-C₆-aminoalkyl-, —OC(O)(C₁-C₆alkyl), C₁-C₆-carboxyamidoalkyl-, NO₂, and C₁-C₉heterocyclyl-, each C₁-C₉heterocyclyl-optionally substituted with C₁-C₆alkyl; C₂-C₁₀ alkenyl; C₆-C₁₄aryl; C₃-C₈cycloalkyl; C₁-C₉heteroaryl; or C₁-C₆heterocyclylalkyl group optionally substituted with from 1 to 3 substituents independently selected from halogen, —NH₂, (C₁-C₆alkyl)NH—, (C₁-C₆alkyl)(C₁-C₆alkyl)N—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, O₁—C₆alkyl, —C(O)OH, —C(O)O(C₁-C₆alkyl), —C(O)(C₁-C₆alkyl), 4- to 7-membered monocyclic heterocycle, C₆-C₁₄aryl C₁-C₉heteroaryl, C₁-C₆heterocyclylalkyl, and C₃-C₈cycloalkyl;
or R⁵ and R¹⁹ taken together with the atoms connecting them form a fused C₅-C₈ heterocyclic ring containing 2-3 hetero atoms selected from N, O, and S, and optionally substituted with halogen, hydroxy, O—C₁-C₆ alkoxy, CN, ═O, C₁-C₆alkyl, NO₂, NH₂, NHC₁-C₆alkyl, N(C₁-C₆alkyl)₂, C(O)C₁-C₆alkyl, CO₂C₁-C₆alkyl, CONH₂, CONHC₁-C₆alkyl, or CON(C₁-C₆alkyl)₂; and
R¹¹ is H or C₁-C₆alkyl.

2. A compound of claim 1, wherein A is oxygen.

3. A compound of claim 1, wherein R² is H.

4. A compound of claim 1, wherein R⁴ is H.

5. A compound of claim 1, wherein R⁵ is H.

6. A compound of claim 1, wherein R⁶ is C₆-C₁₄aryl, optionally independently substituted with from 1 to 3 substituents as specified in claim 1.

7. A compound of claim 1, wherein R⁷ is H.

8. A compound of claim 1, wherein R⁸ is H.

9. A compound of claim 1, wherein R²⁰ is C(O)R¹².

10. A compound of claim 1, wherein R²⁰ is CO₂R¹².

11. A compound of claim 1, wherein R²⁰ is CONR¹²R¹³.

12. A compound of claim 1, wherein R¹² and R¹³ taken together with the N they are attached to form a 3-10 membered heterocyclyl with 1-3 hetero atoms selected from N, O and S, wherein the 3-10 membered heterocyclyl is optionally substituted as defined for claim 1 herein.

13. A compound of claim 1, wherein R²⁰ is P(O)(OR¹²)(OR¹³).

14. A compound of claim 1, wherein R²⁰ is P(O)R¹²(OR¹³).

15. A compound of claim 1, wherein R¹⁰ is C₁-C₆alkyl optionally substituted as defined in claim 1.

16. A compound of claim 1, wherein R¹⁰ is methyl.

17. A compound of claim 1, wherein R¹¹ is H.

18. A compound of claim 1, wherein R⁵═R⁷═R⁸═H and R¹⁰ is CH₃.

19. A compound of claim 1, wherein R⁶ is C₆-C₁₄aryl, optionally independently substituted with from 1 to 3 substituents as specified in claim 1, D is C—R⁶, E is C—R⁹, and R¹¹ is H.

20. A compound of claim 1, wherein R⁶ is C₆-C₁₄aryl, optionally independently substituted with from 1 to 3 substituents as specified in claim 1, D is C—R⁶, E is N, and R¹¹ is H.

21. A compound of claim 1, wherein R², R⁴, R⁵, R⁷, R⁸, R¹¹H, R⁶ is C₆-C₁₄aryl, optionally independently substituted with from 1 to 3 substituents as specified in claim 1, D is C—R⁶, E is C—R⁹, and R¹⁰ is CH₃.

22. A compound of claim 1, wherein R², R⁴, R⁵, R⁷, R⁸, R¹¹H, R⁶ is C₆-C₁₄aryl, optionally independently substituted with from 1 to 3 substituents as specified in claim 1, D is C—R⁶, E is N, and R¹⁰ is CH₃.

23. A compound of claim 1, wherein R¹ and R³ are each OR²⁰ and R² and R⁴ are each H.

24. A compound of claim 23, wherein each R²⁰ is CO₂R¹², wherein each R¹² is C₁-C₆alkyl optionally substituted as defined in claim 1 or each R¹² is C₆-C₁₄ aryl optionally substituted as defined in claim 1.

25. A compound of claim 23, wherein each R²⁰ is CONR¹²R¹³, wherein R¹² and R¹³ are each C₆-C₁₄ aryl or each C₁-C₆alkyl wherein each alkyl or aryl is optionally substituted as defined for claim 1.

26. A compound of claim 23, wherein each R² is CONR¹²R¹³, wherein one of R¹² and R¹³ is C₆-C₁₄ aryl and the other of R¹² and R¹³ is C₁-C₆alkyl wherein the aryl or alkyl is optionally substituted as defined for claim 1

27. A compound of claim 26, wherein the one of R¹² and R¹³ that is alkyl is substituted with a C₁-C₆alkyloxycarbonyl.

28. A compound of claim 23, wherein each R²⁰ is CONR¹²R¹³, wherein one of R¹² and R¹³ is hydrogen and the other of R¹² and R¹³ is C₁-C₆alkyl wherein the alkyl is optionally substituted as defined for claim 1.

29. A compound of claim 28, wherein the one of R¹² and R¹³ that is alkyl is substituted with a C₁-C₆alkyloxycarbonyl.

30. A compound of claim 23, wherein each R²⁰ is CONR¹²R¹³ and R¹² and R¹³ taken together with the N they are attached to form a 3-10 membered heterocyclyl with 1-3 hetero atoms selected from N, O and S, wherein the 3-10 membered heterocyclyl is optionally substituted as defined for claim 1.

31. The compound of claim 23, wherein each R²⁰ is COR¹², wherein each R¹² is C₆-C₁₄ aryl or each R¹² is C₁-C₆alkyl or each R¹² is C₂-C₈ heterocyclyl wherein the alkyl or aryl or heterocyclyl is optionally substituted as defined in claim 1.

32. The compound of claim 23, wherein each R²⁰ is P(O)(OR¹²)(OR¹³), wherein R¹² and R¹³ are each C₁-C₆alkyl optionally substituted as defined in claim 1 or each hydrogen.

33. The compound of claim 1, wherein R³ is OR²⁰ and R¹, R² and R⁴ are each H or OH, provided that no more than one of R¹, R², and R⁴ can be OH.

34. The compound of claim 33, wherein R²⁰ is COR¹², wherein R¹² is C₁-C₆alkyl or C₆-C₁₄ aryl wherein the alkyl or aryl is optionally substituted as defined in claim 1.

35. The compound of claim 33, wherein R²⁰ is COOR¹², wherein R¹² is C₆-C₁₄ aryl optionally substituted as defined in claim 1.

36. The compound of claim 33, wherein R²⁰ is CONR¹²R¹³, wherein R¹² and R¹³ are each C₁-C₆alkyl or each C₆-C₁₄ aryl wherein each alkyl or aryl is optionally substituted as defined in claim 1.

37. The compound of claim 33, wherein R²⁰ is CONR¹²R¹³, wherein one of R¹² and R¹³ is hydrogen and the other of R¹² and R¹³ is C₁-C₆alkyl optionally substituted as defined in claim 1.

38. The compound of claim 33, wherein R²⁰ is P(O)(OR¹²)(OR¹³), wherein R¹² and R¹³ are each hydrogen.

39. The compound of claim 33, wherein R²⁰ is P(O)R¹²(OR¹³), wherein R¹² is C₆-C₁₄ aryl optionally substituted as defined in claim 1 and R¹³ is hydrogen.

40. The compound of claim 1, wherein R¹ is OR²⁰ and R³, R² and R⁴ are each H or OH, provided that no more than one of R², R³, and R⁴ can be OH.

41. The compound of claim 40, wherein R²⁰ is COR¹², wherein R¹² is C₁-C₆alkyl or C₆-C₁₄ aryl wherein the alkyl or aryl is optionally substituted as defined in claim 1.

42. The compound of claim 40, wherein R²⁰ is COOR¹², wherein R¹² is C₆-C₁₄ aryl optionally substituted as defined in claim 1.

43. The compound of claim 40, wherein R²⁰ is CONR¹²R¹³, wherein R¹² and R¹³ are each C₁-C₆alkyl or each C₆-C₁₄ aryl wherein the alkyl or aryl is optionally substituted as defined in claim 1.

44. The compound of claim 40, wherein R²⁰ is CONR¹²R¹³, wherein one of R¹² and R¹³ is hydrogen and the other of R¹² and R¹³ is C₁-C₆alkyl optionally substituted as defined in claim 1.

45. The compound of claim 40, wherein R²⁰ is P(O)(OR¹²)(OR¹³), wherein R¹² and R¹³ are each hydrogen.

46. The compound of claim 40, wherein R²⁰ is P(O)R¹²(OR¹³), wherein R¹² is C₆-C₁₄ aryl optionally substituted as defined in claim 1 and R¹³ is hydrogen.

47. The compound of claim 1, wherein R² is OR²⁰ and R³, R¹ and R⁴ are each H or OH provided that no more than one of R³, R¹, and R⁴ can be OH.

48. The compound of claim 47, wherein R²⁰ is CONR¹²R¹³, wherein one of R¹² and R¹³ is hydrogen and the other of R¹² and R¹³ is C₁-C₆alkyl optionally substituted as defined in claim 1.

49. The compound of claim 1, wherein R¹⁰ is selected from hydrogen and (C₁-C₆)alkyl optionally substituted with di(C₁-C₆)alkylamino or with C₁-C₉ heterocyclyl optionally substituted with C₁-C₆alkyl.

50. The compound of claim 49, wherein C₁-C₉heterocyclyl is piperazinyl, which is optionally substituted with C₁-C₆alkyl.

51. The compound of claim 1, wherein R⁷ is selected from hydrogen and (C₁-C₆)alkoxy.

52. The compound of claim 1, wherein R⁵ is selected from hydrogen, (C₆-C₁₄)aryl, and C₁-C₆alkyl.

53. The compound of claim 1, wherein R⁶ is selected from hydrogen, (C₆-C₁₄)aryl, and C₁-C₉ heterocyclyl.

54. The compound of claim 53, wherein C₁-C₉heterocyclyl is (8-oxa-3-azabicyclo[3.2.1]oct-3-yl).

55. The compound of claim 1, wherein the compound is a compound of formula 2 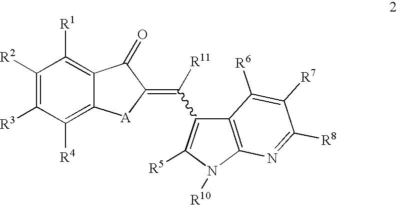
or a geometric isomer thereof or a pharmaceutically acceptable salt thereof, wherein A, R¹-R⁸, R¹⁰, and R¹¹ are as defined in claim 1.

56. The compound of claim 1, wherein the compound is a compound of formula 3 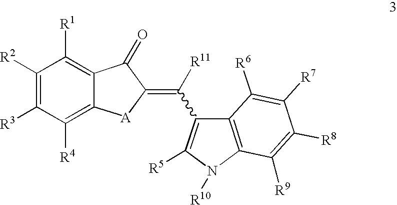
or a geometric isomer thereof or a pharmaceutically acceptable salt thereof, wherein A and R¹-R¹¹ are as defined in claim 1.

57. The compound of claim 1, wherein the geometric isomer is the (Z) isomer.

58. The compound of claim 55, wherein the geometric isomer is the (Z) isomer.

59. The compound of claim 56, wherein the geometric isomer is the (Z) isomer.

60. A compound selected from the group consisting of:
(2Z)-2-({1-[3-(dimethylamino)propyl]-5-methoxy-1H-indol-3-yl}methylene)-3-oxo-2,3-dihydro-1-benzofuran-4,6-diyldimorpholine-4-carboxylate;
(2Z)-2-({1-[3-(dimethylamino)propyl]-5-methoxy-1H-indol-3-yl}methylene)-3-oxo-2,3-dihydro-1-benzofuran-4,6-diylbis[methyl(phenyl)carbamate];
(2Z)-2-[(5-methoxy-2-phenyl-1H-indol-3-yl)methylene]-3-oxo-2,3-dihydro-1-benzofuran-4,6-diyl diacetate;
(2Z)-2-({1-[3-(dimethylamino)propyl]-5-methoxy-1H-indol-3-yl}methylene)-3-oxo-2,3-dihydro-1-benzofuran-4,6-diyl bis(diisopropylcarbamate);
(2Z)-2-({1-[3-(dimethylamino)propyl]-5-methoxy-1H-indol-3-yl}methylene)-3-oxo-2,3-dihydro-1-benzofuran-4,6-diyl diisopropyl biscarbonate;
(2Z)-2-[(5-methoxy-2-phenyl-1H-indol-3-yl)methylene]-3-oxo-2,3-dihydro-1-benzofuran-4,6-diyl dibenzoate;
diisopropyl (2Z)-2-[(5-methoxy-2-phenyl-1H-indol-3-yl)methylene]-3-oxo-2,3-dihydro-1-benzofuran-4,6-diyl biscarbonate;
(2Z)-2-({1-[3-(dimethylamino)propyl]-5-methoxy-1H-indol-3-yl}methylene)-3-oxo-2,3-dihydro-1-benzofuran-4,6-diyl bis(dimethylcarbamate);
(2Z)-2-({1-[3-(dimethylamino)propyl]-5-methoxy-1H-indol-3-yl}methylene)-3-oxo-2,3-dihydro-1-benzofuran-4,6-diyl bis(3-methylbutanoate);
(2Z)-2-[(5-methoxy-2-phenyl-1H-indol-3-yl)methylene]-3-oxo-2,3-dihydro-1-benzofuran-4,6-diyl bis(2,2-dimethylpropanoate);
(2Z)-2-[(5-methoxy-2-phenyl-1H-indol-3-yl)methylene]-3-oxo-2,3-dihydro-1-benzofuran-4,6-diyl bis(diphenylcarbamate);
(2Z)-2-({5-methoxy-2-methyl-1-[2-(4-methylpiperazin-1-yl)ethyl]-1H-indol-3-yl}methylene)-3-oxo-2,3-dihydro-1-benzofuran-4,6-diyl bis(dimethylcarbamate);
(2Z)-2-({1-[3-(dimethylamino)propyl]-5-methoxy-1H-indol-3-yl}methylene)-3-oxo-2,3-dihydro-1-benzofuran-4,6-diyl dimethyl biscarbonate;
(2Z)-2-({1-[3-(dimethylamino)propyl]-5-methoxy-1H-indol-3-yl}methylene)-3-oxo-2,3-dihydro-1-benzofuran-4,6-diyl bis(2,2-dimethylpropanoate);
(2Z)-2-({1-[3-(dimethylamino)propyl]-5-methoxy-1H-indol-3-yl}methylene)-3-oxo-2,3-dihydro-1-benzofuran-4,6-diyl diacetate;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-4,6-diyl dibenzoate;
(2Z)-2-[(5-methoxy-2-phenyl-1H-indol-3-yl)methylene]-3-oxo-2,3-dihydro-1-benzofuran-6-yl benzoate;
(2Z)-2-[(5-methoxy-2-phenyl-1H-indol-3-yl)methylene]-3-oxo-2,3-dihydro-1-benzofuran-6-yl dimethylcarbamate;
(2Z)-2-[(5-methoxy-2-phenyl-1H-indol-3-yl)methylene]-3-oxo-2,3-dihydro-1-benzofuran-6-yl phenyl carbonate;
(2Z)-2-[(5-methoxy-2-phenyl-1H-indol-3-yl)methylene]-3-oxo-2,3-dihydro-1-benzofuran-6-yl diphenylcarbamate;
(2Z)-2-({1-[3-(dimethylamino)propyl]-5-methoxy-1H-indol-3-yl}methylene)-3-oxo-2,3-dihydro-1-benzofuran-4,6-diyl bis(diphenylcarbamate);
(2Z)-2-[(5-methoxy-2-phenyl-1H-indol-3-yl)methylene]-3-oxo-2,3-dihydro-1-benzofuran-4,6-diyl dimethyl biscarbonate;
(2Z)-2-[(5-methoxy-2-phenyl-1H-indol-3-yl)methylene]-3-oxo-2,3-dihydro-1-benzofuran-4,6-diyl diphenyl biscarbonate;
(2Z)-2-({1-[3-(dimethylamino)propyl]-5-methoxy-1H-indol-3-yl}methylene)-3-oxo-2,3-dihydro-1-benzofuran-4,6-diyl dibenzoate;
ethyl N-({[(2Z)-2-({1-[3-(dimethylamino)propyl]-5-methoxy-1H-indol-3-yl}methylene)-3-oxo-2,3-dihydro-1-benzofuran-6-yl]oxy}carbonyl)glycinate;
(2Z)-2-[(5-methoxy-2-phenyl-1H-indol-3-yl)methylene]-3-oxo-2,3-dihydro-1-benzofuran-6-yl hydrogen phenylphosphonate;
(2Z)-2-[(5-methoxy-2-phenyl-1H-indol-3-yl)methylene]-3-oxo-2,3-dihydro-1-benzofuran-6-yl dihydrogen phosphate;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-4,6-diyl bis(diisopropylcarbamate);
(2Z)-2-({5-methoxy-2-methyl-1-[2-(4-methylpiperazin-1-yl)ethyl]-1H-indol-3-yl}methylene)-3-oxo-2,3-dihydro-1-benzofuran-4,6-diyl bis(diphenylcarbamate);
(2Z)-2-[(5-methoxy-2-phenyl-1H-indol-3-yl)methylene]-3-oxo-2,3-dihydro-1-benzofuran-4,6-diyl bis(dimethylcarbamate);
(2Z)-2-[(5-methoxy-2-phenyl-1H-indol-3-yl)methylene]-3-oxo-2,3-dihydro-1-benzofuran-4,6-diyl bis(diisopropylcarbamate);
tetraethyl (2Z)-2-[(5-methoxy-2-phenyl-1H-indol-3-yl)methylene]-3-oxo-2,3-dihydro-1-benzofuran-4,6-diyl bis(phosphate);
(2Z)-2-({1-[3-(dimethylamino)propyl]-5-methoxy-1H-indol-3-yl}methylene)-3-oxo-2,3-dihydro-1-benzofuran-4,6-diyl tetraethyl bis(phosphate);
diethyl (2Z)-2-[(5-methoxy-2-phenyl-1H-indol-3-yl)methylene]-3-oxo-2,3-dihydro-1-benzofuran-6-yl phosphate;
diethyl 2,2′-[[(2Z)-2-({1-[3-(dimethylamino)propyl]-5-methoxy-1H-indol-3-yl}methylene)-3-oxo-2,3-dihydro-1-benzofuran-4,6-diyl]bis(oxycarbonylimino)]diacetate;
(2Z)-2-({1-[3-(dimethylamino)propyl]-5-methoxy-1H-indol-3-yl}methylene)-3-oxo-2,3-dihydro-1-benzofuran-4,6-diylbis(4-methylpiperazine-1-carboxylate);
(2Z)-2-({1-[3-(dimethylamino)propyl]-5-methoxy-1H-indol-3-yl}methylene)-3-oxo-2,3-dihydro-1-benzofuran-4,6-diyl bis(4-benzylpiperazine-1-carboxylate);
(2Z)-2-[(5-methoxy-2-phenyl-1H-indol-3-yl)methylene]-3-oxo-2,3-dihydro-1-benzofuran-4,6-diyl bis[dihydrogen (phosphate)];
(2Z)-6-hydroxy-2-({5-methoxy-2-methyl-1-[2-(4-methylpiperazin-1-yl)ethyl]-1H-indol-3-yl}methylene)-3-oxo-2,3-dihydro-1-benzofuran-4-yl dimethylcarbamate;
(2Z)-2-({1-[3-(dimethylamino)propyl]-5-methoxy-1H-indol-3-yl}methylene)-6-hydroxy-3-oxo-2,3-dihydro-1-benzofuran-4-yl dimethylcarbamate;
(2Z)-4-hydroxy-2-({5-methoxy-2-methyl-1-[2-(4-methylpiperazin-1-yl)ethyl]-1H-indol-3-yl}methylene)-3-oxo-2,3-dihydro-1-benzofuran-6-yl dimethylcarbamate;
(2Z)-2-({1-[3-(dimethylamino)propyl]-5-methoxy-1H-indol-3-yl}methylene)-4-hydroxy-3-oxo-2,3-dihydro-1-benzofuran-6-yl dimethylcarbamate;
(2Z)-2-[(1-methyl-4-phenyl-1H-pyrrolo[2,3-b]pyridin-3yl)methylene]-3-oxo-2,3-dihydro-1-benzofuran-4,6-diyl bis(dimethylcarbamate);
(2Z)-2-{[1-methyl-4-(8-oxa-3-azabicyclo[3.2.1]oct-3-yl)-1H-pyrrolo[2,3-b]pyridin-3-yl]methylene}-3-oxo-2,3-dihydro-1-benzofuran-4-yl dimethylcarbamate;
(2Z)-2-({5-methoxy-1-[2-(4-methylpiperazin-1-yl)ethyl]-1H-indol-3-yl}methylene)-3-oxo-2,3-dihydro-1-benzofuran-5-yl methylcarbamate;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-6-ylmethyl(phenyl)carbamate;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-6-yl diisopropylcarbamate; and
(2Z)-2-[(1-methyl-4-phenyl-1H-indol-3-yl)methylene]-3-oxo-2,3-dihydro-1-benzofuran-4,6-diyl bis(dimethylcarbamate);
or a pharmaceutically acceptable salt thereof.

61. A composition comprising a compound of claim 1 and a pharmaceutically acceptable carrier.

62. The composition of claim 61, wherein the pharmaceutically acceptable carrier suitable for oral administration and the composition comprises an oral dosage form.

63. A composition comprising a compound of claim 1; a second compound selected from the group consisting of a topoisomerase I inhibitor, a MEK 1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab and lavendustin A; and a pharmaceutically acceptable carrier.

64. The composition of claim 63, wherein the second compound is Avastin.

65. A method of treating a PI3K-related disorder, comprising administering to a mammal in need thereof a compound of claim 1 in an amount effective to treat a PI3K-related disorder.

66. The method of claim 65, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

67. The method of claim 66, wherein the PI3K-related disorder is cancer.

68. The method of claim 67, wherein the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

69. A method of treating an mTOR-related disorder, comprising administering to a mammal in need thereof a compound of claim 1 in an amount effective to treat an mTOR-related disorder.

70. The method of claim 69, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

71. The method of claim 70, wherein the mTOR-related disorder is cancer.

72. The method of claim 71, wherein the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

73. A method of treating advanced renal cell carcinoma, comprising administering to a mammal in need thereof a compound of claim 1 in an amount effective to treat advanced renal cell carcinoma.

74. A method of treating acute lymphoblastic leukemia, comprising administering to a mammal in need thereof a compound of claim 1 in an amount effective to treat acute lymphoblastic leukemia.

75. A method of treating acute malignant melanoma, comprising administering to a mammal in need thereof a compound of claim 1 in an amount effective to treat malignant melanoma.

76. A method of treating soft-tissue or bone sarcoma, comprising administering to a mammal in need thereof a compound of claim 1 in an amount effective to treat soft-tissue or bone sarcoma.

77. A method of treating a cancer selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer comprising administering to a mammal in need thereof the composition of claim 64 in an amount effective to treat the cancer.

78. A method of inhibiting mTOR in a subject, comprising administering to a subject in need thereof a compound of claim 1 in an amount effective to inhibit mTOR.

79. A method of inhibiting PI3K in a subject, comprising administering to a subject in need thereof a compound of claim 1 in an amount effective to inhibit PI3K.

80. A method of inhibiting mTOR and PI3K together in a subject, comprising administering to a subject in need thereof a compound of claim 1 in an amount effective to inhibit mTOR and PI3K.

81. A method of synthesizing a compound of claim 1 comprising
a) reacting a compound of the formula 4 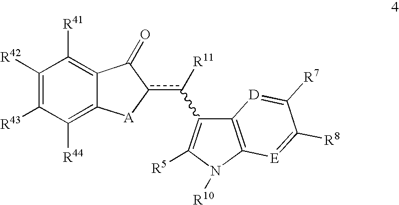
wherein A, R¹¹, R⁵, R¹⁰, D, E, R⁷ and R⁸ are defined as in claim 1, and R⁴¹, R⁴², R⁴³, and R⁴⁴ are each independently H; C₁-C₆alkoxy optionally substituted with from 1 to 3 substituents independently selected from —NH₂, (C₁-C₆alkyl)NH—, and (C₁-C₆alkyl)(C₁-C₆alkyl)N—; C₁-C₆alkyl; (C₁-C₆alkoxy)carbonyl; R¹²R¹³N—; R¹²R¹³NC(O)NH—; R¹²C(O)NH—; R¹⁴OC(O)NH—; halo; or hydroxyl;
wherein R¹², R¹³, and R¹⁴ are defined as in claim 1, and
wherein at least one of R⁴¹—R⁴⁴ is hydroxyl,
with a compound of the formula R²⁰—X, wherein R²⁰ is defined as in claim 1 and X is a leaving group,
to form a compound of claim 1;
b) optionally reacting with an acid to form a pharmaceutically acceptable salt of the compound of claim 1 or geometric isomer thereof.
